SEQUENCE LISTING (80 sequences)

SEQ ID NO: 1 - DNA, 57 bp, synthetic construct
  misc_feature 1..57: note = Synthesized polynucleotide encoding signal peptide
  source 1..57: mol_type = other DNA
atggagttcggcctgagctgggtgttcctggtggccctgttcaggggcgtgcagtgc

SEQ ID NO: 2 - protein, 20 aa, synthetic construct
  REGION 1..20: note = Synthesized signal peptide sequence
MEFGLSWVFLVALFRGVQCD

SEQ ID NO: 3 - protein, 20 aa, synthetic construct
  REGION 1..20: note = human GM-CSF leader sequence
MYRMQLLSCIALSLALVTNS

SEQ ID NO: 4 - protein, 20 aa, synthetic construct
  REGION 1..20: note = Polypeptide sequence of human GM-CSFR leader sequence
MYRMQLLSCIALSLALVTNS

SEQ ID NO: 5 - DNA, 15 bp, synthetic construct
  misc_feature 1..15: note = Synthesized polynucleotide encoding 4GlySer linker peptide
  source 1..15: mol_type = other DNA
ggcggcggcggcagc

SEQ ID NO: 6 - protein, 5 aa, synthetic construct
  REGION 1..5: note = Synthesized 4GlySer linker peptide sequence
GGGGS

SEQ ID NO: 7 - DNA, 45 bp, synthetic construct
  misc_feature 1..45: note = Synthesized polynucleotide encoding 3x 4GlySer linker peptide
  source 1..45: mol_type = other DNA
ggcggcggcggcagcggcggcggcggcagcggcggcggcggcagc

SEQ ID NO: 8 - protein, 15 aa, synthetic construct
  REGION 1..15: note = Synthesized 3x 4GlySer linker peptide sequence
GGGGSGGGGSGGGGS

SEQ ID NO: 9 - DNA, 37 bp, synthetic construct
  misc_feature 1..37: note = Synthesized polynucleotide encoding 4x 2GlySer linker peptide
  source 1..37: mol_type = other DNA
gggcggcagcggcggcagcggcggcagcggcggcagc

SEQ ID NO: 10 - protein, 12 aa, synthetic construct
  REGION 1..12: note = Synthesized 4x 2GlySer linker peptide sequence
GGSGGSGGSGGS

SEQ ID NO: 11 - DNA, 18 bp, synthetic construct
  misc_feature 1..18: note = Synthesized polynucleotide encoding 6-His tag
  source 1..18: mol_type = other DNA
caccaccaccaccaccac

SEQ ID NO: 12 - protein, 6 aa, synthetic construct
  REGION 1..6: note = Synthesized 6-His tag sequence
HHHHHH

SEQ ID NO: 13 - DNA, 66 bp, synthetic construct
  misc_feature 1..66: note = Synthesized polynucleotide encoding T2A linker
  source 1..66: mol_type = other DNA
cgaagaaaacgcgaggggaggggctccctgctcacctgtggtgatgtcgaagagaaccctgggccg

SEQ ID NO: 14 - protein, 22 aa, synthetic construct
  REGION 1..22: note = Synthesized T2A linker linker sequence
RRKREGRGSLLTCGDVEENPGP

SEQ ID NO: 15 - DNA, 333 bp, synthetic construct
  misc_feature 1..333: note = Synthesized polynucleotide encoding anti-CD19 light chain sequence
  source 1..333: mol_type = other DNA
gacatccagctgacccagagccccgccagcctggccgtgagcctgggccagagggccaccatcagctgcaaggccagccagagcgtggactacgacggcgacagctacctgaactggtaccagcagatccccggccagccccccaagctgctgatctacgacgccagcaacctggtgagcggcatcccccccaggttcagcggcagcggcagcggcaccgacttcaccctgaacatccaccccgtggagaaggtggacgccgccacctaccactgccagcagagcaccgaggacccctggaccttcggcggcggcaccaagctggagatcaag

SEQ ID NO: 16 - protein, 111 aa, synthetic construct
  REGION 1..111: note = Synthesized anti-CD19 light chain sequence
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 17 - DNA, 372 bp, synthetic construct
  misc_feature 1..372: note = Synthesized polynucleotide encoding anti-CD19 heavy chain sequence
  source 1..372: mol_type = other DNA
caggtgcagctgcagcagagcggcgccgagctggtgaggcccggcagcagcgtgaagatcagctgcaaggccagcggctacgccttcagcagctactggatgaactgggtgaagcagaggcccggccagggcctggagtggatcggccagatctggcccggcgacggcgacaccaactacaacggcaagttcaagggcaaggccaccctgaccgccgacgagagcagcagcaccgcctacatgcagctgagcagcctggccagcgaggacagcgccgtgtacttctgcgccaggagggagaccaccaccgtgggcaggtactactacgccatggactactggggccagggcaccaccgtgaccgtgagcagc

SEQ ID NO: 18 - protein, 124 aa, synthetic construct
  REGION 1..124: note = Synthesized anti-CD19 heavy chain sequence
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 19 - DNA, 318 bp, synthetic construct
  misc_feature 1..318: note = Synthesized polynucleotide encoding anti-CD3 light chain sequence
  source 1..318: mol_type = other DNA
gacatccagctgacccagagccccgccatcatgagcgccagccccggcgagaaggtgaccatgacctgcagggccagcagcagcgtgagctacatgaactggtaccagcagaagagcggcaccagccccaagaggtggatctacgacaccagcaaggtggccagcggcgtgccctacaggttcagcggcagcggcagcggcaccagctacagcctgaccatcagcagcatggaggccgaggacgccgccacctactactgccagcagtggagcagcaaccccctgaccttcggcgccggcaccaagctggagctgaag

SEQ ID NO: 20 - protein, 106 aa, synthetic construct
  REGION 1..106: note = Synthesized anti-CD3 light chain sequence
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 21 - DNA, 357 bp, synthetic construct
  misc_feature 1..357: note = Synthesized polynucleotide encoding anti-CD3 heavy chain sequence
  source 1..357: mol_type = other DNA
gacatcaagctgcagcagagcggcgccgagctggccaggcccggcgccagcgtgaagatgagctgcaagaccagcggctacaccttcaccaggtacaccatgcactgggtgaagcagaggcccggccagggcctggagtggatcggctacatcaaccccagcaggggctacaccaactacaaccagaagttcaaggacaaggccaccctgaccaccgacaagagcagcagcaccgcctacatgcagctgagcagcctgaccagcgaggacagcgccgtgtactactgcgccaggtactacgacgaccactactgcctggactactggggccagggcaccaccctgaccgtgagcagc

SEQ ID NO: 22 - protein, 119 aa, synthetic construct
  REGION 1..119: note = Synthesized anti-CD3 heavy chain sequence
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 23 - DNA, 489 bp, Homo sapiens
  source 1..489: mol_type = other DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttag

SEQ ID NO: 24 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 25 - DNA, 987 bp, Homo sapiens
  source 1..987: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 26 - protein, 329 aa, Homo sapiens
PMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 27 - DNA, 762 bp, Homo sapiens
  source 1..762: mol_type = other DNA
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctag

SEQ ID NO: 28 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 29 - DNA, 297 bp, Homo sapiens
  source 1..297: mol_type = other DNA
atgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttag

SEQ ID NO: 30 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 31 - DNA, 426 bp, Homo sapiens
  source 1..426: mol_type = other DNA
atggagaccgataccctgctcttgtgggttttgcttctttgggtgccaggatctacaggtgatgaagaagaattgcagatcatccaaccagacaaatccgtactcgtggccgcaggagagaccgctaccctcagatgtaccatcacttctctcttccccgttggccccatccagtggtttcgaggcgcaggaccaggacgagtgcttatttacaatcaacgacagggcccattcccaagagtgacaacagtatccgataccaccaagcgcaataatatggactttagcattagaatcggcaacataacacccgctgacgccggtacatactattgtattaaatttcgaaagggctcaccagacgacgtggaatttaagtcaggggccggaaccgaactctcagttagagcaaaaccttctgctagc

SEQ ID NO: 32 - protein, 142 aa, Homo sapiens
METDTLLLWVLLLWVPGSTGDEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSAS

SEQ ID NO: 33 - DNA, 2124 bp, Homo sapiens
  source 1..2124: mol_type = other DNA
atgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccgggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagccgctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactag

SEQ ID NO: 34 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 35 - DNA, 324 bp, synthetic construct
  misc_feature 1..324: note = Synthesized polynucleotide encoding PD-L1 light chain Fv
  source 1..324: mol_type = other DNA
gatatccagatgacacagagcccatcatctctgtctgcaagcgtaggagaccgagtcaccattacatgcagagcctcccaagacgtttccacagcagtggcctggtatcagcaaaaacctggtaaggcgcccaagcttctcatctattcagccagttttctgtatagcggcgttcccagccgattctctggctctggatccggcacggactttactttgacaatttcctctcttcagcccgaagattttgcaacctactactgtcagcaatatctctaccatccagccacattcggacagggcaccaaagtcgaaatcaaaaga

SEQ ID NO: 36 - protein, 108 aa, synthetic construct
  REGION 1..108: note = Synthesized PD-L1 light chain Fv sequence
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 37 - DNA, 354 bp, synthetic construct
  misc_feature 1..354: note = Synthesized polynucleotide encoding PD-L1 heavy chain Fv
  source 1..354: mol_type = other DNA
gaagtgcaactcgttgaaagcggaggagggcttgtccaacctggcgggtcactgcggttgagctgcgccgcaagcggattcaccttctcagactcttggatccattgggtgcgccaggctcccggaaaaggcttggaatgggttgcttggatttcaccgtatggcggttccacatactacgctgacagcgttaagggtcgattcaccatctctgcagatacttcaaaaaacacagcctaccttcagatgaatagtttgcgcgccgaggacacagcggtttattattgtgcccgaagacattggcccggcggtttcgactactgggggcaaggtacgttggtgactgtgagcgcc

SEQ ID NO: 38 - protein, 118 aa, synthetic construct
  REGION 1..118: note = Synthesized PD-L1 heavy chain Fv sequence
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 39 - DNA, 699 bp, Homo sapiens
  source 1..699: mol_type = other DNA
gtagatgaagcaaaatcttgtgacaaaacccatacctgcccaccatgcccagccccagaacttcttggcggaccctctgtcttccttttccctccgaagcccaaggataccctgatgatcagccgaaccccggaggtaacatgtgtggtggtcgatgttagccatgaggatcctgaagtcaaatttaactggtatgtagacggtgttgaggtgcacaacgctaaaactaagcccagggaggagcagtacaactcaacctatcgcgtcgtatctgtgcttaccgtcctgcatcaagactggctcaatggtaaggaatataaatgtaaagtgagtaacaaggcactgccagcacctatcgaaaaaaccatctcaaaggcgaagggacagcccagggaaccccaggtctatactctgccaccttctcgggatgaattgaccaagaaccaagttagcctgacatgtctggtgaaaggtttctatccaagcgatatagctgtcgagtgggagtccaatggccaacctgagaacaattataagaccaccccacccgttctggacagcgacggatcctttttcctgtactcaaaactcactgtcgataaatcaagatggcaacaaggcaacgtttttagctgtagcgtgatgcacgaagcacttcataatcactatacacagaagtcactctctctttctccagga

SEQ ID NO: 40 - protein, 233 aa, Homo sapiens
VDEAKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - DNA, 69 bp, Homo sapiens
  source 1..69: mol_type = other DNA
gtagatgaagcaaaatcttgtgacaaaacccatacctgcccaccatgcccagccccagaacttcttggc

SEQ ID NO: 42 - protein, 23 aa, Homo sapiens
VDEAKSCDKTHTCPPCPAPELLG

SEQ ID NO: 43 - DNA, 1572 bp, synthetic construct
  misc_feature 1..1572: note = Synthesized polynucleotide encoding CD19-CD3 bi-specific T-cell engager construct
  source 1..1572: mol_type = other DNA
atggagttcggcctgagctgggtgttcctggtggccctgttcaggggcgtgcagtgcgacatccagctgacccagagccccgccagcctggccgtgagcctgggccagagggccaccatcagctgcaaggccagccagagcgtggactacgacggcgacagctacctgaactggtaccagcagatccccggccagccccccaagctgctgatctacgacgccagcaacctggtgagcggcatcccccccaggttcagcggcagcggcagcggcaccgacttcaccctgaacatccaccccgtggagaaggtggacgccgccacctaccactgccagcagagcaccgaggacccctggaccttcggcggcggcaccaagctggagatcaagggcggcggcggcagcggcggcggcggcagcggcggcggcggcagccaggtgcagctgcagcagagcggcgccgagctggtgaggcccggcagcagcgtgaagatcagctgcaaggccagcggctacgccttcagcagctactggatgaactgggtgaagcagaggcccggccagggcctggagtggatcggccagatctggcccggcgacggcgacaccaactacaacggcaagttcaagggcaaggccaccctgaccgccgacgagagcagcagcaccgcctacatgcagctgagcagcctggccagcgaggacagcgccgtgtacttctgcgccaggagggagaccaccaccgtgggcaggtactactacgccatggactactggggccagggcaccaccgtgaccgtgagcagcggcggcggcggcagcgacatcaagctgcagcagagcggcgccgagctggccaggcccggcgccagcgtgaagatgagctgcaagaccagcggctacaccttcaccaggtacaccatgcactgggtgaagcagaggcccggccagggcctggagtggatcggctacatcaaccccagcaggggctacaccaactacaaccagaagttcaaggacaaggccaccctgaccaccgacaagagcagcagcaccgcctacatgcagctgagcagcctgaccagcgaggacagcgccgtgtactactgcgccaggtactacgacgaccactactgcctggactactggggccagggcaccaccctgaccgtgagcagcgtggagggcggcagcggcggcagcggcggcagcggcggcagcggcggcgtggacgacatccagctgacccagagccccgccatcatgagcgccagccccggcgagaaggtgaccatgacctgcagggccagcagcagcgtgagctacatgaactggtaccagcagaagagcggcaccagccccaagaggtggatctacgacaccagcaaggtggccagcggcgtgccctacaggttcagcggcagcggcagcggcaccagctacagcctgaccatcagcagcatggaggccgaggacgccgccacctactactgccagcagtggagcagcaaccccctgaccttcggcgccggcaccaagctggagctgaagcaccaccaccaccaccactag

SEQ ID NO: 44 - protein, 523 aa, synthetic construct
  REGION 1..523: note = Synthesized CD19-CD3 bi-specific T-cell engager construct
MEFGLSWVFLVALFRGVQCDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 45 - DNA, 1177 bp, synthetic construct
  misc_feature 1..1177: note = Synthesized polynucleotide encoding SIRP1alpha-CD3-SL bi-specific T-cell engager construct
  source 1..1177: mol_type = other DNA
atggagaccgataccctgctcttgtgggttttgcttctttgggtgccaggatctacaggtgatgaagaagaattgcagatcatccaaccagacaaatccgtactcgtggccgcaggagagaccgctaccctcagatgtaccatcacttctctcttccccgttggccccatccagtggtttcgaggcgcaggaccaggacgagtgcttatttacaatcaacgacagggcccattcccaagagtgacaacagtatccgataccaccaagcgcaataatatggactttagcattagaatcggcaacataacacccgctgacgccggtacatactattgtattaaatttcgaaagggctcaccagacgacgtggaatttaagtcaggggccggaaccgaactctcagttagagcaaaaccttctgctagcgacatcaagctgcagcagagcggcgccgagctggccaggcccggcgccagcgtgaagatgagctgcaagaccagcggctacaccttcaccaggtacaccatgcactgggtgaagcagaggcccggccagggcctggagtggatcggctacatcaaccccagcaggggctacaccaactacaaccagaagttcaaggacaaggccaccctgaccaccgacaagagcagcagcaccgcctacatgcagctgagcagcctgaccagcgaggacagcgccgtgtactactgcgccaggtactacgacgaccactactgcctggactactggggccagggcaccaccctgaccgtgagcagcgtggagggcggcagcggcggcagcggcggcagcggcggcagcggcggcgtggacgacatccagctgacccagagccccgccatcatgagcgccagccccggcgagaaggtgaccatgacctgcagggccagcagcagcgtgagctacatgaactggtaccagcagaagagcggcaccagccccaagaggtggatctacgacaccagcaaggtggccagcggcgtgccctacaggttcagcggcagcggcagcggcaccagctacagcctgaccatcagcagcatggaggccgaggacgccgccacctactactgccagcagtggagcagcaaccccctgaccttcggcgccggcaccaagctggagctgaagcaccaccatcatcaccactgag

SEQ ID NO: 46 - protein, 391 aa, synthetic construct
  REGION 1..391: note = Synthesized SIRP1alpha-CD3-SL bi-specific T-cell engager construct
METDTLLLWVLLLWVPGSTGDEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 47 - DNA, 1191 bp, synthetic construct
  misc_feature 1..1191: note = Synthesized polynucleotide encoding SIRP1alpha-CD3-LL bi-specific T-cell engager construct
  source 1..1191: mol_type = other DNA
atggagaccgataccctgctcttgtgggttttgcttctttgggtgccaggatctacaggtgatgaagaagaattgcagatcatccaaccagacaaatccgtactcgtggccgcaggagagaccgctaccctcagatgtaccatcacttctctcttccccgttggccccatccagtggtttcgaggcgcaggaccaggacgagtgcttatttacaatcaacgacagggcccattcccaagagtgacaacagtatccgataccaccaagcgcaataatatggactttagcattagaatcggcaacataacacccgctgacgccggtacatactattgtattaaatttcgaaagggctcaccagacgacgtggaatttaagtcaggggccggaaccgaactctcagttagagcaaaaccttctgctagcggcggcggcggcagcgacatcaagctgcagcagagcggcgccgagctggccaggcccggcgccagcgtgaagatgagctgcaagaccagcggctacaccttcaccaggtacaccatgcactgggtgaagcagaggcccggccagggcctggagtggatcggctacatcaaccccagcaggggctacaccaactacaaccagaagttcaaggacaaggccaccctgaccaccgacaagagcagcagcaccgcctacatgcagctgagcagcctgaccagcgaggacagcgccgtgtactactgcgccaggtactacgacgaccactactgcctggactactggggccagggcaccaccctgaccgtgagcagcgtggagggcggcagcggcggcagcggcggcagcggcggcagcggcggcgtggacgacatccagctgacccagagccccgccatcatgagcgccagccccggcgagaaggtgaccatgacctgcagggccagcagcagcgtgagctacatgaactggtaccagcagaagagcggcaccagccccaagaggtggatctacgacaccagcaaggtggccagcggcgtgccctacaggttcagcggcagcggcagcggcaccagctacagcctgaccatcagcagcatggaggccgaggacgccgccacctactactgccagcagtggagcagcaaccccctgaccttcggcgccggcaccaagctggagctgaagcaccaccaccaccaccactag

SEQ ID NO: 48 - protein, 396 aa, synthetic construct
  REGION 1..396: note = Synthesized SIRP1alpha-CD3-LL bi-specific T-cell engager construct
METDTLLLWVLLLWVPGSTGDEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSASGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 49 - DNA, 1545 bp, synthetic construct
  misc_feature 1..1545: note = Synthesized polynucleotide encoding PDL1-CD3 bi-specific T-cell engager construct
  source 1..1545: mol_type = other DNA
atggagttcggcctgagctgggtgttcctggtggccctgttcaggggcgtgcagtgcgacatcaagctgcagcagagcggcgccgagctggccaggcccggcgccagcgtgaagatgagctgcaagaccagcggctacaccttcaccaggtacaccatgcactgggtgaagcagaggcccggccagggcctggagtggatcggctacatcaaccccagcaggggctacaccaactacaaccagaagttcaaggacaaggccaccctgaccaccgacaagagcagcagcaccgcctacatgcagctgagcagcctgaccagcgaggacagcgccgtgtactactgcgccaggtactacgacgaccactactgcctggactactggggccagggcaccaccctgaccgtgagcagcgtggagggcggcagcggcggcagcggcggcagcggcggcagcggcggcgtggacgacatccagctgacccagagccccgccatcatgagcgccagccccggcgagaaggtgaccatgacctgcagggccagcagcagcgtgagctacatgaactggtaccagcagaagagcggcaccagccccaagaggtggatctacgacaccagcaaggtggccagcggcgtgccctacaggttcagcggcagcggcagcggcaccagctacagcctgaccatcagcagcatggaggccgaggacgccgccacctactactgccagcagtggagcagcaaccccctgaccttcggcgccggcaccaagctggagctgaagggcggcggcggcagcgatatccagatgacacagagcccatcatctctgtctgcaagcgtaggagaccgagtcaccattacatgcagagcctcccaagacgtttccacagcagtggcctggtatcagcaaaaacctggtaaggcgcccaagcttctcatctattcagccagttttctgtatagcggcgttcccagccgattctctggctctggatccggcacggactttactttgacaatttcctctcttcagcccgaagattttgcaacctactactgtcagcaatatctctaccatccagccacattcggacagggcaccaaagtcgaaatcaaaagaggcggcggcggcagtggcggcgggggttcaggaggcgggggttctgaagtgcaactcgttgaaagcggaggagggcttgtccaacctggcgggtcactgcggttgagctgcgccgcaagcggattcaccttctcagactcttggatccattgggtgcgccaggctcccggaaaaggcttggaatgggttgcttggatttcaccgtatggcggttccacatactacgctgacagcgttaagggtcgattcaccatctctgcagatacttcaaaaaacacagcctaccttcagatgaatagtttgcgcgccgaggacacagcggtttattattgtgcccgaagacattggcccggcggtttcgactactgggggcaaggtacgttggtgactgtgagcgcccaccaccatcatcaccactga

SEQ ID NO: 50 - protein, 514 aa, synthetic construct
  REGION 1..514: note = Synthesized PDL1-CD3 bi-specific T-cell engager construct
MEFGLSWVFLVALFRGVQCDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAHHHHHH

SEQ ID NO: 51 - DNA, 2244 bp, synthetic construct
  misc_feature 1..2244: note = Synthesized polynucleotide encoding PDL1-CD3-Fc bi-specific T-cell engager construct
  source 1..2244: mol_type = other DNA
atggagttcggcctgagctgggtgttcctggtggccctgttcaggggcgtgcagtgcgacatcaagctgcagcagagcggcgccgagctggccaggcccggcgccagcgtgaagatgagctgcaagaccagcggctacaccttcaccaggtacaccatgcactgggtgaagcagaggcccggccagggcctggagtggatcggctacatcaaccccagcaggggctacaccaactacaaccagaagttcaaggacaaggccaccctgaccaccgacaagagcagcagcaccgcctacatgcagctgagcagcctgaccagcgaggacagcgccgtgtactactgcgccaggtactacgacgaccactactgcctggactactggggccagggcaccaccctgaccgtgagcagcgtggagggcggcagcggcggcagcggcggcagcggcggcagcggcggcgtggacgacatccagctgacccagagccccgccatcatgagcgccagccccggcgagaaggtgaccatgacctgcagggccagcagcagcgtgagctacatgaactggtaccagcagaagagcggcaccagccccaagaggtggatctacgacaccagcaaggtggccagcggcgtgccctacaggttcagcggcagcggcagcggcaccagctacagcctgaccatcagcagcatggaggccgaggacgccgccacctactactgccagcagtggagcagcaaccccctgaccttcggcgccggcaccaagctggagctgaagggcggcggcggcagcgatatccagatgacacagagcccatcatctctgtctgcaagcgtaggagaccgagtcaccattacatgcagagcctcccaagacgtttccacagcagtggcctggtatcagcaaaaacctggtaaggcgcccaagcttctcatctattcagccagttttctgtatagcggcgttcccagccgattctctggctctggatccggcacggactttactttgacaatttcctctcttcagcccgaagattttgcaacctactactgtcagcaatatctctaccatccagccacattcggacagggcaccaaagtcgaaatcaaaagaggcggcggcggcagtggcggcgggggttcaggaggcgggggttctgaagtgcaactcgttgaaagcggaggagggcttgtccaacctggcgggtcactgcggttgagctgcgccgcaagcggattcaccttctcagactcttggatccattgggtgcgccaggctcccggaaaaggcttggaatgggttgcttggatttcaccgtatggcggttccacatactacgctgacagcgttaagggtcgattcaccatctctgcagatacttcaaaaaacacagcctaccttcagatgaatagtttgcgcgccgaggacacagcggtttattattgtgcccgaagacattggcccggcggtttcgactactgggggcaaggtacgttggtgactgtgagcgccgtagatgaagcaaaatcttgtgacaaaacccatacctgcccaccatgcccagccccagaacttcttggcggaccctctgtcttccttttccctccgaagcccaaggataccctgatgatcagccgaaccccggaggtaacatgtgtggtggtcgatgttagccatgaggatcctgaagtcaaatttaactggtatgtagacggtgttgaggtgcacaacgctaaaactaagcccagggaggagcagtacaactcaacctatcgcgtcgtatctgtgcttaccgtcctgcatcaagactggctcaatggtaaggaatataaatgtaaagtgagtaacaaggcactgccagcacctatcgaaaaaaccatctcaaaggcgaagggacagcccagggaaccccaggtctatactctgccaccttctcgggatgaattgaccaagaaccaagttagcctgacatgtctggtgaaaggtttctatccaagcgatatagctgtcgagtgggagtccaatggccaacctgagaacaattataagaccaccccacccgttctggacagcgacggatcctttttcctgtactcaaaactcactgtcgataaatcaagatggcaacaaggcaacgtttttagctgtagcgtgatgcacgaagcacttcataatcactatacacagaagtcactctctctttctccaggacaccaccatcatcaccactga

SEQ ID NO: 52 - protein, 747 aa, synthetic construct
  REGION 1..747: note = Synthesized PDL1-CD3-Fc bi-specific T-cell engager construct
MEFGLSWVFLVALFRGVQCDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAVDEAKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 53 - protein, 707 aa, synthetic construct
  REGION 1..707: note = Synthesized CD19-IL15 bi-specific T-cell engager construct
MEFGLSWVFLVALFRGVQCDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHHRRKREGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 54 - protein, 1149 aa, synthetic construct
  REGION 1..1149: note = Synthesized CD19-IL12 bi-specific T-cell engager construct
MEFGLSWVFLVALFRGVQCDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHHRRKREGRGSLLTCGDVEENPGPMWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASRRKREGRGSLLTCGDVEENPGPPMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 55 - protein, 643 aa, synthetic construct
  REGION 1..643: note = Synthesized CD19-CXCL10 bi-specific T-cell engager construct
MEFGLSWVFLVALFRGVQCDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHHRRKREGRGSLLTCGDVEENPGPMNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 56 - protein, 575 aa, synthetic construct
  REGION 1..575: note = Synthesized SIRP1alpha-IL15-SL bi-specific T-cell engager construct
METDTLLLWVLLLWVPGSTGDEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHHRRKREGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 57 - protein, 580 aa, synthetic construct
  REGION 1..580: note = Synthesized SIRP1alpha-IL15-LL bi-specific T-cell engager construct
METDTLLLWVLLLWVPGSTGDEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSASGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHHRRKREGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 58 - protein, 1017 aa, synthetic construct
  REGION 1..1017: note = Synthesized SIRP1alpha-IL12-SL bi-specific T-cell engager construct
METDTLLLWVLLLWVPGSTGDEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHHRRKREGRGSLLTCGDVEENPGPMWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASRRKREGRGSLLTCGDVEENPGPPMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 59 - protein, 1022 aa, synthetic construct
  REGION 1..1022: note = Synthesized SIRP1alpha-IL12-LL bi-specific T-cell engager construct
METDTLLLWVLLLWVPGSTGDEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSASGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHHRRKREGRGSLLTCGDVEENPGPMWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASRRKREGRGSLLTCGDVEENPGPPMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 60 - protein, 511 aa, synthetic construct
  REGION 1..511: note = Synthesized SIRP1alpha-CXCL10-SL bi-specific T-cell engager construct
METDTLLLWVLLLWVPGSTGDEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHHRRKREGRGSLLTCGDVEENPGPMNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 61 - protein, 516 aa, synthetic construct
  REGION 1..516: note = Synthesized SIRP1alpha-CXCL10-LL bi-specific T-cell engager construct
METDTLLLWVLLLWVPGSTGDEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSASGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHHRRKREGRGSLLTCGDVEENPGPMNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 62 - protein, 698 aa, synthetic construct
  REGION 1..698: note = Synthesized PDL1-CD3-IL15 bi-specific T-cell engager construct
MEFGLSWVFLVALFRGVQCDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAHHHHHHRRKREGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 63 - protein, 1140 aa, synthetic construct
  REGION 1..1140: note = Synthesized PDL1-CD3-IL12 bi-specific T-cell engager construct
MEFGLSWVFLVALFRGVQCDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAHHHHHHRRKREGRGSLLTCGDVEENPGPMWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASRRKREGRGSLLTCGDVEENPGPPMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 64 - protein, 634 aa, synthetic construct
  REGION 1..634: note = Synthesized PDL1-CD3-CXCL10 bi-specific T-cell engager construct
MEFGLSWVFLVALFRGVQCDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAHHHHHHRRKREGRGSLLTCGDVEENPGPMNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 65 - protein, 1120 aa, synthetic construct
  REGION 1..1120: note = Synthesized SIRP1alpha-MMP9-SL bi-specific T-cell engager construct
METDTLLLWVLLLWVPGSTGDEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHHRRKREGRGSLLTCGDVEENPGPMSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 66 - protein, 1125 aa, synthetic construct
  REGION 1..1125: note = Synthesized SIRP1alpha-MMP9-LL bi-specific T-cell engager construct
METDTLLLWVLLLWVPGSTGDEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSASGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHHRRKREGRGSLLTCGDVEENPGPMSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 67 - DNA, 2814 bp, synthetic construct
  misc_feature 1..2814: note = Synthesized polynucleotide encoding SIRP1alpha-PDL1-CD3-Fc-SL bi-specific T-cell engager construct
  source 1..2814: mol_type = other DNA
atggaaaccgatacacttctgttgtgggtgctgctgctgtgggtccctggttcaacaggcgattatccctacgatgtgcccgactacgcaggcgctcagccagctgatgatatccagatgacacagagcccatcatctctgtctgcaagcgtaggagaccgagtcaccattacatgcagagcctcccaagacgtttccacagcagtggcctggtatcagcaaaaacctggtaaggcgcccaagcttctcatctattcagccagttttctgtatagcggcgttcccagccgattctctggctctggatccggcacggactttactttgacaatttcctctcttcagcccgaagattttgcaacctactactgtcagcaatatctctaccatccagccacattcggacagggcaccaaagtcgaaatcaaaagaggcggcggcggcagtggcggcgggggttcaggaggcgggggttctgaagtgcaactcgttgaaagcgtaggagggcttgtccaacctggcgggtcactgcggttgagctgcgccgcaagcggattcaccttctcagactcttggatccattgggtgcgccaggctcccggaaaaggcttggaatgggttgcttggatttcaccgtatggcggttccacatactacgctgacagcgttaagggtcgattcaccatctctgcagatacttcaaaaaacacagcctaccttcagatgaatagtttgcgcgccgaggacacagcggtttattattgtgccctaagacattggcccggcggtttcgactactgggggcaaggtacgttggtgactgtgagcgccgtagatgaagcaaaatcttgtgacaaaacccatacctgcccaccatgcccagccccagaacttcttggcgtaccctctgtcttccttttccctccgaagcccaaggataccctgatgatcagccgaaccccggaggtaacatgtgtggtggtcgatgttagccatgaggatcctgaagtcaaatttaactggtatgtagacggtgttgaggtgcacaacgctaaaactaagcccagggaggagcagtacaactcaacctatcgcgtcgtatctgtgcttaccgtcctgcatcaagactggctcaatggtaaggaatataaatgtaaagtgagtaacaaggcactgccagcacctatcgaaaaaaccatctcaaaggcgaagggacagcccagggaaccccaggtctatactctgcaaccttctcgggatgaattgaccaagaaccaagttagcctgacatgtctggtgaaaggtttctatccaagcgatatagctgtcgagtgggagtccaatggccaacctgagaacaattataagaccaccccacccgttctggacagcgacggatcctttttcctgtactcaaaactcactgtcgataaatcaagatggcaacaaggcaacgtttttagctgtagcgtgatgcacgaagcacttcataatcactatacacagaagtcactctctctttctccaggaaaggttgacgaacagaaattgatatccgaggaagatctcaataggaggaagagagaaggcagggggagccttctcacttgcggcgatgtcgaggaaaatccggggcctatggagaccgataccctgctcttgtgggttttgcttctttgggtgccaggatctacaggtgatgaagaagaattgcagatcatccaaccagacaaatccgtactcgtggccgcaggagagaccgctaccctcagatgtaccatcacttctctcttccccgttggccccatccagtggtttcgaggcgcaggaccaggacgagtgcttatttacaatcaacgacagggcccattcccaagagtgacaacagtatccgataccaccaagcgcaataatatggactttagcattagaatcggcaacataacacccgctgacgccggtacatactattgtattaaatttcgaaagggctcaccagacgacgtggaatttaagtcaggggccggaaccgaactctcagttagagcaaaaccttctgctagcgacatcaagctgcagcagagcggcgccgagctggccaggcccggcgccagcgtgaagatgagctgcaagaccagcggctacaccttcaccaggtacaccatgcactgggtgaagcagaggcccggccagggcctggagtggatcggctacatcaaccccagcaggggctacaccaactacaaccagaagttcaaggacaaggccaccctgaccaccgacaagagcagcagcaccgcctacatgcagctgagcagcctgaccagcgaggacagcgccgtgtactactgcgccaggtactacgacgaccactactgcctggactactggggccagggcaccaccctgaccgtgagcagcgtggagggcggcagcggcggcagcggcggcagcggcggcagcggcggcgtggacgacatccagctgacccagagccccgccatcatgagcgccagccccggcgagaaggtgaccatgacctgcagggccagcagcagcgtgagctacatgaactggtaccagcagaagagcggcaccagccccaagaggtggatctacgacaccagcaaggtggccagcggcgtgccctacaggttcagcggcagcggcagcggcaccagctacagcctgaccatcagcagcatggaggccgaggacgccgccacctactactgccagcagtggagcagcaaccccctgacctccggcgccggcaccaagctggagctgaagcaccaccatcatcaccactga

SEQ ID NO: 68 - protein, 937 aa, synthetic construct
  REGION 1..937: note = Synthesized SIRP1alpha-PDL1-CD3-Fc-SL bi-specific T-cell engager construct
METDRLLLWVLLLWVPGSTGDYPYDVPDYAGAQPADDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAVDEAKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDEQKLISEEDLNRRKREGRGSLLTCGDVEENPGPMETDRLLLWVLLLWVPGSTGDEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSASDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 69 - DNA, 2829 bp, synthetic construct
  misc_feature 1..2829: note = Synthesized polynucleotide encoding SIRP1alpha-PDL1-CD3-Fc-LL bi-specific T-cell engager construct
  source 1..2829: mol_type = other DNA
atggaaaccgatacacttctgttgtgggtgctgctgctgtgggtccctggttcaacaggcgattatccctacgatgtgcccgactacgcaggcgctcagccagctgatgatatccagatgacacagagcccatcatctctgtctgcaagcgtaggagaccgagtcaccattacatgcagagcctcccaagacgtttccacagcagtggcctggtatcagcaaaaacctggtaaggcgcccaagcttctcatctattcagccagttttctgtatagcggcgttcccagccgattctctggctctggatccggcacggactttactttgacaatttcctctcttcagcccgaagattttgcaacctactactgtcagcaatatctctaccatccagccacattcggacagggcaccaaagtcgaaatcaaaagaggcggcggcggcagtggcggcgggggttcaggaggcgggggttctgaagtgcaactcgttgaaagcgtaggagggcttgtccaacctggcgggtcactgcggttgagctgcgccgcaagcggattcaccttctcagactcttggatccattgggtgcgccaggctcccggaaaaggcttggaatgggttgcttggatttcaccgtatggcggttccacatactacgctgacagcgttaagggtcgattcaccatctctgcagatacttcaaaaaacacagcctaccttcagatgaatagtttgcgcgccgaggacacagcggtttattattgtgccctaagacattggcccggcggtttcgactactgggggcaaggtacgttggtgactgtgagcgccgtagatgaagcaaaatcttgtgacaaaacccatacctgcccaccatgcccagccccagaacttcttggcgtaccctctgtcttccttttccctccgaagcccaaggataccctgatgatcagccgaaccccggaggtaacatgtgtggtggtcgatgttagccatgaggatcctgaagtcaaatttaactggtatgtagacggtgttgaggtgcacaacgctaaaactaagcccagggaggagcagtacaactcaacctatcgcgtcgtatctgtgcttaccgtcctgcatcaagactggctcaatggtaaggaatataaatgtaaagtgagtaacaaggcactgccagcacctatcgaaaaaaccatctcaaaggcgaagggacagcccagggaaccccaggtctatactctgcaaccttctcgggatgaattgaccaagaaccaagttagcctgacatgtctggtgaaaggtttctatccaagcgatatagctgtcgagtgggagtccaatggccaacctgagaacaattataagaccaccccacccgttctggacagcgacggatcctttttcctgtactcaaaactcactgtcgataaatcaagatggcaacaaggcaacgtttttagctgtagcgtgatgcacgaagcacttcataatcactatacacagaagtcactctctctttctccaggaaaggttgacgaacagaaattgatatccgaggaagatctcaataggaggaagagagaaggcagggggagccttctcacttgcggcgatgtcgaggaaaatccggggcctatggagaccgataccctgctcttgtgggttttgcttctttgggtgccaggatctacaggtgatgaagaagaattgcagatcatccaaccagacaaatccgtactcgtggccgcaggagagaccgctaccctcagatgtaccatcacttctctcttccccgttggccccatccagtggtttcgaggcgcaggaccaggacgagtgcttatttacaatcaacgacagggcccattcccaagagtgacaacagtatccgataccaccaagcgcaataatatggactttagcattagaatcggcaacataacacccgctgacgccggtacatactattgtattaaatttcgaaagggctcaccagacgacgtggaatttaagtcaggggccggaaccgaactctcagttagagcaaaaccttctgctagcggcggcggcggcagcgacatcaagctgcagcagagcggcgccgagctggccaggcccggcgccagcgtgaagatgagctgcaagaccagcggctacaccttcaccaggtacaccatgcactgggtgaagcagaggcccggccagggcctggagtggatcggctacatcaaccccagcaggggctacaccaactacaaccagaagttcaaggacaaggccaccctgaccaccgacaagagcagcagcaccgcctacatgcagctgagcagcctgaccagcgaggacagcgccgtgtactactgcgccaggtactacgacgaccactactgcctggactactggggccagggcaccaccctgaccgtgagcagcgtggagggcggcagcggcggcagcggcggcagcggcggcagcggcggcgtggacgacatccagctgacccagagccccgccatcatgagcgccagccccggcgagaaggtgaccatgacctgcagggccagcagcagcgtgagctacatgaactggtaccagcagaagagcggcaccagccccaagaggtggatctacgacaccagcaaggtggccagcggcgtgccctacaggttcagcggcagcggcagcggcaccagctacagcctgaccatcagcagcatggaggccgaggacgccgccacctactactgccagcagtggagcagcaaccccctgaccttcggcgccggcaccaagctggagctgaagcaccaccaccaccaccactag

SEQ ID NO: 70 - protein, 942 aa, synthetic construct
  REGION 1..942: note = Synthesized SIRP1alpha-PDL1-CD3-Fc-LL bi-specific T-cell engager construct
METDRLLLWVLLLWVPGSTGDYPYDVPDYAGAQPADDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAVDEAKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDEQKLISEEDLNRRKREGRGSLLTCGDVEENPGPMETDRLLLWVLLLWVPGSTGDEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSASGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 71 - protein, 107 aa, synthetic construct
  REGION 1..107: note = anti-FAP light chain sequence
QIVLTQSPAIMSASPGEKVTMTCSASSGVNFMHWYQQKSGTSPKRWIFDTSKLASGVPARFSGSGSGTSYSLTISSMEAEDAATYYCQQWSFNPPTFGGGTKLEIKR

SEQ ID NO: 72 - protein, 117 aa, synthetic construct
  REGION 1..117: note = anti-FAP heavy chain sequence
QVQLQQSGAELARPGASVNLSCKASGYTFTNNGINWLKQRTGQGLEWIGEIYPRSTNTLYNEKFKGKATLTADRSSNTAYMELRSLTSEDSAVYFCARTLTAPFAFWGQGTLVTVSA

SEQ ID NO: 73 - protein, 242 aa, synthetic construct
  REGION 1..242: note = anti-FAP scFv sequence
QVQLQQSGAELARPGASVNLSCKASGYTFTNNGINWLKQRTGQGLEWIGEIYPRSTNTLYNEKFKGKATLTADRSSNTAYMELRSLTSEDSAVYFCARTLTAPFAFWGQGTLVTVSAGSTSGSGKPGSGEGSTKGQIVLTQSPAIMSASPGEKVTMTCSASSGVNFMHWYQQKSGTSPKRWIFDTSKLASGVPARFSGSGSGTSYSLTISSMEAEDAATYYCQQWSFNPPTFGGGTKLEIKR

SEQ ID NO: 74 - DNA, 1545 bp, synthetic construct
  misc_feature 1..1545: note = Synthetic polynucleotide sequence encoding anti-FAP - anti-CD3 BiTE sequence
  source 1..1545: mol_type = other DNA
atggttttgcttgtgaccagcctcctgctctgtgaactgcctcatcctgcattcctgttgatcccacaggtgcagctccagcagagtggcgcagagctcgctcgcccaggcgcttctgtgaatctgagttgtaaggcctccggatatacttttacgaacaacggcatcaactggctgaagcagcggaccggccagggcctggagtggatcggcgaaatatacccccggtccacaaacactctctataacgagaagtttaagggcaaagcaactctgaccgcggacaggtcctctaacacagcctatatggagctgagaagcttgacgagtgaggactccgctgtctatttttgcgcccgaactctgaccgctccttttgctttttggggccagggcacgctcgtgaccgtaagtgcgggctccactagcggttccggcaaacctggcagcggagaaggcagcaccaaagggcagatcgtcctgacgcagtctccagccatcatgagcgcctcacccggcgaaaaggtgaccatgacctgctcagcctcttctggtgtgaatttcatgcactggtaccagcaaaaaagtgggacctcccctaaaaggtggatcttcgataccagcaaactggcttctggcgttcccgcaaggtttagcggctctggttccggcacatcatacagcctgacgatcagcagcatggaggcagaagacgcagctacctattactgccagcaatggagctttaacccacctactttcggaggaggaacaaagctggaaataaaaagaggtggtggtggatcagaggtgcagctggtggagtctgggggaggcttggtgcagcctggaaagtccctgaaactctcctgtgaggcctctggattcaccttcagcggctatggcatgcactgggtccgccaggctccagggagggggctggagtcggtcgcatacattactagtagtagtattaatatcaaatatgctgacgctgtgaaaggccggttcaccgtctccagagacaatgccaagaacttactgtttctacaaatgaacattctcaagtctgaggacacagccatgtactactgtgcaagattcgactgggacaaaaattactggggccaaggaaccatggtcaccgtctcctcaggtggtggtggatcaggtggaggcggaagtggaggtggcggatccgacatccagatgacccagtctccatcatcactgcctgcctccctgggagacagagtcactatcaattgtcaggccagtcaggacattagcaattatttaaactggtaccagcagaaaccagggaaagctcctaagctcctgatctattatacaaataaattggcagatggagtcccatcaaggttcagtggcagtggttctgggagagattcttctttcactatcagcagcctggaatccgaagatattggatcttattactgtcaacagtattataactatccgtggacgttcggacctggcaccaagctggaaatcaaacggcaccaccatcatcaccactga

SEQ ID NO: 75 - protein, 512 aa, synthetic construct
  REGION 1..512: note = anti-FAP - anti-Cd3 BiTE sequence
MYRMQLLSCIALSLALVTNSQVQLQQSGAELARPGASVNLSCKASGYTFTNNGINWLKQRTGQGLEWIGEIYPRSTNTLYNEKFKGKATLTADRSSNTAYMELRSLTSEDSAVYFCARTLTAPFAFWGQGTLVTVSAGSTSGSGKPGSGEGSTKGQIVLTQSPAIMSASPGEKVTMTCSASSGVNFMHWYQQKSGTSPKRWIFDTSKLASGVPARFSGSGSGTSYSLTISSMEAEDAATYYCQQWSFNPPTFGGGTKLEIKRGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKRHHHHHH

SEQ ID NO: 76 - protein, 116 aa, synthetic construct
  REGION 1..116: note = anti-mouse CD3 heavy chain sequence
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSS

SEQ ID NO: 77 - protein, 108 aa, synthetic construct
  REGION 1..108: note = anti-mouse CD3 light chain
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKR

SEQ ID NO: 78 - protein, 239 aa, synthetic construct
  REGION 1..239: note = anti-mouse CD3 scFv
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKR

SEQ ID NO: 79 - protein, 18 aa, synthetic construct
  REGION 1..18: note = Synthetic linker sequence
GSTSGSGKPGSGEGSTKG

SEQ ID NO: 80 - protein, 92 aa, synthetic construct
  REGION 1..92: note = CCL4 sequence
MKLCVTVLSLLMLVAAFCSPALSAPMGSDPPTACCFSYTARKLPRNFVVDYYETSSLCSQPAVVFQTKRSKQVCADPSESWVQEYVYDLELN

(19) United States
(12) Patent Application Publication
(10) Pub. No.: US 2024/0207334 A1
(43) Pub. Date: Jun. 27, 2024

(54) ONCOLYTIC VIRAL DELIVERY OF THERAPEUTIC POLYPEPTIDES

(71) Applicant: Virogin Biotech Canada Ltd., Richmond (CA)

(72) Inventors: Mitchell H. FINER, Cambridge, MA (US); Luke EVNIN, Cambridge, MA (US)

(21) Appl. No.: 18/513,432

(22) Filed: Nov. 17, 2023

Related U.S. Application Data
(63) Continuation of application No. 16/958,371, filed on Jun. 26, 2020, now Pat. No. 11,865,081.
(60) Provisional application No. 62/611,941, filed on Dec. 29, 2017.

(51) Int. Cl. A61K 35/763; A61K 9/00; A61P 35/00; C07K 14/54; C07K 16/28; C12N 9/64

(52) U.S. Cl. CPC A61K 35/763; A61P 35/00; C07K 14/5434; C07K 14/5443; C07K 16/2803; C12N 9/6416; A61K 9/0019; C12Y 304/24035

(57) ABSTRACT

Described herein are oncolytic viruses comprising one or more nucleic acids encoding an engager molecule. In some embodiments, the oncolytic viruses comprise one or more nucleic acids encoding an engager molecule and one or more therapeutic molecules. Pharmaceutical compositions containing the oncolytic virus and methods of treating cancer using the oncolytic viruses are further provided herein.

### DESCRIPTION OF THE TEXT FILED SUBMITTED ELECTRONICALLY

The contents of the text filed submitted electronically herewith are incorporated herein by reference in their entirety: A computer readable format copy of the Sequence Listing (file name: ONCR_011_001WO_ST25.txt; date generated: Dec. 21, 2018; file size: 208 kilobytes).

### BACKGROUND OF THE INVENTION

Patients with certain hematologic and solid tumors remain in need of new therapies. The use of bispecific antibodies to direct cytotoxic T cells to tumor cells, and chimeric antigen receptors (CARs) to engineer antigen specificity onto an immune effector cell are being demonstrated to provide a therapeutic benefit. Also, oncolytic virus technologies are useful additions to the current standard of care of solid tumors, expected to have a safety profile and the ability to infect, replicate in, and lyse tumor cells. However, the antitumor efficacy of the bispecific antibodies, CARs and oncolytic virus are suboptimal, demonstrating the continued need for further advances of oncology, antibodies, and oncolytic virus therapy.

### SUMMARY OF THE INVENTION

In some embodiments, the present disclosure provides an oncolytic virus comprising a first recombinant nucleic acid encoding a polypeptide comprising an effector domain specific for an antigen expressed on an effector cell and an targeting domain specific for an antigen expressed on a target cell, wherein the target antigen is fibroblast activation protein (FAP). In some embodiments, the present disclosure provides an oncolytic virus comprising one or more micro RNA (miR) target sequences inserted into a locus of one or more genes required for viral replication, and further comprising a a first nucleic acid sequence encoding a polypeptide comprising an effector domain specific for an antigen expressed on an effector cell and a targeting domain that binds to a target antigen expressed on a target cell, wherein the target antigen is fibroblast activation protein (FAP). In some embodiments, the one or more miR target sequences is recognized by miR-10b, miR-17, miR-21, miR-106a, miR-125b, miR-145, miR-146a, miR-146b, miR-155, miR-96, miR-182, miR-183, miR-221, miR-222, or miR-1247-5p. In some embodiments, the one or more miR target sequences is recognized by a plurality of microRNAs selected from miR-10b, miR-17, miR-21, miR-106a, miR-125b, miR-145, miR-146a, miR-146b, miR-155, miR-96, miR-182, miR-183, miR-221, miR-222, and miR-1247-5p.

In some embodiments, the oncolytic virus is derived from herpes simplex virus-1 (HSV-1). In some embodiments, the oncolytic virus is capable of preferential replication in a tumor cell.

In some embodiments, the recombinant nucleic acid further comprises a second nucleic acid sequence encoding an immune modulator polypeptide selected from the group consisting of a cytokine, a costimulatory molecule, an immune checkpoint polypeptide, an anti-angiogenesis factor, and a matrix metalloprotease (MMP). In some embodiments, the cytokine is selected from IL-15, IL-12, CXCL10, and CCL4. In some embodiments, the immune checkpoint polypeptide comprises i) an inhibitor of PD-1, PDL-1, CTLA-4, LAG3, TIM3, neuropilin, or CCR4; ii) an agonist of GITR, OX-40, or CD28; or iii) a combination of i) and ii). In some embodiments, the MMP is MMP9. In some embodiments, the cytokine is selected from IL-15, IL-12, CCL4, and CXCL10.

In some embodiments, the engager polypeptide is a bipartite polypeptide and is comprised of an antibody, an antibody domain, a human immunoglobulin heavy chain variable domain, a dual-variable-domain antibody (DVD-Ig), a Tandab, a diabody, a flexibody, a dock-and-lock antibody, a Scorpion polypeptide, a single chain variable fragment (scFv), a BiTE, a DuoBody, an Fe-engineered IgG, an Fcab, a Mab2, or DART polypeptide.

In some embodiments, the first and second nucleic acid sequences are expressed from a single promoter sequence. In some embodiments, the recombinant nucleic acid is a multicistronic sequence. In some embodiments, the multicistronic sequence is a bicistronic sequence or a tricistronic sequence. In some embodiments, the multicistronic sequence comprises a picornavirus-2a-like sequence, and wherein the first and second nucleic acid sequences are expressed from a single promoter sequence present in the recombinant nucleic acid. In some embodiments, the first and second nucleic acids have a size of 7.2-38 kb.

In some embodiments, the effector cell is a T cell or an NKT cell, an NK cell, or a macrophage. In some embodiments, the antigen expressed on the effector cell is CD3 CD4, CD5, CD8, CD16, CD28, CD40, CD134, CD137, or NKG2D.

In some embodiments, the present disclosure provides an oncolytic virus comprising a first recombinant nucleic acid encoding a polypeptide comprising an effector domain specific for an antigen expressed on an effector cell and a targeting domain specific for an antigen expressed on a target cell, wherein the effector cell antigen is CD3 and the target antigen is fibroblast activation protein (FAP). In some embodiments, the engager molecule comprises a polypeptide sequence that is at least 90%, 95%, 96%, 97%, 98%, or 99% identical to SEQ ID NO: 75. In some embodiments, the engager molecule comprises the polypeptide sequence of SEQ ID NO: 75. In some embodiments, the engager molecule consists of the polypeptide sequence of SEQ ID NO: 75. In some embodiments, the first recombinant nucleic acid is at least 95%, 96%, 97%, 98%, or 99% identical to SEQ ID NO: 74. In some embodiments, the first recombinant nucleic acid comprises SEQ ID NO: 74. In some embodiments, the first recombinant nucleic acid consists of SEQ ID NO: 74.

In some embodiments, the oncolytic virus further comprises a second recombinant nucleic acid encoding a therapeutic molecule. In some embodiments, the therapeutic molecule is IL-12, IL-15, CXCL10, or MMP9. In some embodiments, the therapeutic molecule is IL-12 and wherein the recombinant nucleic acid sequence encodes a polypeptide sequence that is at least 90%, 95%, 96%, 97%, 98%, or 99% identical to SEQ ID NO: 54. In some embodiments, the therapeutic molecule is IL-15 and wherein the recombinant nucleic acid sequence encodes a polypeptide sequence that is at least 90%, 95%, 96%, 97%, 98%, or 99% identical to SEQ ID NO: 53. In some embodiments, the therapeutic molecule is CXCL10 and wherein the recombinant nucleic acid sequence encodes a polypeptide sequence that is at least 90%, 95%, 96%, 97%, 98%, or 99% identical to SEQ ID NO: 55. In some embodiments, the therapeutic molecule is CCL4 and wherein the recombinant nucleic acid sequence encodes a polypeptide sequence that is at least 90%, 95%, 96%, 97%, 98%, or 99% identical to SEQ ID NO: 80. In some embodiments, the therapeutic molecule is MMP9 and wherein the recombinant nucleic acid sequence encodes a polypeptide sequence that is at least 90%, 95%, 96%, 97%, 98%, or 99% identical to SEQ ID NO: 34.

In some embodiments, the oncolytic virus further comprises one or more additional recombinant nucleic acid sequences encoding two or more therapeutic molecules. In some embodiments, the two or more therapeutic molecules comprise two or more of CCL4, CXCL20, and IL-12. In some embodiments, the two or more therapeutic molecules comprise each of CCL4, CXCL20, and IL-12.

In some embodiments, the oncolytic virus is a pseudotyped oncolytic virus. In some embodiments, the pseudotyped oncolytic virus comprises one or more modified glycoproteins involved in viral entry into a host cell. In some embodiments, the one or more modified glycoproteins comprise one or more amino acid mutations. In some embodiments, the pseudotyped oncolytic virus comprises a non-viral protein present on the viral surface. In some embodiments, the non-viral protein binds to a protein expressed on the cell-surface of a target cell. In some embodiments, the protein expressed on the cell-surface of the target cell is not associated with a cancer. In some embodiments, the pseudotyped oncolytic virus possesses an altered tropism relative to a non-pseudotyped virus. In some embodiments, the pseudotyped oncolytic virus yields reduced toxicity and/or reduced entry of non-tumor cells or tissue relative to a non-pseudotyped virus. In some embodiments, the pseudotyped oncolytic virus yields increased toxicity and/or increased entry of tumor cells or cancerous tissue relative to a non-pseudotyped virus.

In some embodiments, the oncolytic virus is administered to the subject intravenously. In some embodiments, the oncolytic virus is selected from or is derived from an adenovirus, a herpes simplex virus 1 (HSV1), a myxoma virus, a reovirus, a poliovirus, a vesicular stomatitis virus (VSV), a measles virus (MV), a lassa virus (LASV), or a Newcastle disease virus (NDV).

In some embodiments, the present disclosure provides a pharmaceutical composition comprising an oncolytic virus described herein. In some embodiments, the composition is capable of being administered intravenously. In some embodiments, the oncolytic virus induces an immune response. In some embodiments, the immune response is selectively cytotoxic to a target cell. In some embodiments, the target cell is derived from a solid tumor or a hematologic cancer.

In some embodiments, the present disclosure provides a method of treating a cancer in a subject in need thereof, comprising administering a therapeutically effective amount of an oncolytic virus described herein or a pharmaceutical composition thereof. In some embodiments, the method further comprises administering one or more additional therapies to the subject in need thereof. In some embodiments, the one or more additional therapies comprise surgery, radiation, chemotherapy, immunotherapy, hormone therapy, or a combination thereof.

In some embodiments, the present disclosure provides a method of treating one or more tumors in a subject in need thereof comprising administering a therapeutically effective amount of an oncolytic virus described herein or a pharmaceutical composition thereof to a patient, wherein the one or more tumors express a tumor antigen.

In some embodiments, the present disclosure provides a method of delivering an engager polypeptide and a therapeutic polypeptide to a tumor site comprising administering to a patient in need thereof an oncolytic virus described herein or a pharmaceutical composition thereof.

### BRIEF DESCRIPTION OF THE DRAWINGS

FIG. 1 illustrates an amino acid sequence of a CD19-CD3 bipartite polypeptide comprising a first single chain variable fragment (scFv) directed against CD19 linked to a second scFv directed against CD3.

FIG. 2 illustrates an amino acid sequence of a CD19-CD3-IL15 construct encoded by a bicistronic gene. The first gene encodes a bipartite polypeptide comprising a first scFv directed against CD19 linked to a second scFv directed against CD3. A second gene encoding IL-15 is linked to the bipartite gene sequence by a T2A self-cleaving polypeptide linker.

FIG. 3 illustrates an amino acid sequence of a CD19-CD3-IL12 construct encoded by a multicistronic gene. The first gene encodes a bipartite polypeptide comprising a first scFv directed against CD19 linked to a second scFv directed against CD3. A second gene encoding the p35 subunit of IL-12 is linked to the bipartite gene sequence by a T2A self-cleaving polypeptide linker and a third gene encoding the p40 subunit of IL-12 is linked by a T2A self-cleaving polypeptide linker.

FIG. 4 illustrates an amino acid sequence of a CD19-CD3-CXCL10 construct encoded by a bicistronic gene. The first gene encodes a bipartite polypeptide comprising a first scFv directed against CD19 linked to a second scFv directed against CD3. A second gene encoding CXCL10 is linked to the bipartite gene sequence by a T2A self-cleaving polypeptide linker.

FIG. 5 illustrates an amino acid sequence of a SIRP1α-CD3 bipartite polypeptide comprising a first protein comprising the first 120 amino acids of SIRP1α linked by a single amino acid linker to an scFv directed against CD3.

FIG. 6 illustrates an amino acid sequence of a SIRP1α-CD3-LL bipartite polypeptide comprising a first protein comprising the first 120 amino acids of SIRP1α linked by a G4S motif linker to an scFv directed against CD3.

FIG. 7 illustrates an amino acid sequence of a SIRP1α-CD3-1L15 construct encoded by a bicistronic gene. The first gene encodes a bipartite polypeptide comprising the first 120 amino acids of SIRP1α linked by a single amino acid linker to an scFv directed against CD3. A second gene encoding IL-15 is linked to the bipartite gene sequence by a T2A self-cleaving polypeptide linker.

FIG. 8 illustrates an amino acid sequence of a SIRP1α-CD3-IL15-LL construct encoded by a bicistronic gene. The first gene encodes a bipartite polypeptide comprising the first 120 amino acids of SIRP1α linked by a G4S motif linker to an scFv directed against CD3. A second gene encoding IL-15 is linked to the bipartite gene sequence by a T2A self-cleaving polypeptide linker.

FIG. 9 illustrates an amino acid sequence of a SIRP1α-CD3-IL12 construct encoded by a multicistronic gene. The first gene encodes a bipartite polypeptide comprising the first 120 amino acids of SIRP1α linked by a single amino acid linker to an scFv directed against CD3. A second gene encoding the p35 subunit of IL-12 is linked to the bipartite gene sequence by a T2A self-cleaving polypeptide linker and a third gene encoding the p40 subunit of IL-12 is linked by a T2A self-cleaving polypeptide linker.

FIG. 10 illustrates an amino acid sequence of a SIRP1α-CD3-IL12-LL construct encoded by a multicistronic gene. The first gene encodes a bipartite polypeptide comprising the first 120 amino acids of SIRP1α linked by a G4S motif linker to an scFv directed against CD3. A second gene encoding the p35 subunit of IL-12 is linked to the bipartite gene sequence by a T2A self-cleaving polypeptide linker and a third gene encoding the p40 subunit of IL-12 is linked by a T2A self-cleaving polypeptide linker.

FIG. 11 illustrates an amino acid sequence of a SIRP1α-CD3-CXCL10 construct encoded by a bicistronic gene. The first gene encodes a bipartite polypeptide comprising the first 120 amino acids of SIRP1α linked by a single amino acid linker to an scFv directed against CD3. A second gene encoding CXCL10 is linked to the bipartite gene sequence by a T2A self-cleaving polypeptide linker.

FIG. 12 illustrates an amino acid sequence of a SIRP1α-CD3-CXCL10-LL construct encoded by a bicistronic gene. The first gene encodes a bipartite polypeptide comprising the first 120 amino acids of SIRP1α linked by a G4S motif linker to an scFv directed against CD3. A second gene encoding CXCL10 is linked to the bipartite gene sequence by a T2A self-cleaving polypeptide linker.

FIG. 13 illustrates an amino acid sequence of a PDL1-CD3 bipartite polypeptide comprising a first scFv directed against PDL1 linked to a second scFv directed against CD3.

FIG. 14 illustrates an amino acid sequence of a PDL1-CD3-IL15 construct encoded by a bicistronic gene. The first gene encodes a bipartite polypeptide comprising a first scFv directed against PDL1 linked to a second scFv directed against CD3. A second gene encoding IL-15 is linked to the bipartite gene sequence by a T2A self-cleaving polypeptide linker.

FIG. 15 illustrates an amino acid sequence of a PDL1-CD3-IL12 construct encoded by a multicistronic gene. The first gene encodes a bipartite polypeptide comprising a first scFv directed against PDL1 linked to a second scFv directed against CD3. A second gene encoding the p35 subunit of IL-12 is linked to the bipartite gene sequence by a T2A self-cleaving polypeptide linker and a third gene encoding the p40 subunit of IL-12 is linked by a T2A self-cleaving polypeptide linker.

FIG. 16 illustrates an amino acid sequence of a PDL1-CD3-CXCL10 construct encoded by a bicistronic gene. The first gene encodes a bipartite polypeptide comprising a first scFv directed against PDL1 linked to a second scFv directed against CD3. A second gene encoding CXCL10 is linked to the bipartite gene sequence by a T2A self-cleaving polypeptide linker.

FIG. 17 illustrates an amino acid sequence of a PDL1-CD3-Fc tripartite polypeptide comprising a first scFv directed against CD3, linked by a G4S motif linker to a second scFv directed against PDL1, which is in turn linked to the CH2-CH3 domain of human IgG1 by an IgG1 hinge.

FIG. 18 illustrates an amino acid sequence of a SIRP1α-CD3-MMP9-SL construct encoded by a bicistronic gene (FIG. 18A) and an amino acid sequence of a SIRP1α-CD3-MMP9-LL construct encoded by a bicistronic gene (FIG. 18B).

FIG. 19A-FIG. 19C illustrate the binding of CD19-CD3 BiTE constructs (FIG. 19A), SIRP1α-CD3 BiTE constructs (FIG. 19B), and PDL1-CD3-Fc tripartite T cell engagers (FIG. 19C) CD3⁺ T cells.

FIG. 20 illustrates the quantification of the T cell engager construct binding shown in FIG. 19.

FIG. 21 illustrates the CD3-specific binding of CD19-CD3 BiTE constructs (FIG. 21A), SIRP1α-CD3 BiTE constructs (FIG. 21B), and PDL1-CD3-Fc tripartite T cell engagers (FIG. 21C) through the use of an anti-CD3 antibody, OKT3.

FIG. 22 illustrates the specificity of the CD47-binding SIRP1α arm of a SIRP1α-CD3 BiTE construct.

FIG. 23A-FIG. 23B illustrate the binding of CD19-CD3 and SIRP1α-CD3 BiTE constructs (FIG. 23A) to Raji cells (CD19⁺CD47⁺). % binding is quantified in FIG. 23B.

FIG. 24A-FIG. 24B illustrate the binding of CD19-CD3 and SIRP1α-CD3 BiTE constructs (FIG. 24A) to U2OS cells (CD19⁻CD47⁺). % binding is quantified in FIG. 24B.

FIG. 25A-FIG. 25B illustrate the binding of CD19-CD3 and SIRP1α-CD3 BiTE constructs (FIG. 25A) to GBM30-luc cells (CD19⁻CD47T). % binding is quantified in FIG. 25B.

FIG. 26A-FIG. 26B illustrate the binding of CD19-CD3 and SIRP1α-CD3 BiTE constructs (FIG. 26A) to U251 cells (CD19⁻CD47). % binding is quantified in FIG. 26B.

FIG. 27A-FIG. 27C illustrate the binding of PDL1-Fc-CD3 tripartite T cell engagers to U251 cells. The binding of the PDL1-Fc-CD3 constructs (FIG. 27B) is compared to the binding of an anti-PDL1 antibody (FIG. 27A). Binding was not mediated by FcγRs, as U251 cells do not express FcγRI, FcγRII, or FcγRIII (FIG. 27C).

FIG. 28 illustrates CD19-CD3 BiTE, SIRP1α-CD3 BiTE, and PDL1-CD3-Fc tripartite T cell engager-mediated T cell-dependent cytotoxicity (TDCC) of Raji cells.

FIG. 29 illustrates CD19-CD3 BiTE and PDL1-CD3-Fc tripartite T cell engager-mediated TDCC of THP1 cells.

FIG. 30 illustrates CD19-CD3 BiTE and PDL1-CD3-Fc tripartite T cell engager-mediated TDCC of U251 cells.

FIG. 31 illustrates SIRP1α-CD3 BiTE-mediated TDCC of 293F cells compared to an osteopontin-fusion control construct.

FIG. 32 illustrates expression of SIRP1α-CD3 BiTE constructs from oncolytic-HSV vectors. Expression of SIRP1α-CD3 BiTE constructs with short linkers (Lanes 1-4 and ONCR085 in lanes 5-6, shown in FIG. 5) and SIRP1α-CD3 BiTE constructs with long linkers (ONCR087 in lanes 7-8, shown in FIG. 6) are shown.

FIG. 33 illustrates expression of PDL1-CD3-Fc BiTE constructs from oncolytic-HSV vectors. Purified PDL1-CD3-Fc BiTE protein is shown in lanes 1-4. Concentrated viral supernatants are shown in lanes 5-6.

FIG. 34A-FIG. 34B illustrates TDCC of U251 cells by virally produced SIRP1α-CD3, SIRP1α-CD3-LL, and PDL1-CD3-Fc BiTE constructs. Photographs of U251 cell cultures after incubation with the indicated BiTE constructs and CD8⁺ T cells are shown in FIG. 34A. Activity of virally produced BiTE constructs, measured by % of cell killing and quantified by flow cytometry, is shown in FIG. 34B.

FIG. 35 illustrates that Amicon ultrafiltration effectively removes virus from samples, as determined by Western blotting with polyclonal anti-HSV antibody, and indicated that BiTE-killing is due to the BiTE and not viral infection.

FIG. 36 illustrates a cartoon representation of the production of a pseudotyped oncolytic virus and a recombinant oncolytic virus and infection of a target cell by the respective pseudotyped oncolytic virus and the recombinant oncolytic virus.

FIG. 37 illustrates an amino acid sequence of a SIRP1α-CD3-PDL1-Fc (SL) construct encoded by a bicistronic gene wherein the first gene encodes an anti-PDL1 scFv linked to an IgG1 FEc domain and the second gene encodes a bipartite polypeptide comprising the first 120 amino acids of SIRP1a linked by a single amino acid linker to an scFv directed against CD3.

FIG. 38 illustrates an amino acid sequence of a SIRP1α-CD3-PDL1-Fc (LL) construct encoded by a bicistronic gene wherein the first gene encodes an anti-PDL1 scFv linked to an IgG1 Fc domain and the second gene encodes a bipartite polypeptide comprising the first 120 amino acids of SIRP1α linked by a G4S motif linker to an scFv directed against CD3.

FIG. 39 illustrates a schematic of a SIRP1α-CD3-PDL1-Fc expression plasmid. Two plasmid constructs, one for SIRP1α-CD3-PDL1-Fc (SL) and one for SIRP1α-CD3-PDL1-Fc (LL) were generated.

FIG. 40 illustrates purification of the SIRP1α-CD3 BiTE (SL), SIRP1α-CD3 BiTE (LL), and the anti-PDL1-Fc compounds from supernatants of transfected 293 T cells. FIG. 40A shows purification of anti-PDL1-Fc compounds assessed by Coomassie. FIG. 40B illustrates purification of SIRP1α-CD3 BiTE compounds as assessed by Western Blot using an anti-His detection antibody.

FIG. 41 shows results of a PD1/PDL1 blockade assay. A schematic of the assay is shown in FIG. 41A-FIG. 41B. The results of the PD1/PDL1 blockade assay using the anti-PDL1-Fc compound produced from 293 cells transfected are shown in FIG. 41C.

FIG. 42 shows tumor growth inhibition after treatment with oHSV expressing a FAP-CD3 BiTE (ONCR-148) and combined treatment with ONCR-148 and an oHSV expressing CCL4, CXCL10, and IL-12 (ONCR-153).

### DETAILED DESCRIPTION OF THE INVENTION

The present disclosure provides novel engineered oncolytic viruses, in particular oncolytic viruses that produce multipartite polypeptides and/or other therapeutic polypeptides for the treatment of cancers including solid tumors (e.g., advanced solid tumors) and hematologic malignancies. In some embodiments, the multipartite and/or therapeutic polypeptides produced by the oncolytic viruses described herein mediate and/or enhance the anti-tumor effects of the oncolytic viruses, such as by effector cell-mediated lysis of target cells (e.g., tumor cells). The oncolytic viruses described herein may have multiple (e.g. dual) modes of action, including effector cell-mediated cytolysis of target cells as a result of the expression of multipartite polypeptides, and viral-mediated destruction of target cells. The present disclosure further provides therapeutic compositions comprising the engineered oncolytic viruses and methods of use in the treatment of solid tumors and hematologic malignancies.

### Overview

In some embodiments, the present invention provides oncolytic viruses, compositions thereof, and methods of use for the treatment of cancer. The oncolytic viruses provided herein comprise recombinant nucleic acids that encode engager polypeptides and/or other therapeutic molecules. Typically, the engager polypeptides function as effector cell engagers and generally comprise a first domain directed against a cell surface molecule expressed on an effector cell (e.g., an effector domain) and a second domain directed against a target cell antigen (e.g., a targeting domain) or other cell-surface molecule (e.g., an activation domain). In some embodiments, the engager molecules encoded by the oncolytic viruses described herein are bipartite, tripartite or multipartite polypeptides (e.g., comprising one or multiple engager domains, one or multiple targeting domains, or one or multiple activation domains, and optionally one or multiple other functional domains). In some embodiments, the oncolytic viruses described herein are further engineered by pseudotyping or other recombinant technology in order to modulate the tropism of the virus to result in a viral infection specific for tumor cells and/or surrounding tumor stroma and/or for other beneficial purposes.

Also provided are methods of treating cancer, comprising the step of delivering to human subject in need thereof a therapeutically effective amount of the oncolytic viruses described herein or pharmaceutical compositions thereof. Such methods optionally include the step of delivering to the human subject an additional cancer therapy, such as surgery, radiation, chemotherapy, immunotherapy, hormone therapy, or a combination thereof.

### Definitions

As used herein, the singular forms “a,” “an,” or “the” include plural references unless the context clearly dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 30%, 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein the specification, “subject” or “subjects” or “individuals” include, but are not limited to, mammals such as humans or non-human mammals, including domesticated, agricultural or wild, animals, as well as birds, and aquatic animals. In some embodiments, subjects are livestock such as cattle, sheep, goats, cows, swine, and the like; poultry such as chickens, ducks, geese, turkeys, and the like; and domesticated animals such as dogs and cats. In some embodiments (e.g., particularly in research contexts) subjects are rodents (e.g., mice, rats, hamsters), rabbits, primates, or swine such as inbred pigs and the like. In particular embodiments, the subject is a human. “Patients” are subjects suffering from or at risk of developing a disease, disorder, or condition or otherwise in need of the compositions and methods provided herein. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, “treating” or “treatment” refers to any indicia of success in the treatment or amelioration of a disease or condition, particularly cancer. Treating or treatment may be performed in vitro and/or in vivo, and may comprise delivering an oncolytic virus, or composition thereof, described herein to a patient or subject in need thereof. In some embodiments, treating includes, for example, reducing, delaying or alleviating the severity of one or more symptoms of the disease or condition, and/or reducing the frequency with which symptoms of a disease, defect, disorder, or adverse condition are experienced by a subject or patient. Herein, “treat or prevent” is used herein to refer to a method that results in some level of treatment or amelioration of the disease or condition, and contemplates a range of results directed to that end, including but not restricted to prevention of the condition entirely.

As used herein, “preventing” refers to the prevention of a disease or condition, e.g., tumor formation, in a patient or subject and may also be referred to as “prophylactic treatment.” Prevention of disease development can refer to complete prevention of the symptoms of disease, a delay in disease onset, or a lessening of the severity of the symptoms in a subsequently developed disease. As a non-limiting illustrative example, if an individual at risk of developing a tumor or other form of cancer is treated with the methods of the present invention and does not later develop the tumor or other form of cancer, then the disease has been prevented, at least over a period of time, in that individual.

The terms “therapeutically effective amount” and “therapeutically effective dose” are used interchangeably herein and refer to the amount of an oncolytic viruses or composition thereof that is sufficient to provide a beneficial effect or to otherwise reduce a detrimental non-beneficial event (e.g. an amount or dose sufficient to treat a disease). The exact amount or dose of an oncolytic virus comprised within a therapeutically effective amount or therapeutically effective dose will depend on variety of factors including: the purpose of the treatment; the weight, sex, age, and general health of the subject or patient; the route of administration; the timing of administrations; and the nature of the disease to be treated. The therapeutically effective amount for a given subject or patient is ascertainable by one skilled in the art using known techniques (see, e.g. Lieberman, Pharmaceutical Dosage Forms (vols. 1-3, 1992); Lloyd, The Art, Science and Technology of Pharmaceutical Compounding (1999); and Pickar, Dosage Calculations (1999)).

“Pseudotype” refers to a virus particle, wherein a portion of the virus particle (e.g., the envelope or capsid) comprises one or more heterologous proteins, such as viral proteins derived from a heterologous virus or non-viral proteins, and/or one or more modified viral coat proteins. Non-viral proteins (also referred to as “targeting moieties”) may include antibodies and antigen-binding fragments thereof.

The term “targeting moiety” refers herein to a heterologous protein linked to a virus particle that is capable of binding to a protein on the cell surface of a selected cell type in order to direct interaction between the virus particle and the selected cell type. The targeting moiety may be covalently or non-covalently linked and is generally linked to an envelope protein, e.g., E1, E2, or E3. Representative targeting moieties include antibodies, antigen binding fragments thereof, and receptor ligands. A viral “envelope” protein, or “Env” protein, refers to any polypeptide sequence that resides on the surface lipid bilayer of a virion and functions to mediate the adsorption to and viral entry of host cells susceptible to infection.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In some embodiments, the vector is a virus (i.e., a viral vector or oncolytic viral vector) and the transferred nucleic acid sequence is a recombinant nucleic acid sequence encoding an engager molecule and/or a therapeutic molecule. A viral vector may sometimes be referred to as a “recombinant virus” or a “virus.” The terms “oncolytic virus” and “oncolytic vector” are used interchangeably herein.

“Nucleic acid genome” or “viral genome” refers to the nucleic acid component of a virus particle, which encodes the genome of the virus particle including any proteins required for replication and/or integration of the genome. In some embodiments, a viral genome acts as a viral vector and may comprise a heterologous gene operably linked to a promoter. The promoter may be either native or heterologous to the gene and may be viral or non-viral in origin. The viral genomes described herein may be based on any virus, may be an RNA or DNA genome, and may be either single stranded or double stranded. Preferably, the nucleic acid genome is from the family Rhabdoviridae.

“Retroviral vectors,” as used herein, refer to viral vectors based on viruses of the Retroviridae family. In their wild-type (WT) form, retroviral vectors typically contain a nucleic acid genome.

The term “antibody fragment or derivative thereof” includes polypeptide sequences containing at least one CDR and capable of specifically binding to a target antigen. The term further relates to single chain antibodies, or fragments thereof, synthetic antibodies, antibody fragments, such as a Camel Ig, Ig NAR, Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), and single-domain antibody (sdAb, nanobody), etc., or a chemically modified derivative of any of these. In some embodiments, antibodies or their corresponding immunoglobulin chain(s) are further modified by using, for example, amino acid deletion(s), insertion(s), substitution(s), addition(s), and/or recombination(s) and/or any other modification(s) (e.g. posttranslational and chemical modifications, such as glycosylation and phosphorylation), either alone or in combination. Methods for introducing such modifications in the DNA sequence underlying the amino acid sequence of an immunoglobulin chain are well known to the person skilled in the art.

The term “single-chain” as used in accordance with the present disclosure refers to the covalent linkage of two or more polypeptide sequences, preferably in the form of a co-linear amino acid sequence encoded by a single nucleic acid molecule.

The terms “binding to” and “interacting with” are used interchangeably herein and refer to the interaction of at least two “antigen-interaction-sites” with each other. An “antigen-interaction-site” refers to a motif of a polypeptide (e.g., an antibody or antigen binding fragment thereof) capable of specific interaction with an antigen or a group of antigens. The binding/interaction is also understood to define a “specific interaction” or “specific binding.”

The terms “specific binding” or “specific interaction” refer to an antigen-interaction-site that is capable of specifically interacting with and/or binding to at least two amino acids of a target molecule as defined herein. The term relates to the ability of the antigen-interaction-site to discriminate between the specific regions (e.g. epitopes) of the target molecules defined herein such that it does not, or essentially does not, cross-react with polypeptides of similar structures. In some embodiments, the epitopes are linear. In some embodiments, the epitopes are conformational epitopes, a structural epitope, or a discontinuous epitope consisting of two regions of the human target molecules or parts thereof. In context of this disclosure, a conformational epitope is defined by two or more discrete amino acid sequences separated in the primary sequence which come together on the surface of the folded protein. Specificity and/or cross-reactivity of a panel of antigen bindings construct under investigation can be tested, for example, by assessing binding of the panel of the constructs to the polypeptide of interest as well as to a number of more or less (structurally and/or functionally) closely related polypeptides under conventional conditions (See e.g., Harlow and Lane, Antibodies: A Laboratory Manual, Cold Spring Harbor Laboratory Press, 1988 and Using Antibodies: A Laboratory Manual, Cold Spring Harbor Laboratory Press, 1999). Only those constructs that bind to the polypeptide/protein of interest and do not, or essentially do not, bind to any of the other polypeptides are considered specific for the polypeptide/protein of interest. Examples of specific interactions of an antigen-interaction-site with a specific antigen include the interaction of ligands which induce a signal upon binding to its specific receptor, the specificity of a ligand for its receptor, such as cytokines that bind to specific cytokine receptors, and the binding of an antigen binding site of an antibody to an antigenic epitope, among others.

In some instances, the specific interaction of the antigen-interaction-site with a specific antigen results in the initiation of a signal, e.g. due to the induction of a change of the conformation of the antigen, oligomerization of the antigen, etc. In some embodiments, specific binding encompasses a “key-lock-principle.” Therefore in some embodiments, specific motifs in the amino acid sequence of the antigen-interaction-site interact with specific motifs in the antigen and bind to each other as a result of their primary, secondary or tertiary structure, or as the result of secondary modifications of said structure. In some embodiments, the specific interaction of the antigen-interaction-site with its specific antigen results in a simple binding of the site to the antigen.

### Oncolytic Viruses

Oncolytic viruses are able to infect, replicate in, and lyse tumor cells, and are further capable of spreading to other tumor cells in successive rounds of replication. While past oncolytic virus therapy has shown promise in preclinical models and clinical studies, anti-tumor efficacy of these oncolytic virus, such as vaccinia, has been suboptimal. For example, these viruses demonstrated limited viral spread throughout the tumor and/or limited activation of anti-tumor T cell responses within the tumor. Therefore, the present disclosure provides oncolytic viruses that 1) facilitate tumor infiltration and activation of effector cells (e.g., T cells), and/or 2) effectively lyse tumor cells that are not infected the virus (also known as by-stander killing).

In some embodiments, the oncolytic viruses described herein have one or more of the following advantageous properties:
(a) the viruses are oncolytic and have a particularly high oncolytic activity compared to other previously described oncolytic viruses;
(b) the viruses replicate preferentially in tumor cells and have a particularly high replication capability compared to other oncolytic viruses;
(c) the viruses infect actively dividing cells as well as resting cells;
(d) the viruses induce a strong innate, humoral, and cellular immune response;
(e) the viruses replicate purely cytoplasmatically, i.e., they cannot integrate into the host cell genome or recombine into replication-competent viruses;
(f) one or more of the native viral glycoproteins are interchangeable with a heterologous envelope protein.

### Pseudotyped Oncolytic Viruses

In some embodiments, the present invention provides pseudotyped oncolytic viruses. In general, viruses have natural host cell populations that they infect most efficiently. For example, retroviruses have limited natural host cell ranges, while adenoviruses and adeno-associated viruses are able to efficiently infect a relatively broader range of host cells, although some cell types are refractory to infection by these viruses. The proteins on the surface of a virus (e.g., envelope proteins or capsid proteins) meditate attachment to and entry into a susceptible host cell and thereby determine the tropism of the virus, i.e., the ability of a particular virus to infect a particular cell or tissue type. In some embodiments, the oncolytic viruses described herein comprise a single type of protein on the surface of the virus. For example, retroviruses and adeno-associated viruses have a single protein coating their membrane. In some embodiments, the oncolytic viruses described herein comprise more than one type of protein on the surface of the virus. For example, adenoviruses are coated with both an envelope protein and fibers that extend away from the surface of the virus.

The proteins on the surface of the virus can bind to cell-surface molecules such as heparin sulfate, thereby localizing the virus to the surface of the potential host cell. The proteins on the surface of the virus can also mediate interactions between the virus and specific protein receptors expressed on a host cell that induce structural changes in the viral protein in order to mediate viral entry. Alternatively, interactions between the proteins on the surface of the virus and cell receptors can facilitate viral internalization into endosomes, wherein acidification of the endosomal lumen induces refolding of the viral coat. In either case, viral entry into potential host cells requires a favorable interaction between at least one molecule on the surface of the virus and at least one molecule on the surface of the cell.

In some embodiments, the oncolytic viruses described herein comprise a viral coat (e.g., a viral envelop or viral capsid), wherein the proteins present on the surface of the viral coat (e.g., viral envelop proteins or viral capsid proteins) modulate recognition of a potential target cell for viral entry. In some instances, this process of determining a potential target cell for entry by a virus is referred to as host tropism. In some embodiments, the host tropism is cellular tropism, wherein viral recognition of a receptor occurs at a cellular level, or tissue tropism, wherein viral recognition of cellular receptors occurs at a tissue level. In some instances, the viral coat of a virus recognizes receptors present on a single type of cell. In other instances, the viral coat of a virus recognizes receptors present on multiple cell types (e.g., 2, 3, 4, 5, 6 or more different cell types). In some instances, the viral coat of a virus recognizes cellular receptors present on a single type of tissue. In other instances, the viral coat of a virus recognizes cellular receptors present on multiple tissue types (e.g., 2, 3, 4, 5, 6 or more different tissue types).

In some embodiments, the oncolytic viruses described herein are pseudotyped in order to limit or control the viral tropism (i.e., to reduce the number of different cell or tissue types that the oncolytic virus is capable of infecting). In some embodiments, the pseudotyped oncolytic viruses described herein are pseudotyped in order to expand the viral tropism (i.e., to increase the number of different cell or tissue types that the pseudotyped oncolytic virus is capable of infecting).

In some embodiments, a pseudotyped oncolytic virus comprises a viral coat comprising one or more amino acid mutations in one or more endogenous viral coat proteins (e.g., one or more amino acid insertions, deletions, and/or substitutions). In some embodiments, a pseudotyped virus comprises a viral coat comprising one or more heterologous viral proteins, or wherein one or more of the endogenous viral coat proteins have been replaced or modified. In some embodiments, a pseudotyped oncolytic virus comprises a viral coat that has been modified to incorporate coat proteins from a heterologous virus. In some embodiments, a pseudotyped oncolytic viruses comprises a viral coat wherein the viral coat of a first virus is exchanged with a viral coat of second, wherein the viral coat of the second virus is allows the pseudotyped oncolytic virus to infect a particular cell or tissue type.

In some embodiments, a pseudotyped oncolytic virus comprises a viral coat comprising one or more heterologous, non-viral proteins, such as a targeting moiety. In some embodiments, the non-viral targeting moiety may include antibodies, antigen-binding fragments thereof, and/or receptor ligands. In some embodiments, the viral coat of a pseudotyped oncolytic virus comprises one or more chimeric proteins. In some embodiments, the chimeric proteins are comprised of parts of an endogenous viral protein necessary for incorporation into the virion, as well proteins or nucleic acids designed to interact with specific host cell proteins (e.g., targeting moieties).

The non-viral targeting moieties may be any moiety capable of binding to a corresponding receptor or ligand on the surface of a selected cell type, thereby directing the interaction between the virus particle and the selected cell type. In some instances, any cell surface biological material known in the art (e.g., a protein, lipid, or carbohydrate) or yet to be identified that is differentially expressed or otherwise present on a particular cell or tissue type (e.g., a tumor or tumor cell, or tumor associated stroma or stromal cell) may be used as a potential target for the oncolytic viruses the present invention. In particular embodiments, the cell surface material is a protein. In some embodiments, the targeting moiety binds cell surface antigens indicative of a disease, such as a cancer (e.g., breast, lung, ovarian, prostate, colon, lymphoma, leukemia, melanoma, and others); an autoimmune disease (e.g. myasthenia gravis, multiple sclerosis, systemic lupus erythymatosis, rheumatoid arthritis, diabetes mellitus, and others); an infectious disease, including infection by HIV, HCV, HBV, CMV, and HPV; and a genetic disease including sickle cell anemia, cystic fibrosis, Tay-Sachs, J3-thalassemia, neurofibromatosis, polycystic kidney disease, hemophilia, etc. In certain embodiments, the targeting moiety targets a cell surface antigen specific to a particular cell or tissue type, e.g., cell-surface antigens present in neural, lung, kidney, muscle, vascular, thyroid, ocular, breast, ovarian, testis, or prostate tissue.

Exemplary antigens and cell surface molecules for targeting include, e.g. P-glycoprotein, Her2/Neu, erythropoietin (EPO), epidermal growth factor receptor (EGFR), vascular endothelial growth factor receptor (VEGF-R), cadherin, carcinoembryonic antigen (CEA), CD4, CD8, CD19, CD20, CD33, CD34, CD45, CD117 (c-kit), CD133, HLA-A. HLA-B, HLA-C, chemokine receptor 5 (CCR5), ABCG2 transporter, ovarian cancer antigen CA125, immunoglobulins, integrins, prostate specific antigen (PSA), prostate stem cell antigen (PSCA), dendritic cell-specific intercellular adhesion molecule 3-grabbing nonintegrin (DC-SIGN), thyroglobulin, granulocyte-macrophage colony stimulating factor (GM-CSF), myogenic differentiation promoting factor-I (MyoD-1), Leu-7 (CD57), LeuM-1, cell proliferation-associated human nuclear antigen defined by the monoclonal antibody Ki-67 (Ki-67), viral envelope proteins, IV gp120, transferrin receptor, etc.

In some embodiments, the viral coat comprises a capsid. In some instances, the capsid is assembled from oligomeric protein subunits termed protomers. In some embodiments, the capsid is assembled from one type of protomer or protein, or is assembled from two, three, four, or more types of protomers or proteins. In some embodiments, the viral coat comprises a viral envelope. In some instances, the viral envelope comprises a phospholipid bilayer and proteins such as proteins obtained from a host membrane. In some embodiments, the viral envelope further comprises glycoproteins for recognition and attachment to a receptor expressed by a host cell. In some embodiments, the pseudotyped oncolytic viruses described herein comprise one or more heterologous glycoproteins (GPs) or one or more endogenous GPs that have been modified (i.e., by amino acid mutation). In some embodiments, the pseudotyped oncolytic viruses described herein comprise one or more GPs derived from VSV. In some embodiments, the pseudotyped oncolytic viruses described herein comprise one or more GPs derived from lyssavirus, lymphocytic choriomeningitis virus, alphavirus GPs (e.g., RRV and SFV GPs), sindbis virus, filovirus, and baculovirus (e.g., GP64).

In some embodiments, the pseudotyped oncolytic viruses described herein demonstrate i) altered tropism relative to non-pseudotyped virus; and/or ii) reduction or elimination of a non-beneficial effect, such as infectivity of healthy cells or tissue; and/or iii) increase of a beneficial effect, such as infectivity of cancerous cells or tissue. For example, in some embodiments a pseudotyped virus demonstrates reduced toxicity or reduced infection of non-tumor cells or non-tumor tissue as compared to a non-pseudotyped virus. In some embodiments, a pseudotyped virus is capable of infecting a cell or tissue type that the corresponding non-pseudotyped virus is not capable of infecting. In some embodiments, a pseudotyped virus is capable of preferentially infecting a cell or tissue type compared to a non-pseudotyped virus.

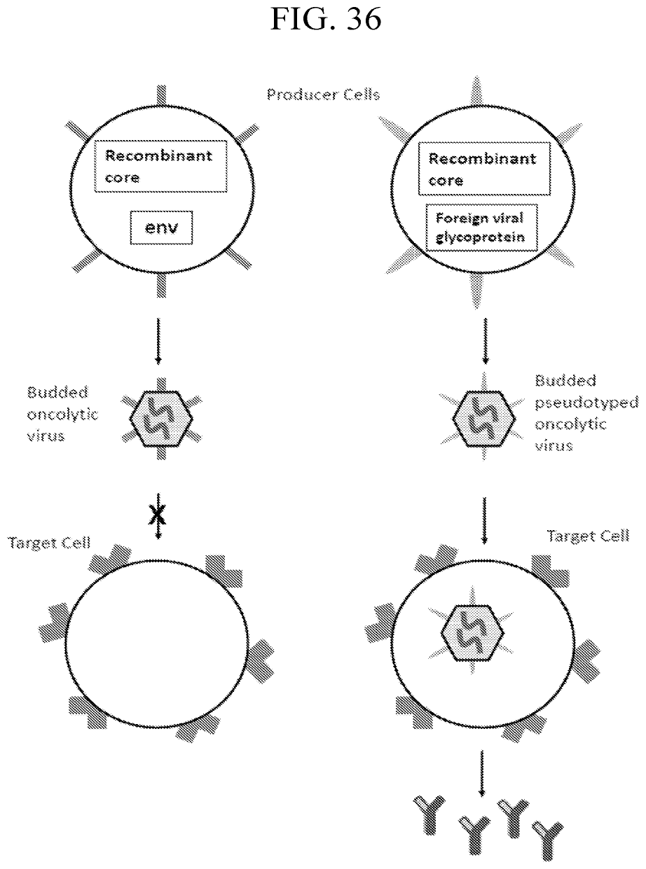

In some embodiments, the pseudotyped oncolytic viruses provided herein are capable of selectively entering, replicating in, and/or lysing tumor cells. Such an embodiment is illustrated in FIG. 36, wherein the pseudotyped oncolytic virus gains entry to the target cell due to the incorporation of viral glycoproteins derived from a different (i.e., heterologous) virus that allow for entry of the pseudotyped oncolytic virus into the target cell. In contrast, the non-pseudotyped oncolytic virus is unable to gain entry into the target cell due to the non-permissive nature of the envelope proteins. In some instances, the ability of a pseudotyped oncolytic virus to selectively enter, replicate in, and/or lyse a tumor cells is due to a reduced or otherwise ineffective cellular interferon (IFN) response. In some embodiments, the pseudotyped oncolytic viruses produce an engager molecule and/or a therapeutic molecule, such as an immune modulating polypeptide, that interferes or impairs the cellular IFN response, thereby enhancing the replication of the pseudotyped or engineered virus.

The pseudotyped oncolytic viruses described herein may be derived from a variety of viruses, non-limiting examples of which include vaccinia virus, adenovirus, herpes simplex virus 1 (HSV1), myxoma virus, reovirus, poliovirus, vesicular stomatitis virus (VSV), measles virus (MV), lassa virus (LASV) and Newcastle disease virus (NDV). In some embodiments, the pseudotyped oncolytic viruses described herein can infect substantially any cell type. An exemplary lentivirus for use in oncolytic therapy is Simian immunodeficiency virus coated with the envelope proteins, G-protein (GP), from VSV. In some instances, this virus is referred to as VSV G-pseudotyped lentivirus, and is known to infect an almost universal set of cells.

In some embodiments, the present invention provides recombinant vesicular stomatitis viruses (VSV) and VSV vectors. The VSV genome includes five genes, 1, m, n, p and g, which encode the proteins L, M, N, P and G and are essential for the reproduction of the virus. N is a nucleoprotein which packages the VSV genomic RNA. The VSV genome is replicated as RNA-protein complex and L and P together form a polymerase complex which replicates the VSV genome and transcribes the VSV mRNA. M is a matrix protein which provides structural support between the lipid envelope and nucleocapsid and is important for particle sprouting at the cell membrane. G is the envelope protein which is incorporated in the viral envelope and is essential for the infectivity and tropism of the virus.

In some embodiments, the pseudotyped oncolytic viruses of the present invention are VSV viruses pseudotyped against healthy brain cells, i.e., neurons and exhibit considerably reduced toxicity. Since neurotropism is a dose-limiting factor in all applications of oncolytic VSV, the use of the vector according to some embodiments of the present invention is that they are used for all tumors types of solid tumors.

In some embodiments, the pseudotyped VSV vectors have one or more key attributes including: (i) the VSV is not cell-toxic; (ii) the vectors are concentrated by ultracentrifugation without loss of infectivity; and (iii) the vectors show a tropism for tumor cells, whereas neurons and other non-tumor cells are infected inefficiently. To increase the safety during the use of replicable viruses in therapeutic uses, some embodiments of the present invention provide a vector system which ensures that replication, oncolysis and the production of VSV viruses takes place only in cells which are infected by at least two replication-deficient, mutually complementing vectors.

In some embodiments, the genetic material (e.g., the viral coat protein or the core genetic material) for generating a pseudotyped oncolytic virus is obtained from a DNA virus, an RNA virus, or from both virus types. In some embodiments, a DNA virus is a single-stranded (ss) DNA virus, a double-stranded (ds) DNA virus, or a DNA virus that contains both ss and ds DNA regions. In some embodiments, an RNA virus is a single-stranded (ss) RNA virus or a double-stranded (ds) RNA virus. In some embodiments, an ssRNA virus is further classified into a positive-sense RNA virus or a negative-sense RNA virus.

In some instances, the genetic material for generating a pseudotyped oncolytic virus is obtained from a dsDNA virus of any one of the following families: Myoviridae, Podoviridae, Siphoviridae, Alloherpesviridae, Herpesviridae, Malacoherpesviridae, Tipothrixviridae, Rudiviridae, Adenoviridae, Ampullaviridae, Ascoviridae, AsfAviridae, Baculoviridae, Bicaudaviridae, Clavaviridae, Corticoviridae, Fuselloviridae, Globuloviridae, Guttaviridae, Hytrosaviridae, Iridoviridae, Marseilleviridae, Mimiviridae, Nimaviridae, Pandoraviridae, Papillomaviridae, Phycodnaviridae, Plasmaviridae, Polydnaviruses, Polyomaviridae, Poxviridae, Sphaerolipoviridae, or Tectiviridae.

In some cases, the genetic material for generating a pseudotyped oncolytic virus is obtained from a ssDNA virus of any one of the following families: Anelloviridae, Bacillariodnaviridae, Bidnaviridae, Circoviridae, Geminiviridae, Inoviridae, Microviridae, Nanoviridae, Parvoviridae, or Spiraviridae.

In some embodiments, the genetic material for generating a pseudotyped oncolytic virus is obtained from a DNA virus that contains both ssDNA and dsDNA regions. In some cases, the DNA virus is from the group pleolipoviruses. In some cases, the pleolipoviruses include Haloarcula hispanica pleomorphic virus 1, Halogeometricum pleomorphic virus 1, Halorubrum pleomorphic virus 1, Halorubrum pleomorphic virus 2, Halorubrum pleomorphic virus 3, or Halorubrum pleomorphic virus 6.

In some cases, the genetic material for generating a pseudotyped oncolytic virus is obtained from a dsRNA virus of any one of the following families: Birnaviridae, Chrysoviridae, Cystoviridae, Endornaviridae, Hypoviridae, Megavirnaviridae, Partitiviridae, Picobirnaviridae, Reoviridae, Rotavirus or Totiviridae.

In some instances, the genetic material for generating a pseudotyped oncolytic virus is obtained from a positive-sense ssRNA virus of any one of the following families: Alphaflexiviridae, Alphateiraviridae, Alvernaviridae, Arteriviridae, Astroviridae, Barnaviridae, Betaflexiviridae, Bromoviridae, Caliciviridae, Carmotetraviridae, Closteroviridae, Coronaviridae, Dicistroviridae, Flaviviridae, Gannaflexiviridae, Iflaviridae, Leviviridae, Luteoviridae, Marnaviridae, Mesoniviridae, Narnaviridae, Nodaviridae, Permutotetraviridae, Picornaviridae, Potyviridae, Roniviridae, Secoviridae, Togaviridae, Tombusviridae, Tymoviridae, or Virgaviridae.

In some cases, the genetic material for generating a pseudotyped oncolytic virus is obtained from a negative-sense ssRNA virus of any one of the following families: Bornaviridae, Filoviridae, Paramyxoviridae, Rhabdoviridae, Nyamiviridae, Arenaviridae, Bunyaviridae, Ophioviridae, or Orthonyxoviridae.

In some instances, the genetic material for generating a pseudotyped oncolytic virus is obtained from oncolytic DNA viruses that comprise capsid symmetry that is isocahedral or complex. In some cases, isosahedral oncolytic DNA viruses are naked or comprise an envelope. Exemplary families of oncolytic DNA viruses include the Adenoviridae (for example, Adenovirus, having a genome size of 36-38 kb), Herpesviridae (for example, HSV1, having a genome size of 120-200 kb), and Poxviridae (for example, Vaccinia virus and myxoma virus, having a genome size of 130-280 kb).

In some cases, the genetic material for generating a pseudotyped oncolytic virus is obtained from oncolytic RNA viruses include those having icosahedral or helical capsid symmetry. In some cases, icosahedral oncolytic viruses are naked without envelope and include Reoviridae (for example, Reovirus, having a genome of 22-27 kb) and Picornaviridae (for example, Poliovirus, having a genome size of 7.2-8.4 kb). In other cases, helical oncolytic RNA viruses are enveloped and include Rhabdoviridae (for example, VSV, having genome size of 13-16 kb) and Paramyxoviridae (for example MV and NDV, having genome sizes of 16-20 kb).

In some instances, the genetic material for generating a pseudotyped oncolytic virus is obtained from a virus such as Abelson leukemia virus, Abelson murine leukemia virus, Abelson's virus, Acute laryngotracheobronchitis virus, Adelaide River virus, Adeno associated virus group, Adenovirus, African horse sickness virus, African swine fever virus, AIDS virus, Aleutian mink disease parvovirus, Alpharetrovirus, Alphavirus, ALV related virus, Amapari virus, Aphthovirus, Aquareovirus, Arbovirus, Arbovirus C, arbovirus group A, arbovirus group B, Arenavirus group, Argentine hemorrhagic fever virus, Argentine hemorrhagic fever virus, Arterivirus, Astrovirus, Ateline herpesvirus group, Aujezky's disease virus, Aura virus, Ausduk disease virus, Australian bat lyssavirus, Aviadenovirus, avian erythroblastosis virus, avian infectious bronchitis virus, avian leukemia virus, avian 23apulose virus, avian lymphomatosis virus, avian myeloblastosis virus, avian paramyxovirus, avian pneumoencephalitis virus, avian reticuloendotheliosis virus, avian sarcoma virus, avian type C retrovirus group, Avihepadnavirus, Avipoxvirus, B virus, B19 virus, Babanki virus, baboon herpesvirus, baculovirus, Barmah Forest virus, Bebaru virus, Berrimah virus, Betaretrovirus, Birnavirus, Bittner virus, BK virus, Black Creek Canal virus, bluetongue virus, Bolivian hemorrhagic fever virus, Boma disease virus, border disease of sheep virus, borna virus, bovine alphaherpesvirus 1, bovine alphaherpesvirus 2, bovine coronavirus, bovine ephemeral fever virus, bovine immunodeficiency virus, bovine leukemia virus, bovine 23apulose virus, bovine mammillitis virus, bovine papillomavirus, bovine 23apulos stomatitis virus, bovine parvovirus, bovine syncytial virus, bovine type C oncovirus, bovine viral diarrhea virus, Buggy Creek virus, bullet shaped virus group, Bunyamwera virus supergroup, Bunyavirus, Burkitt's lymphoma virus, Bwamba Fever, CA virus, Calicivirus, California encephalitis virus, camelpox virus, canarypox virus, canid herpesvirus, canine coronavirus, canine distemper virus, canine herpesvirus, canine minute virus, canine parvovirus, Cano Delgadito virus, caprine arthritis virus, caprine encephalitis virus, Caprine Herpes Virus, Capripox virus, Cardiovirus, caviid herpesvirus 1, Cercopithecid herpesvirus 1, cercopithecine herpesvirus 1, Cercopithecine herpesvirus 2, Chandipura virus, Changuinola virus, channel catfish virus, Charleville virus, chickenpox virus, Chikungunya virus, chimpanzee herpesvirus, chub reovirus, chum salmon virus, Cocal virus, Coho salmon reovirus, coital exanthema virus, Colorado tick fever virus, Coltivirus, Columbia SK virus, common cold virus, contagious eethyma virus, contagious pustular dermatitis virus, Coronavirus, Corriparta virus, coryza virus, cowpox virus, coxsackie virus, CPV (cytoplasmic polyhedrosis virus), cricket paralysis virus, Crimean-Congo hemorrhagic fever virus, croup associated virus, Cryptovirus, Cypovirus, Cytomegalovirus, cytomegalovirus group, cytoplasmic polyhedrosis virus, deer papillomavirus, deltaretrovirus, dengue virus, Densovirus, Dependovirus, Dhori virus, diploma virus, Drosophila C virus, duck hepatitis B virus, duck hepatitis virus 1, duck hepatitis virus 2, duovirus, Duvenhage virus, Deformed wing virus DWV, eastern equine encephalitis virus, eastern equine encephalomyelitis virus, EB virus, Ebola virus, Ebola-like virus, echo virus, echovirus, echovirus 10, echovirus 28, echovirus 9, ectromelia virus, EEE virus, EIA virus, ETA virus, encephalitis virus, encephalomyocarditis group virus, encephalomyocarditis virus, Enterovirus, enzyme elevating virus, enzyme elevating virus (LDH), epidemic hemorrhagic fever virus, epizootic hernorrhagic disease virus, Epstein-Barr virus, equid alphaherpesvirus 1, equid alphaherpesvirus 4, equid herpesvirus 2, equine abortion virus, equine arteritis virus, equine encephalosis virus, equine infectious anemia virus, equine morbillivirus, equine rhinopneumnonitis virus, equine rhinovirus, Eubenangu virus, European elk papillomavirus, European swine fever virus, Everglades virus, Eyach virus, felid herpesvirus 1, feline calicivirus, feline fibrosarcoma virus, feline herpesvirus, feline immunodeficiency virus, feline infectious peritonitis virus, feline leukemia/sarcoma virus, feline leukemia virus, feline panleukopenia virus, feline parvovirus, feline sarcoma virus, feline syncytial virus, Filovirus, Flanders virus, Flavivirus, foot and mouth disease virus, Fort Morgan virus, Four Corners hantavirus, fowl adenovirus 1, fowlpox virus, Friend virus, Gammaretrovirus, GB hepatitis virus, GB virus, German measles virus, Getah virus, gibbon ape leukemia virus, glandular fever virus, goatpox virus, golden 24apulo virus, Gonometa virus, goose parvovirus, granulosis virus, Gross' virus, ground squirrel hepatitis B virus, group A arbovirus, Guanarito virus, guinea pig cytomegalovirus, guinea pig type C virus, Hantaan virus, Hantavirus, hard clam reovirus, hare fibroma virus, HCMV (human cytomegalovirus), hemadsorption virus 2, henagglutinating virus of Japan, hemorrhagic fever virus, 24apulo virus, Henipaviruses, Hepadnavirus, hepatitis A virus, hepatitis B virus group, hepatitis C virus, hepatitis D virus, hepatitis delta virus, hepatitis E virus, hepatitis F virus, hepatitis G virus, hepatitis nonA nonB virus, hepatitis virus, hepatitis virus (nonhuman), hepatoencephalomyelitis reovirus 3, Hepatovirus, heron hepatitis B virus, herpes B virus, herpes simplex virus, herpes simplex virus I, herpes simplex virus 2, herpesvirus, herpesvirus 7, Herpesvirus ateles, Herpesvirus hominis, Herpesvirus infection, Herpesvirus saimiri, Herpesvirus suis, Herpesvirus varicellae, Highlands J virus, Hirame rhabdovirus, hog cholera virus, human adenovirus 2, human alphaherpesvirus 1, human alphaherpesvirus 2, human alphaherpesvirus 3, human B lymphotropic virus, human betaherpesvirus 5, human coronavirus, human cytomegalovirus group, human foamy virus, human gammaherpesvirus 4, human gammaherpesvirus 6, human hepatitis A virus, human herpesvirus 1 group, human herpesvirus 2 group, human herpesvirus 3 group, human herpesvirus 4 group, human herpesvirus 6, human herpesvirus 8, human immunodeficiency virus, human immuniodeficiency virus 1, human immunodeficiency virus 2, human papillomavirus, human T cell leukemia virus, human T cell leukemia virus I, human T cell leukemia virus II, human T cell leukemia virus III, human T cell lymphomna virus L, human T cell lymphoma virus II, human T cell lymphotropic virus type 1, human T cell lymphotropic virus type 2, human T lymphotropic virus 1, human T lymnphotropic virus II, human T lymnphotropic virus III, Ichnovirus, infantile gastroenteritis virus, infectious bovine rhinotracheitis virus, infectious haematopoietic necrosis virus, infectious pancreatic necrosis virus, influenza virus A, influenza virus B, influenza virus C, influenza virus D, influenza virus pr8, insect iridescent virus, insect virus, iridovirus, Japanese B virus, Japanese encephalitis virus, JC virus, Junin virus, Kaposi's sarcoma-associated herpesvirus, Kemerovo virus, Kilham's rat virus, Klamath virus, Kolongo virus, Korean hemorrhagic fever virus, kamba virus, Kysanur forest disease virus, Kyzylagach virus, La Crosse virus, lactic dehydrogenase elevating virus, lactic dehydrogenase virus, Lagos bat virus, Langur virus, lapine parvovirus, Lassa fever virus, Lassa virus, latent rat virus, LCM virus, Leaky virus, Lentivirus, Leporipoxvirus, leukemia virus, leukovirus, lumpy skin disease virus, lymphadenopathy associated virus, Lymphocryptovirus, lymphocytic choriomeningitis virus, lymphoproliferative virus group, Machupo virus, mad itch virus, mammalian type B oncovirus group, mammalian type B retroviruses, mammalian type C retrovirus group, mammalian type D retroviruses, mammary tumor virus, Mapuera virus, Marburg virus, Marburg-like virus, Mason Pfizer monkey virus, Mastadenovirus, Mayaro virus, ME virus, measles virus, Menangle virus, Mengo virus, Mengovirus, Middelburg virus, milkers nodule virus, mink enteritis virus, minute virus of mice, MLV related virus, MN virus, Mokola virus, Molluscipoxvirus, Molluscum contagiosum virus, monkey B virus, monkeypox virus, Mononegavirales, Morbillivirus, Mount Elgon bat virus, mouse cytomegalovirus, mouse encephalomyelitis virus, mouse hepatitis virus, mouse K virus, mouse leukemia virus, mouse mammary tumor virus, mouse minute virus, mouse pneumonia virus, mouse poliomyelitis virus, mouse polyomavirus, mouse sarcoma virus, iousepox virus, Mozambique virus, Mucanbo virus, mucosal disease virus, mumps virus, murid betaherpesvirus 1, murid cytomegalovirus 2, murine cytomegalovirus group, murine encephalomyelitis virus, murine hepatitis virus, murine leukemia virus, murine nodule inducing virus, murine polyornavirus, murine sarcoma virus, Muromegalovirus, iurray Valley encephalitis virus, rnyxorna virus, Myxovirus, Myxovirus multiforme, Myxovirus parotitidis, Nairobi sheep disease virus, Nairovirus, Nanirnavirus, Nariva virus, Ndumo virus, Neethling virus, Nelson Bay virus, neurotropic virus, New World Arenavirus, newborn pneumonitis virus, Newcastle disease virus, Nipah virus, noncytopathogenic virus, Norwalk virus, nuclear polyhedrosis virus (NPV), nipple neck virus, O‘nyong’nyong virus, Ockelbo virus, oncogenic virus, oncogenic viruslike particle, oncornavirus, Orbivirus, Orf virus, Oropouche virus, Orthohepadnavirus, Orthomyxovirus, Orthopoxvirus, Orthoreovirus, Orungo, ovine papillomavirus, ovine catarrhal fever virus, owl monkey herpesvirus, Palyam virus, Papillomavirus, Papillornavirus sylvilagi, Papovavirus, parainfluenza virus, parainfluenza virus type 1, parainfluenza virus type 2, parainfluenza virus type 3, parainfluenza virus type 4, Paramyxovirus, Parapoxvirus, paravaccinia virus, Parvovirus, Parvovirus B19, parvovirus group, Pestivirus, Phlebovirus, phocine distemper virus, Picodnavirus, Picornavirus, pig cytomegalovirus-pigeonpox virus, Piry virus, Pixuna virus, pneumonia virus of mice, Pneumovirus, poliomyelitis virus, poliovirus, Polydnavirus, polyhedral virus, polyoma virus, Polyomavirus, Polyomavirus bovis, Polyomavirus cercopitheci, Polyomavirus hominis 2, Polyomavirus naccacae 1, Polyomavirus muris 1, Polyomavirus muris 2, Polyornavirus papionis 1, Polyomavirus papionis 2, Polyonavirus sylvilagi, Pongine herpesvirus 1, porcine epidemic diarrhea virus, porcine hemagglutinating encephalomyelitis virus, porcine parvovirus, porcine transmissible gastroenteritis virus, porcine type C virus, pox virus, poxvirus, poxvirus variolae, Prospect Hill virus, Provirus, pseudocowpox virus, pseudorabies virus, psittacinepox virus, quailpox virus, rabbit fibroma virus, rabbit kidney vaculolating virus, rabbit papillomavirus, rabies virus, raccoon parvovirus, raccoonpox virus, Ranikhet virus, rat cytoiegalovirus, rat parvovirus, rat virus, Rauscher's virus, recombinant vaccinia virus, recombinant virus, reovirus, reovirus 1, reovirus 2, reovirus 3, reptilian type C virus, respiratory infection virus, respiratory syncytial virus, respiratory virus, reticuloendotheliosis virus, Rhabdovirus, Rhabdovirus carpia, Rhadinovirus, Rhinovirus, Rhizidiovirus, Rift Valley fever virus, Riley's virus, rinderpest virus, RNA tumor virus, Ross River virus, Rotavirus, rougeole virus, Rous sarcoma virus, rubella virus, rubeola virus, Rubivirus, Russian autumn encephalitis virus, SA 11 simian virus, SA2 virus, Sabia virus, Sagiyama virus, Saimirine herpesvirus 1, salivary gland virus, sandfly fever virus group, Sandjimba virus, SAPS virus, SDAV (sialodacryoadenitis virus), sealpox virus, Semliki Forest Virus, Seoul virus, sheeppox virus, Shope fibroma virus, Shope papilloma virus, simian foamy virus, simian hepatitis A virus, simian human immunodeficiency virus, simian immunodeficiency virus, simian parainfluenza virus, simian T cell lymphotrophic virus, simian virus, simian virus 40, Simplexvirus, Sin Nombre virus, Sindbis virus, smallpox virus, South American hemorrhagic fever viruses, sparrowpox virus, Spumavirus, squirrel fibroma virus, squirrel monkey retrovirus, SSV I virus group, STIL (simian T lymphotropic virus) type I, STLV (simian T lymphotropic virus) type 11, STLV (simian T lymphotropic virus) type III, stomatitis 27apulose virus, submaxillary virus, suid alphaherpesvirus 1, suid herpesvirus 2, Suipoxvirus, swamp fever virus, swinepox virus, Swiss mouse leukemia virus, TAC virus, Tacaribe complex virus, Tacaribe virus, Tanapox virus, Taterapox virus, Tench reovirus, Theiler's encephalonyelitis virus, Theiler's virus, Thogoto virus, Thottapalayam virus, Tick borne encephalitis virus, Tioman virus, Togavirus, Torovirus, tumor virus, Tupaia virus, turkey rhinotracheitis virus, turkeypox virus, type C retroviruses, type D oncovirus, type D retrovirus group, ulcerative disease rhabdovirus, tina virus, Uukuniemi virus group, vaccinia virus, vacuolating virus, varicella zoster virus, Varicellovirus, Varicola virus, variola major virus, variola virus, Vasin Gishu disease virus, VEE virus, Venezuelan equine encephalitis virus, Venezuelan equine encephalomyelitis virus, Venezuelan hemorrhagic fever virus, vesicular stomatitis virus, Vesiculovirus, Vilyuisk virus, viper retrovirus, viral haemorrhagic septicemia virus, Visna Maedi virus, Visna virus, volepox virus, VSV (vesicular stomatitis virus), Wallal virus, Warrego virus, wart virus, WEE virus, West Nile virus, western equine encephalitis virus, western equine encephalomyelitis virus, Whataroa virus, Winter Vomiting Virus, woodchuck hepatitis B virus, woolly monkey sarcoma virus, wound tumor virus, WRSV virus, Yaba monkey tumor virus, Yaba virus, Yatapoxvirus, yellow fever virus, and the Yug Bogdanovac virus.

### Methods of Producing Pseudotyped Oncolytic Viruses

In some instances, a pseudotyped oncolytic virus described herein is generated using methods well known in the art. In some instances, the methods involve one or more transfection steps and one or more infection steps. In some instances, a cell line such as a mammalian cell line, an insect cell line, or a plant cell line is infected with a pseudotyped oncolytic virus described herein to produce one or more viruses. Exemplary mammalian cell lines include: 293A cell line, 293FT cell line, 293F cells, 293 H cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, T-REx™-HeLa cell line, 3T6, A549, A9, AtT-20, BALB/3T3, BHK-21, BHL-100, BT, Caco-2, Chang, Clone 9, Clone M-3, COS-1, COS-3, COS-7, CRFK, CV-1, D-17, Daudi, GH1, GH3, H9, HaK, HCT-15, HEp-2, HL-60, HT-1080, HT-29, HUVEC, I-10, IM-9, JEG-2, Jensen, K-562, KB, KG-1, L2, LLC-WRC 256, McCoy, MCF7, VERO, WI-38, WISH, XC, or Y-1. Exemplary insect cell lines include Drosophila S2 cells, Sf9 cells, Sf21 cells, High Five™ cells, or expresSF+® cells. Exemplary plant cell lines include algae cells such as for example Phaeocystis pouchetii.

Any method known to one skilled in the art is used for large scale production of recombinant oncolytic vectors and vector constructs, such as pseudotyped oncolytic vectors. For example, master and working seed stocks can be prepared under GMP conditions in qualified primary CEFs or by other methods. In some instances, cells are plated on large surface area flasks, grown to near confluency, and infected at selected MOI. The produced virus can then be purified. In some cases, cells are harvested and intracellular virus is released by mechanical disruption. In some embodiments, cell debris is removed by large-pore depth filtration and/or host cell DNA is digested with an endonuclease. In some cases, virus particles are subsequently purified and concentrated by tangential-flow filtration, followed by diafiltration. The resulting concentrated virus can formulated by dilution with a buffer containing one or more stabilizers, filled into vials, and lyophilized. Compositions and formulations can be stored for later use. In some embodiments, a lyophilized virus is reconstituted by addition of one or more diluents.

### MicroRNA-Attenuated Oncolytic Viruses

In some embodiments, the present invention provides oncolytic viruses, wherein one or more micro-RNA (miR) target sequences are inserted into a locus of one or more viral genes required for viral replication. The terms “microRNA” and “miRNA” and “miR” are used interchangeably herein and refer to small non-coding endogenous RNAs that regulate gene expression by directing their target messenger RNAs (mRNA) for degradation or translational repression. miRs are differentially expressed in a broad array of disease states, including multiple types of cancer. In some aspects, cancer cells of a given cancer type or tissue demonstrate differential expression of miRs compared to non-cancerous control cells. As used herein, the term “oncomiR” refers to miRs that are associated (either positively or negatively) with carcinogenesis, malignant transformation, and/or metastasis. In some aspects, the expression level of a particular oncomiR is associated with the development or maintenance of a particular cancer. Such miRs are referred to herein as “oncogenic miRs.” In some embodiments, the expression of a particular oncomiR is associated with the prevention and/or delay of carcinogenesis and/or metastasis. Such oncomiRs are referred to herein as “tumor-suppressor miRs” or “tumor-suppressive miRs,” as their expression prevents or suppresses the development of cancer.

One aspect of the invention comprises a recombinant oncolytic virus (or viral vector) comprising a plurality of copies of one or more tumor-suppressive miRNA target sequences inserted into a locus of one or more viral genes required for viral replication. In certain embodiments, a recombinant oncolytic virus may comprise tumor-suppressive miRNA target sequences inserted into a locus of at least one, at least two, at least three, at least four, at least five, at least six, at least seven, at least eight, at least nine, or at least ten viral genes required for viral replication. Tumor-suppressor miRNAs expressed in normal (non-cancerous) cells can bind to such target sequences and suppress expression of the viral gene containing the miRNA target sequence, thereby limiting viral replication in healthy, non-cancerous cells. Such recombinant oncolytic viruses and/or vectors are referred to herein as “miR-attenuated” or “replication-restricted” as they result in reduced or attenuated viral replication in cell that express a tumor-suppressive miR capable of binding to the incorporated tumor-suppressive miR target sequence compared to cells that do not express, or have reduced expression of, the tumor-suppressive miR. By incorporating tumor-suppressive miRNAs into key genes required for viral replication, viral replication can be conditionally suppressed in normal diploid cells expressing the tumor-suppressive miRNAs and can proceed normally in cells that do not express the tumor-suppressive miRNAs. In such embodiments, healthy, non-cancerous cells are protected from the normal cells from lytic effects of infection by the recombinant viral vector. Exemplary miR-attenuated oncolytic viruses are described in PCT Publication No. WO 2017/132552, which is incorporated by reference in its entiriety. In some embodiments, one or more target sequences for miR-1, miR-9, miR-10b, miR-17, miR-21, miR-30a, miR-96, miR-106a, miR-122, miR-124, miR-125b, miR-126, miR-127, miR-128, miR-133a, miR-137, miR-143, miR-145, miR-146a, miR-146b, miR-148a, miR-150, miR-155, miR-182, miR-183, miR-204, miR-206, miR-208a, miR-208b, miR-218, miR-219a, miR-221, miR-222, miR-378g, miR-378i, miR-499, miR-885, miR-1247-5p, or miR-4284.

### Engager Molecules

In some embodiments, the oncolytic viruses described herein comprise a polynucleotide sequence that encodes an engager molecule, e.g., an engager polypeptide. The engager molecules of the present invention comprise at least two domains each capable of binding to a different cell surface molecule.

In some embodiments, the engager molecule is a bipartite polypeptide (i.e., comprises 2 domains each binding to a different cell surface protein). In some embodiments, the engager molecule is a tripartite or multipartite polypeptide (i.e., comprises 3 or more domains each binding to a different cell surface protein). In such embodiments, the engager molecule comprises one or more effector domains and one or more activation or targeting domains. In some embodiments, the engager molecules further comprise one or more dimerization or trimerization domains. In some embodiments, the engager molecule is a tripartite polypeptide comprising (i) an effector domain, (ii) an activation or targeting domain, and (iii) an additional domain e.g., an additional effector domain, an additional activation domain, an additional targeting domain, a therapeutic molecule domain (such as a cytokine, a co-stimulatory domain, an immune checkpoint inhibitor, or an anti-angiogenic factor, described infra), or a combination thereof. In some embodiments, the engager is a first polypeptide provided within the oncolytic virus with a second polypeptide having one or more other domains, e.g., one or more of a cytokine, a co-stimulatory domain, a domain that inhibits negative regulatory molecules of T-cell activation, or a combination thereof. In some embodiments, the first polypeptide and the second polypeptide are encoded in the same vector (e.g., viral vector). In some embodiments, the first polypeptide and the second polypeptide are encoded in different vectors (e.g., viral vectors). In specific embodiments, the cytokine is IL-15, IL-2, and/or IL-7. In other specific embodiments, the co-stimulatory domain is CD27, CD80, CD83, CD86, CD134, or CD137. In other specific embodiments, the domain that inhibits negative regulatory molecules of T-cell activation is PD-1, PDL1, CTLA4, or B7-H4.

In some embodiments, the engager molecules of the present invention are in one of the following formats: DVD-Ig, Fab-scFv-Fc, Morrison heterodimer (2+1), Fc-DART, Azymertric™ bispecific Abs, Binanbody, multi-DARPin, an antibody, an antibody domain, a human immunoglobulin heavy chain variable domain, a dual-variable-domain antibody (DVD-Ig), a Tandab, a diabody, a flexibody, a dock-and-lock antibody, a single chain variable fragment (scFv), a BiTE, a DuoBody, an Fc-engineered IgG, an Fcab, a Mab2, or DART polypeptide.

In some embodiments, the engager molecule comprises an antibody or or an antigen-binding fragment thereof e.g., a single chain variable fragment (scFv), an Fab, an Fv, a minibody, or a diabody. In some embodiments, the engager molecule is a is a trifunctional antibody, an Fab2, a bi-specific scFv such as a bi-specific T-cell engager (BiTE), a bivalent minibody, a bispecific diabody, a DuoBody, or an Mab2. In certain embodiments, the engager molecule is a bipartite T cell engager (BiTE) or a tripartite T cell engager (TiTE). In some embodiments, these antibody-derived fragments or derivatives may be modified by chemical, biochemical, or molecular biological methods. In some instances, the antibodies or antigen-binding fragments thereof used in the construction of the engager molecules described herein are humanized or deimmunized constructs. Methods for the humanization and/or deimmunization of polypeptides and, in particular, antibody constructs are known to the person skilled in the art. Corresponding methods are known in the art and described, inter alia, in laboratory manuals (see Sambrook et al., Molecular Cloning: A Laboratory Manual; Cold Spring Harbor Laboratory Press, 2nd edition 1989 and 3rd edition 2001; Gerhardt et al., Methods for General and Molecular Bacteriology, ASM Press, 1994; Lefkovits, Immunology Methods Manual: The Comprehensive Sourcebook of Techniques; Academic Press, 1997; Golemis, Protein-Protein Interactions: A Molecular Cloning Manual; Cold Spring Harbor Laboratory Press, 2002). In some embodiments, the domains of the engager molecule comprise a ligand, a peptide, a peptide that recognize and interacts with a cell surface receptor.

In some embodiments, two or more of the domains of an engager molecule are linked by a linker. In some instances, the linker is of any suitable length, and such a parameter is routinely optimized in the art. For example, linkers are of a length and sequence sufficient to ensure that each of the domains can, independently from one another, retain their differential binding specificities. The term “peptide linker” refers to an amino acid sequence by which the amino acid sequences of a first domain and a second domain of a defined construct are linked together. In some instance, one technical feature of such peptide linker is that said peptide linker does not comprise any polymerization activity and/or does not promote formation of secondary structures. Such peptide linkers are known in the art and described, for example, in Dall'Acqua et al. (Biochem. (1998) 37, 9266-9273); Cheadle et al. (Mol Immunol (1992) 29, 21-30); and Raag and Whitlow (FASEB (1995) 9(1), 73-80). In some embodiments, the peptide linkers of the present invention comprise less than 5 amino acids, less than 4 amino acids, less than 3 amino acids, less than 2 amino acids, or 1 amino acid. In some embodiments, the peptide linker is a single amino acid linker. In such embodiments, the single amino acid is typically a glycine (Gly). In some embodiments, peptide linkers that also do not promote any secondary structures are preferred. Methods for preparing fused, operatively-linked constructs and their expression in mammalian or bacterial cells are well-known in the art (See e.g., International PCT Publication No. WO 99/54440; Ausubel, Current Protocols in Molecular Biology, Green Publishing Associates and Wiley Interscience, N.Y. 1989 and 1994; and Sambrook et al., Molecular Cloning: A Laboratory Manual, Cold Spring Harbor Laboratory Press, Cold Spring Harbor, New York, 2001).

In some embodiments, the engager molecule is a single chain bi-specific antibody construct comprising two antibody-derived binding domains. In such embodiments, one of the binding domains comprises variable regions (or parts thereof) of the heavy chain (V_{H}) and light chain (V_{L}) of an antibody capable of specifically binding to/interacting with a first cell surface molecule and the second binding domain comprises variable regions (or parts thereof) of the heavy chain (V_{H}) and light chain (V_{L}) of an antibody capable of specifically binding to/interacting with a second cell surface molecule. In particular embodiments, each of the two antibody-derived binding domains comprise at least one complementary determining region (CDR), particularly a CDR3. In some embodiments, the single chain bi-specific antibody construct is a diabody. In some embodiments, the single chain bispecific antibody construct is a single chain bispecific scFv, wherein the two scFvs are connected by a peptide or linker sequence. In some embodiments, a single chain bispecific scFv further comprises a signal peptide to allow for secretion from cells. Bispecific single chain molecules are known in the art and are described in International PCT Publication No. WO 99/54440; Mack, J. Immunol. (1997), 158, 3965-3970; Mack, PNAS, (1995), 92, 7021-7025; Kufer, Cancer Immunol Immunother, (1997), 45, 193-197; Loftier, Blood, (2000), 95, 6, 2098-2103; and Bruhl, J. Immunol., (2001), 166, 2420-2426.

In some embodiments, the molecular format of the polynucleotide encoding a single chain bi-specific scFv polypeptide comprises a nucleic acid sequence encoding a signal peptide (such as the signal sequences of SEQ ID NO: 2 or 4) followed by two or more scFv regions (e.g., a first scFv and a second scFv) each comprising one V_{H} and one V_{L} chain linked together. In specific embodiments, the two or more antibody-derived regions are scFvs and are linked by a peptide linker to form a single chain bi-specific scFv construct. In some embodiments, the bi-specific scFv is a tandem bi-scFv or a diabody. Single chain bispecific scFvs can be arranged in different formats from N-terminus to C-terminus, including the following, wherein “L” is a linker: V_{H}1-L-V_{L}1-L-V_{H}2 L-V_{L}2; V_{L}1-L-V_{H}1-L-V_{H}2-L-V_{L}2; V_{H}-L-V_{L}-L-V_{L}2-L-V_{H}2; V_{L}-L-V_{H}1-L-V_{L}2-L-V_{H}2; V_{H}2-L-V_{L}2-L-V_{H}1-L-V_{L}1; V_{L}2-L-V_{H}2-L-V_{H}1-L-V_{L}1; V_{H}2-L-V_{L}2-Ly-V_{L}1-L-V_{H}1; V_{L}2-L-V_{H}2-L-V_{L}1-L-V_{H}1; V_{H}1-L-V_{H}2-L-V_{L}2-L-V_{L}1; V_{L}2-L-V_{L}1-L-V_{H}1-L-V_{H}2; V_{H}2-L-V_{H}1-L-V_{L}1-L-V_{L}2; or V_{L}1-L-V_{L}2-L-V_{H}2 L-V_{H}I.

In some embodiments, the engager molecules of the present invention specifically bind to/interact with a particular conformational/structural epitope(s) of cell-surface molecules. Accordingly, specificity in some instances is determined experimentally by methods known in the art and methods as disclosed and described herein. Such methods comprise, but are not limited to Western blots, enzyme-linked immunosorbent assay (ELISA), radioimmunoassay (RIA), radioimmtinoprecipitation (RIP), electrochemiluminescence (ECL), immunoradiometric assay (IRMA), enzyme immunoassay (EIA), and peptide scans.

In some embodiments, the engager polypeptides comprise an effector domain and a targeting domain or an activation domain. In some embodiments, the respective domains are in any order from N-terminus to C-terminus. For example, in some embodiments, the engager molecule comprises an N-terminal effector domain and a C-terminal targeting domain. In some embodiments, the engager molecule may comprise an N-terminal targeting domain and a C-terminal effector domain. In some embodiments, the engager molecule may comprise an N-terminal effector domain and a C-terminal activation domain. In some embodiments, the engager molecule may comprise an N-terminal activation domain and a C-terminal effector domain. In some embodiments, the engager molecule comprises multiple (e.g., 2, 3, 4, 5 or more) targeting domains to allow targeting of multiple antigens on target cells. In some embodiments, the engager molecule comprises multiple (e.g., 2, 3, 4, 5 or more) effector domains. In some embodiments, the engager molecule comprises multiple (e.g., 2, 3, 4, 5 or more) activation domains. In some embodiments of the disclosure, the engager molecule comprises additional domains for the isolation and/or preparation of recombinantly produced constructs, such as a tag or a label. The tag or label may be a short peptide sequence, such as a histidine tag (SEQ ID NO: 12), or may be a tag or label that is capable of being imaged, such as fluorescent or radioactive label.

In some embodiments, the engager polypeptides comprise an effector domain and a targeting domain or an activation domain. An “effector domain” recognizes and binds to a molecule present on the surface of an effector cell. As used herein, the term “effector cell” refers to any mammalian cell type that is capable of mediating an immune response, including facilitating the death of a target cell. In particular embodiments, the effector cells of the present invention are immune cells, such as a T cell, a B cell, an innate lymphocyte, a natural killer (NK) cell, a natural killer T cell (NKT), a granulocyte (e.g., a neutrophil, basophil, mast cell, or eosinophil), a macrophage, a monocyte, or a dendritic cell. Exemplary effector cell types include T cells, NK cells, NKT cells, and macrophages. The skilled artisan will recognize that the nature of the cell surface molecule recognized by the effector domain may vary according to the nature of the effector cell, although different groups of effector cells may share expression of certain types of cell surface molecules. For example, T cells express different surface receptors than NK cells or macrophages. As an illustrative example, CD3 is a receptor expressed by T-cells that is not expressed by NK cells or macrophages, whereas CD1, CD16, NKG2D, and/or NKp30 are receptors expressed by NK cells that are not expressed by T cells. Therefore, in some instances, engager molecules that activate T-cells have a different effector domain than engager molecules that activate NK cells, macrophages, NKT cells, or other types of effector cells.

In some embodiments, the effector cell is a T cell. The T-cell repertoire is comprised of numerous sub-types of T cell, cytotoxic T cells (Tc or CTL), memory T cells, helper T cells (e.g., Th1, Th2, Th17, Th9, and/or Th22 cells), suppressor T cells (e.g., regulator T cells (Tregs)), mucosal-associated invariant T cells, and γδ T cells. In some instances, one or more surface receptors expressed by one T cell subtype are not expressed by another T cell subtype. In some instances, one or more surface receptors expressed by one T cell subtype are expressed by at least one other T cell subtype. In some instances, one or more surface receptors expressed by one T cell subtype are generally expressed by all, or most, T cell subtypes. For example, CD3 is a signaling component of the T cell receptor (TCR) complex and is expressed in multiple T cell subtypes.

In some embodiments, the effector cell is a T cell and the effector domain of the engager molecule binds to one or more components of CD3 (e.g., CD3γ, CD3δ, CD3ε or CD3ξ), CD2, CD4, CD5, CD6, CD7, CD8, CD25, CD27, CD28, CD30, CD38, CD40, CD57, CD69, CD70, CD73, CD81, CD82, CD96, CD134, CD137, CD152, CD278 and/or LIGHT (also known as TNFSF14). In some embodiments, the effector cell is an NKT-cell and the effector domain of the engager molecule binds to one or more components of CD3 or an invariant TCR. In some embodiments, the effector cell is an NK cell and the effector domain of the engager molecule binds to CD16, CD94/NKG2 (e.g., NKG2D), NKp30, NKp44, NKp46, or killer activation receptors (KARs). Additional cell-surface molecules that can be expressed on lymphocytes and can be bound by effector domains of engager molecules include CD48, expressed by T cells and NK cells.

In some embodiments, binding of an effector domain to a molecule present on the surface of an effector cell results in activation of the effector cell. In some embodiments, activation of an effector cell results in one or more of the following: (i) increased proliferation of the effector cell; (ii) changes in the expression or activity of one or more cell surface proteins of the effector cell; (iii) change in expression or activity of one or more intracellular proteins expressed by the effector cell; (iv) changes in the amount or nature of factors produced and/or secreted by the effector cell, such as cytokines, chemokines or reactive oxygen species; (v) changes in the morphology of the effector cell; (vi) changes in the chemotactic potential of the effector cell, such as through increased or decreased expression of one or more chemokine receptors; (vii) changes in the functional activity of the effector cell, such as increased cytolytic activity and/or increased phagocytic activity; and/or (viii) increased cytotoxicity against a target cell. Activation of an effector cell, or population of effector cells, can be determined by any means known in the art. For example, changes in proliferation, protein expression, production, or secretion can be determined by flow cytometry, Western blot, ELISA, immunohistochemistry, immunoprecipitation, or immunofluorescence and changes in cell morphology can be determined by numerous types of microscopy known in the art.

In some embodiments, the engager polypeptides comprise an effector domain and an activation domain. An “activation domain” of an engager molecule binds to a protein expressed on the cell-surface and results in cell activation. In some embodiments, the cell-surface protein recognized by the activation domain and the cell-surface protein recognized by the effector domain are both expressed on an effector cell. However, the cell-surface protein recognized by the activation domain is distinct from the the cell-surface protein recognized by the effector domain. In particular embodiments, the activation domain binds to a cell surface protein that is a negative regulator of effector cell function (e.g., an immune checkpoint molecule or other inhibitory molecule). Exemplary cell-surface antigen for targeting by an activation domain include 2B4, A2aR, B7H3 (CD276), B7H4, CD2, CD28, CD30, CD40, CD47, CD70, CD80, CD86, CD137, CD160, CD226, DR3, GAL9, GITR, HAVCR2, HVEM, IDO1, ID02, ICOS, LAIRI, LIGHT, MARCO, PS, SLAM, TIGHT, VISTA, VTCN1, PD1, PDL1, PDL2, CTLA4, TIM2, LAG3, BTLA, KIR, TIGIT, OX40, FITR, CD27, SLAMF7, and CD200. In certain embodiments, engager molecules comprising an activation domain and an effector domain enhance the activation of the effector cell and thereby facilitate the destruction of one or more bystander target cells by the effector cell.

In some embodiments, the engager polypeptides comprise an effector domain and a targeting domain. A “targeting domain” of an engager molecule binds to a cell-surface molecule expressed by a target cell. As used herein, the term “target cell” refers to a mammalian cell that should be killed, attacked, destroyed, and/or controlled. In particular, target cells are cells that are in some way altered compared to a normal cell of the same cell type, such as a cancerous cell, a bacterially-infected cell, a virally-infected cell, a fungally-infected cell, and/or an autoimmune cell. In particular embodiments, the target cells of the present invention are cancerous cells (e.g., tumor cells). In some embodiments, binding of an engager molecule to a target cell and an effector cell brings the effector cell in close proximity to the target cell and thereby facilitates the destruction of the target cell by the effector cell. Destruction (i.e., death) of a target cell can be determined by any means known in the art, such as flow cytometry (e.g., by AnnexinV, propidium iodide, or other means), cell counts, and/or microscopy to determine the cellular morphology of the target cells.

In some embodiments, the targeting domain of an engager molecule brings a target cell (e.g., tumor cell) into the vicinity of an effector cell via interaction between the targeting domain and surface antigens expressed by the target cell (e.g., target cell antigens). In some embodiments, the target-cell antigen is a tumor antigen. In some embodiments, a tumor antigen is a tumor-specific antigen (TSA), and is expressed only by tumor cells. In some embodiments, the target cell angien is a tumor-associated antigen (TAA), and is expressed by tumor cells and one or more types of normal cells or non-tumor cells. In some cases, TSA is also present in one or more types of normal cells or non-tumor cells, but is predominantly expressed by tumor cells. In some instances, a tumor antigen (e.g., TSA or TAA) is present in one cancer type. In some instances, a tumor antigen is present in multiple cancer types. In one embodiment, a tumor antigen is expressed on a blood cancer cell. In another embodiment, a tumor antigen is expressed on a cell of a solid tumor. In some embodiments, the solid tumor is a glioblastoma, a non-small cell lung cancer, a lung cancer other than a non-small cell lung cancer, breast cancer, prostate cancer, pancreatic cancer, liver cancer, colon cancer, stomach cancer, a cancer of the spleen, skin cancer, a brain cancer other than a glioblastoma, a kidney cancer, a thyroid cancer, or the like. In more specific embodiments, a tumor antigen is expressed by a tumor cell in an individual.

In certain embodiments, the targeting domain of an engager molecule specifically binds a tumor-associated antigen (TAA) or a tumor-specific antigen (TSA). In certain embodiments, the targeting domain comprises an antibody or an antibody fragment or an antigen-binding fragment or portion thereof, such as for example, a monoclonal antibody, Fv, a scFv, Fab, minibody, or diabody that is specific for a TAA or TSA. In certain embodiments, the targeting domain of the engager is an scFv that is specific for a TAA or TSA. In a specific embodiment, the TAA or TSA is expressed on a cancer cell. In one embodiment, the TAA or TSA is expressed on a blood cancer cell. In another embodiment, the TAA or TSA is expressed on a cell of a solid tumor. In some embodiments, the solid tumor is a glioblastoma, a non-small cell lung cancer, a lung cancer other than a non-small cell lung cancer, breast cancer, prostate cancer, pancreatic cancer, liver cancer, colon cancer, stomach cancer, a cancer of the spleen, skin cancer, a brain cancer other than a glioblastoma, a kidney cancer, a thyroid cancer, or the like. In more specific embodiments, the TAA or TSA is expressed by a tumor cell in an individual.

Exemplary tumor antigens (e.g., TSAs or TAAs) include, but are not limited to, 5T4, 8119, ABCG2, absent in melanoma 2 (AIM2), acetylglucosaminyltransferase-V (GnT-V, β1,6-N), alphafetoprotein (AFP), the transferrin receptor, antigen recognized by T cells (ART)-1, ART4, B7-H3, B7-H4, B7-H6, BCMA, BMI1 polycomb ring finger oncogene (BMI1), CA-125 (also known as MUC16), carbonic anhydrase (CA)-6, CA-9 (also known as CAIX), cadherin, calcium binding tyrosine-(Y)-phosphorylation regulated (CABYR), cancer/testis antigen 2 (CTAG2), carcinoembryonic antigen (CEA), cathepsin B, caveolin-1 (Cav-1), CCR8, CD10 (also known as neprilysin, membrane metallo-endopeptidase (MME), neutral endopeptidase (NEP), and common acute lymphoblastic leukemia antigen (CALLA)), CD117 (c-kit), CD123, CD133, CD138, CD15, CD171, CD19, CD20, CD21, CD22, CD30, CD33, CD38, CD4, CD44, CD44v6, CD44v7/8, CD45, CD70, CD74, CD79b, CD8, CEACAM5, cell proliferation-associated human nuclear antigen defined by the monoclonal antibody Ki-67 (Ki-67), chemokine receptor 5 (CCR5), CD74, CSAG family member 2 (CSAG2), chondroitin sulfate proteoglycan 4 (CSPG4, also known as MCSP), cyclin B, cyclooxygenase-2 (COX-2), cysteine-rich secretory protein 3 (CRISP3), dendritic cell-specific intercellular adhesion molecule 3-grabbing nonintegrin (DC-SIGN), dihydrofolate reductase (DHFR), EGP2, EGP40, enhancer of zeste homolog 2 (EZH2), EpCAM, EPH receptor A2 (EphA2), EphA2/epithelial kinase (EphA2/Eck), epidermal growth factor receptor variant III (EGFRvIII), epithelial calcium-dependent adhesion (E-cadherin), epithelial tumor antigen (ETA), ERBB3, ErbB3/4, ERBB4, erythropoietin (EPO), fibroblast activation protein (FAP), far upstream binding element 1 (FUBP1), FAR, folate binding proteins (FBP including Folate receptor alpha (FRu)), ferritin heavy polypeptide 1 testis-specific expression (FTHL17), fetal AchR, FGFR3, fos-related antigen 1 (Fra-1/Fosl 1), G antigen 1 (GAGE1), Ganglioside/GD2, GD3, glioma-associated oncogene homlog 1 (Glil), glycoprotein 100 (GP100), Glypican-3, GPA33, granulocyte-macrophage colony stimulating factor (GM-CSF), H60, HIV gp120, HLA-A, HLA-A2, HLA-AI, HLA-B, HLA-C, HMW-MAA, human epidermal growth factor receptor 2 (Her2/Neu), human Ku heterodimer proteins subunits (u70/80), human LI cell adhesion molecule (LICAM), IL-15R, IL-1R2, IL-1Ru, immunoglobulins, integrins, interleukin-13 receptor subunit alpha-2 (IL-13Ra2), kappa light chain, lactate dehydrogenase C (LDHC), lambda light chain, LAMP-1 (also known as CD107b), layilin (LAYN), Leu-7 (CD57), LeuM-1, Lewis-Y, LIV-1, Livin (also known as BIRC7), melanoma antigen family A (MAGEA) 1, MAGEA3, MAGEA4, melanoma antigen family B 6 (MAGEB6), melanoma antigen recognized by T cells (MART-1), Mesothelin, mitogen-activated protein kinase I (MAPK1), mucin (MUC)-1, multidrug resistance protein 3 (MRP-3), myogenic differentiation promoting factor-1 (MyoD-1), sodium-dependent phosphate transport protein 2A (NaPi2a), neural cell adhesion molecule (NCAM), necrotic regions of tumors, nectin 4, Nestin, New York esophageal squamous cell carcinoma 1 (NY-ESO-1), NKG2D ligands (including the murine NKGD2 ligands Rae-1α, Rae-1β, Rae-1γ, and Rae-1δ, and human NKG2D ligands MHC Class I polypeptide-related sequence A (MICA) and MICB, and ULBP1, ULBP2, ULBP3, ULBP4, ULBP5, and ULBP6), NLR family, nuclear proliferation-associated antigen of antibody Ki67 (Ki67), oligodendrocyte transcription factor (OLIG2), oncofetal variants of fibronectin, p5³, P-Cadherin, PDZ binding kinase (PB), P-glycoprotein, preferentially expressed antigen in melanoma (PRAME), prospero homeobox protein I (PROXI), prostate specific antigen (PSA), prostate stem cell antigen (PSCA), prostate stem cell antigen (PSCA), PSC1, PSMA, pyrin domain containing 4 (NLRP4), Ras, ROR1, SART2, SART3, sex determining region Y-box (SOX)-2, SOX10, SOX11, SLITRK6, sperm protein associated with the nucleus X-linked family member A1 (SPANXAI), squamous cell carcinoma antigen recognized by T cells (SART)-I, synovial sarcoma X breakpoint 2 (SSX2), SSX4, SSX5, STEAP1, Survivin, TAG72, TEM1, TEM8, tenascin, testis specific 10 (TSGA10), testis-specific serine kinase 6 (TSSK6), thyroglobulin, TMEM97, TRP-2, tubby like protein (TULP2), tyrosinase, tyrosine related protein (TRP)-1, urokinase-type plasminogen activator receptor (UPAR), VEGF receptors (including VEGFR-R2, also known as KDR), VEGF, viral envelope proteins, v-raf murine sarcoma viral oncogene homolog B1 (BRAF), Wilms' tumor protein 1 (WT-1), X antigen family member 2 (XAGE2), zinc finger protein 165 (ZNF165), avj36 integrin, and β-catenin.

### Ephrin Type-A Receptor 2 (EphA2)

In some embodiments, the target antigen is ephrin type-A receptor 2 (EPHA2, also known as ARCC2, CTPA, CTPP1, and ECK). The EPHA2 protein is a member of the ephrin receptor subfamily of the protein-tyrosine kinase family. Receptors in this subfamily generally comprise a single kinase domain and an extracellular region comprising a Cys-rich domain and 2 fibronectin type III repeats; embodiments of the antibodies of the disclosure target any of these domains. Exemplary human EphA2 nucleic sequences are provided in GenBank® Accession No. NM_004431.5 and NM_001329090.1, and exemplary human EphA2 polypeptide sequences are in GenBank® Accession No. NP_004422.2 and NP_001316019.1.

The Eph family, the largest group among tyrosine kinase receptor families, is comprised of the EphA (EphA1-10) or EphB (EphB1-6) subclasses of receptors classified by sequence homology and binding affinity for Ephrin (Eph receptor interacting protein) ligands. The human EphA2 gene is located on chromosome 1, encodes a receptor tyrosine kinase of 976 amino acids with an apparent molecular weight of 130 kDa and has a 90% amino acid sequence homology to the mouse EphA2. The Eph family contains an extracellular conserved N-terminal ligand-binding domain followed by a cysteine-rich domain with an epidermal growth factor-like motif and two fibronectin type-III repeats. The extracellular motif is followed by a membrane spanning region and a cytoplasmic region that encompasses a juxtamembrane region, a tyrosine kinase domain, a sterile alpha motif (SAM), and a post synaptic domain (disc large and zona occludens protein (PDZ) domain-binding motif). EphA2 shows 25-35% sequence homologies with other Eph receptors, and the tyrosine residues are conserved within the juxtamembrane and kinase domain.

EphA2 mRNA expression is observed in the skin, bone marrow, thymus, uterus, testis, prostate, urinary bladder, kidney, small intestine, colon, spleen, liver, lung, and brain. EphA2 expression in the colon, skin, kidney and lung is increased over ten-fold relative to the bone marrow. EphA2 is also expressed during gastrulation in the ectodermal cells and early embryogenesis in the developing hind brain. In the skin, EphA2 is present in keratinocytes of epidermis and hair follicles but not in dermal cells (fibroblasts, vascular cells and inflammatory cells). EphA2 is also expressed in proliferating mammary glands in female mice at puberty and differentially expressed during the estrous cycle. EphA2 is also overexpressed in several cancers, such as breast cancer, gastric cancer, melanoma, ovarian cancer, lunch cancer, gliomas, urinary bladder cancer, prostate cancer, esophageal cancer, renal cancer, colon cancer, and vulvar cancers. In particular, a high level of EphA2 is detected in malignant cancer-derived cell lines and advanced forms of cancer. In light of the EphA2 overexpression in pre-clinical models and clinical specimens of many different types of cancer, the increased level of EphA2 expression is informative in both the prediction of cancer outcomes and in the clinical management of cancer. The differential expression of EphA2 in normal cells compared to cancer cells also signifies its importance as a therapeutic target.

### Human Epidermal Growth Factor Receptor 2 (HER2)

In some embodiments, the target antigen is human Epidermal Growth Factor Receptor 2 (HER2, also referred to as ERBB2, NEU, NGL, HER2, TKR1, CD340, HER-2, MLN 19, HER-2/neu). HER2 is a member of the epidermal growth factor receptor family having tyrosine kinase activity and is encoded by the HER2 gene in the epidermal growth factor receptor (EFR/ErbB) family. Dimerization of the receptor results in the autophosphorylation of tyrosine residues within the cytoplasmic domain of the receptors and initiates a variety of signaling pathways leading to cell proliferation and tumorigenesis. Amplification or overexpression of HER2 is associated with a multitude of different cancers and can serve as a prognostic and predictive biomarker. For example, amplification or overexpression of HER2 occurs in approximately 15-30% of breast cancers and 10-30% of gastric/gastroesophageal cancers. Overexpression of the HER2 protein and amplification of the HER2 gene was found in 23% and 27%, respectively, of 200 resected tumors in a gastric cancer study. Further, in a study of 260 gastric cancers, HER2 overexpression was an independent negative prognostic factor and HER2 staining intensity correlated with tumor size, serosal invasion, and lymph node metastases. Additional studies have reported HER2 overexpression in 0-83% of esophageal cancers, with a tendency towards higher rates of positivity in adenocarcinoma (10-83%) compared to squamous cell carcinomas (0-56%). HER2 overexpression has also been seen in ovarian cancers, endometrial cancers, bladder cancer, lung cancer, colon cancer, and head and neck cancer. For example, overexpression of HER2 has been reported in 20-30% patients with ovarian cancer. In endometrial serous carcinoma, the reported rates of HER2 overexpression range between 14% and 80% with HER2 amplification ranging from 21% to 47%.

Exemplary human HER2 nucleic sequences are provided in GenBank® Accession Nos. NM_004448.3, NM_001005862.2, NM_001289936.1, NM_001289937.1, and NM_001289938.1. Exemplary human HER2 polypeptide sequences are provided in in GenBank® Accession Nos. NP_004439.2, NP_001005862.1, NP_001276865.1, NP_001276866.1, and NP_001276867.1.

### Disialoganglioside GD2

In some embodiments, the target antigen is the disialoganglioside GD2. GD2 is a sialic acid-containing glycosphingolipid expressed primarily on the cell surface. The function of this carbohydrate antigen is not completely understood; however, it is thought to play an important role in the attachment of tumor cells to extracellular matrix proteins. GD2 expression in normal fetal and adult tissues is primarily restricted to the central nervous system, peripheral nerves, and skin melanocytes, although GD2 expression has been described in the stromal component of some normal tissues and white pulp of the spleen. In malignant cells, GD2 is uniformly expressed in neuroblastomas and most melanomas and to a variable degree in a variety of other tumors, including bone and soft-tissue sarcomas, small cell lung cancer, and brain tumors. GD2 is present and concentrated on cell surfaces, with the two hydrocarbon chains of the ceramide moiety embedded in the plasma membrane and the oligosaccharides located on the extracellular surface, where they present points of recognition for extracellular molecules or surfaces of neighboring cells. Because of the relatively tumor-selective expression combined with its localization on the cell surface, GD2 is an attractive target for tumor-specific antibody therapy. Exemplarily antigen binding domains for targeting GD2 are known in the art, for example those described in Navid et al., Curr Cancer Drug Targets. 2010; 10(2):200-209, and the monoclonal Dinutuximab antibody (Ploessl et al., Ann Pharmacother. 2016; (50(5): 416-422).

### Fibroblast Activation Protein (FAP)

In some embodiments, the target antigen is fibroblast activation protein (FAP, also known as FAPA, SIMP, DPPIV, and FAPalpha). FAP is a 170 kDa membrane-bound gelatinase expressed during embryonic development (Niedermeyer et al., Intl J Dev Biol. 2001; 45(2): 445-7 and in adults in pancreatic alpha cells (Busek et al., Histochemistry and Cell Biology. 2015; 143(5): 497-504), in multipotent bone marrow stromal cells (Bae et al., British Journal of Haematology. 2008; 142(5): 827-30), and uterine stroma (Dolznig et al, Cancer Immunity. 2005; 5:10). FAP expression is seen on activated stromal fibroblasts of more than 90% of all human carcinomas (Pure et al., Oncogene. 2018; 37 (32): 4343-4357; Busek et al., Frontiers in Bioscience. 2018; 23: 1933-1968).

Exemplary human FAP nucleic sequences are provided in GenBank® Accession Nos. NM_001291807.2 and NM_004460.5. Exemplary human FAP polypeptide sequences are provided in in GenBank® Accession Nos. NP_001278736.1 and NP_004451.2. Exemplarily antigen binding domains for targeting FAP are known in the art, including the monoclonal antibody sibrotuzumab (Scott et al., Clin Cancer Res. 2003; 9(5):1639-47; Hofheinz et al., Onkologie. 2003; 26(1):44-8) as well as those described in Tran el al., J Exp Med. 2013 Jun. 3; 210(6):1125-35; Garin-Chesa el al., PNAS 1990; 87(18): 7235-7239; Santos et al., J Clin Invest 119:3613-25 (2009); Goscinski et al., Ultrastruct Pathol 32:89-96 (2008); Lo et al., JCI Insight, 2017; 2(19); Lakins et al., Nat Commun, 2018; 9(1):948; U.S. Pat. No. 6,455,677; US Patent Application Publication No. 2014-0370019; International PCT Publication Nos. WO 1999/057151, 2001/068708, WO 2007/077173, WO 2012/020006, WO WO 2014/055442, and WO 2016/055432. Exemplary anti-FAP scFv sequences are provided in SEQ ID NO: 73.

### Therapeutic Molecules

In some embodiments, the oncolytic viruses described herein comprise a nucleic acid sequence that encodes an engager molecule and one or more additional nucleic acid sequences that encode one or more therapeutic molecules. As used herein, a “therapeutic molecule” refers to a molecule that enhances the therapeutic efficacy of an oncolytic virus described herein. In general, the therapeutic molecules described herein are proteins, nucleic acids, or a combination thereof. Exemplary therapeutic molecules include cytokines, chemokines, antibodies or antigen binding fragments thereof, proteases, RNA polynucleotides, and DNA polynucleotides.

In some embodiments, the therapeutic molecule is capable of increasing or enhancing the therapeutic efficacy of an oncolytic virus described herein by stimulating, or activating, a cellular immune response. In some embodiments, the therapeutic molecule is capable of increasing or enhancing the therapeutic efficacy of an oncolytic virus described herein by antagonizing a suppressive or regulatory immune response. In some embodiments, reduction of a suppressive immune response occurs in a tumor microenvironment. In some instances, reduction of a suppressive immune response by the therapeutic molecule enhances the oncolytic effects of an oncolytic virus described herein. In some embodiments, the therapeutic molecule further reduces immunoregulatory T cell activity in a subject treated with an oncolytic virus described herein. In some embodiments, the therapeutic molecule modulates or impairs the production level of a protein at a nucleic acid level or at a protein level, or disrupts a protein function.

In some embodiments, the nucleic acid sequence encoding the engager molecule and the nucleic acid sequence encoding one or more therapeutic molecules are comprised within the same vector or virus. In some embodiments, the nucleic acid sequence encoding the engager molecule and the nucleic acid sequence encoding one or more therapeutic molecules are comprised in different vectors or viruses. In some embodiments, the vector is a viral vector. In some embodiments, the therapeutic molecule is a polypeptide. In some instances, the polypeptide is an immune modulator polypeptide. In some cases, the immune modulator polypeptide is a cytokine, a co-stimulatory domain, a domain that inhibits negative regulatory molecules of T-cell activation (e.g., an immune checkpoint inhibitor), or a combination thereof.

In some embodiments, the immune modulator polypeptide modulates the activity of one or more cell types, such as regulatory T cells (Tregs), myeloid-derived suppressor cells (MDSCs), dendritic cells, and/or T cells. Exemplary Treg modulatory polypeptides include CCR4, Helios, TIGIT, GITR, neuropilin, neuritin, CD103, CTLA-4, ICOS, and Swap70. Exemplary MDSC modulatory polypeptides include TGF-βR1, GM-CSF, INFγ, interleukins such as IL-β, IL-1F2, IL-6, IL-10, IL-12, IL-13, IL-6, IL-6Rα, IL-6/IL-6R complex, TGF-β1, M-CSF, Prostaglandin E2/PGE2, Prostaglandin E Synthase 2, S100A8, and VEGF. Exemplary dendritic-cell directed modulatory polypeptides include GM-CSF and/or IL-13. Exemplary T cell-directed modulatory polypeptides include IL-12, OX-40, GITR, CD28, or IL-28, or an antibody that agonizes a pathway comprising IL-12, OX-40, GITR, CD28, or IL-28.

In other embodiments, the therapeutic polypeptides modulate the fibrotic stroma. Exemplary fibrotic stromal polypeptides include fibroblast activation protein-alpha (FAP). In some embodiments, the therapeutic polypeptide is a protease. In particular embodiments, the protease is capable of altering the extracellular matrix, particularly the extracellular matrix within a tumor microenvironment. Exemplary proteases include matrixmetalloproteases (MMP), such as MMP9, collagenases, and elastases.

### Cytokines as Therapeutic Molecules

In some cases, the immune modulator polypeptide is a cytokine. Cytokines are a category of small proteins between about 5-20 kDa that are involved in cell signaling and include chemokines, interferons (INF), interleukins (IL), and tumor necrosis factors (TNF), among others. Chemokines play a role as a chemoattractant to guide the migration of cells and are classified into four subfamilies: CXC, CC, CX3C, and XC. Exemplary chemokines include chemokines from the CC subfamily, such as CCL1, CCL2 (MCP-1), CCL3, CCL4, CCL5 (RANTES), CCL6, CCL7, CCL8, CCL9 (or CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, and CCL28; the CXC subfamily, such as CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, and CXCL17; the XC subfamily, such as XCL1 and XCL2; and the CX3C subfamily, such as CX3CLI.

Interferons (IFNs) comprise Type I IFNs (e.g. IFN-α, IFN-β, IFN-ε, IFN-κ, and IFN-ω), Type II IFNs (e.g. IFN-γ), and Type III IFNs. In some embodiments, IFN-α is further classified into about 13 subtypes including IFNA1, IFNA2, IFNA4, IFNA5, IFNA6, IFNA7, IFNA8, IFNA10, IFNA13, IFNA14, IFNA16, IFNA17, and IFNA21. Interleukins are a broad class of cytokine that promote the development and differentiation of immune cells, including T and B cells, and other hematopoietic cells. Exemplary interleukins include IL-1, IL-2, IL-3, IL-4, IL-5, TL-6, IL-7, IL-8 (CXCL8), IL-9, IL-10, IL-11, TL-12, IL-13, IL-14, IL-I5, IL-16, IL-7, IL-18, IL-19, IL-20, IL-21, IL-22, TL-23, TL-24, IL-25, IL-26, IL-27, IL-28, IL-29, IL-30, IL-31, IL-32, IL-33, IL-35, and IL-36. Tumor necrosis factors (TNFs) are a group of cytokines that modulate apoptosis. In some instances, there are about 19 members within the TNF family, including, not limited to, TNFα, lymphotoxin-alpha (LT-α), lymphotoxin-beta (LT-β), T cell antigen gp39 (CD40L, also called CD154), CD27L, CD30L, FASL, 4-1BBL, OX40L, and TNF-related apoptosis inducing ligand (TRAIL).

In some embodiments, an oncolytic virus comprises a nucleic acid sequence that encodes an engager and an additional nucleic acid sequence that encodes a cytokine. In some embodiments, the cytokine selected from IL-15, IL-12, CXCL0, and CCL4.

### Co-Stimulatory Domains as Therapeutic Molecules

In some embodiments, the immune modulator polypeptide is a co-stimulatory domain. In some cases, the co-stimulatory domain enhances antigen-specific cytotoxicity. In some cases, the co-stimulatory domain further enhances cytokine production. In some embodiments, the co-stimulatory domain comprises CD27, CD28, CD70, CD80, CD83, CD86, CD134 (OX-40), CD134L (OX-40L), CD137 (41BB), CD137L (41BBL), or CD224.

In some embodiments, an oncolytic virus comprises a nucleic acid sequence that encodes an engager and an additional nucleic acid sequence that encodes a co-stimulatory domain. In some embodiments, the co-stimulatory domain is selected from CD27, CD28, CD80, CD83, CD86, CD134, CD134L, CD137, CD137L, or CD224.

### Immune Checkpoint Polypeptides as Therapeutic Molecules

In some embodiments, the immune modulator polypeptide is an immune checkpoint polypeptide. In some embodiments, the immune modulator polypeptide is an immune checkpoint inhibitor polypeptide that inhibits a negative regulatory molecule of T-cell activation. Immune checkpoint inhibitor bind to immune checkpoint molecules, which are a group of molecules on the cell surface of CD4 and CD8 T cells. In some instances, these molecules effectively serve as “brakes” to down-modulate or inhibit an anti-tumor immune response. An immune checkpoint inhibitor refers to any molecule that modulates or inhibits the activity of an immune checkpoint molecule. In some instances, immune checkpoint inhibitors include antibodies, antibody-derivatives (e.g., Fab fragments, scFvs, minobodies, diabodies), antisense oligonucleotides, siRNA, aptamers, or peptides.

Exemplary immune checkpoint molecules include, but are not limited to, programmed death-ligand 1 (PDL1, also known as B7-H1, CD274), PD-L2 (B7-DC, CD273), programmed death 1 (PD-1), CTLA-4, LAG3, TIM3 (HAVCR2), A2aR, B7H1, B7113 (CD276), B7H4 (VTCN1), BTLA, CD27, CD70, GAL9, HVEM, IDO1, IDO2, KIR, LAIR1, LIGHT, TIGIT, and VISTA.

In some embodiments, an oncolytic virus comprises a nucleic acid sequence that encodes an engager molecule and an additional nucleic acid sequence that encodes an immune checkpoint inhibitor. In some embodiments, the immune checkpoint inhibitor reduces the expression or activity of one or more immune checkpoint molecules. In some embodiments, the immune checkpoint inhibitor reduces the interaction between an immune checkpoint molecule and its ligand (e.g., reduced the interaction between PD-1 and PDL1). In some embodiments, the immune checkpoint inhibitor inhibits one or more of PDL1, PD-1, CTLA-4, PD-L2, LAG3, TIM3 (HAVCR2), A2aR, B7H1, B7H3 (CD276), B7H4 (VTCN1), BTLA, CD27, CD70, GAL9, HVEM, IDO1, ID02, KIR, LAIRI, LIGHT, TIGIT, and VISTA.

In some embodiments, the immune checkpoint inhibitor is an inhibitor of PDL1. In some embodiments, the immune checkpoint inhibitor is an antibody (e.g., a monoclonal antibody or antigen-binding fragments thereof, or a humanized or chimeric antibody or antigen-binding fragments thereof) against PDL1. In some embodiments, the inhibitor of PDLT reduces the expression or activity of PDL1. In some embodiments, the inhibitor of PDL1 reduces the interaction between PD-1 and PDL1. Exemplary inhibitors of PDL1 include anti-PDL1 antibodies, RNAi molecules (e.g., anti-PDL1 RNAi), antisense molecules (e.g., an anti-PDL1 antisense RNA), or dominant negative proteins (e.g., a dominant negative PDL1 protein). Exemplary anti-PDL1 antibodies includes clone EH12; MPDL3280A (Genentech, RG7446); anti-mouse PDL1 antibody Clone 10F.9G2 (BioXcell, Cat #BE0101); anti-PDL1 monoclonal antibody MDX-1105 (BMS-936559 and BMS-935559 from Bristol-Meyers Squibb; MSB0010718C; mouse anti-PDL1 Clone 29E.2A3; and AstraZeneca's MEDI4736. In some embodiments, the anti-PDL1 antibody is an anti-PDL1 antibody disclosed in International PCT Publication Nos. WO 2013/079174; WO 2010/036959; WO 2013/056716; WO 2007/005874; WO 2010/089411; WO 2010/077634; WO 2004/004771; WO 2006/133396; WO 2013/09906; WO 2012/145493; WO 2013/181634; U.S. Patent Application Publication No. 20140294898; or Chinese Patent Application Publication No. CN 101104640.

In some embodiments, the PDL1 inhibitor is a nucleic acid inhibitor of PDL1 expression. In some embodiments, the PDL1 inhibitor is one disclosed in International PCT Publication Nos. WO 2011/127180 or WO 2011/000841.

In some embodiments, an oncolytic virus comprises a nucleic acid sequence that encodes an engager molecule and an additional nucleic acid sequence that encodes PDL1 inhibitor selected from EH12, Genentech's MPDL3280A (RG7446); Anti-mouse PDL1 antibody Clone 10F.9G2 (Cat #BE0101) from BioXcell; anti-PDL1 monoclonal antibody MDX-1105 (BMS-936559) and BMS-935559 from Bristol-Meyer's Squibb; MSB0010718C; mouse anti-PDL1 Clone 29E.2A3; and AstraZeneca's MEDI4736.

In some embodiments, the immune checkpoint inhibitor is an inhibitor of PD-L2. In some embodiments, the inhibitor of PD-L2 is an antibody (e.g., a monoclonal antibody or fragments, or a humanized or chimeric antibody or fragments thereof) against PD-L2. In some embodiments, the inhibitor of PD-L2 reduces the expression or activity of PD-L2. In other embodiments, the inhibitor of PD-L2 reduces the interaction between PD-1 and PD-L2. Exemplary inhibitors of PD-L2 include antibodies (e.g., an anti-PD-L2 antibody), RNAi molecules (e.g., an anti-PD-L2 RNAi), antisense molecules (e.g., an anti-PD-L2 antisense RNA), or dominant negative proteins (e.g., a dominant negative PD-L2 protein). In some embodiments, the PD-L2 inhibitor is GlaxoSmithKline's AMP-224 (Amplimmune). In some embodiments, the PD-L2 inhibitor is rHIgM12B7. In some embodiments, an oncolytic virus comprises a nucleic acid sequence that encodes an engager molecule and an additional nucleic acid sequence that encodes a PD-L2 inhibitor. In some embodiments, the PD-L2 inhibitor is selected from AMP-224 (Amplimmune) or rHIgM12B7.

In some embodiments, the immune checkpoint inhibitor is an inhibitor of PD1. In some embodiments, the inhibitor of PDL1 is an antibody (e.g., a monoclonal antibody or fragments, or a humanized or chimeric antibody or fragments thereof) against PD-1. Exemplary antibodies against PD-1 include: anti-mouse PD-1 antibody Clone J43 (Cat #BE0033-2) from BioXcell; anti-mouse PD-1 antibody Clone RMP1-14 (Cat #BE0146) from BioXcell; mouse anti-PD-1 antibody Clone EH12; Merck's MK-3475 anti-mouse PD-1 antibody (Keytruda, pembrolizumab, lambrolizumab); and AnaptysBio's anti-PD-1 antibody, known as ANBO11; antibody MDX-1 106 (ONO-4538); Bristol-Myers Squibb's human IgG4 monoclonal antibody nivolumab (Opdivo®, BMS-936558, MDX1106); AstraZeneca's AMP-514, and AMP-224; and Pidilizumab (CT-011), CureTech Ltd.

In some embodiments, an oncolytic virus comprises a nucleic acid sequence that encodes an engager molecule and an additional nucleic acid sequence that encodes a PD1 inhibitor selected from ANBO11; antibody MDX-1106 (ONO-4538); Bristol-Myers Squibb's human IgG4 monoclonal antibody nivolumab (Opdivo®, BMS-936558, MDX1106); AstraZeneca's AMP-514, and AMP-224; and Pidilizumab (CT-011).

In some embodiments, the immune checkpoint inhibitor is an inhibitor of CTLA-4. In some embodiments, the inhibitor of CTLA-4 is an antibody (e.g., a monoclonal antibody or fragments, or a humanized or chimeric antibody or fragments thereof) against CTLA-4. In one embodiment, the anti-CTLA-4 antibody blocks the binding of CTLA-4 to CD80 (B7-1) and/or CD86 (B7-2) expressed on antigen presenting cells. Exemplary antibodies against CTLA-4 include ipilimumab (also known as Yervoy®, MDX-010, BMS-734016 and MDX-101, Bristol Meyers Squibb); anti-CTLA4 antibody clone 9H10 from Millipore; tremelimumab (CP-675,206, ticilimumab, Pfizer); and anti-CTLA4 antibody clone BNI3 from Abcam.

In some embodiments, the anti-CTLA-4 antibody is one disclosed in any of International PCT Publication Nos. WO 2001/014424; WO 2004/035607; WO 2003/086459; WO 2012/120125; WO 2000/037504; WO 2009/100140; WO 2006/09649; WO 2005/092380; WO 2007/123737; WO 2006/029219; WO 2010/0979597; WO 2006/12168; WO 1997/020574 U.S. Patent Application Publication No. 2005/0201994; or European Patent Application Publication No. EP 1212422. Additional CTLA-4 antibodies are described in U.S. Pat. Nos. 5,811,097; 5,855,887; 5,977,318; 6,051,227; 6,682,736; 6,984,720; 7,109,003; 7,132,281; International PCT Publication Nos. WO 01/14424 and WO 00/37504; and in U.S. Patent Application Publication Nos. 2002/0039581 and 2002/086014. In some embodiments, the anti-CTLA-4 antibody is one disclosed in any of International PCT Publication Nos. WO 1998/42752; U.S. Pat. Nos. 6,682,736 and 6,207,156; Hurwitz et al, Proc. Natl. Acad. Sci. USA, 95(17): 10067-10071 (1998); Camacho et al, J Clin Oncol, 22(145): Abstract No. 2505 (2004) (antibody CP-675206); Mokyr et al, Cancer Res., 58:5301-5304 (1998). In some embodiments, the CTLA-4 inhibitor is a CTLA-4 ligand as disclosed in International PCT Publication No. WO 1996/040915.

In some embodiments, the CTLA-4 inhibitor is a nucleic acid inhibitor of CTLA-4 expression, such as an RNAi molecule. In some embodiments, anti-CTLA4 RNAi molecules take the form of those described in any of International PCT Publication Nos. WO 1999/032619 and WO 2001/029058; U.S. Patent Application Publication Nos. 2003/0051263, 2003/0055020, 2003/0056235, 2004/265839, 2005/0100913, 2006/0024798, 2008/0050342, 2008/0081373, 2008/0248576, and 2008/055443; and/or U.S. Pat. Nos. 6,506,559; 7,282,564; 7,538,095; and 7,560,438. In some instances, the anti-CTLA4 RNAi molecules are double stranded RNAi molecules, such as those disclosed in European Patent No. EP 1309726. In some instances, the anti-CTLA4 RNAi molecules are double stranded RNAi molecules, such as those described in U.S. Pat. Nos. 7,056,704 and 7,078,196. In some embodiments, the CTLA4 inhibitor is an aptamer, such as those described in International PCT Publication No. WO 2004/081021, such as Del 60 or M9-14 del 55. Additionally, in some embodiments, the anti-CTLA4 RNAi molecules of the present invention are RNA molecules, such as those described in U.S. Pat. Nos. 5,898,031, 6,107,094, 7,432,249, and 7,432,250, and European Application No. EP 0928290.

In some embodiments, an oncolytic virus comprises a nucleic acid sequence that encodes an engager molecule, and an additional nucleic acid sequence that encodes a CTLA-4 inhibitor. In some embodiments, the CTLA-4 inhibitor is selected from ipilimumab (also known as Yervoy®, MDX-010, BMS-734016 and MDX-101); anti-CTLA4 Antibody, clone 9H10 from Millipore; Pfizer's tremelimumab (CP-675,206, ticilimumab); and anti-CTLA4 antibody clone BNI3 from Abcam.

In some embodiments, the immune checkpoint inhibitor is an inhibitor of LAG3 (CD223). In some embodiments, the inhibitor of LAG3 is an antibody (e.g., a monoclonal antibody or fragments, or a humanized or chimeric antibody or fragments thereof) against LAG3. In additional embodiments, an antibody against LAG3 blocks the interaction of LAG3 with major histocompatibility complex (MHC) class II molecules. Exemplary antibodies against LAG3 include: anti-Lag-3 antibody clone eBioC9B7W (C9B7W) from eBioscience; anti-Lag3 antibody LS-B2237 from LifeSpan Biosciences; IMP321 (ImmuFact) from Immutep; anti-Lag3 antibody BMS-986016; and the LAG-3 chimeric antibody A9H12. In some embodiments, the anti-LAG3 antibody is an anti-LAG3 antibody disclosed in International PCT Publication Nos. WO 2010/019570; WO 2008/132601; or WO 2004/078928.

In some embodiments, an oncolytic virus comprises a nucleic acid sequence that encodes an engager molecule and an additional nucleic acid sequence that encodes LAG3 inhibitor. In some embodiments, the LAG3 inhibitor is selected from anti-Lag-3 antibody clone eBioC9B7W (C9B7W) from eBioscience; anti-Lag3 antibody LS-B2237 from LifeSpan Biosciences; IMP321 (ImmuFact) from Immutep; anti-Lag3 antibody BMS-986016; and the LAG-3 chimeric antibody A9H12.

In some embodiments, the immune checkpoint inhibitor is an inhibitor of TIM3. In some embodiments, the inhibitor of TIM3 is an antibody (e.g., a monoclonal antibody or fragments, or a humanized or chimeric antibody or fragments thereof) against TIM3 (also known as HAVCR2). In additional embodiments, an antibody against TIM3 blocks the interaction of TIM3 with galectin-9 (Gal9). In some embodiments, the anti-TIM3 antibody is an anti-TIM3 antibody disclosed in International PCT Publication Nos. WO 2013/006490; WO 2011/55607; WO 2011/159877; or WO 2001/17057. In another embodiment, a TIM3 inhibitor is a TIM3 inhibitor disclosed in International PCT Publication No. WO 2009/052623. In some embodiments, an oncolytic virus comprises a nucleic acid sequence that encodes an engager molecule and an additional nucleic acid sequence that encodes TIM3 inhibitor. In some embodiments, the TIM3 inhibitor is an antibody that blocks the interaction of TIM3 with Gal9.

In some embodiments, the immune checkpoint inhibitor is an inhibitor of B7-H3. In some embodiments, the inhibitor of B7-H3 is an antibody (e.g., a monoclonal antibody or fragments, or a humanized or chimeric antibody or fragments thereof) against B7-H3. In some embodiments, the inhibitor of B7-H3 is MGA271 (MacroGenics). In some embodiments, an oncolytic virus comprises a nucleic acid sequence that encodes an engager molecule and an additional nucleic acid sequence that encodes a B7-H3 inhibitor. In some embodiments, the B7-H3 inhibitor is MGA271.

In some embodiments, the immune checkpoint polypeptide is an agonist of a cell surface molecule expressed on an effector cell (i.e., an immune checkpoint activator). In some embodiments, immune checkpoint activators include antibodies, antibody-derivatives (e.g., Fab fragments, scFvs, minobodies, diabodies), antisense oligonucleotides, siRNA, aptamers, or peptides. In some embodiments, the immune checkpoint activator activates a cell surface molecule selected from 2B4, CD16 (FcγRIII), CD28, CD30, CD40, CD80, CD86, CD137, CD160, CD226, CD276, DR3, GITR, inducible T cell costimulatory (ICOS), macrophage receptor with collageneous structure (MARCO), OX-40, phosphatidylserine (PS) receptor, SLAM receptors (including CD2), and CD16 (FcγRIII). In some embodiments, an oncolytic virus comprises a nucleic acid sequence that encodes an engager molecule and an additional nucleic acid sequence that encodes an immune checkpoint activator. In some embodiments, the immune checkpoint activator increases the expression or activity of one or more activating cell surface molecules. In some embodiments, the immune activator activates one or more of 2B4, CD16 (FcγRIII), CD28, CD30, CD40, CD80, CD86, CD137, CD160, CD226, CD276, DR3, GITR, ICOS, MARCO, OX-40, PS receptor, SLAM receptors (including CD2), and CD16 (FcγRIII).

### Anti-Angiogenic Factors as Therapeutic Molecules

In some embodiments, the therapeutic molecule is a polypeptide such as an anti-angiogenic factor. Angiogenesis or neovascularization is the formation of new microvessels from an established vascular network. In some instances, the angiogenic process involves communications from multiple cell types such as endothelial cells (EC) and circulating endothelial progenitor cells, pericytes, vascular smooth muscle cells, stromal cells, including stem cells, and parenchymal cells. These communications or interactions occur through secreted factors such as VEGF, fibroblast growth factor (FGF), platelet-derived growth factor (PDGF), or angiopoietins. In some instances, an anti-angiogenic factor is a polypeptide that disrupts one or more of the interactions of the cell types: endothelial cells (EC) and circulating endothelial progenitor cells, pericytes, vascular smooth muscle cells, stromal cells, including stem cells, and parenchymal cells. In some instances, an anti-angiogenic factor is a polypeptide that disrupts one or more of the interactions of secreted factors such as VEGF, fibroblast growth factor (FGF), platelet-derived growth factor (PDGF) or angiopoietins.

In other embodiments, provided are oncolytic viruses comprising nucleic acids that encode therapeutic polypeptides that modulate regulatory T cells. In some instances, regulatory T cells maintain the tolerance to self-antigens and in some instances abrogate autoimmune. In some cases, Treg supresses or downregulates induction and proliferation of effector T cells. Exemplary Treg modulatory polypeptides include CCR4, Helios, TIGIT, GITR, neuropilin, neuritin, CD103, CTLA-4, ICOS, and Swap70.

In other embodiments, provided are oncolytic viruses comprising nucleic acids that encode therapeutic polypeptides that modulate myeloid-derived suppressor cells (MDSCs). MDSCs are a heterogenous population of immune cells from the myeloid lineage (a cluster of different cell types that originate from bone marrow stem cells), to which also includes dendritic cells, macrophages and neutrophils. In some instances, myeloid cells interact with T cells to regulate the T cell's function. Exemplary MDSC modulatory polypeptides include TGF-βR1, GM-CSF, IFN-γ, Interleukins (e.g., IL-β, IL-1F2, IL-6, IL-10, IL-12, IL-13, IL-6, IL-6Rα, IL-6/IL-6R complex, TGF-β1, M-CSF, Prostaglandin E2/PGE2, Prostaglandin E Synthase 2, S100A8, and VEGF.

In other embodiments, provided are oncolytic viruses comprising nucleic acids that encode therapeutic polypeptides that modulate the fibrotic stroma. In some embodiments, fibrosis occurs in response to inflammation, either chronic or recurrent. Over time, the repeated bouts of inflammation irritate and scar the tissue, causing buildups of fibrous tissue. In some instances, if enough fibrous material develops, it turns into stromal fibrosis.

### Nucleic Acid Polymers as Therapeutic Molecules

In some embodiments, the therapeutic molecule is a nucleic acid polymer. In some instances, the nucleic acid polymer is a RNA polymer. In some instances, the RNA polymer is an antisense polymer those sequence is complementary to a microRNA (miRNA or miR) target sequence. In some instances, the RNA polymer is a microRNA polymer. In some embodiments, the RNA polymer comprises a DNA-directed RNAi (ddRNAi) sequence, which enables in vivo production of short hairpin RNAs (shRNAs).

In some embodiments, a microRNA polymer is a short non-coding RNA that is expressed in different tissue and cell types which suppresses the expression of a target gene. For example, miRNAs are transcribed by RNA polymerase II as part of the capped and polyadenylated primary transcripts (pri-miRNAs). In some instances, the primary transcript is cleaved by the Drosha ribonuclease III enzyme to produce an approximately 70-nt stem-loop precursor miRNA (pre-miRNA), which is further cleaved by the cytoplasmic Dicer ribonuclease to generate the mature miRNA and antisense miRNA star (miRNA*) products. In some instances, the mature miRNA is incorporated into a RNA-induced silencing complex (RISC), which recognizes target mRNAs through imperfect base pairing with the miRNA and in some instances results in translational inhibition or destabilization of the target mRNA.

In some instances, dysregulated microRNA expression is correlated with one or more types of cancer. In some embodiments, the microRNA is referred to as an oncomiR. In some instances, the dysregulated microRNA expression is an elevated expression. In some instances, the elevated expression level of microRNA correlates to one or more types of cancer. For example, overexpression of microRNA-155 (miR-155) has been observed in cancers such as Burkitt lymphoma, or laryngeal squamous cell carcinoma (LSCC) and overexpression of microRNA-21 (miR-21) has been observed in breast cancer. In some embodiments, exemplary microRNAs with an elevated expression level include, but are not limited to, miR-10 family (e.g., miR-10b), miR-17, miR-21, miR-106 family (e.g., miR-106a), miR-125 family (e.g., miR-125b), miR-145, miR-146 family (e.g., miR-146a, miR-146b), miR-155, miR-96, miR-182, miR-183, miR-221, miR-222, and miR-1247-5p.

In some instances, the nucleic acid polymer is an antisense polymer those sequence complements an oncomiR. In some instances, the nucleic acid polymer is an antisense polymer those sequence complements an oncomiR that is characterized with an overexpression. In some instances, the nucleic acid polymer is an antisense polymer those sequence complements a microRNA target sequence. In some instances, the nucleic acid polymer is an antisense polymer those sequence complements a microRNA target sequence that is characterized with an overexpression. In some instances, the therapeutic molecule is an antisense polymer those sequence complements a microRNA target sequence. In some instances, the therapeutic molecule is an antisense polymer those sequence complements a microRNA target sequence that is characterized with an overexpression. In some instances, the overexpression level is relative to the endogenous expression level of the microRNA.

In some instances, the dysregulated microRNA expression is a reduced expression. In some instances, the reduced expression level of microRNA correlates to one or more types of cancer. For example, a depleted level of miR-31 has been observed in both human and mouse metastatic breast cancer cell lines. In some embodiments, exemplary microRNAs with reduced expression levels include, but are not limited to, miR-31, miR-34 family (e.g., miR34a, miR-34b, and miR-34c), miR-101, miR-126, miR-145, miR-196a, and the miR-200 family.

In some instances, the nucleic acid polymer is an oncomiR. In some instances, the oncomiR is equivalent to an endogeous oncomiR wherein the endogeous oncomiR is characterized with a reduced expression level. In some instances, the nucleic acid polymer is a microRNA polymer. In some instances, the therapeutic molecule is a microRNA polymer. In some instances, the microRNA is equivalent to an endogeous microRNA polymer wherein the endogenous microRNA is characterized with a reduced expression level.

As described above, in some instances the RNA polymer comprises a DNA-directed RNAi (ddRNAi) sequence. In some instances, a ddRNAi construct encoding a shRNA is packaged into a viral vector such as a viral vector of a oncolytic virus described herein. In some instances upon entry into the target cell (e.g., a tumor cell), the viral genome is processed to produce the encoded shRNAs. The shRNAs are then processed by endogenous host systems and enter the RNAi pathway to modulate or silence the desired gene target. In some instances, the gene target is a gene that is overexpressed in a cancer type. In some instances, the gene target is a gene that is overexpressed in a solid tumor. In some instances, the gene target is a gene that is overexpressed in a hematologic cancer. Exemplary genes that are overexpressed in cancer include, but are not limited to, TP53, human epidermal growth factor receptor 2 (HER2), mucin 1-cell surface associated (MIUC1), human pituitary tumour-transforming gene 1 (hPPTG1), prostate and breast cancer overexpressed gene 1 protein (PBOVI), and the like.

In some instances, the nucleic acid polymer comprises a ddRNAi sequence. In some instances, the nucleic acid polymer is comprises a ddRNAi sequence which targets a gene that is overexpressed in a cancer. In some instances, the therapeutic molecule comprises a ddRNAi sequence. In some instances, the therapeutic molecule comprises a ddRNAi sequence which targets a gene that is overexpressed in a cancer.

### Exemplarily Oncolytic Viruses and Engager Molecules

In some embodiments, the oncolytic viruses described herein comprise a polynucleotide sequence encoding an engager molecule. In some embodiments, the engager molecule is a bi-specific antibody construct comprising an effector domain and a targeting domain. In some embodiments, the engager molecule is a bi-specific antibody construct comprising an effector domain and an activation domain. In some embodiments, the engager molecule is a tripartite polypeptide comprising (i) an effector domain, (ii) an activation or targeting domain, and (iii) an additional domain e.g., an additional effector domain, an additional activation domain, an additional targeting domain, a therapeutic molecule domain (such as a cytokine, a co-stimulatory domain, an immune checkpoint inhibitor, or an anti-angiogenic factor), or a combination thereof.

In some embodiments, the oncolytic viruses described herein comprise a first polynucleotide sequence encoding an engager molecule and a second polynucleotide encoding a therapeutic molecule. In some embodiments, the first polypeptide and the second polynucleotide sequences are comprised in the same vector (e.g., in the same oncolytic virus). In some embodiments, the first polypeptide and the second polypeptide are encoded in different vectors (e.g., in different oncolytic viruses).

In some embodiments, the oncolytic viruses described herein comprise a first polynucleotide sequence encoding an engager molecule comprising an effector domain and a targeting domain. In some such embodiments, the effector domain binds to one or more components of CD3. In some such embodiments, the targeting domain binds to a target antigen selected from FAP and CD19. In some embodiments, the oncolytic viruses described herein comprise a first polynucleotide sequence encoding an engager molecule comprising an effector domain and an activation domain. In some such embodiments, the effector domain binds to one or more components of CD3. In some such embodiments, the activation domain binds to a cell-surface molecule selected from CD47, PD1, and PDL1. In some embodiments, the oncolytic viruses described herein comprise a first polynucleotide sequence encoding an engager molecule comprising an effector domain and a targeting or activation domain and a second polynucleotide sequence encoding a therapeutic molecule. In some such embodiments, the therapeutic molecule is selected from a cytokine, a co-stimulatory domain, and an immune checkpoint inhibitor. In some embodiments, the cytokine is selected from IL-15, IL-2, IL-7, IL-12, CCL4, and CXCL10. In some embodiments, the co-stimulatory molecule is selected from a molecule that binds to and activates a cell-surface molecule selected from CD27, CD80, CD83, CD86, CD134, or CD137. In some embodiments, the immune checkpoint inhibitor molecule is selected from a molecule that binds to and inhibits a cell surface molecule selected from PD-1, PDL1, CTLA4, and B7-H4.

In some embodiments, the oncolytic viruses described herein comprise a polynucleotide sequence encoding an engager molecule comprising an effector domain, wherein the effector domain comprises an anti-CD3 scFv. In some embodiments, the anti-CD3 scFv comprises a light chain variable fragment comprising an amino acid sequence that is at least 80%, at least, 85%, at least 90%, at least 95%, at least 96%, at least 97%, at least 98%, or at least 99% identical to SEQ ID NO: 20 or 77 and a heavy chain variable fragment comprising an amino acid sequence that is at least 80%, at least, 85%, at least 90%, at least 95%, at least 96%, at least 97%, at least 98%, or at least 99% identical to SEQ ID NO: 22 or 76. In some embodiments, the anti-CD3 scFv comprises a light chain variable fragment comprising an amino acid sequence that is 100% identical to SEQ ID NO: 20 or 77 and a heavy chain variable fragment that is 100% identical to SEQ ID NO: 22 or 76. In some embodiments, the anti-CD3 scFv comprises a light chain variable fragment comprising SEQ ID NO: 20 or 77 and a heavy chain variable fragment comprising SEQ ID NO: 22 or 76. In some embodiments, the anti-CD3 scFv comprises a light chain variable fragment consisting of SEQ ID NO: 20 or 77 and a heavy chain variable fragment consisting of SEQ ID NO: 22 or 76.

In some embodiments, the anti-CD3 scFv comprises a light chain variable fragment and a heavy chain variable fragment, wherein the nucleic acid sequence encoding the light chain variable fragment is at least 80%, at least, 85%, at least 90%, at least 95%, at least 96%, at least 97%, at least 98%, or at least 99% identical to SEQ ID NO: 19 and wherein the nucleic acid sequence encoding the heavy chain variable fragment is at least 80%, at least, 85%, at least 90%, at least 95%, at least 96%, at least 97%, at least 98%, or at least 99% identical to SEQ ID NO: 21. In some embodiments, the nucleic acid sequence encoding the light chain variable fragment is 100% identical to the nucleic acid sequence of SEQ ID NO: 19 and the nucleic acid sequence encoding the heavy chain variable fragment is 100% identical SEQ ID NO: 21. In some embodiments, the nucleic acid sequence encoding the light chain variable fragment comprises SEQ ID NO: 19 and the nucleic acid sequence encoding the heavy chain variable fragment comprises SEQ ID NO: 21. In some embodiments, the nucleic acid sequence encoding the light chain variable fragment consists of SEQ ID NO: 19 and the nucleic acid sequence encoding the heavy chain variable fragment consists of SEQ ID NO: 21.

In some embodiments, the oncolytic viruses described herein comprise a polynucleotide sequence encoding an engager molecule comprising a targeting domain, wherein the targeting domain comprises an anti-CD19 scFv. In some embodiments, the anti-CD19 scFv comprises a light chain variable fragment comprising an amino acid sequence that is at least 80%, at least, 85%, at least 90%, at least 95%, at least 96%, at least 97%, at least 98%, or at least 99% identical to SEQ ID NO: 16 and a heavy chain variable fragment comprising an amino acid sequence that is at least 80%, at least, 85%, at least 90%, at least 95%, at least 96%, at least 97%, at least 98%, or at least 99% identical to SEQ ID NO: 18. In some embodiments, the anti-CD19 scFv comprises a light chain variable fragment comprising an amino acid sequence that is 100% identical to SEQ ID NO: 16 and a heavy chain variable fragment that is 100% identical to SEQ ID NO: 18. In some embodiments, the anti-CD19 scFv comprises a light chain variable fragment comprising SEQ ID NO: 16 and a heavy chain variable fragment comprising SEQ ID NO: 18. In some embodiments, the anti-CD19 scFv comprises a light chain variable fragment consisting of SEQ ID NO: 16 and a heavy chain variable fragment consisting of SEQ ID NO: 18.

In some embodiments, the anti-CD19 scFv comprises a light chain variable fragment and a heavy chain variable fragment, wherein the nucleic acid sequence encoding the light chain variable fragment is at least 80%, at least, 85%, at least 90%, at least 95%, at least 96%, at least 97%, at least 98%, or at least 99% identical to SEQ ID NO: 15 and the nucleic acid sequence encoding the heavy chain variable fragment is at least 80%, at least, 85%, at least 90%, at least 95%, at least 96%, at least 97%, at least 98%, or at least 99% identical to SEQ ID NO: 17. In some embodiments, the the nucleic acid sequence encoding the light chain variable fragment is 100% identical to SEQ ID NO: 15 and the nucleic acid sequence encoding the heavy chain variable fragment is 100% identical to SEQ ID NO: 17. In some embodiments, the the nucleic acid sequence encoding the light chain variable fragment nucleic acid sequence comprises SEQ ID NO: 15 and the nucleic acid sequence encoding the heavy chain variable fragment comprises SEQ ID NO: 17. In some embodiments, the nucleic acid sequence encoding the light chain variable fragment nucleic acid sequence consists of SEQ ID NO: 15 and the nucleic acid sequence encoding the heavy chain variable fragment nucleic acid sequence consists of SEQ ID NO: 17.

In some embodiments, the oncolytic viruses described herein comprise a polynucleotide sequence encoding an engager molecule comprising a targeting domain, wherein the targeting domain comprises an anti-FAP scFv. In some embodiments, the anti-FAP scFv comprises a light chain variable fragment comprising an amino acid sequence that is at least 80%, at least, 85%, at least 90%, at least 95%, at least 96%, at least 97%, at least 98%, or at least 99% identical to SEQ ID NO: 71 and a heavy chain variable fragment comprising an amino acid sequence that is at least 80%, at least, 85%, at least 90%, at least 95%, at least 96%, at least 97%, at least 98%, or at least 99% identical to SEQ ID NO: 72. In some embodiments, the anti-FAP scFv comprises a light chain variable fragment comprising an amino acid sequence that is 100% identical to SEQ ID NO: 71 and a heavy chain variable fragment that is 100% identical to SEQ ID NO: 72. In some embodiments, the anti-FAP scFv comprises a light chain variable fragment comprising the amino acid sequence of SEQ ID NO: 71 and a heavy chain variable fragment comprising the amino acid sequence of SEQ ID NO: 72. In some embodiments, the anti-FAP scFv comprises a light chain variable fragment consisting of the amino acid sequence of SEQ ID NO: 71 and a heavy chain variable fragment consisting of the amino acid sequence of of SEQ ID NO: 72. In some embodiments, the amino acid sequence of the anti-FAP scFv is at least 80%, at least, 85%, at least 90%, at least 95%, at least 96%, at least 97%, at least 98%, or at least 99% identical to SEQ ID NO: 75. In some embodiments, the amino acid sequence of the anti-FAP scFv is 100% identical to SEQ ID NO: 75. In some embodiments, the amino acid sequence of the anti-FAP scFv comprises SEQ ID NO: 75. In some embodiments, the amino acid sequence of the anti-FAP scFv consists of SEQ ID NO: 75.

In some embodiments, the polynucleotide sequence encoding the anti-FAP scFv comprises a nucleic acid sequence that is at least 80%, at least, 85%, at least 90%, at least 95%, at least 96%, at least 97%, at least 98%, or at least 99% identical to SEQ ID NO: 74. In some embodiments, the polynucleotide sequence encoding the anti-FAP scFv comprises a nucleic acid sequence that is 100% identical to SEQ ID NO: 74. In some embodiments, the polynucleotide sequence encoding the anti-FAP scFv comprises SEQ ID NO: 74. In some embodiments, the polynucleotide sequence encoding the anti-FAP scFv consists of SEQ ID NO: 74.

In some embodiments, the oncolytic viruses described herein comprise a polynucleotide sequence encoding an engager molecule comprising an activation domain, wherein the activation domain comprises an anti-PDL1 scFv. In some embodiments, the anti-PDL1 scFv comprises a light chain variable fragment comprising an amino acid sequence that is at least 80%, at least, 85%, at least 90%, at least 95%, at least 96%, at least 97%, at least 98%, or at least 99% identical to SEQ ID NO: 36 and a heavy chain variable fragment comprising an amino acid sequence that is at least 80%, at least, 85%, at least 90%, at least 95%, at least 96%, at least 97%, at least 98%, or at least 99% identical to SEQ ID NO: 38. In some embodiments, the anti-PDL1 scFv comprises a light chain variable fragment comprising an amino acid sequence that is 100% identical to SEQ ID NO: 36 and a heavy chain variable fragment that is 100% identical to SEQ ID NO: 38. In some embodiments, the anti-PDL1 scFv comprises a light chain variable fragment comprising the amino acid sequence of SEQ ID NO: 36 and a heavy chain variable fragment comprising the amino acid sequence of SEQ ID NO: 38. In some embodiments, the anti-PDL1 scFv comprises a light chain variable fragment consisting of the amino acid sequence of SEQ ID NO: 36 and a heavy chain variable fragment consisting of the amino acid sequence of of SEQ ID NO: 38.

In some embodiments, the anti-PDL1 scFv comprises a light chain variable fragment and a heavy chain variable fragment, wherein the nucleic acid sequence encoding the light chain variable fragment is at least 80%, at least, 85%, at least 90%, at least 95%, at least 96%, at least 97%, at least 98%, or at least 99% identical to SEQ ID NO: 35 and the nucleic acid sequence encoding the heavy chain variable fragment is at least 80%, at least, 85%, at least 90%, at least 95%, at least 96%, at least 97%, at least 98%, or at least 99% identical to SEQ ID NO: 37. In some embodiments, the nucleic acid sequence encoding the light chain variable fragment nucleic acid sequence is 100% identical to SEQ ID NO: 35 and the nucleic acid sequence encoding the heavy chain variable fragment is 100% identical to SEQ ID NO: 37. In some embodiments, the nucleic acid sequence encoding the light chain variable fragment comprises SEQ ID NO: 35 and the nucleic acid sequence encoding the heavy chain variable fragment comprises SEQ ID NO: 37. In some embodiments, the nucleic acid sequence encoding the light chain variable fragment consists of SEQ ID NO: 35 and the nucleic acid sequence encoding the heavy chain variable fragment nucleic acid sequence consists of SEQ ID NO: 37.

In some embodiments, the oncolytic viruses described herein comprise a polynucleotide sequence encoding an engager molecule comprising an activation domain comprising a SIRP1α polypeptide fragment. In some embodiments, the SIRP1α polypeptide fragment comprises an amino acid sequence that is at least 80%, at least, 85%, at least 90%, at least 95%, at least 96%, at least 97%, at least 98%, or at least 99% identical to SEQ ID NO: 32. In some embodiments, the SIRP1α polypeptide fragment comprises an amino acid sequence that is 100% identical to SEQ ID NO: 32. In some embodiments, the SIRP1α polypeptide fragment comprises SEQ ID NO: 32. In some embodiments, the SIRP1α polypeptide fragment consists of SEQ ID NO: 32.

In some embodiments, the nucleic acid sequence encoding the SIRP1α polypeptide fragment is at least 80%, at least, 85%, at least 90%, at least 95%, at least 96%, at least 97%, at least 98%, or at least 99% identical to SEQ ID NO: 31. In some embodiments, the nucleic acid sequence encoding the SIRP1α polypeptide fragment is 100% identical to SEQ ID NO: 31. In some embodiments, the nucleic acid sequence encoding the SIRP1α polypeptide fragment comprises SEQ ID NO: 31. In some embodiments, the nucleic acid sequence encoding the SIRP1α polypeptide fragment consists of SEQ ID NO: 31.

In some embodiments, the oncolytic viruses described herein comprise a first nucleic acid sequence encoding an engager molecule and a second nucleic acid sequence encoding a therapeutic molecule. In some embodiments, the therapeutic molecule is IL-15 and comprises an amino acid sequence that is at least 80%, at least 85%, at least 90%, at least 95%, at least 96%, at least 97%, at least 98%, or at least 99% identical to SEQ ID NO: 24. In some embodiments, the therapeutic molecule is IL-15 and comprises an amino acid sequence that is 100% identical to SEQ ID NO: 24. In some embodiments, the therapeutic molecule is IL-15 and comprises the amino acid sequence of SEQ ID NO: 24. In some embodiments, the therapeutic molecule is IL-15 and consists of the amino acid sequence of SEQ ID NO: 24. In some embodiments, the therapeutic molecule is IL-15 and is encoded by a nucleic acid sequence that is at least 80%, at least 85%, at least 90%, at least 95%, at least 96%, at least 97%, at least 98%, or at least 99% identical to SEQ ID NO: 23. In some embodiments, the therapeutic molecule is IL-15 and is encoded by a nucleic acid sequence that 100% identical to SEQ ID NO: 23. In some embodiments, the therapeutic molecule is IL-15 and is encoded by a nucleic acid sequence comprising SEQ ID NO: 23. In some embodiments, the therapeutic molecule is IL-15 and is encoded by a nucleic acid sequence consisting of SEQ ID NO: 23.

In some embodiments, the oncolytic viruses described herein comprise a first nucleic acid sequence encoding an engager molecule and a second nucleic acid sequence encoding a therapeutic molecule. In some embodiments, the therapeutic molecule is IL-12 p35 and/or IL-12 p40 and comprises an amino acid sequence that is at least 80%, at least 85%, at least 90%, at least 95%, at least 96%, at least 97%, at least 98%, or at least 99% identical to SEQ ID NO: 26 or SEQ ID NO: 28, respectively. In some embodiments, the therapeutic molecule is IL-12 p³5 and/or IL-12 p40 and comprises an amino acid sequence that is 100% identical to SEQ ID NO: 26 or SEQ ID NO: 28, respectively. In some embodiments, the therapeutic molecule is IL-12 p³⁵ and/or IL-12 p40 and comprises the amino acid sequence of SEQ ID NO: 26 or SEQ ID NO: 28, respectively. In some embodiments, the therapeutic molecule is IL-12 p35 and/or IL-12 p40 and consists of the amino acid sequence of SEQ ID NO: 26 or SEQ ID NO: 28, respectively. In some embodiments, the therapeutic molecule is IL-12 p³⁵ and/or IL-12 p40 and is encoded by a nucleic acid sequence that is at least 80%, at least 85%, at least 90%, at least 95%, at least 96%, at least 97%, at least 98%, or at least 99% identical to SEQ ID NO: 25 or SEQ ID NO: 27, respectively. In some embodiments, the therapeutic molecule is IL-12 p35 and/or IL-12 p40 and is encoded by a nucleic acid sequence that 100% identical to SEQ ID NO: 25 or SEQ ID NO: 27, respectively. In some embodiments, the therapeutic molecule is IL-12 p35 and/or IL-12 p40 and is encoded by a nucleic acid sequence comprising SEQ ID NO: 25 or SEQ ID NO: 27, respectively. In some embodiments, the therapeutic molecule is IL-12 p35 and/or IL-12 p40 and is encoded by a nucleic acid sequence consisting of SEQ ID NO: 25 or SEQ ID NO: 27, respectively.

In some embodiments, the oncolytic viruses described herein comprise a first nucleic acid sequence encoding an engager molecule and a second nucleic acid sequence encoding a therapeutic molecule. In some embodiments, the therapeutic molecule is CXCL10 and comprises an amino acid sequence that is at least 80%, at least 85%, at least 90%, at least 95%, at least 96%, at least 97%, at least 98%, or at least 99% identical to SEQ ID NO: 30. In some embodiments, the therapeutic molecule is CXCL10 and comprises an amino acid sequence that is 100% identical to SEQ ID NO: 30. In some embodiments, the therapeutic molecule is CXCL10 and comprises the amino acid sequence of SEQ ID NO: 30. In some embodiments, the therapeutic molecule is CXCL10 and consists of the amino acid sequence of SEQ ID NO: 30. In some embodiments, the therapeutic molecule is CXCL10 and is encoded by a nucleic acid sequence that is at least 80%, at least 85%, at least 90%, at least 95%, at least 96%, at least 97%, at least 98%, or at least 99% identical to SEQ ID NO: 29. In some embodiments, the therapeutic molecule is CXCL10 and is encoded by a nucleic acid sequence that 100% identical to SEQ ID NO: XX29X. In some embodiments, the therapeutic molecule is CXCL10 and is encoded by a nucleic acid sequence comprising SEQ ID NO: 29. In some embodiments, the therapeutic molecule is CXCL10 and is encoded by a nucleic acid sequence consisting of SEQ ID NO: 29.

In some embodiments, the oncolytic viruses described herein comprise a first nucleic acid sequence encoding an engager molecule and a second nucleic acid sequence encoding a therapeutic molecule. In some embodiments, the therapeutic molecule is CCL4 and comprises an amino acid sequence that is at least 80%, at least 85%, at least 90%, at least 95%, at least 96%, at least 97%, at least 98%, or at least 99% identical to SEQ ID NO: 80. In some embodiments, the therapeutic molecule is CCL4 and comprises an amino acid sequence that is 100% identical to SEQ ID NO: 80. In some embodiments, the therapeutic molecule is CCL4 and comprises the amino acid sequence of SEQ ID NO: 80. In some embodiments, the therapeutic molecule is CCL4 and consists of the amino acid sequence of SEQ ID NO: 80.

In some embodiments, the oncolytic viruses described herein comprise a first nucleic acid sequence encoding an engager molecule and a second nucleic acid sequence encoding a therapeutic molecule. In some embodiments, the therapeutic molecule is MMP9 and comprises an amino acid sequence that is at least 80%, at least 85%, at least 90%, at least 95%, at least 96%, at least 97%, at least 98%, or at least 99% identical to SEQ ID NO: 34. In some embodiments, the therapeutic molecule is MMP9 and comprises an amino acid sequence that is 100% identical to SEQ ID NO: 34. In some embodiments, the therapeutic molecule is MMP9 and comprises the amino acid sequence of SEQ ID NO: 34. In some embodiments, the therapeutic molecule is MMP9 and consists of the amino acid sequence of SEQ ID NO: 34. In some embodiments, the therapeutic molecule is MMP9 and is encoded by a nucleic acid sequence that is at least 80%, at least 85%, at least 90%, at least 95%, at least 96%, at least 97%, at least 98%, or at least 99% identical to SEQ ID NO: 33. In some embodiments, the therapeutic molecule is MMP9 and is encoded by a nucleic acid sequence that 100% identical to SEQ ID NO: 33. In some embodiments, the therapeutic molecule is MMP9 and is encoded by a nucleic acid sequence comprising SEQ ID NO: 33. In some embodiments, the therapeutic molecule is IMP9 and is encoded by a nucleic acid sequence consisting of SEQ ID NO: 33.

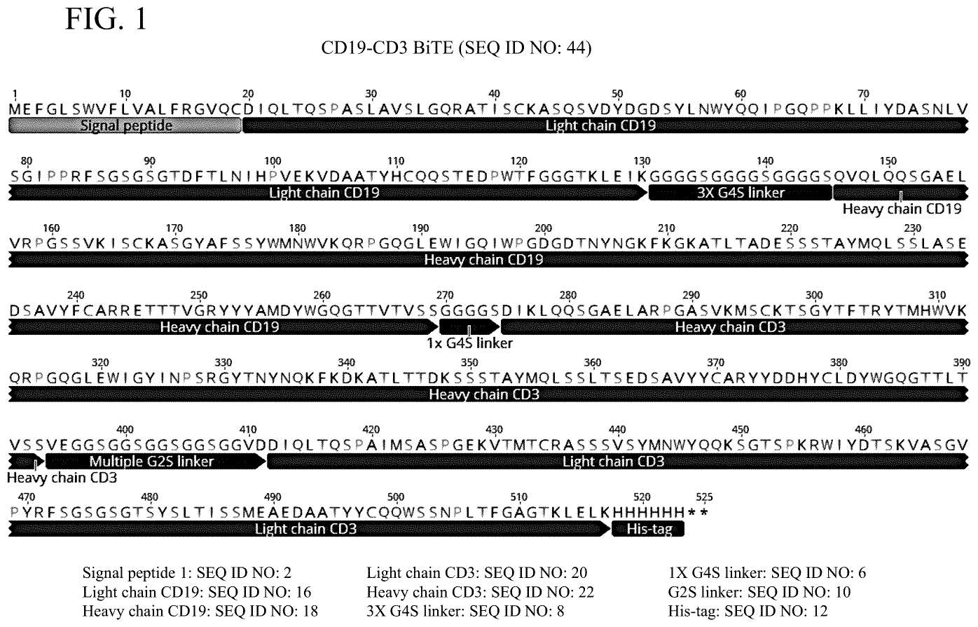

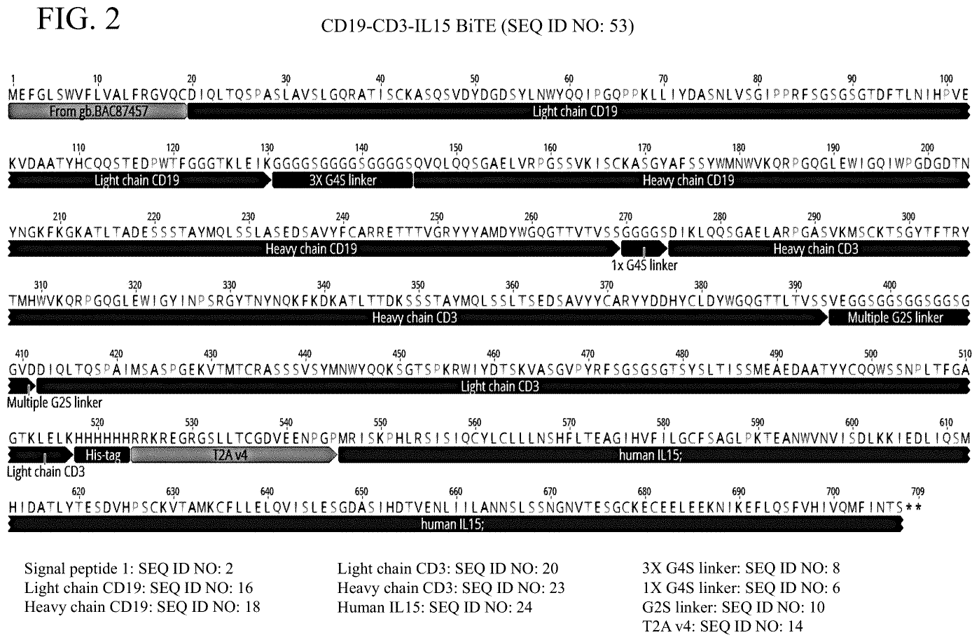

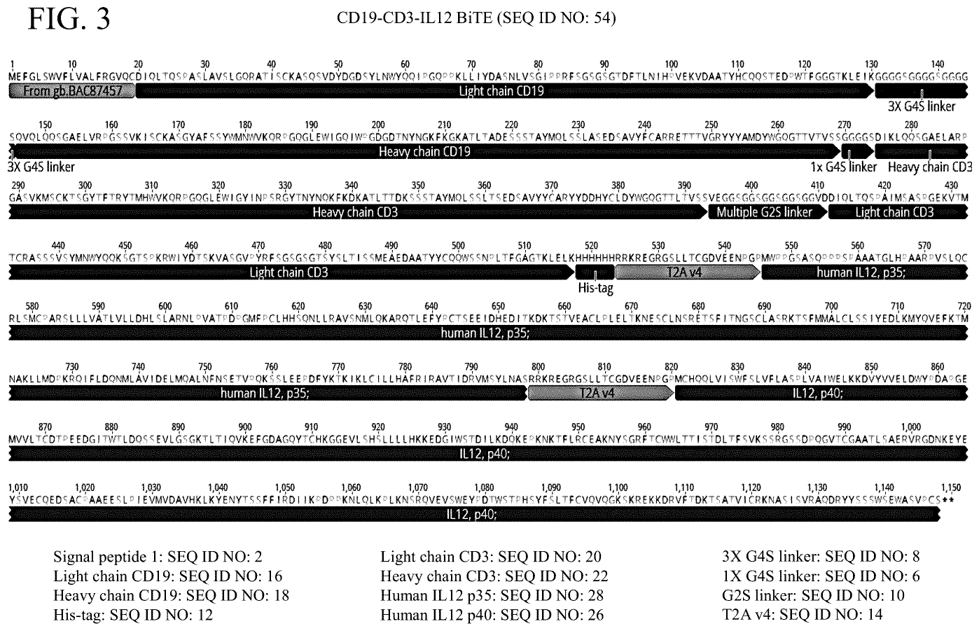

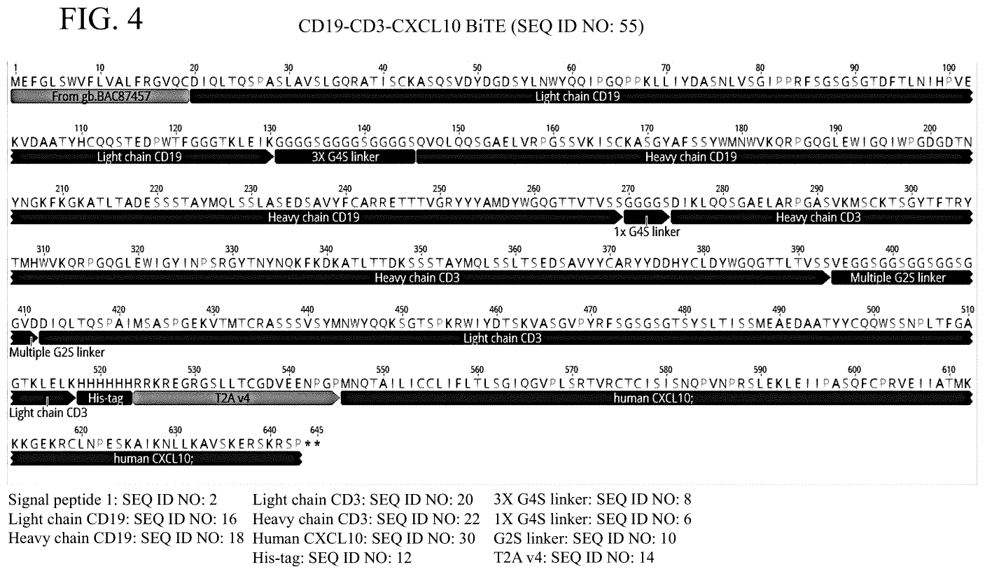

In some embodiments, the oncolytic viruses described herein comprise a polynucleotide sequence encoding an engager molecule comprising an effector domain comprising an scFv that binds to CD3 and targeting domain comprising an scFv that binds to CD19, referred to herein as a CD19-CD3 BiTE, or a CD19 BiTE. A schematic of an exemplary CD19-CD3 BiTE is shown in FIG. 1 (SEQ ID NO: 44). In such embodiments, the anti-CD3 scFv and the anti-CD19 scFv are linked together by a G4S linker (SEQ ID NO: 6). In some embodiments, the oncolytic viruses described herein comprise a first nucleic acid sequence encoding a CD19-CD3 BiTE and a second nucleic acid sequence encoding a therapeutic molecule such as IL-15, IL-12, CXCL10, or CCL4. In some embodiments, the nucleic acid sequences encoding the CD19-CD3 BiTE and the therapeutic molecule are separate nucleic acid sequences and the CD19-CD3 BiTE and the therapeutic molecule are encoded as separate proteins. In some embodiments, the oncolytic viruses described herein comprise a bicistronic or multicistronic nucleic acid sequence, wherein a first nucleic acid sequence encodes a CD19-CD3 BiTE and a second nucleic acid sequence encodes a therapeutic molecule such as IL-15 (FIG. 2, SEQ ID NO: 53), IL-12 (FIG. 3, SEQ ID NO: 54), or CXCL10 (FIG. 4, SEQ ID NO: 55). In such embodiments, the CD19-CD3 BiTE (e.g., SEQ ID NO: 44) is linked to the therapeutic molecule, e.g., IL-15 (SEQ ID NO: 24), IL-12 p35 (SEQ ID NO: 28), IL-12 p40 (SEQ ID NO: 26), and/or CXCL10 (SEQ ID NO: 30), by a T2A self-cleaving peptide linker (SEQ ID NO: 14).

In some embodiments, the oncolytic viruses described herein comprise a polynucleotide sequence encoding an engager molecule comprising an effector domain comprising an scFv that binds to CD3 and an activation domain comprising an scFv that binds to FAP, referred to herein as a FAP-CD3 BiTE, or a FAP BiTE (SEQ ID NO: 75). In such embodiments, the anti-CD3 scFv and the anti-FAP scFv are linked together by a G4S linker (SEQ ID NO: 6). In some embodiments, the oncolytic viruses described herein comprise a first nucleic acid sequence encoding a FAP-CD3 BiTE and a second nucleic acid sequence encoding a therapeutic molecule such as IL-15, IL-12, CXCL10, or CCL4. In some embodiments, the nucleic acid sequences encoding the FAP-CD3 BiTE and the therapeutic molecule are separate nucleic acid sequences and the FAP-CD3 BiTE and the therapeutic molecule are encoded as separate proteins. In some embodiments, the oncolytic viruses described herein comprise a bicistronic or multicistronic nucleic acid sequence, wherein a first nucleic acid sequence encodes a FAP-CD3 BiTE and a second nucleic acid sequence encodes a therapeutic molecule such as IL-15, IL-12, CXCL10, or CCL4. In some such embodiments, the encoded FAP-CD3 BiTE (e.g., SEQ ID NO: 74) is linked to the therapeutic molecule, e.g., IL-15 (SEQ ID NO: 24), IL-12 p35 (SEQ ID NO: 28), IL-12 p40 (SEQ ID NO: 26), CXCL10 (SEQ ID NO: 30), and/or CCL4 (SEQ ID NO: 80) by a T2A self-cleaving peptide linker (SEQ ID NO: 14).

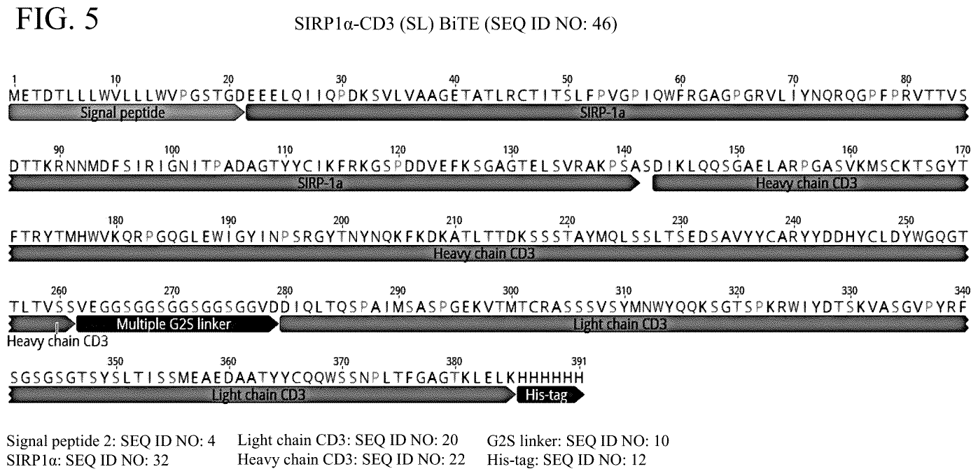

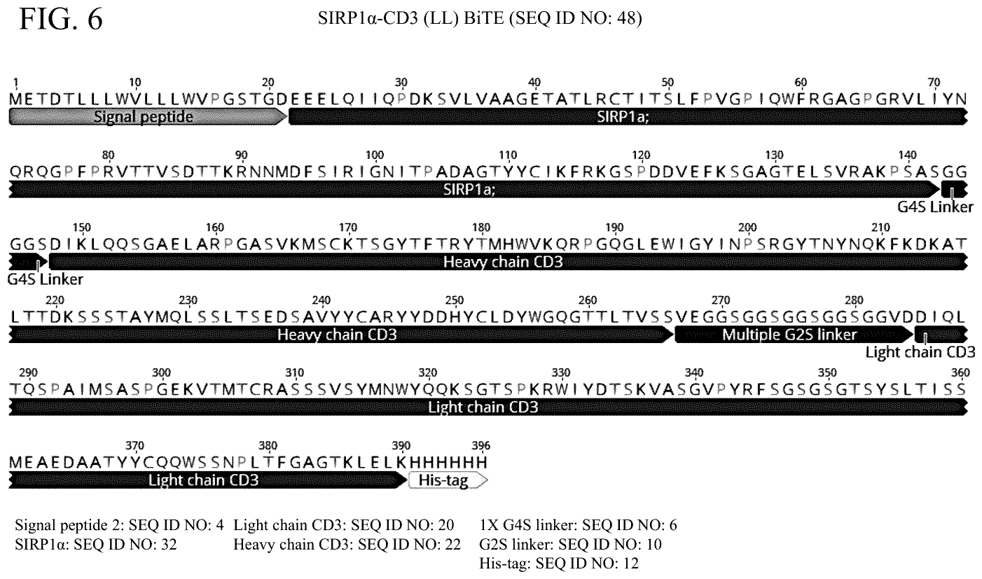

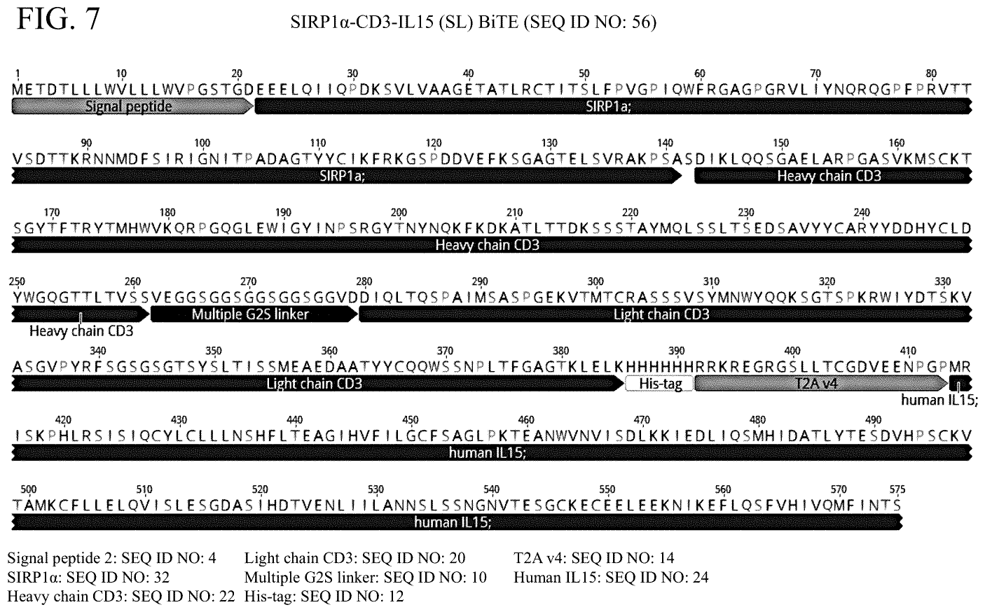

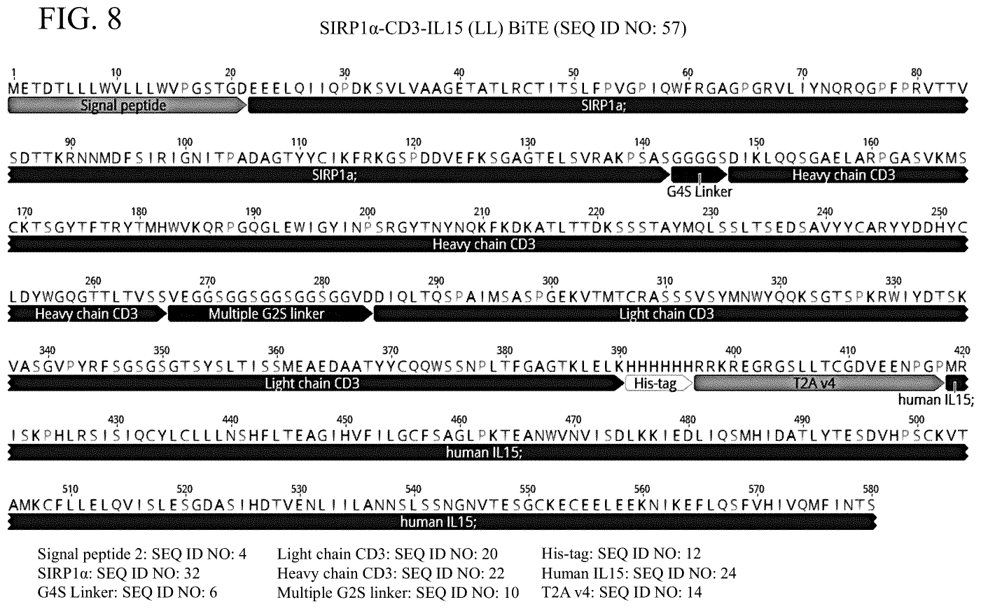

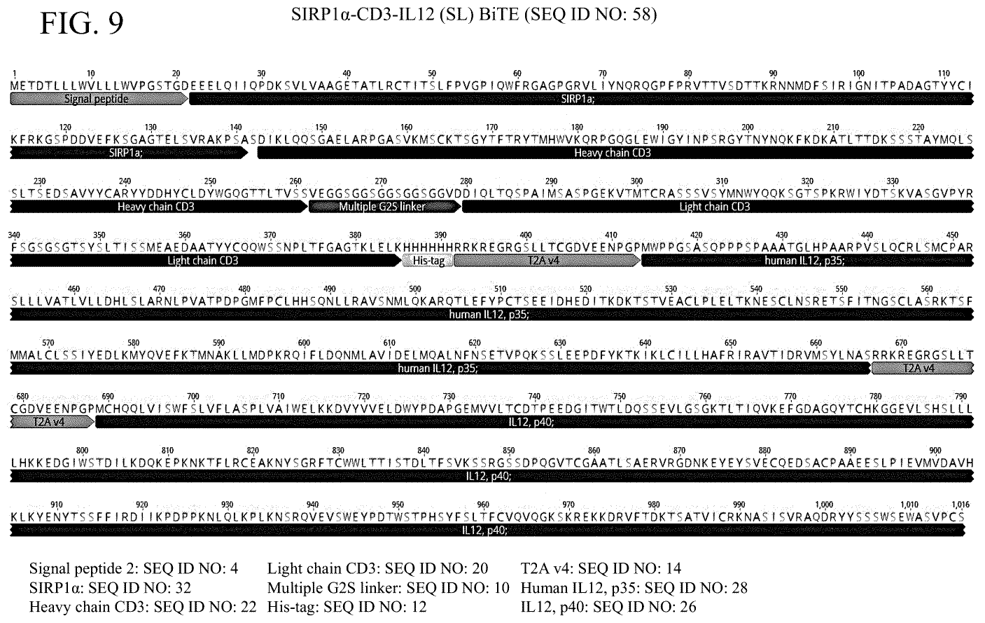

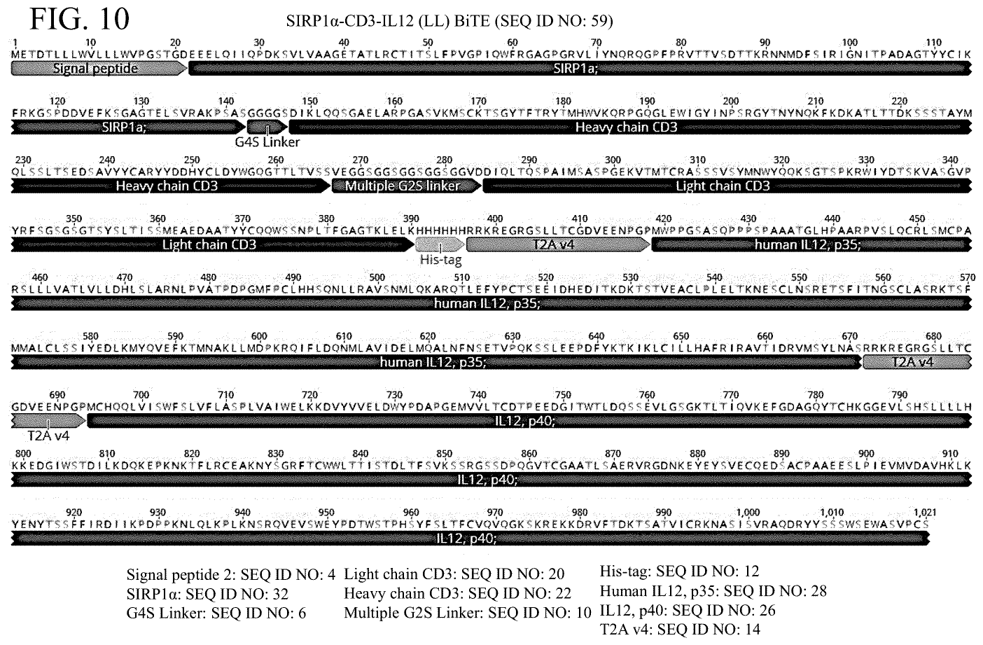

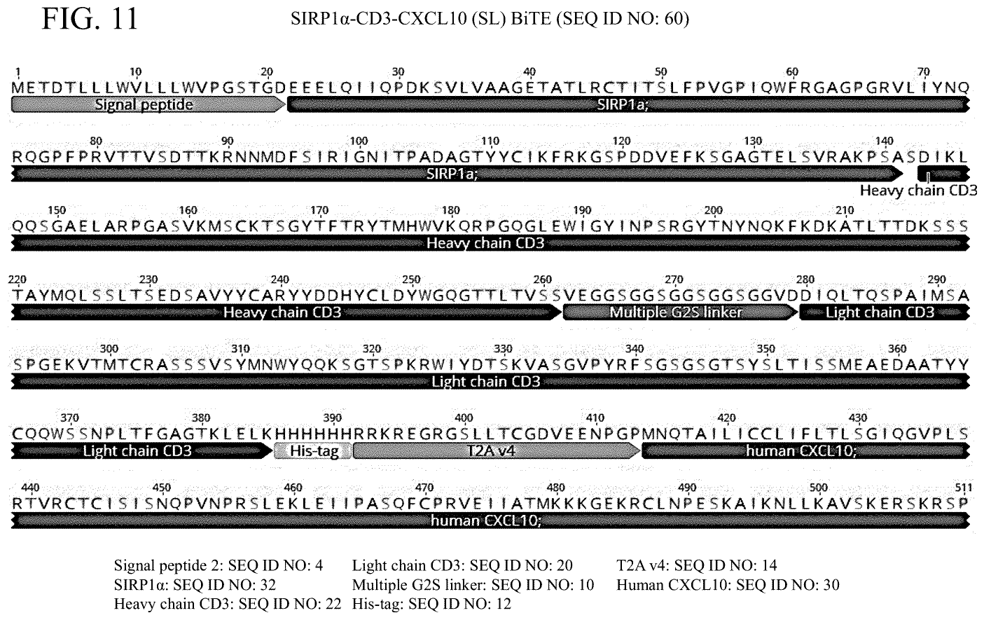

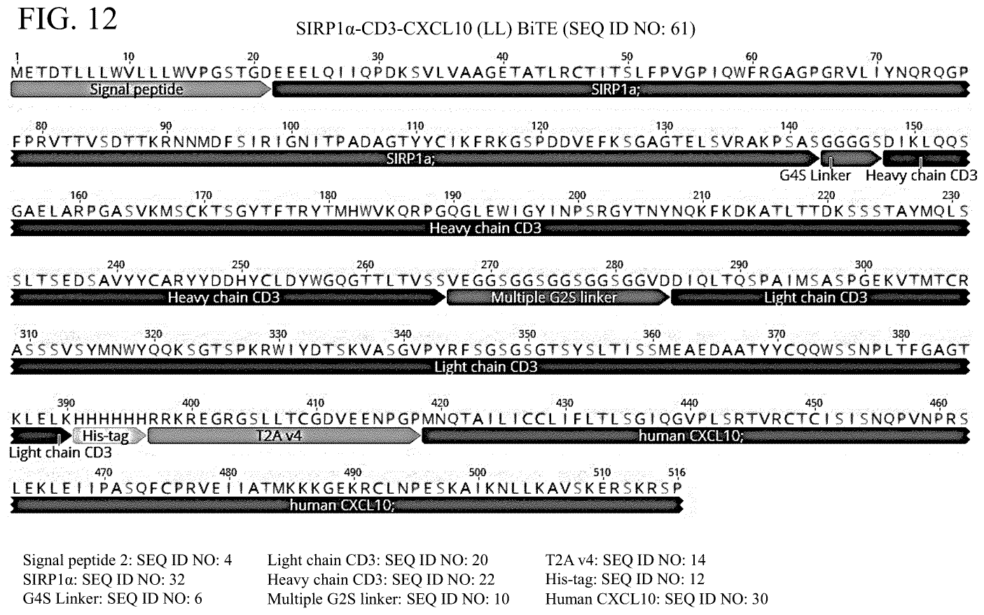

In some embodiments, the oncolytic viruses described herein comprise a polynucleotide sequence encoding an engager molecule comprising an effector domain comprising an scFv that binds to CD3 and an activation domain comprising a SIRP1α polypeptide fragment that binds to CD47 (SEQ ID NO: 32), referred to herein as an SIRP1α-CD3 BiTE or a SIRP1α BiTE. A schematic of an exemplary SIRP1α-CD3 BiTE is shown in FIG. 5 (SIRP1α-CD3 (SL), SEQ ID NO: 46) and FIG. 6 (SIRP1α-CD3 (LL), SEQ ID NO: 48). In some embodiments, the anti-CD3 scFv and the SIRP1α peptide fragment are linked together by a single amino acid linker, or a “short linker” (SL) (e.g., SIRP1α-CD3 (SL) as shown in FIG. 5). In some embodiments, the anti-CD3 scFv and the SIRP1α peptide fragment are linked together by G4S linker, or a “long linker” (LL) (e.g., SIRP1α-CD3 (LL) as shown in FIG. 6). In some embodiments, the oncolytic viruses described herein comprise a first nucleic acid sequence encoding a SIRP1α-CD3 BiTE and a second nucleic acid sequence encoding a therapeutic molecule such as IL-15, IL-12, CXCL10, or CCL4. In some embodiments, the nucleic acid sequences encoding the SIRP1α-CD3 BiTE and the therapeutic molecule are separate nucleic acid sequences and the FAP-CD3 BiTE and the therapeutic molecule are encoded as separate proteins. In some embodiments, the oncolytic viruses described herein comprise a bicistronic or multicistronic nucleic acid sequence, wherein a first nucleic acid sequence encodes a SIRP1α-CD3 BiTE and a second nucleic acid sequence encodes a therapeutic molecule such as IL-15 (FIG. 7, SEQ ID NO: 56 and FIG. 8, SEQ ID NO: 57), IL-12 (FIG. 9, SEQ ID NO: 58 and FIG. 10, SEQ ID NO: 59), or CXCL10 (FIG. 11, SEQ ID NO: 60 and FIG. 12, SEQ ID NO: 61). In such embodiments, the SIRP1α-CD3 BiTE (e.g., SEQ ID NO: 46 or SEQ ID NO: 48) is linked to the therapeutic molecule, e.g., IL-15 (SEQ ID NO: 24), IL-12 p35 (SEQ ID NO: 28), IL-12 p40 (SEQ ID NO: 26), and/or CXCL10 (SEQ ID NO: 30), by a T2A self-cleaving peptide linker (SEQ ID NO: 14).

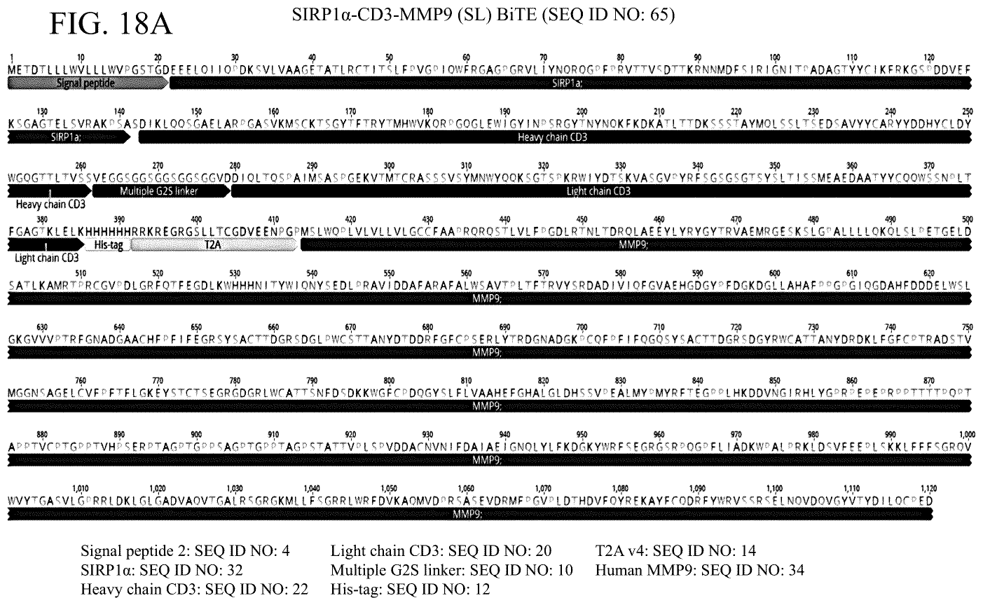

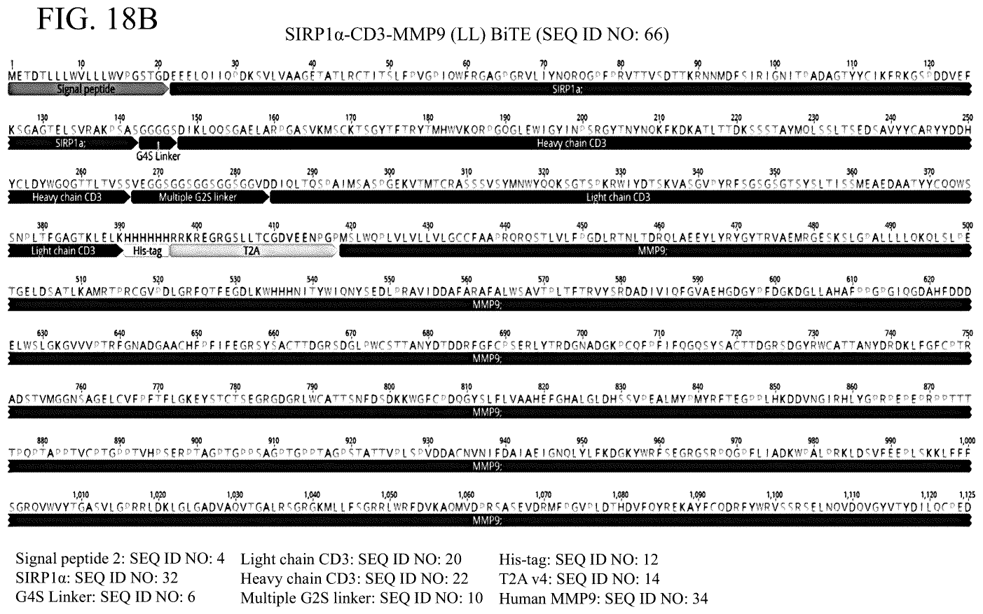

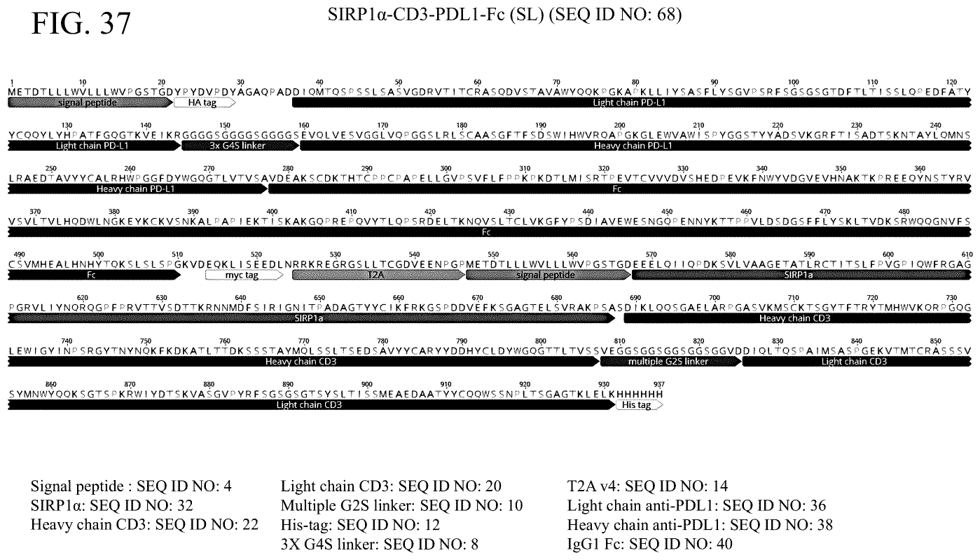

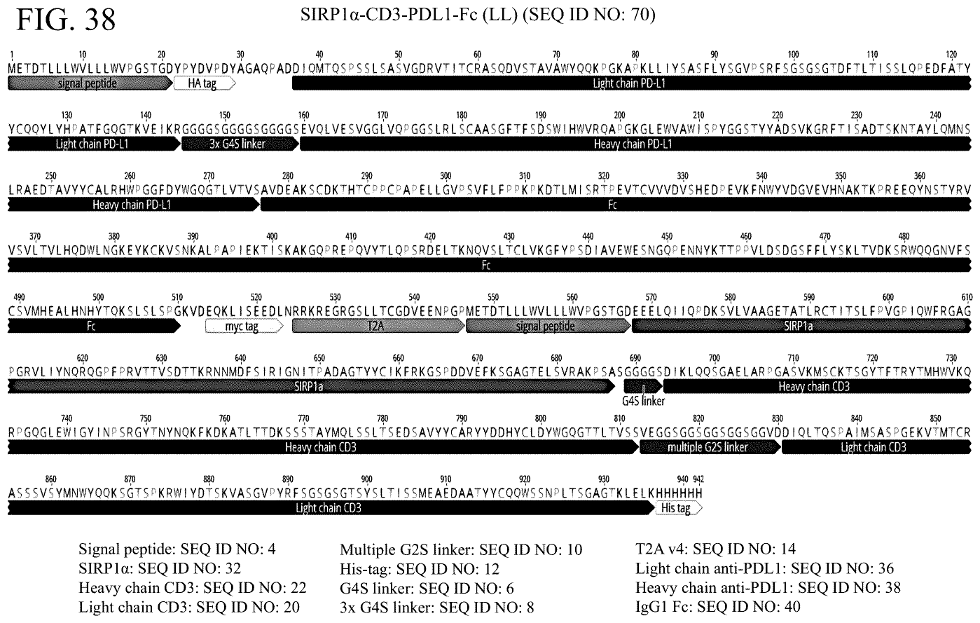

In some embodiments, the oncolytic viruses described herein comprise a bicistronic or multicistronic nucleic acid sequence, wherein a first nucleic acid sequence encodes a SIRP1α-CD3 BiTE and a second nucleic acid sequence encodes a therapeutic molecule such as MMP9 (FIG. 18A, SEQ ID NO: 65 and FIG. 18B, SEQ ID NO: 66). In such embodiments, the SIRP1α-CD3 BiTE (e.g., SEQ ID NO: 65 or 66) is linked to the MMP9 polypeptide (SEQ ID NO: 34) by a T2A self-cleaving peptide linker (SEQ ID NO: 14). In some embodiments, the oncolytic viruses described herein comprise a bicistronic or multicistronic nucleic acid sequence, wherein a first nucleic acid sequence encodes a SIRP1α-CD3 BiTE and a second nucleic acid sequence encodes a therapeutic molecule comprising an anti-PDL1 scFv linked to an IgG1 Fc domain (e.g., comprises an IgG1 CH2-CH3-Hinge, SEQ ID NO: 40), such as the SIRP1α-CD3-PDL1-Fc (SL) construct shown in FIG. 37 (SEQ ID NO: 68) or the SIRP1α-CD3-PDL1-Fc (LL) construct show in FIG. 38 (SEQ ID NO: 70).

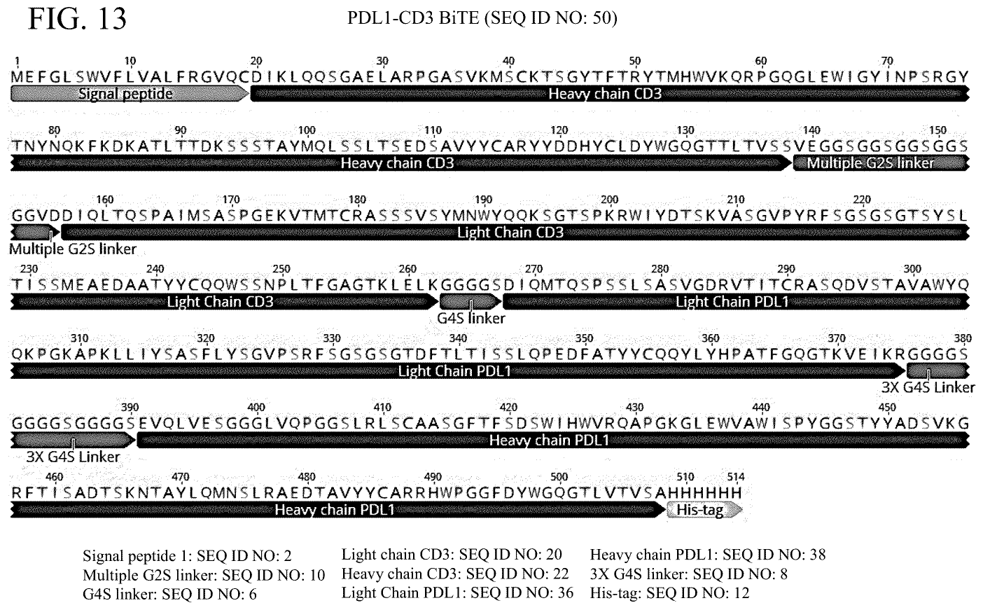

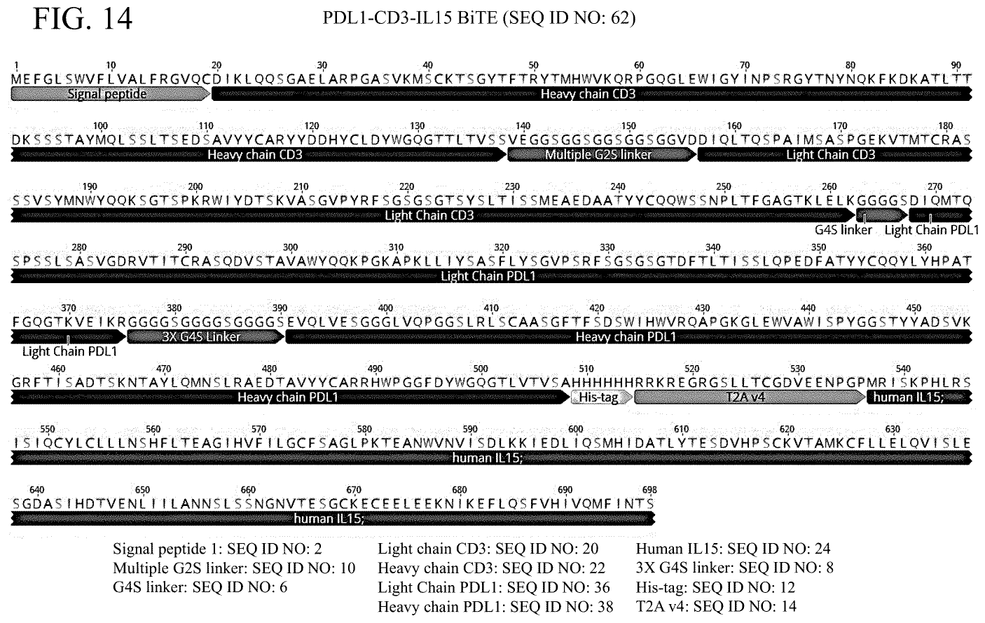

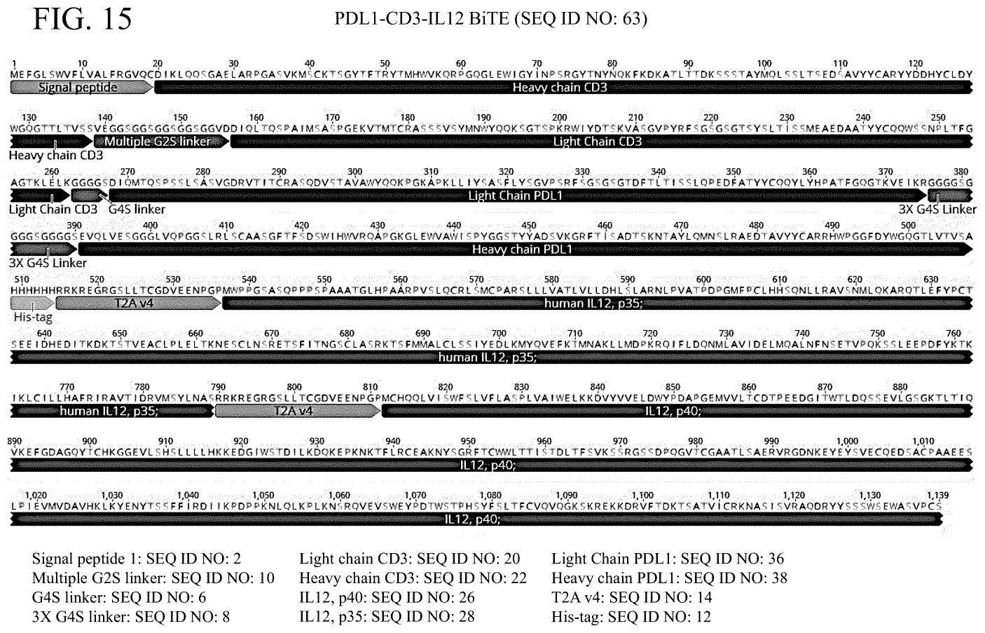

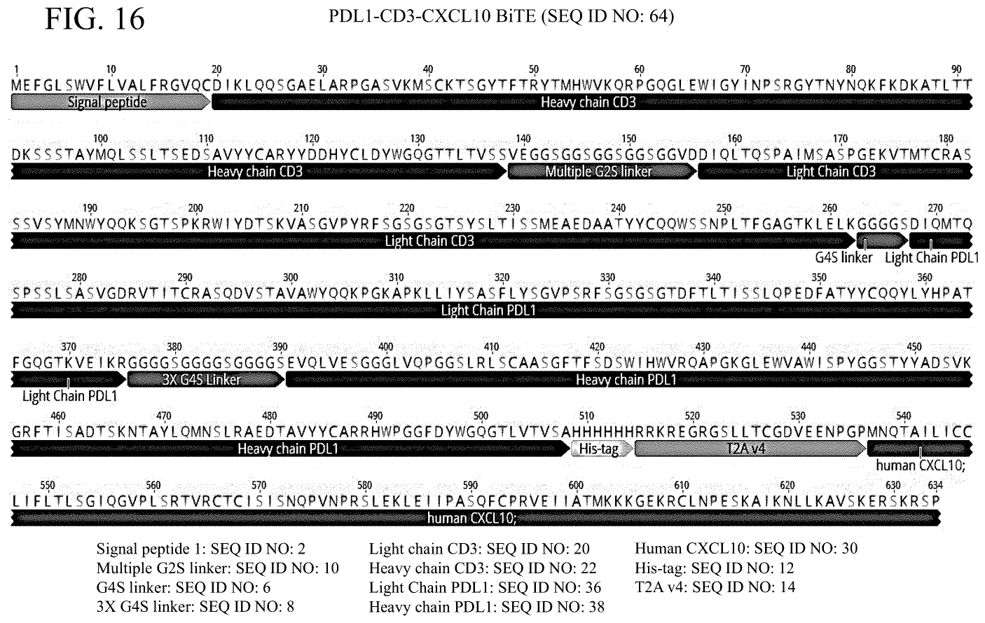

In some embodiments, the oncolytic viruses described herein comprise a polynucleotide sequence encoding an engager molecule comprising an effector domain comprising an scFv that binds to CD3 and an activation domain comprising an scFv that binds to PDL1, referred to herein as an PDL1-CD3 BiTE or a PDL1 BiTE. Exemplary PDL1-CD3 BiTEs are shown in FIG. 13 (SEQ ID NO: 50). In some embodiments, the anti-CD3 scFv and the anti-PDL1 scFv are linked together by G4S linker (SEQ ID NO: 6). In some embodiments, the oncolytic viruses described herein comprise a first nucleic acid sequence encoding a PDL1-CD3 BiTE and a second nucleic acid sequence encoding a therapeutic molecule such as IL-15, IL-12, CXCL10, or CCL4. In some embodiments, the nucleic acid sequences encoding the PDL1-CD3 BiTE and the therapeutic molecule are separate nucleic acid sequences and the PDL1-CD3 BiTE and the therapeutic molecule are encoded as separate proteins. In some embodiments, the oncolytic viruses described herein comprise a bicistronic or multicistronic nucleic acid sequence, wherein a first nucleic acid sequence encodes a PDL1-CD3 BiTE and a second nucleic acid sequence encodes a therapeutic molecule such as IL-15 (FIG. 14, SEQ ID NO: 62), IL-12 (FIG. 15, SEQ ID NO: 63), or CXCL10 (FIG. 16, SEQ ID NO: 64). In such embodiments, the SIRP1α-CD3 BiTE (e.g., SEQ ID NO: 50) is linked to the therapeutic molecule, e.g., IL-15 (SEQ ID NO: 24), IL-12 p35 (SEQ ID NO: 28), IL-12 p40 (SEQ ID NO: 26), and/or CXCL10 (SEQ ID NO: 30), by a T2A self-cleaving peptide linker (SEQ ID NO: 14).

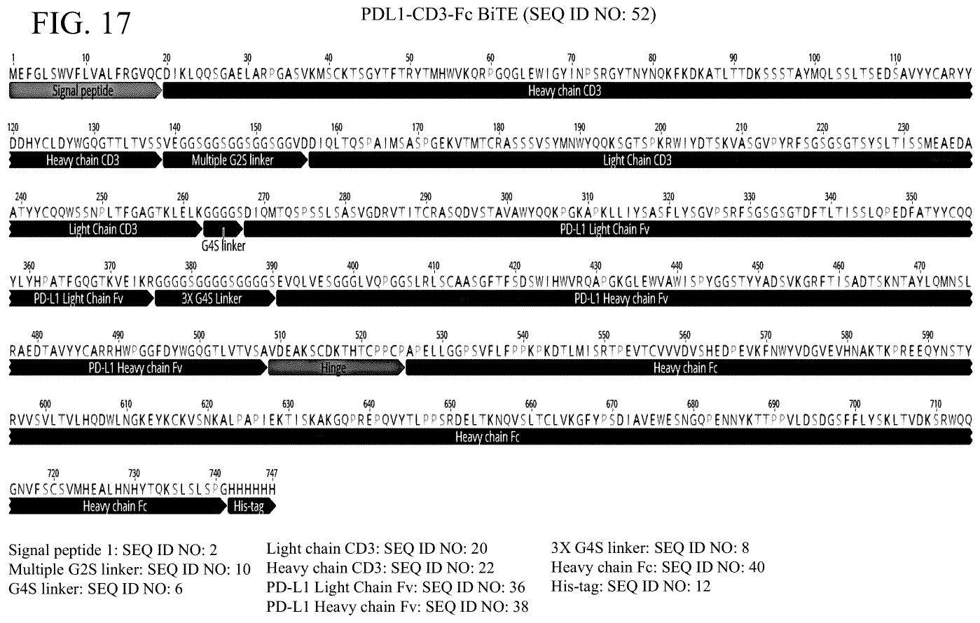

In some embodiments, oncolytic viruses described herein comprise a polynucleotide sequence encoding an engager molecule wherein the engager molecule is a tripartite engager molecule and comprises an effector domain comprising an scFv that binds to CD3, an activation domain comprising an scFv that binds to PDL1, and a third domain comprising an IgG1 Fc domain (e.g., comprises an IgG1 CH2-CH3-Hinge, SEQ ID NO: 40) and capable of binding to one or more FcγRs, referred to herein as an PDL1-CD3-Fc tripartite T cell engager, or TiTE, or a PDL1 TiTE. A schematic of an exemplary PDL1-CD3-Fc TiTE is shown in FIG. 17 (SEQ ID NO: 52).

The amino acid sequences of exemplary engager molecules and therapeutic molecules are shown in Table 1.

TABLE 1
Amino acid sequences of exemplary engager molecules

| BiTE | Amino Acid Sequence | SEQ ID |
|---|---|---|
| CD19-CD3 | MEFGLSWVFLVALFRGVQCDIQLTQSPASLAVSLGQRATISCKASQSVDYDGDSYLN | 44 |
| | WYQQIPGQPPKLLIYDASNLVSGIPPRFSGSGSGTDFTLNIHPVEKVDAATYHCQQS | |
| | TEDPWTFGGGTKLEIKGGGGSGGGGSGGGGSQVQLQQSGAELVRPGSSVKISCKASG | |
| | YAFSSYWMNWVKQRPGQGLEWIGQIWPGDGDTNYNGKFKGKATLTADESSSTAYMQL | |
| | SSLASEDSAVYFCARRETTTVGRYYYAMDYWGQGTTVTVSSGGGGSDIKLQQSGAEL | |
| | ARPGASVKMSCKTSGYTFTRYTMHWVKQRPGQGLEWIGYINPSRGYTNYNQKFKDKA | |
| | TLTTDKSSSTAYMQLSSLTSEDSAVYYCARYYDDHYCLDYWGQGTTLTVSSVEGGSG | |
| | GSGGSGGSGGVDDIQLTQSPAIMSASPGEKVTMTCRASSSVSYMNWYQQKSGTSPKR | |
| | WIYDTSKVASGVPYRFSGSGSGTSYSLTISSMEAEDAATYYCQQWSSNPLTFGAGTK | |
| | LELKHHHHHH- | |
| SIRP1α- | METDTLLLWVLLLWVPGSTGDEEELQIIQPDKSVLVAAGETATLRCTITSLFPVGPI | 46 |
| CD3-SL | QWFRGAGPGRVLIYNQRQGPFPRVTTVSDTTKRNNMDFSIRIGNITPADAGTYYCIK | |
| | FRKGSPDDVEFKSGAGTELSVRAKPSASDIKLQQSGAELARPGASVKMSCKTSGYTF | |
| | TRYTMHWVKQRPGQGLEWIGYINPSRGYTNYNQKFKDKATLTTDKSSSTAYMQLSSL | |
| | TSEDSAVYYCARYYDDHYCLDYWGQGTTLTVSSVEGGSGGSGGSGGSGGVDDIQLTQ | |
| | SPAIMSASPGEKVTMTCRASSSVSYMNWYQQKSGTSPKRWIYDTSKVASGVPYRFSG | |
| | SGSGTSYSLTISSMEAEDAATYYCQQWSSNPLTFGAGTKLELKHHHHHH- | |
| SIRP1α- | METDTLLLWVLLLWVPGSTGDEEELQIIQPDKSVLVAAGETATLRCTITSLFPVGPI | 48 |
| CD3-LL | QWFRGAGPGRVLIYNQRQGPFPRVTTVSDTTKRNNMDFSIRIGNITPADAGTYYCIK | |
| | FRKGSPDDVEFKSGAGTELSVRAKPSASGGGGSDIKLQQSGAELARPGASVKMSCKT | |
| | SGYTFTRYTMHWVKQRPGQGLEWIGYINPSRGYTNYNQKFKDKATLTTDKSSSTAYM | |
| | QLSSLTSEDSAVYYCARYYDDHYCLDYWGQGTTLTVSSVEGGSGGSGGSGGSGGVDD | |
| | IQLTQSPAIMSASPGEKVTMTCRASSSVSYMNWYQQKSGTSPKRWIYDTSKVASGVP | |
| | YRFSGSGSGTSYSLTISSMEAEDAATYYCQQWSSNPLTFGAGTKLELKHHHHHH- | |
| PDL1-CD3 | MEFGLSWVFLVALFRGVQCDIKLQQSGAELARPGASVKMSCKTSGYTFTRYTMHWVK | 50 |
| | QRPGQGLEWIGYINPSRGYTNYNQKFKDKATLTTDKSSSTAYMQLSSLTSEDSAVYY | |
| | CARYYDDHYCLDYWGQGTTLTVSSVEGGSGGSGGSGGSGGVDDIQLTQSPAIMSASP | |
| | GEKVTMTCRASSSVSYMNWYQQKSGTSPKRWIYDTSKVASGVPYRFSGSGSGTSYSL | |
| | TISSMEAEDAATYYCQQWSSNPLTFGAGTKLELKGGGGSDIQMTQSPSSLSASVGDR | |
| | VTITCRASQDVSTAVAWYQQKPGKAPKLLIYSASFLYSGVPSRFSGSGSGTDFTLTI | |
| | SSLQPEDFATYYCQQYLYHPATFGQGTKVEIKRGGGGSGGGGSGGGGSEVQLVESGG | |
| | GLVQPGGSLRLSCAASGFTFSDSWIHWVRQAPGKGLEWVAWISPYGGSTYYADSVKG | |
| | RFTISADTSKNTAYLQMNSLRAEDTAVYYCARRHWPGGFDYWGQGTLVTVSAHHHHH | |
| | H- | |
| PDIA-CD3- | MEFGLSWVFLVALFRGVQCDIKLQQSGAELARPGASVKMSCKTSGYTFTRYTMHWVK | 52 |
| Fc | QRPGQGLEWIGYINPSRGYTNYNQKFKDKATLTTDKSSSTAYMQLSSLTSEDSAVYY | |
| | CARYYDDHYCLDYWGQGTTLTVSSVEGGSGGSGGSGGSGGVDDIQLTQSPAIMSASP | |
| | GEKVTMTCRASSSVSYMNWYQQKSGTSPKRWIYDTSKVASGVPYRFSGSGSGTSYSL | |
| | TISSMEAEDAATYYCQQWSSNPLTFGAGTKLELKGGGGSDIQMTQSPSSLSASVGDR | |
| | VTITCRASQDVSTAVAWYQQKPGKAPKLLIYSASFLYSGVPSRFSGSGSGTDFTLTI | |
| | SSLQPEDFATYYCQQYLYHPATFGQGTKVEIKRGGGGSGGGGSGGGGSEVQLVESGG | |
| | GLVQPGGSLRLSCAASGFTFSDSWIHWVRQAPGKGLEWVAWISPYGGSTYYADSVKG | |
| | RFTISADTSKNTAYLQMNSLRAEDTAVYYCARRHWPGGFDYWGQGTLVTVSAVDEAK | |
| | SCDKTHTCPPCPAPELLGGPSVFLFPPKPKDTLMISRTPEVTCVVVDVSHEDPEVKF | |
| | NWYVDGVEVHNAKTKPREEQYNSTYRVVSVLTVLHQDWLNGKEYKCKVSNKALPAPI | |
| | EKTISKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLSLSPG | |
| | HHHHHH- | |
| CD19-CD3- | MEFGLSWVFLVALFRGVQCDIQLTQSPASLAVSLGQRATISCKASQSVDYDGDSYLN | 53 |
| IL15 | WYQQIPGQPPKLLIYDASNLVSGIPPRFSGSGSGTDFTLNIHPVEKVDAATYHCQQS | |
| | TEDPWTFGGGTKLEIKGGGGSGGGGSGGGGSQVQLQQSGAELVRPGSSVKISCKASG | |
| | YAFSSYWMNWVKQRPGQGLEWIGQIWPGDGDTNYNGKFKGKATLTADESSSTAYMQL | |
| | SSLASEDSAVYFCARRETTTVGRYYYAMDYWGQGTTVTVSSGGGGSDIKLQQSGAEL | |
| | ARPGASVKMSCKTSGYTFTRYTMHWVKQRPGQGLEWIGYINPSRGYTNYNQKFKDKA | |
| | TLTTDKSSSTAYMQLSSLTSEDSAVYYCARYYDDHYCLDYWGQGTTLTVSSVEGGSG | |
| | GSGGSGGSGGVDDIQLTQSPAIMSASPGEKVTMTCRASSSVSYMNWYQQKSGTSPKR | |
| | WIYDTSKVASGVPYRFSGSGSGTSYSLTISSMEAEDAATYYCQQWSSNPLTFGAGTK | |
| | LELKHHHHHHRRKREGRGSLLTCGDVEENPGPMRISKPHLRSISIQCYLCLLLNSHF | |
| | LTEAGIHVFILGCFSAGLPKTEANWVNVISDLKKIEDLIQSMHIDATLYTESDVHPS | |
| | CKVTAMKCFLLELQVISLESGDASIHDTVENLIILANNSLSSNGNVTESGCKECEEL | |
| | EEKNIKEFLQSFVHIVQMFINTS- | |
| CD19-CD3- | MEFGLSWVFLVALFRGVQCDIQLTQSPASLAVSLGQRATISCKASQSVDYDGDSYLN | 54 |
| IL12 | WYQQIPGQPPKLLIYDASNLVSGIPPRFSGSGSGTDFTLNIHPVEKVDAATYHCQQS | |
| | TEDPWTFGGGTKLEIKGGGGSGGGGSGGGGSQVQLQQSGAELVRPGSSVKISCKASG | |
| | YAFSSYWMNWVKQRPGQGLEWIGQIWPGDGDTNYNGKFKGKATLTADESSSTAYMQL | |
| | SSLASEDSAVYFCARRETTTVGRYYYAMDYWGQGTTVTVSSGGGGSDIKLQQSGAEL | |
| | ARPGASVKMSCKTSGYTFTRYTMHWVKQRPGQGLEWIGYINPSRGYTNYNQKFKDKA | |
| | TLTTDKSSSTAYMQLSSLTSEDSAVYYCARYYDDHYCLDYWGQGTTLTVSSVEGGSG | |
| | GSGGSGGSGGVDDIQLTQSPAIMSASPGEKVTMTCRASSSVSYMNWYQQKSGTSPKR | |
| | WIYDTSKVASGVPYRFSGSGSGTSYSLTISSMEAEDAATYYCQQWSSNPLTFGAGTK | |
| | LELKHHHHHHRRKREGRGSLLTCGDVEENPGPMWPPGSASQPPPSPAAATGLHPAAR | |
| | PVSLQCRLSMCPARSLLLVATLVLLDHLSLARNLPVATPDPGMFPCLHHSQNLLRAV | |
| | SNMLQKARQTLEFYPCTSEEIDHEDITKDKTSTVEACLPLELTKNESCLNSRETSFI | |
| | TNGSCLASRKTSFMMALCLSSIYEDLKMYQVEFKTMNAKLLMDPKRQIFLDQNMLAV | |
| | IDELMQALNFNSETVPQKSSLEEPDFYKTKIKLCILLHAFRIRAVTIDRVMSYLNAS | |
| | RRKREGRGSLLTCGDVEENPGPPMCHQQLVISWFSLVFLASPLVAIWELKKDVYVVE | |
| | LDWYPDAPGEMVVLTCDTPEEDGITWTLDQSSEVLGSGKTLTIQVKEFGDAGQYTCH | |
| | KGGEVLSHSLLLLHKKEDGIWSTDILKDQKEPKNKTFLRCEAKNYSGRFTCWWLTTI | |
| | STDLTFSVKSSRGSSDPQGVTCGAATLSAERVRGDNKEYEYSVECQEDSACPAAEES | |
| | LPIEVMVDAVHKLKYENYTSSFFIRDIIKPDPPKNLQLKPLKNSRQVEVSWEYPDTW | |
| | STPHSYFSLTFCVQVQGKSKREKKDRVFTDKTSATVICRKNASISVRAQDRYYSSSW | |
| | SEWASVPCS- | |
| CD19-CD3- | MEFGLSWVFLVALFRGVQCDIQLTQSPASLAVSLGQRATISCKASQSVDYDGDSYLN | 55 |
| CXCL10 | WYQQIPGQPPKLLIYDASNLVSGIPPRESGSGSGTDFTLNIHPVEKVDAATYHCQQS | |
| | TEDPWTFGGGTKLEIKGGGGSGGGGSGGGGSQVQLQQSGAELVRPGSSVKISCKASG | |
| | YAFSSYWMNWVKQRPGQGLEWIGQIWPGDGDTNYNGKFKGKATLTADESSSTAYMQL | |
| | SSLASEDSAVYFCARRETTTVGRYYYAMDYWGQGTTVTVSSGGGGSDIKLQQSGAEL | |
| | ARPGASVKMSCKTSGYTFTRYTMHWVKQRPGQGLEWIGYINPSRGYTNYNQKFKDKA | |
| | TLTTDKSSSTAYMQLSSLTSEDSAVYYCARYYDDHYCLDYWGQGTTLTVSSVEGGSG | |
| | GSGGSGGSGGVDDIQLTQSPAIMSASPGEKVTMTCRASSSVSYMNWYQQKSGTSPKR | |
| | WIYDTSKVASGVPYRFSGSGSGTSYSLTISSMEAEDAATYYCQQWSSNPLTFGAGTK | |
| | LELKHHHHHHRRKREGRGSLLTCGDVEENPGPMNQTAILICCLIFLTLSGIQGVPLS | |
| | RTVRCTCISISNQPVNPRSLEKLEIIPASQFCPRVEIIATMKKKGEKRCLNPESKAI | |
| | KNLLKAVSKERSKRSP- | |
| SIRP1α- | METDTLLLWVLLLWVPGSTGDEEELQIIQPDKSVLVAAGETATLRCTITSLFPVGPI | 56 |
| CD3-IL15 | QWFRGAGPGRVLIYNQRQGPFPRVTTVSDTTKRNNMDFSIRIGNITPADAGTYYCIK | |
| (SL) | FRKGSPDDVEFKSGAGTELSVRAKPSASDIKLQQSGAELARPGASVKMSCKTSGYTF | |
| | TRYTMHWVKQRPGQGLEWIGYINPSRGYTNYNQKFKDKATLTTDKSSSTAYMQLSSL | |
| | TSEDSAVYYCARYYDDHYCLDYWGQGTTLTVSSVEGGSGGSGGSGGSGGVDDIQLTQ | |
| | SPAIMSASPGEKVTMTCRASSSVSYMNWYQQKSGTSPKRWIYDTSKVASGVPYRFSG | |
| | SGSGTSYSLTISSMEAEDAATYYCQQWSSNPLTFGAGTKLELKHHHHHHRRKREGRG | |
| | SLLTCGDVEENPGPMRISKPHLRSISIQCYLCLLLNSHFLTEAGIHVFILGCFSAGL | |
| | PKTEANWVNVISDLKKIEDLIQSMHIDATLYTESDVHPSCKVTAMKCFLLELQVISL | |
| | ESGDASIHDTVENLIILANNSLSSNGNVTESGCKECEELEEKNIKEFLQSFVHIVQM | |
| | FINTS- | |
| SIRP1α- | METDTLLLWVLLLWVPGSTGDEEELQIIQPDKSVLVAAGETATLRCTITSLFPVGPI | 57 |
| CD3-IL15 | QWFRGAGPGRVLIYNQRQGPFPRVTTVSDTTKRNNMDFSIRIGNITPADAGTYYCIK | |
| (LL) | FRKGSPDDVEFKSGAGTELSVRAKPSASGGGGSDIKLQQSGAELARPGASVKMSCKT | |
| | SGYTFTRYTMHWVKQRPGQGLEWIGYINPSRGYTNYNQKFKDKATLTTDKSSSTAYM | |
| | QLSSLTSEDSAVYYCARYYDDHYCLDYWGQGTTLTVSSVEGGSGGSGGSGGSGGVDD | |
| | IQLTQSPAIMSASPGEKVTMTCRASSSVSYMNWYQQKSGTSPKRWIYDTSKVASGVP | |
| | YRFSGSGSGTSYSLTISSMEAEDAATYYCQQWSSNPLTFGAGTKLELKHHHHHHRRK | |
| | REGRGSLLTCGDVEENPGPMRISKPHLRSISIQCYLCLLLNSHFLTEAGIHVFILGC | |
| | FSAGLPKTEANWVNVISDLKKIEDLIQSMHIDATLYTESDVHPSCKVTAMKCFLLEL | |
| | QVISLESGDASIHDTVENLIILANNSLSSNGNVTESGCKECEELEEKNIKEFLQSFV | |
| | HIVQMFINTS- | |
| SIRP1α- | METDTLLLWVLLLWVPGSTGDEEELQIIQPDKSVLVAAGETATLRCTITSLFPVGPI | 58 |
| C3-IL12) | QWFRGAGPGRVLIYNQRQGPFPRVTTVSDTTKRNNMDFSIRIGNITPADAGTYYCIK | |
| (SL) | FRKGSPDDVEFKSGAGTELSVRAKPSASDIKLQQSGAELARPGASVKMSCKTSGYTF | |
| | TRYTMHWVKQRPGQGLEWIGYINPSRGYTNYNQKFKDKATLTTDKSSSTAYMQLSSL | |
| | TSEDSAVYYCARYYDDHYCLDYWGQGTTLTVSSVEGGSGGSGGSGGSGGVDDIQLTQ | |
| | SPAIMSASPGEKVTMTCRASSSVSYMNWYQQKSGTSPKRWIYDTSKVASGVPYRFSG | |
| | SGSGTSYSLTISSMEAEDAATYYCQQWSSNPLTFGAGTKLELKHHHHHHRRKREGRG | |
| | SLLTCGDVEENPGPMWPPGSASQPPPSPAAATGLHPAARPVSLQCRLSMCPARSLLL | |
| | VATLVLLDHLSLARNLPVATPDPGMFPCLHHSQNLLRAVSNMLQKARQTLEFYPCTS | |
| | EEIDHEDITKDKTSTVEACLPLELTKNESCLNSRETSFITNGSCLASRKTSFMMALC | |
| | LSSIYEDLKMYQVEFKTMNAKLLMDPKRQIFLDQNMLAVIDELMQALNFNSETVPQK | |
| | SSLEEPDFYKTKIKLCILLHAFRIRAVTIDRVMSYLNASRRKREGRGSLLTCGDVEE | |
| | NPGPPMCHQQLVISWFSLVFLASPLVAIWELKKDVYVVELDWYPDAPGEMVVLTCDT | |
| | PEEDGITWTLDQSSEVLGSGKTLTIQVKEFGDAGQYTCHKGGEVLSHSLLLLHKKED | |
| | GIWSTDILKDQKEPKNKTFLRCEAKNYSGRFTCWWLTTISTDLTFSVKSSRGSSDPQ | |
| | GVTCGAATLSAERVRGDNKEYEYSVECQEDSACPAAEESLPIEVMVDAVHKLKYENY | |
| | TSSFFIRDIIKPDPPKNLQLKPLKNSRQVEVSWEYPDTWSTPHSYFSLTFCVQVQGK | |
| | SKREKKDRVFTDKTSATVICRKNASISVRAQDRYYSSSWSEWASVPCS- | |
| SIRP1α- | METDTLLLWVLLLWVPGSTGDEEELQIIQPDKSVLVAAGETATLRCTITSLFPVGPI | 59 |
| CD3-IL12 | QWFRGAGPGRVLIYNQRQGPFPRVTTVSDTTKRNNMDFSIRIGNITPADAGTYYCIK | |
| (LL) | FRKGSPDDVEFKSGAGTELSVRAKPSASGGGGSDIKLQQSGAELARPGASVKMSCKT | |
| | SGYTFTRYTMHWVKQRPGQGLEWIGYINPSRGYTNYNQKFKDKATLTTDKSSSTAYM | |
| | QLSSLTSEDSAVYYCARYYDDHYCLDYWGQGTTLTVSSVEGGSGGSGGSGGSGGVDD | |
| | IQLTQSPAIMSASPGEKVTMTCRASSSVSYMNWYQQKSGTSPKRWIYDTSKVASGVP | |
| | YRFSGSGSGTSYSLTISSMEAEDAATYYCQQWSSNPLTFGAGTKLELKHHHHHHRRK | |
| | REGRGSLLTCGDVEENPGPMWPPGSASQPPPSPAAATGLHPAARPVSLQCRLSMCPA | |
| | RSLLLVATLVLLDHLSLARNLPVATPDPGMFPCLHHSQNLLRAVSNMLQKARQTLEF | |
| | YPCTSEEIDHEDITKDKTSTVEACLPLELTKNESCLNSRETSFITNGSCLASRKTSF | |
| | MMALCLSSIYEDLKMYQVEFKTMNAKLLMDPKRQIFLDQNMLAVIDELMQALNFNSE | |
| | TVPQKSSLEEPDFYKTKIKLCILLHAFRIRAVTIDRVMSYLNASRRKREGRGSLLTC | |
| | GDVEENPGPPMCHQQLVISWFSLVFLASPLVAIWELKKDVYVVELDWYPDAPGEMVV | |
| | LTCDTPEEDGITWTLDQSSEVLGSGKTLTIQVKEFGDAGQYTCHKGGEVLSHSLLLL | |
| | HKKEDGIWSTDILKDQKEPKNKTFLRCEAKNYSGRFTCWWLTTISTDLTFSVKSSRG | |
| | SSDPQGVTCGAATLSAERVRGDNKEYEYSVECQEDSACPAAEESLPIEVMVDAVHKL | |
| | KYENYTSSFFIRDIIKPDPPKNLQLKPLKNSRQVEVSWEYPDTWSTPHSYFSLTFCV | |
| | QVQGKSKREKKDRVFTDKTSATVICRKNASISVRAQDRYYSSSWSEWASVPCS- | |
| SIRP1α- | METDTLLLWVLLLWVPGSTGDEEELQIIQPDKSVLVAAGETATLRCTITSLFPVGPI | 60 |
| CD3- | QWFRGAGPGRVLIYNQRQGPFPRVTTVSDTTKRNNMDFSIRIGNITPADAGTYYCIK | |
| CXCL10 | FRKGSPDDVEFKSGAGTELSVRAKPSASDIKLQQSGAELARPGASVKMSCKTSGYTF | |
| (SL) | TRYTMHWVKQRPGQGLEWIGYINPSRGYTNYNQKFKDKATLTTDKSSSTAYMQLSSL | |
| | TSEDSAVYYCARYYDDHYCLDYWGQGTTLTVSSVEGGSGGSGGSGGSGGVDDIQLTQ | |
| | SPAIMSASPGEKVTMTCRASSSVSYMNWYQQKSGTSPKRWIYDTSKVASGVPYRFSG | |
| | SGSGTSYSLTISSMEAEDAATYYCQQWSSNPLTFGAGTKLELKHHHHHHRRKREGRG | |
| | SLLTCGDVEENPGPMNQTAILICCLIFLTLSGIQGVPLSRTVRCTCISISNQPVNPR | |
| | SLEKLEIIPASQFCPRVEIIATMKKKGEKRCLNPESKAIKNLLKAVSKERSKRSP- | |
| SIRP1α- | METDTLLLWVLLLWVPGSTGDEEELQIIQPDKSVLVAAGETATLRCTITSLFPVGPI | 61 |
| CD3- | QWFRGAGPGRVLIYNQRQGPFPRVTTVSDTTKRNNMDFSIRIGNITPADAGTYYCIK | |
| CXCL10 | FRKGSPDDVEFKSGAGTELSVRAKPSASGGGGSDIKLQQSGAELARPGASVKMSCKT | |
| (LL) | SGYTFTRYTMHWVKQRPGQGLEWIGYINPSRGYTNYNQKFKDKATLTTDKSSSTAYM | |
| | QLSSLTSEDSAVYYCARYYDDHYCLDYWGQGTTLTVSSVEGGSGGSGGSGGSGGVDD | |
| | IQLTQSPAIMSASPGEKVTMTCRASSSVSYMNWYQQKSGTSPKRWIYDTSKVASGVP | |
| | YRFSGSGSGTSYSLTISSMEAEDAATYYCQQWSSNPLTFGAGTKLELKHHHHHHRRK | |
| | REGRGSLLTCGDVEENPGPMNQTAILICCLIFLTLSGIQGVPLSRTVRCTCISISNQ | |
| | PVNPRSLEKLEIIPASQFCPRVEIIATMKKKGEKRCLNPESKAIKNLLKAVSKERSK | |
| | RSP- | |
| PDL1-CD3- | MEFGLSWVFLVALFRGVQCDIKLQQSGAELARPGASVKMSCKTSGYTFTRYTMHWVK | 62 |
| IL15 | QRPGQGLEWIGYINPSRGYTNYNQKFKDKATLTTDKSSSTAYMQLSSLTSEDSAVYY | |
| | CARYYDDHYCLDYWGQGTTLTVSSVEGGSGGSGGSGGSGGVDDIQLTQSPAIMSASP | |
| | GEKVTMTCRASSSVSYMNWYQQKSGTSPKRWIYDTSKVASGVPYRFSGSGSGTSYSL | |
| | TISSMEAEDAATYYCQQWSSNPLTFGAGTKLELKGGGGSDIQMTQSPSSLSASVGDR | |
| | VTITCRASQDVSTAVAWYQQKPGKAPKLLIYSASFLYSGVPSRFSGSGSGTDFTLTI | |
| | SSLQPEDFATYYCQQYLYHPATFGQGTKVEIKRGGGGSGGGGSGGGGSEVQLVESGG | |
| | GLVQPGGSLRLSCAASGFTFSDSWIHWVRQAPGKGLEWVAWISPYGGSTYYADSVKG | |
| | RFTISADTSKNTAYLQMNSLRAEDTAVYYCARRHWPGGFDYWGQGTLVTVSAHHHHH | |
| | HRRKREGRGSLLTCGDVEENPGPMRISKPHLRSISIQCYLCLLLNSHFLTEAGIHVF | |
| | ILGCFSAGLPKTEANWVNVISDLKKIEDLIQSMHIDATLYTESDVHPSCKVTAMKCF | |
| | LLELQVISLESGDASIHDTVENLIILANNSLSSNGNVTESGCKECEELEEKNIKEFL | |
| | QSFVHIVQMFINTS- | |
| PDL1-CD3- | MEFGLSWVFLVALFRGVQCDIKLQQSGAELARPGASVKMSCKTSGYTFTRYTMHWVK | 63 |
| IL12 | QRPGQGLEWIGYINPSRGYTNYNQKFKDKATLTTDKSSSTAYMQLSSLTSEDSAVYY | |
| | CARYYDDHYCLDYWGQGTTLTVSSVEGGSGGSGGSGGSGGVDDIQLTQSPAIMSASP | |
| | GEKVTMTCRASSSVSYMNWYQQKSGTSPKRWIYDTSKVASGVPYRFSGSGSGTSYSL | |
| | TISSMEAEDAATYYCQQWSSNPLTFGAGTKLELKGGGGSDIQMTQSPSSLSASVGDR | |
| | VTITCRASQDVSTAVAWYQQKPGKAPKLLIYSASFLYSGVPSRFSGSGSGTDFTLTI | |
| | SSLQPEDFATYYCQQYLYHPATFGQGTKVEIKRGGGGSGGGGSGGGGSEVQLVESGG | |
| | GLVQPGGSLRLSCAASGFTFSDSWIHWVRQAPGKGLEWVAWISPYGGSTYYADSVKG | |
| | RFTISADTSKNTAYLQMNSLRAEDTAVYYCARRHWPGGFDYWGQGTLVTVSAHHHHH | |
| | HRRKREGRGSLLTCGDVEENPGPMWPPGSASQPPPSPAAATGLHPAARPVSLQCRLS | |
| | MCPARSLLLVATLVLLDHLSLARNLPVATPDPGMFPCLHHSQNLLRAVSNMLQKARQ | |
| | TLEFYPCTSEEIDHEDITKDKTSTVEACLPLELTKNESCLNSRETSFITNGSCLASR | |
| | KTSFMMALCLSSIYEDLKMYQVEFKTMNAKLLMDPKRQIFLDQNMLAVIDELMQALN | |
| | FNSETVPQKSSLEEPDFYKTKIKLCILLHAFRIRAVTIDRVMSYLNASRRKREGRGS | |
| | LLTCGDVEENPGPPMCHQQLVISWFSLVFLASPLVAIWELKKDVYVVELDWYPDAPG | |
| | EMVVLTCDTPEEDGITWTLDQSSEVLGSGKTLTIQVKEFGDAGQYTCHKGGEVLSHS | |
| | LLLLHKKEDGIWSTDILKDQKEPKNKTFLRCEAKNYSGRFTCWWLTTISTDLTFSVK | |
| | SSRGSSDPQGVTCGAATLSAERVRGDNKEYEYSVECQEDSACPAAEESLPIEVMVDA | |
| | VHKLKYENYTSSFFIRDIIKPDPPKNLQLKPLKNSRQVEVSWEYPDTWSTPHSYFSL | |
| | TFCVQVQGKSKREKKDRVFTDKTSATVICRKNASISVRAQDRYYSSSWSEWASVPCS | |
| PDL1-CD3- | MEFGLSWVFLVALFRGVQCDIKLQQSGAELARPGASVKMSCKTSGYTFTRYTMHWVK | 64 |
| CXCL10 | QRPGQGLEWIGYINPSRGYTNYNQKFKDKATLTTDKSSSTAYMQLSSLTSEDSAVYY | |
| | CARYYDDHYCLDYWGQGTTLTVSSVEGGSGGSGGSGGSGGVDDIQLTQSPAIMSASP | |
| | GEKVTMTCRASSSVSYMNWYQQKSGTSPKRWIYDTSKVASGVPYRFSGSGSGTSYSL | |
| | TISSMEAEDAATYYCQQWSSNPLTFGAGTKLELKGGGGSDIQMTQSPSSLSASVGDR | |
| | VTITCRASQDVSTAVAWYQQKPGKAPKLLIYSASFLYSGVPSRFSGSGSGTDFTLTI | |
| | SSLQPEDFATYYCQQYLYHPATFGQGTKVEIKRGGGGSGGGGSGGGGSEVQLVESGG | |
| | GLVQPGGSLRLSCAASGFTFSDSWIHWVRQAPGKGLEWVAWISPYGGSTYYADSVKG | |
| | RFTISADTSKNTAYLQMNSLRAEDTAVYYCARRHWPGGFDYWGQGTLVTVSAHHHHH | |
| | HRRKREGRGSLLTCGDVEENPGPMNQTAILICCLIFLTLSGIQGVPLSRTVRCTCIS | |
| | ISNQPVNPRSLEKLEIIPASQFCPRVEIIATMKKKGEKRCLNPESKAIKNLLKAVSK | |
| | ERSKRSP- | |
| SIRP1α- | METDTLLLWVLLLWVPGSTGDEEELQIIQPDKSVLVAAGETATLRCTITSLFPVGPI | 65 |
| CD3-MMP9 | QWFRGAGPGRVLIYNQRQGPFPRVTTVSDTTKRNNMDFSIRIGNITPADAGTYYCIK | |
| (SL) | FRKGSPDDVEFKSGAGTELSVRAKPSASDIKLQQSGAELARPGASVKMSCKTSGYTF | |
| | TRYTMHWVKQRPGQGLEWIGYINPSRGYTNYNQKFKDKATLTTDKSSSTAYMQLSSL | |
| | TSEDSAVYYCARYYDDHYCLDYWGQGTTLTVSSVEGGSGGSGGSGGSGGVDDIQLTQ | |
| | SPAIMSASPGEKVTMTCRASSSVSYMNWYQQKSGTSPKRWIYDTSKVASGVPYRFSG | |
| | SGSGTSYSLTISSMEAEDAATYYCQQWSSNPLTFGAGTKLELKHHHHHHRRKREGRG | |
| | SLLTCGDVEENPGPMSLWQPLVLVLLVLGCCFAAPRQRQSTLVLFPGDLRTNLTDRQ | |
| | LAEEYLYRYGYTRVAEMRGESKSLGPALLLLQKQLSLPETGELDSATLKAMRTPRCG | |
| | VPDLGRFQTFEGDLKWHHHNITYWIQNYSEDLPRAVIDDAFARAFALWSAVTPLTFT | |
| | RVYSRDADIVIQFGVAEHGDGYPFDGKDGLLAHAFPPGPGIQGDAHFDDDELWSLGK | |
| | GVVVPTRFGNADGAACHFPFIFEGRSYSACTTDGRSDGLPWCSTTANYDTDDRFGFC | |
| | PSERLYTRDGNADGKPCQFPFIFQGQSYSACTTDGRSDGYRWCATTANYDRDKLFGF | |
| | CPTRADSTVMGGNSAGELCVFPFTFLGKEYSTCTSEGRGDGRLWCATTSNFDSDKKW | |
| | GFCPDQGYSLFLVAAHEFGHALGLDHSSVPEALMYPMYRFTEGPPLHKDDVNGIRHL | |
| | YGPRPEPEPRPPTTTTPQPTAPPTVCPTGPPTVHPSERPTAGPTGPPSAGPTGPPTA | |
| | GPSTATTVPLSPVDDACNVNIFDAIAEIGNQLYLFKDGKYWRFSEGRGSRPQGPFLI | |
| | ADKWPALPRKLDSVFEEPLSKKLFFFSGRQVWVYTGASVLGPRRLDKLGLGADVAQV | |
| | TGALRSGRGKMLLFSGRRLWRFDVKAQMVDPRSASEVDRMFPGVPLDTHDVFQYREK | |
| | AYFCQDRFYWRVSSRSELNQVDQVGYVTYDILQCPED- | |
| SIRP1α- | METDTLLLWVLLLWVPGSTGDEEELQIIQPDKSVLVAAGETATLRCTITSLFPVGPI | 66 |
| CD3-MMP9 | QWFRGAGPGRVLIYNQRQGPFPRVTTVSDTTKRNNMDFSIRIGNITPADAGTYYCIK | |
| (LL) | FRKGSPDDVEFKSGAGTELSVRAKPSASGGGGSDIKLQQSGAELARPGASVKMSCKT | |
| | SGYTFTRYTMHWVKQRPGQGLEWIGYINPSRGYTNYNQKFKDKATLTTDKSSSTAYM | |
| | QLSSLTSEDSAVYYCARYYDDHYCLDYWGQGTTLTVSSVEGGSGGSGGSGGSGGVDD | |
| | IQLTQSPAIMSASPGEKVTMTCRASSSVSYMNWYQQKSGTSPKRWIYDTSKVASGVP | |
| | YRFSGSGSGTSYSLTISSMEAEDAATYYCQQWSSNPLTFGAGTKLELKHHHHHHRRK | |
| | REGRGSLLTCGDVEENPGPMSLWQPLVLVLLVLGCCFAAPRQRQSTLVLFPGDLRTN | |
| | LTDRQLAEEYLYRYGYTRVAEMRGESKSLGPALLLLQKQLSLPETGELDSATLKAMR | |
| | TPRCGVPDLGRFQTFEGDLKWHHHNITYWIQNYSEDLPRAVIDDAFARAFALWSAVT | |
| | PLTFTRVYSRDADIVIQFGVAEHGDGYPFDGKDGLLAHAFPPGPGIQGDAHFDDDEL | |
| | WSLGKGVVVPTRFGNADGAACHFPFIFEGRSYSACTTDGRSDGLPWCSTTANYDTDD | |
| | RFGFCPSERLYTRDGNADGKPCQFPFIFQGQSYSACTTDGRSDGYRWCATTANYDRD | |
| | KLFGFCPTRADSTVMGGNSAGELCVFPFTFLGKEYSTCTSEGRGDGRLWCATTSNFD | |
| | SDKKWGFCPDQGYSLFLVAAHEFGHALGLDHSSVPEALMYPMYRFTEGPPLHKDDVN | |
| | GIRHLYGPRPEPEPRPPTTTTPQPTAPPTVCPTGPPTVHPSERPTAGPTGPPSAGPT | |
| | GPPTAGPSTATTVPLSPVDDACNVNIFDAIAEIGNQLYLEKDGKYWRFSEGRGSRPQ | |
| | GPFLIADKWPALPRKLDSVFEEPLSKKLFFFSGRQVWVYTGASVLGPRRLDKLGLGA | |
| | DVAQVTGALRSGRGKMLLFSGRRLWRFDVKAQMVDPRSASEVDRMFPGVPLDTHDVF | |
| | QYREKAYFCQDRFYWRVSSRSELNQVDQVGYVTYDILQCPED- | |
| SIRP1α- | METDRLLLWVLLLWVPGSTGDYPYDVPDYAGAQPADDIQMTQSPSSLSASVGDRVTI | 68 |
| CD3-PDL1- | TCRASQDVSTAVAWYQQKPGKAPKLLIYSASFLYSGVPSRFSGSGSGTDFTLTISSL | |
| Fc(SL) | QPEDFATYYCQQYLYHPATFGQGTKVEIKRGGGGSGGGGSGGGGSEVQLVESGGGLV | |
| | QPGGSLRLSCAASGETFSDSWIHWVRQAPGKGLEWVAWISPYGGSTYYADSVKGRFT | |
| | ISADTSKNTAYLQMNSLRAEDTAVYYCARRHWPGGFDYWGQGTLVTVSAVDEAKSCD | |
| | KTHTCPPCPAPELLGGPSVFLFPPKPKDTLMISRTPEVTCVVVDVSHEDPEVKFNWY | |
| | VDGVEVHNAKTKPREEQYNSTYRVVSVLTVLHQDWLNGKEYKCKVSNKALPAPIEKT | |
| | ISKAKGQPREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLSLSPGKVD | |
| | EQKLISEEDLNRRKREGRGSLLTCGDVEENPGPMETDRLLLWVLLLWVPGSTGDEEE | |
| | LQIIQPDKSVLVAAGETATLRCTITSLFPVGPIQWFRGAGPGRVLIYNQRQGPFPRV | |
| | TTVSDTTKRNNMDFSIRIGNITPADAGTYYCIKFRKGSPDDVEFKSGAGTELSVRAK | |
| | PSASDIKLQQSGAELARPGASVKMSCKTSGYTFTRYTMHWVKQRPGQGLEWIGYINP | |
| | SRGYTNYNQKFKDKATLTTDKSSSTAYMQLSSLTSEDSAVYYCARYYDDHYCLDYWG | |
| | QGTTLTVSSVEGGSGGSGGSGGSGGVDDIQLTQSPAIMSASPGEKVTMTCRASSSVS | |
| | YMNWYQQKSGTSPKRWIYDTSKVASGVPYRFSGSGSGTSYSLTISSMEAEDAATYYC | |
| | QQWSSNPLTFGAGTKLELKHHHHHH- | |
| SIRP1α- | METDRLLLWVLLLWVPGSTGDYPYDVPDYAGAQPADDIQMTQSPSSLSASVGDRVTI | 70 |
| CD3-PDL1- | TCRASQDVSTAVAWYQQKPGKAPKLLIYSASFLYSGVPSRFSGSGSGTDFTLTISSL | |
| Fc(LL) | QPEDFATYYCQQYLYHPATFGQGTKVEIKRGGGGSGGGGSGGGGSEVQLVESGGGLV | |
| | QPGGSLRLSCAASGETFSDSWIHWVRQAPGKGLEWVAWISPYGGSTYYADSVKGRFT | |
| | ISADTSKNTAYLQMNSLRAEDTAVYYCARRHWPGGFDYWGQGTLVTVSAVDEAKSCD | |
| | KTHTCPPCPAPELLGGPSVFLFPPKPKDTLMISRTPEVTCVVVDVSHEDPEVKFNWY | |
| | VDGVEVHNAKTKPREEQYNSTYRVVSVLTVLHQDWLNGKEYKCKVSNKALPAPIEKT | |
| | ISKAKGQPREPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSLSLSPGKVD | |
| | EQKLISEEDLNRRKREGRGSLLTCGDVEENPGPMETDRLLLWVLLLWVPGSTGDEEE | |
| | LQIIQPDKSVLVAAGETATLRCTITSLFPVGPIQWFRGAGPGRVLIYNQRQGPFPRV | |
| | TTVSDTTKRNNMDFSIRIGNITPADAGTYYCIKFRKGSPDDVEFKSGAGTELSVRAK | |
| | PSASGGGGSDIKLQQSGAELARPGASVKMSCKTSGYTFTRYTMHWVKQRPGQGLEWI | |
| | GYINPSRGYTNYNQKFKDKATLTTDKSSSTAYMQLSSLTSEDSAVYYCARYYDDHYC | |
| | LDYWGQGTTLTVSSVEGGSGGSGGSGGSGGVDDIQLTQSPAIMSASPGEKVTMTCRA | |
| | SSSVSYMNWYQQKSGTSPKRWIYDTSKVASGVPYRFSGSGSGTSYSLTISSMEAEDA | |
| | ATYYCQQWSSNPLTFGAGTKLELKHHHHHH- | |
| FAP-CD3 | MYRMQLLSCIALSLALVTNSQVQLQQSGAELARPGASVNLSCKASGYTFTNNGINWL | 75 |
| | KQRTGQGLEWIGEIYPRSTNTLYNEKFKGKATLTADRSSNTAYMELRSLTSEDSAVY | |
| | FCARTLTAPFAFWGQGTLVTVSAGSTSGSGKPGSGEGSTKGQIVLTQSPAIMSASPG | |
| | EKVTMTCSASSGVNFMHWYQQKSGTSPKRWIFDTSKLASGVPARFSGSGSGTSYSLT | |
| | ISSMEAEDAATYYCQQWSFNPPTFGGGTKLEIKRGGGGSEVQLVESGGGLVQPGKSL | |
| | KLSCEASGETFSGYGMHWVRQAPGRGLESVAYITSSSINTKYADAVKGRFTVSRDNA | |
| | KNLLFLQMNILKSEDTAMYYCARFDWDKNYWGQGTMVTVSSGGGGSGGGGSGGGGSD | |
| | IQMTQSPSSLPASLGDRVTINCQASQDISNYLNWYQQKPGKAPKLLIYYTNKLADGV | |
| | PSRFSGSGSGRDSSETISSLESEDIGSYYCQQYYNYPWTFGPGTKLEIKRHHHHHH | |

In some embodiments, the oncolytic viruses described herein comprise a nucleic acid sequence selected from SEQ ID NOs: 43, 45, 47, 49, 51, 67, and 69. In some embodiments, the oncolytic viruses described herein comprise a nucleic acid sequence that is at least 80%, at least, 85%, at least 90%, at least 95%, at least 96%, at least 97%, at least 98%, or at least 99% identical to a nucleic acid sequence selected from SEQ ID NOs: 43, 45, 47, 49, 51, 67, and 69. In some embodiments, the oncolytic viruses described herein comprise a nucleic acid sequence that is 100% identical to a nucleic acid sequence selected from SEQ ID NOs: 43, 45, 47, 49, 51, 67, and 69. In some embodiments, the oncolytic viruses described herein consist of a nucleic acid sequence selected from SEQ ID NOs: 43, 45, 47, 49, 51, 67, and 69.

In some embodiments, the oncolytic viruses described herein comprise a nucleic acid sequence encoding an engager molecule and/or therapeutic molecule that is at least 80%, at least, 85%, at least 90%, at least 95%, at least 96%, at least 97%, at least 98%, or at least 99% identical to an amino acid sequence selected from SEQ ID NOs: 44, 46, 48, 50, and 52. In some embodiments, the oncolytic viruses described herein comprise a nucleic acid sequence encoding an engager molecule that is 100% identical to an amino acid sequence selected from SEQ ID NOs: 44, 46, 48, 50, and 52. In some embodiments, the oncolytic viruses described herein comprise a nucleic acid sequence encoding an engager molecule comprising an amino acid sequence selected from SEQ ID NOs: 44, 46, 48, 50, and 52. In some embodiments, the oncolytic viruses described herein comprise a nucleic acid sequence encoding an engager molecule consisting of an amino acid sequence selected from SEQ ID NOs: 44, 46, 48, 50, and 52.

In some embodiments, the oncolytic viruses described herein comprise a nucleic acid sequence encoding an engager molecule and a therapeutic molecule. In some embodiments, the recombinant nucleic acid sequences encode an engager molecule and a therapeutic molecule comprising an amino acid sequence is at least 80%, at least, 85%, at least 90%, at least 95%, at least 96%, at least 97%, at least 98%, or at least 99% identical to an amino acid sequence selected from SEQ ID NOs: 53-66, 68 and 70. In some embodiments, the recombinant nucleic acid sequences encode an engager molecule and a therapeutic molecule comprising an amino acid sequence that is 100% identical to an amino acid sequence selected from SEQ ID NOs: 53-66, 68 and 70. In some embodiments, the recombinant nucleic acid sequences encode an engager molecule and a therapeutic molecule comprising an amino acid sequence consisting of an amino acid sequence selected from SEQ ID NOs: 53-66, 68 and 70.

Table 2: Nucleic sequences of exemplary engager molecules BiTE Nucleic Acid Sequence SEQ ID

TABLE 2
Nucleic sequences of exemplary engager molecules

| BiTE | Nucleic Acid Sequence | SEQ ID |
|---|---|---|
| CD19- | ATGGAGTTCGGCCTGAGCTGGGTGTTCCTGGTGGCCCTGTTCAGGGGCGTGCAGTGC | 43 |
| CD3 | GACATCCAGCTGACCCAGAGCCCCGCCAGCCTGGCCGTGAGCCTGGGCCAGAGGGCC | |
| | ACCATCAGCTGCAAGGCCAGCCAGAGCGTGGACTACGACGGCGACAGCTACCTGAAC | |
| | TGGTACCAGCAGATCCCCGGCCAGCCCCCCAAGCTGCTGATCTACGACGCCAGCAAC | |
| | CTGGTGAGCGGCATCCCCCCCAGGTTCAGCGGCAGCGGCAGCGGCACCGACTTCACC | |
| | CTGAACATCCACCCCGTGGAGAAGGTGGACGCCGCCACCTACCACTGCCAGCAGAGC | |
| | ACCGAGGACCCCTGGACCTTCGGCGGCGGCACCAAGCTGGAGATCAAGGGCGGCGGC | |
| | GGCAGCGGCGGCGGCGGCAGCGGCGGCGGCGGCAGCCAGGTGCAGCTGCAGCAGAGC | |
| | GGCGCCGAGCTGGTGAGGCCCGGCAGCAGCGTGAAGATCAGCTGCAAGGCCAGCGGC | |
| | TACGCCTTCAGCAGCTACTGGATGAACTGGGTGAAGCAGAGGCCCGGCCAGGGCCTG | |
| | GAGTGGATCGGCCAGATCTGGCCCGGCGACGGCGACACCAACTACAACGGCAAGTTC | |
| | AAGGGCAAGGCCACCCTGACCGCCGACGAGAGCAGCAGCACCGCCTACATGCAGCTG | |
| | AGCAGCCTGGCCAGCGAGGACAGCGCCGTGTACTTCTGCGCCAGGAGGGAGACCACC | |
| | ACCGTGGGCAGGTACTACTACGCCATGGACTACTGGGGCCAGGGCACCACCGTGACC | |
| | GTGAGCAGCGGCGGCGGCGGCAGCGACATCAAGCTGCAGCAGAGCGGCGCCGAGCTG | |
| | GCCAGGCCCGGCGCCAGCGTGAAGATGAGCTGCAAGACCAGCGGCTACACCTTCACC | |
| | AGGTACACCATGCACTGGGTGAAGCAGAGGCCCGGCCAGGGCCTGGAGTGGATCGGC | |
| | TACATCAACCCCAGCAGGGGCTACACCAACTACAACCAGAAGTTCAAGGACAAGGCC | |
| | ACCCTGACCACCGACAAGAGCAGCAGCACCGCCTACATGCAGCTGAGCAGCCTGACC | |
| | AGCGAGGACAGCGCCGTGTACTACTGCGCCAGGTACTACGACGACCACTACTGCCTG | |
| | GACTACTGGGGCCAGGGCACCACCCTGACCGTGAGCAGCGTGGAGGGCGGCAGCGGC | |
| | GGCAGCGGCGGCAGCGGCGGCAGCGGCGGCGTGGACGACATCCAGCTGACCCAGAGC | |
| | CCCGCCATCATGAGCGCCAGCCCCGGCGAGAAGGTGACCATGACCTGCAGGGCCAGC | |
| | AGCAGCGTGAGCTACATGAACTGGTACCAGCAGAAGAGCGGCACCAGCCCCAAGAGG | |
| | TGGATCTACGACACCAGCAAGGTGGCCAGCGGCGTGCCCTACAGGTTCAGCGGCAGC | |
| | GGCAGCGGCACCAGCTACAGCCTGACCATCAGCAGCATGGAGGCCGAGGACGCCGCC | |
| | ACCTACTACTGCCAGCAGTGGAGCAGCAACCCCCTGACCTTCGGCGCCGGCACCAAG | |
| | CTGGAGCTGAAGCACCACCACCACCACCACTAG | |
| SIRP1a- | ATGGAGACCGATACCCTGCTCTTGTGGGTTTTGCTTCTTTGGGTGCCAGGATCTACA | 45 |
| CD3 | GGTGATGAAGAAGAATTGCAGATCATCCAACCAGACAAATCCGTACTCGTGGCCGCA | |
| (SL) | GGAGAGACCGCTACCCTCAGATGTACCATCACTTCTCTCTTCCCCGTTGGCCCCATC | |
| | CAGTGGTTTCGAGGCGCAGGACCAGGACGAGTGCTTATTTACAATCAACGACAGGGC | |
| | CCATTCCCAAGAGTGACAACAGTATCCGATACCACCAAGCGCAATAATATGGACTTT | |
| | AGCATTAGAATCGGCAACATAACACCCGCTGACGCCGGTACATACTATTGTATTAAA | |
| | TTTCGAAAGGGCTCACCAGACGACGTGGAATTTAAGTCAGGGGCCGGAACCGAACTC | |
| | TCAGTTAGAGCAAAACCTTCTGCTAGCGACATCAAGCTGCAGCAGAGCGGCGCCGAG | |
| | CTGGCCAGGCCCGGCGCCAGCGTGAAGATGAGCTGCAAGACCAGCGGCTACACCTTC | |
| | ACCAGGTACACCATGCACTGGGTGAAGCAGAGGCCCGGCCAGGGCCTGGAGTGGATC | |
| | GGCTACATCAACCCCAGCAGGGGCTACACCAACTACAACCAGAAGTTCAAGGACAAG | |
| | GCCACCCTGACCACCGACAAGAGCAGCAGCACCGCCTACATGCAGCTGAGCAGCCTG | |
| | ACCAGCGAGGACAGCGCCGTGTACTACTGCGCCAGGTACTACGACGACCACTACTGC | |
| | CTGGACTACTGGGGCCAGGGCACCACCCTGACCGTGAGCAGCGTGGAGGGCGGCAGC | |
| | GGCGGCAGCGGCGGCAGCGGCGGCAGCGGCGGCGTGGACGACATCCAGCTGACCCAG | |
| | AGCCCCGCCATCATGAGCGCCAGCCCCGGCGAGAAGGTGACCATGACCTGCAGGGCC | |
| | AGCAGCAGCGTGAGCTACATGAACTGGTACCAGCAGAAGAGCGGCACCAGCCCCAAG | |
| | AGGTGGATCTACGACACCAGCAAGGTGGCCAGCGGCGTGCCCTACAGGTTCAGCGGC | |
| | AGCGGCAGCGGCACCAGCTACAGCCTGACCATCAGCAGCATGGAGGCCGAGGACGCC | |
| | GCCACCTACTACTGCCAGCAGTGGAGCAGCAACCCCCTGACCTTCGGCGCCGGCACC | |
| | AAGCTGGAGCTGAAGCACCACCATCATCACCACTGAG | |
| SIRP1a- | ATGGAGACCGATACCCTGCTCTTGTGGGTTTTGCTTCTTTGGGTGCCAGGATCTACA | 47 |
| CD3 | GGTGATGAAGAAGAATTGCAGATCATCCAACCAGACAAATCCGTACTCGTGGCCGCA | |
| (LL) | GGAGAGACCGCTACCCTCAGATGTACCATCACTTCTCTCTTCCCCGTTGGCCCCATC | |
| | CAGTGGTTTCGAGGCGCAGGACCAGGACGAGTGCTTATTTACAATCAACGACAGGGC | |
| | CCATTCCCAAGAGTGACAACAGTATCCGATACCACCAAGCGCAATAATATGGACTTT | |
| | AGCATTAGAATCGGCAACATAACACCCGCTGACGCCGGTACATACTATTGTATTAAA | |
| | TTTCGAAAGGGCTCACCAGACGACGTGGAATTTAAGTCAGGGGCCGGAACCGAACTC | |
| | TCAGTTAGAGCAAAACCTTCTGCTAGCGGCGGCGGCGGCAGCGACATCAAGCTGCAG | |
| | CAGAGCGGCGCCGAGCTGGCCAGGCCCGGCGCCAGCGTGAAGATGAGCTGCAAGACC | |
| | AGCGGCTACACCTTCACCAGGTACACCATGCACTGGGTGAAGCAGAGGCCCGGCCAG | |
| | GGCCTGGAGTGGATCGGCTACATCAACCCCAGCAGGGGCTACACCAACTACAACCAG | |
| | AAGTTCAAGGACAAGGCCACCCTGACCACCGACAAGAGCAGCAGCACCGCCTACATG | |
| | CAGCTGAGCAGCCTGACCAGCGAGGACAGCGCCGTGTACTACTGCGCCAGGTACTAC | |
| | GACGACCACTACTGCCTGGACTACTGGGGCCAGGGCACCACCCTGACCGTGAGCAGC | |
| | GTGGAGGGCGGCAGCGGCGGCAGCGGCGGCAGCGGCGGCAGCGGCGGCGTGGACGAC | |
| | ATCCAGCTGACCCAGAGCCCCGCCATCATGAGCGCCAGCCCCGGCGAGAAGGTGACC | |
| | ATGACCTGCAGGGCCAGCAGCAGCGTGAGCTACATGAACTGGTACCAGCAGAAGAGC | |
| | GGCACCAGCCCCAAGAGGTGGATCTACGACACCAGCAAGGTGGCCAGCGGCGTGCCC | |
| | TACAGGTTCAGCGGCAGCGGCAGCGGCACCAGCTACAGCCTGACCATCAGCAGCATG | |
| | GAGGCCGAGGACGCCGCCACCTACTACTGCCAGCAGTGGAGCAGCAACCCCCTGACC | |
| | TTCGGCGCCGGCACCAAGCTGGAGCTGAAGCACCACCACCACCACCACTAG | |
| PDL1- | ATGGAGTTCGGCCTGAGCTGGGTGTTCCTGGTGGCCCTGTTCAGGGGCGTGCAGTGC | 49 |
| CD3 | GACATCAAGCTGCAGCAGAGCGGCGCCGAGCTGGCCAGGCCCGGCGCCAGCGTGAAG | |
| | ATGAGCTGCAAGACCAGCGGCTACACCTTCACCAGGTACACCATGCACTGGGTGAAG | |
| | CAGAGGCCCGGCCAGGGCCTGGAGTGGATCGGCTACATCAACCCCAGCAGGGGCTAC | |
| | ACCAACTACAACCAGAAGTTCAAGGACAAGGCCACCCTGACCACCGACAAGAGCAGC | |
| | AGCACCGCCTACATGCAGCTGAGCAGCCTGACCAGCGAGGACAGCGCCGTGTACTAC | |
| | TGCGCCAGGTACTACGACGACCACTACTGCCTGGACTACTGGGGCCAGGGCACCACC | |
| | CTGACCGTGAGCAGCGTGGAGGGCGGCAGCGGCGGCAGCGGCGGCAGCGGCGGCAGC | |
| | GGCGGCGTGGACGACATCCAGCTGACCCAGAGCCCCGCCATCATGAGCGCCAGCCCC | |
| | GGCGAGAAGGTGACCATGACCTGCAGGGCCAGCAGCAGCGTGAGCTACATGAACTGG | |
| | TACCAGCAGAAGAGCGGCACCAGCCCCAAGAGGTGGATCTACGACACCAGCAAGGTG | |
| | GCCAGCGGCGTGCCCTACAGGTTCAGCGGCAGCGGCAGCGGCACCAGCTACAGCCTG | |
| | ACCATCAGCAGCATGGAGGCCGAGGACGCCGCCACCTACTACTGCCAGCAGTGGAGC | |
| | AGCAACCCCCTGACCTTCGGCGCCGGCACCAAGCTGGAGCTGAAGGGCGGCGGCGGC | |
| | AGCGATATCCAGATGACACAGAGCCCATCATCTCTGTCTGCAAGCGTAGGAGACCGA | |
| | GTCACCATTACATGCAGAGCCTCCCAAGACGTTTCCACAGCAGTGGCCTGGTATCAG | |
| | CAAAAACCTGGTAAGGCGCCCAAGCTTCTCATCTATTCAGCCAGTTTTCTGTATAGC | |
| | GGCGTTCCCAGCCGATTCTCTGGCTCTGGATCCGGCACGGACTTTACTTTGACAATT | |
| | TCCTCTCTTCAGCCCGAAGATTTTGCAACCTACTACTGTCAGCAATATCTCTACCAT | |
| | CCAGCCACATTCGGACAGGGCACCAAAGTCGAAATCAAAAGAGGCGGCGGCGGCAGT | |
| | GGCGGCGGGGGTTCAGGAGGCGGGGGTTCTGAAGTGCAACTCGTTGAAAGCGGAGGA | |
| | GGGCTTGTCCAACCTGGCGGGTCACTGCGGTTGAGCTGCGCCGCAAGCGGATTCACC | |
| | TTCTCAGACTCTTGGATCCATTGGGTGCGCCAGGCTCCCGGAAAAGGCTTGGAATGG | |
| | GTTGCTTGGATTTCACCGTATGGCGGTTCCACATACTACGCTGACAGCGTTAAGGGT | |
| | CGATTCACCATCTCTGCAGATACTTCAAAAAACACAGCCTACCTTCAGATGAATAGT | |
| | TTGCGCGCCGAGGACACAGCGGTTTATTATTGTGCCCGAAGACATTGGCCCGGCGGT | |
| | TTCGACTACTGGGGGCAAGGTACGTTGGTGACTGTGAGCGCCCACCACCATCATCAC | |
| | CACTGA | |
| PDL1- | ATGGAGTTCGGCCTGAGCTGGGTGTTCCTGGTGGCCCTGTTCAGGGGCGTGCAGTGC | 51 |
| CD3- | GACATCAAGCTGCAGCAGAGCGGCGCCGAGCTGGCCAGGCCCGGCGCCAGCGTGAAG | |
| Fc | ATGAGCTGCAAGACCAGCGGCTACACCTTCACCAGGTACACCATGCACTGGGTGAAG | |
| | CAGAGGCCCGGCCAGGGCCTGGAGTGGATCGGCTACATCAACCCCAGCAGGGGCTAC | |
| | ACCAACTACAACCAGAAGTTCAAGGACAAGGCCACCCTGACCACCGACAAGAGCAGC | |
| | AGCACCGCCTACATGCAGCTGAGCAGCCTGACCAGCGAGGACAGCGCCGTGTACTAC | |
| | TGCGCCAGGTACTACGACGACCACTACTGCCTGGACTACTGGGGCCAGGGCACCACC | |
| | CTGACCGTGAGCAGCGTGGAGGGCGGCAGCGGCGGCAGCGGCGGCAGCGGCGGCAGC | |
| | GGCGGCGTGGACGACATCCAGCTGACCCAGAGCCCCGCCATCATGAGCGCCAGCCCC | |
| | GGCGAGAAGGTGACCATGACCTGCAGGGCCAGCAGCAGCGTGAGCTACATGAACTGG | |
| | TACCAGCAGAAGAGCGGCACCAGCCCCAAGAGGTGGATCTACGACACCAGCAAGGTG | |
| | GCCAGCGGCGTGCCCTACAGGTTCAGCGGCAGCGGCAGCGGCACCAGCTACAGCCTG | |
| | ACCATCAGCAGCATGGAGGCCGAGGACGCCGCCACCTACTACTGCCAGCAGTGGAGC | |
| | AGCAACCCCCTGACCTTCGGCGCCGGCACCAAGCTGGAGCTGAAGGGCGGCGGCGGC | |
| | AGCGATATCCAGATGACACAGAGCCCATCATCTCTGTCTGCAAGCGTAGGAGACCGA | |
| | GTCACCATTACATGCAGAGCCTCCCAAGACGTTTCCACAGCAGTGGCCTGGTATCAG | |
| | CAAAAACCTGGTAAGGCGCCCAAGCTTCTCATCTATTCAGCCAGTTTTCTGTATAGC | |
| | GGCGTTCCCAGCCGATTCTCTGGCTCTGGATCCGGCACGGACTTTACTTTGACAATT | |
| | TCCTCTCTTCAGCCCGAAGATTTTGCAACCTACTACTGTCAGCAATATCTCTACCAT | |
| | CCAGCCACATTCGGACAGGGCACCAAAGTCGAAATCAAAAGAGGCGGCGGCGGCAGT | |
| | GGCGGCGGGGGTTCAGGAGGCGGGGGTTCTGAAGTGCAACTCGTTGAAAGCGGAGGA | |
| | GGGCTTGTCCAACCTGGCGGGTCACTGCGGTTGAGCTGCGCCGCAAGCGGATTCACC | |
| | TTCTCAGACTCTTGGATCCATTGGGTGCGCCAGGCTCCCGGAAAAGGCTTGGAATGG | |
| | GTTGCTTGGATTTCACCGTATGGCGGTTCCACATACTACGCTGACAGCGTTAAGGGT | |
| | CGATTCACCATCTCTGCAGATACTTCAAAAAACACAGCCTACCTTCAGATGAATAGT | |
| | TTGCGCGCCGAGGACACAGCGGTTTATTATTGTGCCCGAAGACATTGGCCCGGCGGT | |
| | TTCGACTACTGGGGGCAAGGTACGTTGGTGACTGTGAGCGCCGTAGATGAAGCAAAA | |
| | TCTTGTGACAAAACCCATACCTGCCCACCATGCCCAGCCCCAGAACTTCTTGGCGGA | |
| | CCCTCTGTCTTCCTTTTCCCTCCGAAGCCCAAGGATACCCTGATGATCAGCCGAACC | |
| | CCGGAGGTAACATGTGTGGTGGTCGATGTTAGCCATGAGGATCCTGAAGTCAAATTT | |
| | AACTGGTATGTAGACGGTGTTGAGGTGCACAACGCTAAAACTAAGCCCAGGGAGGAG | |
| | CAGTACAACTCAACCTATCGCGTCGTATCTGTGCTTACCGTCCTGCATCAAGACTGG | |
| | CTCAATGGTAAGGAATATAAATGTAAAGTGAGTAACAAGGCACTGCCAGCACCTATC | |
| | GAAAAAACCATCTCAAAGGCGAAGGGACAGCCCAGGGAACCCCAGGTCTATACTCTG | |
| | CCACCTTCTCGGGATGAATTGACCAAGAACCAAGTTAGCCTGACATGTCTGGTGAAA | |
| | GGTTTCTATCCAAGCGATATAGCTGTCGAGTGGGAGTCCAATGGCCAACCTGAGAAC | |
| | AATTATAAGACCACCCCACCCGTTCTGGACAGCGACGGATCCTTTTTCCTGTACTCA | |
| | AAACTCACTGTCGATAAATCAAGATGGCAACAAGGCAACGTTTTTAGCTGTAGCGTG | |
| | ATGCACGAAGCACTTCATAATCACTATACACAGAAGTCACTCTCTCTTTCTCCAGGA | |
| | CACCACCATCATCACCACTGA | |
| SIRP1α- | ATGGAAACCGATACACTTCTGTTGTGGGTGCTGCTGCTGTGGGTCCCTGGTTCAACA | 67 |
| CD3- | GGCGATTATCCCTACGATGTGCCCGACTACGCAGGCGCTCAGCCAGCTGATGATATC | |
| PDL1- | CAGATGACACAGAGCCCATCATCTCTGTCTGCAAGCGTAGGAGACCGAGTCACCATT | |
| Fc (SL) | ACATGCAGAGCCTCCCAAGACGTTTCCACAGCAGTGGCCTGGTATCAGCAAAAACCT | |
| | GGTAAGGCGCCCAAGCTTCTCATCTATTCAGCCAGTTTTCTGTATAGCGGCGTTCCC | |
| | AGCCGATTCTCTGGCTCTGGATCCGGCACGGACTTTACTTTGACAATTTCCTCTCTT | |
| | CAGCCCGAAGATTTTGCAACCTACTACTGTCAGCAATATCTCTACCATCCAGCCACA | |
| | TTCGGACAGGGCACCAAAGTCGAAATCAAAAGAGGCGGCGGCGGCAGTGGCGGCGGG | |
| | GGTTCAGGAGGCGGGGGTTCTGAAGTGCAACTCGTTGAAAGCGTAGGAGGGCTTGTC | |
| | CAACCTGGCGGGTCACTGCGGTTGAGCTGCGCCGCAAGCGGATTCACCTTCTCAGAC | |
| | TCTTGGATCCATTGGGTGCGCCAGGCTCCCGGAAAAGGCTTGGAATGGGTTGCTTGG | |
| | ATTTCACCGTATGGCGGTTCCACATACTACGCTGACAGCGTTAAGGGTCGATTCACC | |
| | ATCTCTGCAGATACTTCAAAAAACACAGCCTACCTTCAGATGAATAGTTTGCGCGCC | |
| | GAGGACACAGCGGTTTATTATTGTGCCCTAAGACATTGGCCCGGCGGTTTCGACTAC | |
| | TGGGGGCAAGGTACGTTGGTGACTGTGAGCGCCGTAGATGAAGCAAAATCTTGTGAC | |
| | AAAACCCATACCTGCCCACCATGCCCAGCCCCAGAACTTCTTGGCGTACCCTCTGTC | |
| | TTCCTTTTCCCTCCGAAGCCCAAGGATACCCTGATGATCAGCCGAACCCCGGAGGTA | |
| | ACATGTGTGGTGGTCGATGTTAGCCATGAGGATCCTGAAGTCAAATTTAACTGGTAT | |
| | GTAGACGGTGTTGAGGTGCACAACGCTAAAACTAAGCCCAGGGAGGAGCAGTACAAC | |
| | TCAACCTATCGCGTCGTATCTGTGCTTACCGTCCTGCATCAAGACTGGCTCAATGGT | |
| | AAGGAATATAAATGTAAAGTGAGTAACAAGGCACTGCCAGCACCTATCGAAAAAACC | |
| | ATCTCAAAGGCGAAGGGACAGCCCAGGGAACCCCAGGTCTATACTCTGCAACCTTCT | |
| | CGGGATGAATTGACCAAGAACCAAGTTAGCCTGACATGTCTGGTGAAAGGTTTCTAT | |
| | CCAAGCGATATAGCTGTCGAGTGGGAGTCCAATGGCCAACCTGAGAACAATTATAAG | |
| | ACCACCCCACCCGTTCTGGACAGCGACGGATCCTTTTTCCTGTACTCAAAACTCACT | |
| | GTCGATAAATCAAGATGGCAACAAGGCAACGTTTTTAGCTGTAGCGTGATGCACGAA | |
| | GCACTTCATAATCACTATACACAGAAGTCACTCTCTCTTTCTCCAGGAAAGGTTGAC | |
| | GAACAGAAATTGATATCCGAGGAAGATCTCAATAGGAGGAAGAGAGAAGGCAGGGGG | |
| | AGCCTTCTCACTTGCGGCGATGTCGAGGAAAATCCGGGGCCTATGGAGACCGATACC | |
| | CTGCTCTTGTGGGTTTTGCTTCTTTGGGTGCCAGGATCTACAGGTGATGAAGAAGAA | |
| | TTGCAGATCATCCAACCAGACAAATCCGTACTCGTGGCCGCAGGAGAGACCGCTACC | |
| | CTCAGATGTACCATCACTTCTCTCTTCCCCGTTGGCCCCATCCAGTGGTTTCGAGGC | |
| | GCAGGACCAGGACGAGTGCTTATTTACAATCAACGACAGGGCCCATTCCCAAGAGTG | |
| | ACAACAGTATCCGATACCACCAAGCGCAATAATATGGACTTTAGCATTAGAATCGGC | |
| | AACATAACACCCGCTGACGCCGGTACATACTATTGTATTAAATTTCGAAAGGGCTCA | |
| | CCAGACGACGTGGAATTTAAGTCAGGGGCCGGAACCGAACTCTCAGTTAGAGCAAAA | |
| | CCTTCTGCTAGCGACATCAAGCTGCAGCAGAGCGGCGCCGAGCTGGCCAGGCCCGGC | |
| | GCCAGCGTGAAGATGAGCTGCAAGACCAGCGGCTACACCTTCACCAGGTACACCATG | |
| | CACTGGGTGAAGCAGAGGCCCGGCCAGGGCCTGGAGTGGATCGGCTACATCAACCCC | |
| | AGCAGGGGCTACACCAACTACAACCAGAAGTTCAAGGACAAGGCCACCCTGACCACC | |
| | GACAAGAGCAGCAGCACCGCCTACATGCAGCTGAGCAGCCTGACCAGCGAGGACAGC | |
| | GCCGTGTACTACTGCGCCAGGTACTACGACGACCACTACTGCCTGGACTACTGGGGC | |
| | CAGGGCACCACCCTGACCGTGAGCAGCGTGGAGGGCGGCAGCGGCGGCAGCGGCGGC | |
| | AGCGGCGGCAGCGGCGGCGTGGACGACATCCAGCTGACCCAGAGCCCCGCCATCATG | |
| | AGCGCCAGCCCCGGCGAGAAGGTGACCATGACCTGCAGGGCCAGCAGCAGCGTGAGC | |
| | TACATGAACTGGTACCAGCAGAAGAGCGGCACCAGCCCCAAGAGGTGGATCTACGAC | |
| | ACCAGCAAGGTGGCCAGCGGCGTGCCCTACAGGTTCAGCGGCAGCGGCAGCGGCACC | |
| | AGCTACAGCCTGACCATCAGCAGCATGGAGGCCGAGGACGCCGCCACCTACTACTGC | |
| | CAGCAGTGGAGCAGCAACCCCCTGACCTCCGGCGCCGGCACCAAGCTGGAGCTGAAG | |
| | CACCACCATCATCACCACTGA | |
| SIRP1α- | ATGGAAACCGATACACTTCTGTTGTGGGTGCTGCTGCTGTGGGTCCCTGGTTCAACA | 69 |
| CD3- | GGCGATTATCCCTACGATGTGCCCGACTACGCAGGCGCTCAGCCAGCTGATGATATC | |
| PDL1- | CAGATGACACAGAGCCCATCATCTCTGTCTGCAAGCGTAGGAGACCGAGTCACCATT | |
| Fc (LL) | ACATGCAGAGCCTCCCAAGACGTTTCCACAGCAGTGGCCTGGTATCAGCAAAAACCT | |
| | GGTAAGGCGCCCAAGCTTCTCATCTATTCAGCCAGTTTTCTGTATAGCGGCGTTCCC | |
| | AGCCGATTCTCTGGCTCTGGATCCGGCACGGACTTTACTTTGACAATTTCCTCTCTT | |
| | CAGCCCGAAGATTTTGCAACCTACTACTGTCAGCAATATCTCTACCATCCAGCCACA | |
| | TTCGGACAGGGCACCAAAGTCGAAATCAAAAGAGGCGGCGGCGGCAGTGGCGGCGGG | |
| | GGTTCAGGAGGCGGGGGTTCTGAAGTGCAACTCGTTGAAAGCGTAGGAGGGCTTGTC | |
| | CAACCTGGCGGGTCACTGCGGTTGAGCTGCGCCGCAAGCGGATTCACCTTCTCAGAC | |
| | TCTTGGATCCATTGGGTGCGCCAGGCTCCCGGAAAAGGCTTGGAATGGGTTGCTTGG | |
| | ATTTCACCGTATGGCGGTTCCACATACTACGCTGACAGCGTTAAGGGTCGATTCACC | |
| | ATCTCTGCAGATACTTCAAAAAACACAGCCTACCTTCAGATGAATAGTTTGCGCGCC | |
| | GAGGACACAGCGGTTTATTATTGTGCCCTAAGACATTGGCCCGGCGGTTTCGACTAC | |
| | TGGGGGCAAGGTACGTTGGTGACTGTGAGCGCCGTAGATGAAGCAAAATCTTGTGAC | |
| | AAAACCCATACCTGCCCACCATGCCCAGCCCCAGAACTTCTTGGCGTACCCTCTGTC | |
| | TTCCTTTTCCCTCCGAAGCCCAAGGATACCCTGATGATCAGCCGAACCCCGGAGGTA | |
| | ACATGTGTGGTGGTCGATGTTAGCCATGAGGATCCTGAAGTCAAATTTAACTGGTAT | |
| | GTAGACGGTGTTGAGGTGCACAACGCTAAAACTAAGCCCAGGGAGGAGCAGTACAAC | |
| | TCAACCTATCGCGTCGTATCTGTGCTTACCGTCCTGCATCAAGACTGGCTCAATGGT | |
| | AAGGAATATAAATGTAAAGTGAGTAACAAGGCACTGCCAGCACCTATCGAAAAAACC | |
| | ATCTCAAAGGCGAAGGGACAGCCCAGGGAACCCCAGGTCTATACTCTGCAACCTTCT | |
| | CGGGATGAATTGACCAAGAACCAAGTTAGCCTGACATGTCTGGTGAAAGGTTTCTAT | |
| | CCAAGCGATATAGCTGTCGAGTGGGAGTCCAATGGCCAACCTGAGAACAATTATAAG | |
| | ACCACCCCACCCGTTCTGGACAGCGACGGATCCTTTTTCCTGTACTCAAAACTCACT | |
| | GTCGATAAATCAAGATGGCAACAAGGCAACGTTTTTAGCTGTAGCGTGATGCACGAA | |
| | GCACTTCATAATCACTATACACAGAAGTCACTCTCTCTTTCTCCAGGAAAGGTTGAC | |
| | GAACAGAAATTGATATCCGAGGAAGATCTCAATAGGAGGAAGAGAGAAGGCAGGGGG | |
| | AGCCTTCTCACTTGCGGCGATGTCGAGGAAAATCCGGGGCCTATGGAGACCGATACC | |
| | CTGCTCTTGTGGGTTTTGCTTCTTTGGGTGCCAGGATCTACAGGTGATGAAGAAGAA | |
| | TTGCAGATCATCCAACCAGACAAATCCGTACTCGTGGCCGCAGGAGAGACCGCTACC | |
| | CTCAGATGTACCATCACTTCTCTCTTCCCCGTTGGCCCCATCCAGTGGTTTCGAGGC | |
| | GCAGGACCAGGACGAGTGCTTATTTACAATCAACGACAGGGCCCATTCCCAAGAGTG | |
| | ACAACAGTATCCGATACCACCAAGCGCAATAATATGGACTTTAGCATTAGAATCGGC | |
| | AACATAACACCCGCTGACGCCGGTACATACTATTGTATTAAATTTCGAAAGGGCTCA | |
| | CCAGACGACGTGGAATTTAAGTCAGGGGCCGGAACCGAACTCTCAGTTAGAGCAAAA | |
| | CCTTCTGCTAGCGGCGGCGGCGGCAGCGACATCAAGCTGCAGCAGAGCGGCGCCGAG | |
| | CTGGCCAGGCCCGGCGCCAGCGTGAAGATGAGCTGCAAGACCAGCGGCTACACCTTC | |
| | ACCAGGTACACCATGCACTGGGTGAAGCAGAGGCCCGGCCAGGGCCTGGAGTGGATC | |
| | GGCTACATCAACCCCAGCAGGGGCTACACCAACTACAACCAGAAGTTCAAGGACAAG | |
| | GCCACCCTGACCACCGACAAGAGCAGCAGCACCGCCTACATGCAGCTGAGCAGCCTG | |
| | ACCAGCGAGGACAGCGCCGTGTACTACTGCGCCAGGTACTACGACGACCACTACTGC | |
| | CTGGACTACTGGGGCCAGGGCACCACCCTGACCGTGAGCAGCGTGGAGGGCGGCAGC | |
| | GGCGGCAGCGGCGGCAGCGGCGGCAGCGGCGGCGTGGACGACATCCAGCTGACCCAG | |
| | AGCCCCGCCATCATGAGCGCCAGCCCCGGCGAGAAGGTGACCATGACCTGCAGGGCC | |
| | AGCAGCAGCGTGAGCTACATGAACTGGTACCAGCAGAAGAGCGGCACCAGCCCCAAG | |
| | AGGTGGATCTACGACACCAGCAAGGTGGCCAGCGGCGTGCCCTACAGGTTCAGCGGC | |
| | AGCGGCAGCGGCACCAGCTACAGCCTGACCATCAGCAGCATGGAGGCCGAGGACGCC | |
| | GCCACCTACTACTGCCAGCAGTGGAGCAGCAACCCCCTGACCTTCGGCGCCGGCACC | |
| | AAGCTGGAGCTGAAGCACCACCACCACCACCACTAG | |
| FAP-CD3 | ATGGTTTTGCTTGTGACCAGCCTCCTGCTCTGTGAACTGCCTCATCCTGCATTCCTG | 74 |
| | TTGATCCCACAGGTGCAGCTCCAGCAGAGTGGCGCAGAGCTCGCTCGCCCAGGCGCT | |
| | TCTGTGAATCTGAGTTGTAAGGCCTCCGGATATACTTTTACGAACAACGGCATCAAC | |
| | TGGCTGAAGCAGCGGACCGGCCAGGGCCTGGAGTGGATCGGCGAAATATACCCCCGG | |
| | TCCACAAACACTCTCTATAACGAGAAGTTTAAGGGCAAAGCAACTCTGACCGCGGAC | |
| | AGGTCCTCTAACACAGCCTATATGGAGCTGAGAAGCTTGACGAGTGAGGACTCCGCT | |
| | GTCTATTTTTGCGCCCGAACTCTGACCGCTCCTTTTGCTTTTTGGGGCCAGGGCACG | |
| | CTCGTGACCGTAAGTGCGGGCTCCACTAGCGGTTCCGGCAAACCTGGCAGCGGAGAA | |
| | GGCAGCACCAAAGGGCAGATCGTCCTGACGCAGTCTCCAGCCATCATGAGCGCCTCA | |
| | CCCGGCGAAAAGGTGACCATGACCTGCTCAGCCTCTTCTGGTGTGAATTTCATGCAC | |
| | TGGTACCAGCAAAAAAGTGGGACCTCCCCTAAAAGGTGGATCTTCGATACCAGCAAA | |
| | CTGGCTTCTGGCGTTCCCGCAAGGTTTAGCGGCTCTGGTTCCGGCACATCATACAGC | |
| | CTGACGATCAGCAGCATGGAGGCAGAAGACGCAGCTACCTATTACTGCCAGCAATGG | |
| | AGCTTTAACCCACCTACTTTCGGAGGAGGAACAAAGCTGGAAATAAAAAGAGGTGGT | |
| | GGTGGATCAGAGGTGCAGCTGGTGGAGTCTGGGGGAGGCTTGGTGCAGCCTGGAAAG | |
| | TCCCTGAAACTCTCCTGTGAGGCCTCTGGATTCACCTTCAGCGGCTATGGCATGCAC | |
| | TGGGTCCGCCAGGCTCCAGGGAGGGGGCTGGAGTCGGTCGCATACATTACTAGTAGT | |
| | AGTATTAATATCAAATATGCTGACGCTGTGAAAGGCCGGTTCACCGTCTCCAGAGAC | |
| | AATGCCAAGAACTTACTGTTTCTACAAATGAACATTCTCAAGTCTGAGGACACAGCC | |
| | ATGTACTACTGTGCAAGATTCGACTGGGACAAAAATTACTGGGGCCAAGGAACCATG | |
| | GTCACCGTCTCCTCAGGTGGTGGTGGATCAGGTGGAGGCGGAAGTGGAGGTGGCGGA | |
| | TCCGACATCCAGATGACCCAGTCTCCATCATCACTGCCTGCCTCCCTGGGAGACAGA | |
| | GTCACTATCAATTGTCAGGCCAGTCAGGACATTAGCAATTATTTAAACTGGTACCAG | |
| | CAGAAACCAGGGAAAGCTCCTAAGCTCCTGATCTATTATACAAATAAATTGGCAGAT | |
| | GGAGTCCCATCAAGGTTCAGTGGCAGTGGTTCTGGGAGAGATTCTTCTTTCACTATC | |
| | AGCAGCCTGGAATCCGAAGATATTGGATCTTATTACTGTCAACAGTATTATAACTAT | |
| | CCGTGGACGTTCGGACCTGGCACCAAGCTGGAAATCAAACGGCACCACCATCATCAC | |
| | CACTGA | |

Additional exemplarily embodiments of engager molecules include engager molecules comprising an effector domain comprising an anti-CD3 scFv (e.g., comprised of SEQ ID NOs: 20 and 22) and an activation domain comprising an scFv that binds to a cell surface protein such as CTLA4, TIM3, LAG3, BTLA, MRP, TIGIT, 0X40, or GITR. In some embodiments, the oncolytic viruses described herein comprise a bicistronic or multicistronic nucleic acid sequence, wherein a first nucleic acid sequence encodes an engager molecules comprising an effector domain comprising an anti-CD3 scFv (e.g., comprised of SEQ ID NOs: 20 and 22) and an activation domain comprising an scFv that binds to a cell surface protein such as CTLA4, TIM3, LAG3, BTLA, KIR, TIGIT, OX40, CD47, or GITR, and a second nucleic acid sequence encoding a therapeutic molecule such as IL-15 (SEQ ID NO: 24), IL-12 (SEQ ID NOs: 26 and 28), CXCL10 (SEQ ID NO: 30), or MMP9 (SEQ ID NO: 34). In such embodiments, the engager molecule is linked to the therapeutic molecule polypeptide by a T2A self-cleaving peptide linker (SEQ ID NO: 14).

Additional exemplarily embodiments of engager molecules include engager molecules comprising an effector domain comprising an anti-CD3 scFv (e.g., comprised of SEQ ID NOs: 20 and 22) and a targeting domain comprising an scFv that binds to SLAMF7 (also known as CD319) or CD27 (either the membrane bound form of CD27 or the soluble form of CD27). In some embodiments, the oncolytic viruses described herein comprise a bicistronic or multicistronic nucleic acid sequence, wherein a first nucleic acid sequence encodes an engager molecules comprising an effector domain comprising an anti-CD3 scFv (e.g., comprised of SEQ ID NOs: 20 and 22) and an antigen-recognition domain comprising an scFv that binds to a target cell antigen such as SLAMF7 or CD27, and a second nucleic acid sequence encoding a therapeutic molecule such as IL-15 (SEQ ID NO: 24), IL-12 (SEQ ID NOs: 26 and 28), CXCL10 (SEQ ID NO: 30), or MMP9 (SEQ ID NO: 34). In such embodiments, the engager molecule is linked to the therapeutic molecule polypeptide by a T2A self-cleaving peptide linker (SEQ ID NO: 14).

Additional cell surface proteins that are suitable for target by the engager molecules described herein are shown below in Table 3. Additional proteins that are suitable for use as therapeutic molecules are show below in Table 4.

TABLE 3
Cell-surface proteins suitable for targeting by engager molecules

| | NCBI Reference Sequence |
|---|---|
| Cell-surface protein | (RefSeq) Identifier |
| human SLAMF7 | NP_067004.3 |
| human NKGD2L | NP_079494.1 |
| human CTLA4 | NP_005205.2 |
| human TIM3 | NP_116171.3 |
| human LAG3 | NP_002277.4 |
| human BTLA (isoform | NP_001078826.1; |
| 1 and 2, respectively) | NP_861445.3 |
| human KIR | |
| human TIGIT | NP_776160.2 |
| human OX40 | NP_003318.1 |
| human GITR (isoform | NP_004186.1; |
| 1, 2, 3 respectively) | NP_683699.1; |
| | NP_683700.1 |
| human CD27 | NP_001233.1 |
| human CD40 (isoforms | NP_001241.1; |
| 1-5, respectively) | NP_690593.1; |
| | NP_001289682.1; |
| | NP_001309350.1; |
| | NP_001309351.1 |
| human NKGD2L | NP_079494.1 |
| human CD200 | NP_005935.4 |

TABLE 4
Proteins suitable for use as therapeutic molecules

| | NCBI Reference Sequence |
|---|---|
| Molecule | (RefSeq) Identifier |
| human TNFα | NP_000585.2 |
| human CX3CL1 | NP_002987.1 |
| human CCR4 | NP_005499.1 |
| human CSF-1 | NP_000748.3 |
| human TGFβ | NP_000651.3 |
| human IL-7 | NP_000871.1 |
| human GM-CSF | NP_000749.2 |

In some embodiments, the present invention provides compositions and methods of use for the prevention, treatment, and/or amelioration of a cancerous disease. In some embodiments, the methods described herein comprise administering an effective amount (e.g., a therapeutically effective amount) of an oncolytic virus described herein to a subject in need thereof, wherein the virus expresses an engager molecule or an engager molecule and a therapeutic molecule.

In some embodiments, compositions and methods of the present invention are useful for all stages and types of cancer, including for minimal residual disease, early solid tumor, advanced solid tumor and/or metastatic solid tumor. In some embodiments, compositions and methods of the present invention are used to treat a variety of solid tumors associated with a number of different cancers. The term “solid tumors” refers to relapsed or refractory tumors as well as metastases (wherever located), other than metastases observed in lymphatic cancer.

Exemplarily solid tumors include, but are not limited to, brain and other central nervous system tumors (e.g. tumors of the meninges, brain, spinal cord, cranial nerves and other parts of central nervous system, e.g. glioblastomas or medulla blastoma); head and/or neck cancer; breast tumors; circulatory system tumors (e.g. heart, mediastinum and pleura, and other intrathoracic organs, vascular tumors and tumor-associated vascular tissue); excretory system tumors (e.g. kidney, renal pelvis, ureter, bladder, other and unspecified urinary organs); gastrointestinal tract tumors (e.g. oesophagus, stomach, small intestine, colon, colorectal, recto sigmoid junction, rectum, anus and anal canal), tumors involving the liver and intrahepatic bile ducts, gall bladder, other and unspecified parts of biliary tract, pancreas, other and digestive organs); head and neck; oral cavity (lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx); reproductive system tumors (e.g. vulva, vagina, Cervix uteri, Corpus uteri, uterus, ovary, and other sites associated with female genital organs, placenta, penis, prostate, testis, and other sites associated with male genital organs); respiratory tract tumors (e.g. nasal cavity and middle ear, accessory sinuses, larynx, trachea, bronchus and lung, e.g. small cell lung cancer or non-small cell lung cancer); skeletal system tumors (e.g. bone and articular cartilage of limbs, bone articular cartilage and other sites); skin tumors (e.g. malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneum and peritoneum, eye and adnexa, thyroid, adrenal gland and other endocrine glands and related structures, secondary and unspecified malignant neoplasm of lymph nodes, secondary malignant neoplasm of respiratory and digestive systems and secondary malignant neoplasm of other sites, oligodendroglioma, oligoastrocytoma, astrocytoma, glioblastoma or medulloblastoma or other solid tumor.

In particular embodiments, the solid tumor is a brain tumor. In some instances, the brain tumor includes, but is not limited to, a glioma, in particular ependymoma, oligodendroglioma, oligoastrocytoma, astrocytoma, glioblastoma, or a medulloblastoma.

In some embodiments, compositions and methods of the present invention are used to treat a hematologic cancer. The term “hematologic cancer” refers herein to a cancer of the blood system and includes relapsed or refractory hematologic cancer as well as a metastasized hematologic cancer (wherever located). In some instances, the hematologic cancer is a T-cell malignancy or a B-cell malignancy. Exemplary T-cell malignancies include, but are not limited to, peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

Exemplary B-cell malignancies include, but are not limited to, chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some cases, the hematologic cancer is a relapsed or refractory hematologic cancer. In some cases, the hematologic cancer is a metastasized hematologic cancer.

In some embodiments, the oncolytic virus is engineered to produce a high level of expression of the engager molecule and/or the therapeutic polypeptide prior to the death of the virally-infected cell, e.g., within 1, 2, 3, 4, 5, 6, 7, 8, 9, 10, 11, 12, 13, 14, 15, 16, 17, 18, 19, 20, 21, 22, 23 or 24 hours of infection, or within 2, 3, 4, 5, or 6 days of infection. Expression of the engager molecule and/or the therapeutic polypeptide can be determined by methods known in the art, including Western blot, ELISA, immunoprecipitation, or electrophoresis, among others. In general, a “high level of expression” in reference to a therapeutic molecule refers to a level of expression that is greater than the basal level of expression of a corresponding polypeptide in a cell that is not infected with the oncolytic virus

### Compositions and Routes of Administration

In some embodiments, a therapeutically effective amount of an oncolytic virus or compositions thereof are administered to a subject. In accordance with this disclosure, the term “pharmaceutical composition” relates to a composition for administration to an individual. Administration of the compositions described herein can be local or systemic and can be effected by different ways, e.g., by intravenous, subcutaneous, intraperitoneal, intramuscular, topical or intradermal administration. In some embodiments, compositions disclosed herein are administered by any means known in the art. For example, the compositions described herein may be administered to a subject intravenously, intratumorally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a cream, or in a lipid composition. In particular embodiments, the composition is administered to the individual via infusion or injection. In some embodiments, administration is parenteral, e.g., intravenous. In some embodiments, the oncolytic virus or composition thereof is administered directly to the target site, e.g., by biolistic delivery to an internal or external target site or by catheter to a site in an artery. In particular embodiments, the compositions described herein are administered subcutaneously or intravenously. In some embodiments, the oncolytic viruses or compositions thereof described herein are administered intravenously or intraarterially.

In a preferred embodiment, the compositions described herein are formulated for a particular route of administration, for parenteral, transdermal, intraluminal, intra-arterial, intrathecal, intravenous administration, or for direct injection into a cancer. In some embodiments, the compositions further comprise a pharmaceutically acceptable carrier. “Pharmaceutically or pharmacologically acceptable” refer herein to molecular entities and compositions that do not produce an adverse, allergic or other untoward reaction when administered to an animal, or a human, as appropriate. In some embodiments, the pharmaceutical compositions of the present disclosure further comprise a pharmaceutically acceptable carrier. A “pharmaceutically acceptable carrier” includes any and all solvents, dispersion media, coatings, buffer, stabilizing formulation, antibacterial and antifungal agents, isotonic and absorption delaying agents and the like. Examples of suitable pharmaceutical carriers are well known in the art and include phosphate buffered saline solutions, water, emulsions, such as oil/water emulsions, various types of wetting agents, sterile solutions, etc. Compositions comprising such carriers are formulated by well-known conventional methods. In some embodiments, supplementary active ingredients are also incorporated into the compositions. For human administration, the compositions described herein are met with sterility, pyrogenicity, and general safety and purity standards as required by FDA Office of Biologics standards.

In some embodiments, the compositions described herein comprise a carrier such as a solvent or dispersion medium containing, for example, water, ethanol, polyol (for example, glycerol, propylene glycol, and liquid polyethylene glycol, and the like), suitable mixtures thereof, and vegetable oils. The proper fluidity is maintained, for example, by the use of a coating, such as lecithin, by the maintenance of the required particle size in the case of dispersion and by the use of surfactants. The prevention of the action of microorganisms is brought about by various antibacterial and antifungal agents known in the art. In many cases, it is preferable to include isotonic agents, for example, sugars or sodium chloride. In some embodiments, prolonged absorption of the injectable compositions is brought about by the use in the compositions of agents delaying absorption, for example, aluminum monostearate and gelatin.

In some embodiments, the oncolytic viruses described herein are formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups are derived from inorganic bases such as, for example, sodium, potassium, ammonium, calcium, or ferric hydroxides, and such organic bases as isopropylamine, trimethylamine, histidine, procaine and the like.

Pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In some cases, the form is sterile and is fluid. In some cases, it is stable under the conditions of manufacture and certain storage parameters (e.g. refrigeration and freezing) and is preserved against the contaminating action of microorganisms, such as bacteria and fungi. Aqueous compositions of some embodiments herein include an effective amount of a virus, nucleic acid, therapeutic protein, peptide, construct, stimulator, inhibitor, and the like, dissolved or dispersed in a pharmaceutically acceptable carrier or aqueous medium. Aqueous compositions of vectors expressing any of the foregoing are also contemplated.

In certain embodiments, biological material is extensively dialyzed to remove undesired small molecular weight molecules and/or lyophilized for more ready formulation into a desired vehicle, where appropriate. In some embodiments, the active compounds or constructs are formulated for parenteral administration, e.g., formulated for injection via the intravenous, intramuscular, sub-cutaneous, intralesional, intranasal or intraperitoneal routes. Any route used for vaccination or boost of a subject is used. The preparation of an aqueous composition that contains an active component or ingredient is known to those of skill in the art in light of the present disclosure. Typically, such compositions are prepared as injectables, either as liquid solutions or suspensions; solid forms suitable for use in preparing solutions or suspensions upon the addition of a liquid prior to injection is also prepared; and the preparations are also emulsified.

In some instances, the oncolytic virus is dispersed in a pharmaceutically acceptable formulation for injection. In some embodiments, sterile injectable solutions are prepared by incorporating the active compounds or constructs in the required amount in the appropriate solvent with any of the other ingredients enumerated above, as required, followed by filtered sterilization.

Upon formulation, the compositions described herein are administered in a manner compatible with disease to be treated and the dosage formulation and in such amount as is therapeutically effective. The formulations are easily administered in a variety of dosage forms, such as the type of injectable solutions described above, but also as slow release capsules or microparticles and microspheres and the like.

For parenteral administration in an aqueous solution, for example, the solution is suitably buffered if necessary and the liquid diluent first rendered isotonic with sufficient saline or glucose. These particular aqueous solutions are especially suitable for intravenous, intratumorally, intramuscular, subcutaneous and intraperitoneal administration. In this context, sterile aqueous media that is employed is known to those of skill in the art in light of the present disclosure. For example, one dosage is dissolved in 1 mL of isotonic NaCl solution and either added to 1000 mL of hypodermolysis fluid or injected at the proposed site of infusion.

In addition to the compounds formulated for parenteral administration, such as intravenous, intratumorally, intradermal or intramuscular injection, other pharmaceutically acceptable forms include, e.g., tablets or other solids for oral administration; liposomal formulations; time release capsules; biodegradable and any other form currently used.

In some embodiments, the viruses are encapsulated to inhibit immune recognition and placed at the site of a tumor.

In some instances, preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives are also present such as, for example, antimicrobials, anti-oxidants, chelating agents, and inert gases and the like. In addition, the pharmaceutical composition of the present disclosure might comprise proteinaceous carriers, like, e.g., serum albumin or immunoglobulin, preferably of human origin. It is envisaged that the pharmaceutical composition of the disclosure might comprise, in addition to the proteinaceous bispecific single chain antibody constructs or nucleic acid molecules or vectors encoding the same (as described in this disclosure), further biologically active agents, depending on the intended use of the pharmaceutical composition.

In some embodiments, tumor-infiltrating virus-producing cells which continuously release vectors are formulated for direct implantation into a tumor in order to increase the viral oncolysis and the transfer efficiency of the therapeutic genes.

Intranasal formulations are known in the art and are described in, for example, U.S. Pat. Nos. 4,476,116; 5,116,817; and 6,391,452. Formulations which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these are found in Remington: The Science and Practice of Pharmacy, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are also present. The nasal dosage form is isotonic with nasal secretions.

For administration by inhalation described herein is in a form as an aerosol, a mist or a powder. Pharmaceutical compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator is formulated containing a powder mix of the compound described herein and a suitable powder base such as lactose or starch.

### Therapeutically Effective Amounts and Therapeutic Regimens

In some embodiments, the oncolytic viruses and compositions thereof described herein are administered to a subject at therapeutically effective amount. The therapeutically effective amount will depend on the subject to be treated, the state (e.g., general health) of the subject, the protection desired, the disease to be treated, the route of administration, and/or the nature of the virus. In some embodiments, the person responsible for administration (e.g., an attending physician) will determine the appropriate dose for an individual. As is well known in the medical arts, dosages for any one patient depend upon many factors, including the patient's size, weight, body surface area, age, sex, and general health, the particular compound to be administered, the particular disease to be treated, timing and route of administration, and other drugs being administered concurrently. Therefore, it is expected that for each individual patient, even if the viruses that are administered to the population at large, each patient is monitored for the proper dosage for the individual, and such practices of monitoring a patient are routine in the art.

In some embodiments, the therapeutically effective amount of an oncolytic virus described herein is administered in a single dose. In some embodiments of the present invention, the oncolytic viruses or compositions thereof are administered to a subject at a dose ranging from about 1×10⁺⁵ pfu to about 1×10⁵ pfu (plaque forming units), about 1×10⁺⁸ pfu to about 1×10⁺¹⁵ pfu, about 1×10⁺¹⁰ pfu to about 1×10⁺⁵ pfu, or about 1×10⁺⁸ pfu to about 1×10⁺¹² pfu. For example, in some embodiments, the oncolytic viruses or compositions thereof are administered to a subject at a dose of about 10⁵, 10⁶, 10⁷, 10⁸, 10⁹, 10¹⁰, 10¹¹, 10¹², 10¹³, 10¹⁴, or 10¹⁵ pfu of virus. In some embodiments, the dose depends, on the age of the subject to which a composition is being administered. For example, a lower dose may be required if the subject is juvenile, and a higher dose may be required if the subject is an adult human subject. In certain embodiments, for example, a juvenile subject receives about 1×10⁺⁸ pfu and about 1×10⁺¹⁰ pfu, while an adult human subject receives a dose between about 1×10⁺¹⁰ pfu and about 1×10⁺¹² pfu. In some embodiments, the therapeutically effective amount of an oncolytic virus described herein is administered over the course of two or more doses. In some embodiments, the two or more doses are administered simultaneously (e.g., on the same day or over a short period of time) or at appropriate intervals, for example as two, three, four or more sub-doses per day.

In some embodiments, the oncolytic viruses or compositions thereof described herein are administered to a subject once. In some embodiments, the oncolytic viruses or compositions thereof described herein are administered to a subject more than once. For example, a composition disclosed herein may be administered multiple times, including 1, 2, 3, 4, 5, 6, or more times. In some embodiments, a composition disclosed herein may be administered to a subject on a daily or weekly basis for a time period or on a monthly, bi-yearly, or yearly basis depending on need or exposure to a pathogenic organism or to a condition in the subject (e.g. cancer). In particular embodiments, the oncolytic viruses and compositions thereof are formulated in such a way, and administered in such and amount and/or frequency, that they are retained by the subject for extended periods of time.

In some embodiments, the oncolytic viruses or compositions thereof are administered for therapeutic applications or is administered as a maintenance therapy, such as for example, for a patient in remission. In some embodiments, the oncolytic viruses or compositions thereof are administered once every month, once every 2 months, once every 6 months, once a year, twice a year, three times a year, once every two years, once every three years, or once every five years.

Im some embodiments wherein a patient's status does improve, the oncolytic viruses or compositions thereof may be administered continuously upon the doctor's discretion. In some embodiments, the dose composition is temporarily reduced and/or administration of the composition is temporarily suspended for a certain length of time (i.e., a “drug holiday”). In some embodiments, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

In some embodiments, once improvement of a patient's conditions has occurred, a maintenance dose may be administered if necessary. In some embodiments, the dosage and/or the frequency of administration of the composition is reduced, as a function of the symptoms, to a level at which the improved disease, disorder or condition is retained. In some embodiments, patients may require intermittent treatment on a long-term basis upon any recurrence of symptoms.

In some embodiments, toxicity and therapeutic efficacy of such therapeutic regimens are determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it is expressed as the ratio between LD₅₀ and ED₅₀. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

In some instances, tumor antigen expression levels are evaluated to assess the progress of treatment in a patient, to stratify a patient, and/or to modulate a therapeutic regimen. In some instances, assessment of antigen expression levels include the use of imrmunohistochernistry (IHC) (including semi-quantitative or quantitative IHC) or other antibody-based assays (Western blot, fluorescent immunoassay (FIA), fluorescence in situ hybridization (FISH), radioimmunoassay (RIA), radioimmunoprecipitation (RIP), enzyme-linked imnunosorbent assay (ELISX), immunoassay, immunoradiometric assay, fluoroimmunoassay, chemiluminescent assay, bioluminescent assay, gel electrophoresis), or indirectly by quantitating the transcripts for these genes (e.g. by in situ hybridization, nuclease protection, Northern blot, polymerase chain reaction (PCR) including reverse transcriptase PCR (RT-PCR)). In some instances, cells, for example, lymphocytes, are analyzed using FACs technology or paraffin embedded tumor sections using antibodies.

In some instances, antibodies are used to characterize the protein content of target cells through techniques such as immunohistochemistry, ELISAs and Western blotting. In some cases, this provides a screen e.g. for the presence or absence of a subject likely to respond favorably to oncolytic virus therapy and/or a need for co-administering an immune stimulating agent with an oncolytic virus.

In some embodiments, immunohistochemistry is performed on a sample of tissue from a biopsy. In some cases, the sample is examined fresh or frozen. In some instances, antibodies against antigens presented in the cell are added to the sample on a slide and the antibodies bind wherever the antigens are present. In some embodiments, excess antibody is then washed away. In some cases, the antibodies that remain bound to the cell are further labeled by a secondary antibody for visualization under a microscope.

In some embodiments, test samples are obtained from a subject such as for example, from tissue (e.g. tumor biopsy), cerebrospinal fluid (CSF), lymph, blood, plasma, serum, peripheral blood mononuclear cells (PBMCs), lymph fluid, lymphocytes, synovial fluid and urine. In particular embodiments, the test sample is obtained from CSF or tumor tissue. In other particular embodiments, the test sample is obtained from tumor tissue and e.g. the relative number of CD4⁺ and/or CD8⁺ cells in the sample is determined and/or the level of one or more Th1 and/or Th2 cytokines in the sample is measured e.g. by immunofluorescent staining of fixed and permeabilized cells from the sample with antibodies against the Th1 and/or Th2 cytokines. In other particular embodiments, the test sample is obtained from blood and e.g. the level of one or more Th1 and/or Th2 cytokines in the sample is measured by ELISA.

### Combination Therapy

In some embodiments, the viruses, expression constructs, nucleic acid molecules and/or vectors described herein are administered in combination with another therapeutic agent. In some embodiments, the oncolytic viruses and an additional therapeutic agent are formulated in the same compositions. In such embodiments, the composition may further comprise a pharmaceutically acceptable carrier or excipient. In some embodiments, the oncolytic viruses and an additional therapeutic agent are formulated in separate compositions (e.g., two or more compositions suitable for administration to patient or subject). The disclosure further encompasses co-administration protocols with other cancer therapies, e.g. bispecific antibody constructs, targeted toxins or other compounds, including those which act via immune cells, including T-cell therapy. The clinical regimen for co-administration of the inventive composition(s) encompass(es) co-administration at the same time, before and/or after the administration of the other component. Particular combination therapies include chemotherapy, radiation, surgery, hormone therapy, and/or other types of immunotherapy. In some embodiments, a therapeutically effective amount of a oncolytic virus is administered to a subject in need thereof in combination with an additional therapeutic agent. In some instances, the additional therapeutic agent is a chemotherapeutic agent, a steroid, an immunotherapeutic agent, a targeted therapy, or a combination thereof.

In some embodiments, pharmaceutical compositions are administered in conjunction with an adjuvant therapy. For examples, activating adjuvant treatments are administered prior to, contemporaneous with, or after one or more administrations (e.g., intratumoral injection of the virus). For example, adjuvant therapy includes modulation of Toll-like receptor (TLR) ligands, such as TLR9 activation by DNA molecules comprising CpG sequences, or TLR9 activation (e.g., by RNA ligands). Other adjuvant treatments include agonizing antibodies or other polypeptides (e.g. activation of CD40 or GITR by CD410 Ligand (CD40L) or GTR Ligand (GITRL), respectively). Further, provided are cyclic dinucleotides (e.g., c-di-GMP) that modulate STING, Another activating adjuvant includes interleukins such as IL-33.

In some embodiments, the additional therapeutic agent comprises an agent selected from: bendamustine, bortezomib, lenalidomide, idelalisib (GS-1101), vorinostat, everolimus, panobinostat, temsirolimus, romidepsin, vorinostat, fludarabine, cyclophosphamide, mitoxantrone, pentostatine, prednisone, etopside, procarbazine, and thalidomide.

In some embodiments, the additional therapeutic agent is a multi-agent therapeutic regimen. In some embodiments the additional therapeutic agent comprises the HyperCVAD regimen (cyclophosphamide, vincristine, doxorubicin, dexamethasone alternating with methotrexate and cytarabine). In some embodiments, the HyperCVAD regimen is administered in combination with rituximab.

In some embodiments the additional therapeutic agent comprises the R-CHOP regiment (rituximab, cyclophosphamide, doxorubicin, vincristine, and prednisone). In some embodiments the additional therapeutic agent comprises the FCR regimen (FCR (fludarabine, cyclophosphamide, rituximab). In some embodiments the additional therapeutic agent comprises the FCMR regimen (fludarabine, cyclophosphamide, mitoxantrone, rituximab). In some embodiments the additional therapeutic agent comprises the FMR regimen (fludarabine, mitoxantrone, rituximab). In some embodiments the additional therapeutic agent comprises the PCR regimen (pentostatin, cyclophosphamide, rituximab). In some embodiments the additional therapeutic agent comprises the PEPC regimen (prednisone, etoposide, procarbazine, cyclophosphamide). In some embodiments the additional therapeutic agent comprises radioimmunotherapy with ⁹⁰Y-ibritumomab tiuxetan or ¹³¹I-tositumomab. In some embodiments, the additional therapeutic agent is an autologous stem cell transplant.

In some embodiments, the additional therapeutic agent is selected from: nitrogen mustards such as for example, bendamustine, chlorambucil, chlormethine, cyclophosphamide, ifosfamide, melphalan, prednimustine, trofosfamide; alkyl sulfonates like busulfan, mannosulfan, treosulfan; ethylene imines like carboquone, thiotepa, triaziquone; nitrosoureas like carmustine, fotemustine, lomustine, nimustine, ranimustine, semustine, streptozocin; epoxides such as for example, etoglucid; other alkylating agents such as for example dacarbazine, mitobronitol, pipobroman, temozolomide; folic acid analogues such as for example methotrexate, permetrexed, pralatrexate, raltitrexed; purine analogs such as for example cladribine, clofarabine, fludarabine, mercaptopurine, nelarabine, tioguanine; pyrimidine analogs such as for example azacitidine, capecitabine, carmofur, cytarabine, decitabine, fluorouracil, gemcitabine, tegafur; vinca alkaloids such as for example vinblastine, vincristine, vindesine, vinflunine, vinorelbine; podophyllotoxin derivatives such as for example etoposide, teniposide; colchicine derivatives such as for example demecolcine; taxanes such as for example docetaxel, paclitaxel, paclitaxel poliglumex; other plant alkaloids and natural products such as for example trabectedin; actinomycines such as for example dactinomycin; antracyclines such as for example aclarubicin, daunorubicin, doxorubicin, epirubicin, idarubicin, mitoxantrone, pirarubicin, valrubicin, zorubincin; other cytotoxic antibiotics such as for example bleomycin, ixabepilone, mitomycin, plicamycin; platinum compounds such as for example carboplatin, cisplatin, oxaliplatin, satraplatin; methylhydrazines such as for example procarbazine; sensitizers such as for example aminolevulinic acid, efaproxiral, methyl aminolevulinate, porfimer sodium, temoporfin; protein kinase inhibitors such as for example dasatinib, erlotinib, everolimus, gefitinib, imatinib, lapatinib, nilotinib, pazonanib, sorafenib, sunitinib, temsirolimus; other antineoplastic agents such as for example alitretinoin, altretamine, amzacrine, anagrelide, arsenic trioxide, asparaginase, bexarotene, bortezomib, celecoxib, denileukin diftitox, estramustine, hydroxycarbamide, irinotecan, lonidamine, masoprocol, miltefosein, mitoguazone, mitotane, oblimersen, pegaspargase, pentostatin, romidepsin, sitimagene ceradenovec, tiazofurine, topotecan, tretinoin, vorinostat; estrogens such as for example diethylstilbenol, ethinylestradiol, fosfestrol, polyestradiol phosphate; progestogens such as for example gestonorone, medroxyprogesterone, megestrol; gonadotropin releasing hormone analogs such as for example buserelin, goserelin, leuprorelin, triptorelin; anti-estrogens such as for example fulvestrant, tamoxifen, toremifene; anti-androgens such as for example bicalutamide, flutamide, nilutamide, enzyme inhibitors, aminoglutethimide, anastrozole, exemestane, formestane, letrozole, vorozole; other hormone antagonists such as for example abarelix, degarelix; Immunostimulants such as for example histamine dihydrochloride, mifamurtide, pidotimod, plerixafor, roquinimex, thymopentin; immunosuppressants such as for example everolimus, gusperimus, leflunomide, mycophenolic acid, sirolimus; calcineurin inhibitors such as for example ciclosporin, tacrolimus; other immunosuppressants such as for example azathioprine, lenalidomide, methotrexate, thalidomide; and Radiopharmaceuticals such as for example, iobenguane.

In some embodiments, the additional therapeutic agent is selected from: interferons, interleukins, tumor necrosis factors, growth factors, or the like.

In some embodiments, the additional therapeutic agent is selected from: ancestim, filgrastim, lenograstim, molgramostim, pegfilgrastim, sargramostim; Interferons such as for example IFNα natural, IFN α-2a, IFN α-2b, IFN alfacon-1, IFN α-n1, IFN βnatural, IFN-1a, IFN β-1b, IFN γ, peginterferon α-2a, peginterferon α-2b; interleukins such as for example aldesleukin, oprelvekin; other immunostimulants such as for example BCG vaccine, glatiramer acetate, histamine dihydrochloride, immunocyanin, lentinan, melanoma vaccine, mifamurtide, pegademase, pidotimod, plerixafor, poly I:C, poly ICLC, roquinimex, tasonermin, thymopentin; Immunosuppressants such as for example abatacept, abetimus, alefacept, antilymphocyte immunoglobulin (horse), antithymocyte immunoglobulin (rabbit), eculizumab, efalizumab, everolimus, gusperimus, leflunomide, muromab-CD3, mycophenolic acid, natalizumab, sirolimus; TNFα inhibitors such as for example adalimumab, afelimomab, certolizumab pegol, etanercept, golimumab, infliximab; Interleukin Inhibitors such as for example anakinra, basiliximab, canakinumab, daclizumab, mepolizumab, rilonacept, tocilizumab, ustekinumab; calcineurin inhibitors such as for example ciclosporin, tacrolimus; other immunosuppressants such as for example azathioprine, lenalidomide, methotrexate, thalidomide.

In some embodiments, the additional therapeutic agent is selected from: Adalimumab, Alemtuzumab, Basiliximab, Bevacizumab, Cetuximab, Certolizumab pegol, Daclizumab, Eculizumab, Efalizumab, Gemtuzumab, Ibritumomab tiuxetan, Infliximab, Muromonab-CD3, Natalizumab, Panitumumab, Ranibizumab, Rituximab, Tositumomab, Trastuzumab, or the like, or a combination thereof.

In some embodiments, the additional therapeutic agent is selected from: monoclonal antibodies such as for example alemtuzumab, bevacizumab, catumaxomab, cetuximab, edrecolomab, gemtuzumab, panitumumab, rituximab, trastuzumab; Immunosuppressants, eculizumab, efalizumab, muromab-CD3, natalizumab; TNF alpha Inhibitors such as for example adalimumab, afelimomab, certolizumab pegol, golimumab, infliximab; Interleukin Inhibitors, basiliximab, canakinumab, daclizumab, mepolizumab, tocilizumab, ustekinumab; Radiopharmaceuticals, ibritumomab tiuxetan, tositumomab; additional monoclonal antibodies such as for example abagovomab, adecatumumab, alemtuzumab, anti-CD30 monoclonal antibody Xmab2513, anti-MET monoclonal antibody MetMab, apolizumab, apomab, arcitumomab, basiliximab, bispecific antibody 2B1, blinatumomab, brentuximab vedotin, capromab pendetide, cixutumumab, claudiximab, conatumumab, dacetuzumab, denosumab, eculizumab, epratuzumab, epratuzumab, ertumaxomab, etaracizumab, figitumumab, fresolimumab, galiximab, ganitumab, gemtuzumab ozogamicin, glembatumumab, ibritumomab, inotuzumab ozogamicin, ipilimumab, lexatumumab, lintuzumab, lintuzumab, lucatumumab, mapatumumab, matuzumab, milatuzumab, monoclonal antibody CC49, necitumumab, nimotuzumab, oregovomab, pertuzumab, ramacurimab, ranibizumab, siplizumab, sonepcizumab, tanezumab, tositumomab, trastuzumab, tremelimumab, tucotuzumab celmoleukin, veltuzumab, visilizumab, volociximab, zalutumumab.

In some embodiments, the additional therapeutic agent is selected from: agents that affect the tumor micro-enviroment such as cellular signaling network (e.g. phosphatidylinositol 3-kinase (PI3K) signaling pathway, signaling from the B-cell receptor and the IgE receptor). In some embodiments, the additional therapeutic agent is a PI3K signaling inhibitor or a syc kinase inhibitor. In one embodiment, the syk inhibitor is R788. In another embodiment is a PKCγ inhibitor such as by way of example only, enzastaurin.

Examples of agents that affect the tumor micro-environment include PI3K signaling inhibitor, syc kinase inhibitor, protein kinase inhibitors such as for example dasatinib, erlotinib, everolimus, gefitinib, imatinib, lapatinib, nilotinib, pazonanib, sorafenib, sunitinib, temsirolimus; other angiogenesis inhibitors such as for example GT-111, JI-101, R1530; other kinase inhibitors such as for example AC220, AC480, ACE-041, AMG 900, AP24534, Arry-614, AT7519, AT9283, AV-951, axitinib, AZD1152, AZD7762, AZD8055, AZD8931, bafetinib, BAY 73-4506, BGJ398, BGT226, BI 811283, BI6727, BIBF 1120, BIBW 2992, BMS-690154, BMS-777607, BMS-863233, BSK-461364, CAL-101, CEP-11981, CYC116, DCC-2036, dinaciclib, dovitinib lactate, E7050, EMD 1214063, ENMD-2076, fostamatinib disodium, GSK2256098, GSK690693, TNCB18424, INNO-406, JNJ-26483327, JX-594, KX2-391, linifanib, LY2603618, MGCD265, MK-0457, MK1496, MLN8054, MLN8237, MP470, NMS-1116354, NMS-1286937, ON 01919.Na, OSI-027, OSI-930, Btk inhibitor, PF-00562271, PF-02341066, PF-03814735, PF-04217903, PF-04554878, PF-04691502, PF-3758309, PHA-739358, PLC3397, progenipoietin, R547, R763, ramucirumab, regorafenib, R05185426, SAR103168, SCH 727965, SGI-1176, SGX523, SNS-314, TAK-593, TAK-901, TKI258, TLN-232, TTP607, XL147, XL228, XL281RO5126766, XL418, XL765.

In some embodiments, the additional therapeutic agent is selected from: inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

In some embodiments, the additional therapeutic agent is selected from: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-such as for example growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

In some embodiments, the additional therapeutic agent is selected from: alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, ete.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

In some embodiments, pharmaceutical compositions are administered in conjunction with an adjuvant therapy. For examples, activating adjuvant treatments are administered prior to, contemporaneous with, or after one or more administrations (e.g., intratumoral injection of the virus), For example, adjuvant therapy includes modulation of Toll-like receptor (TLR) ligands, such as TLR9 activation by DNA molecules comprising CpG sequences, or TLR9 activation (e.g., by RNA ligands). Other adjuvant treatments include agonizing antibodies or other polypeptides (e.g., activation of CD40 or GITR by CD40 Ligand (CD40L) or G1TR Ligand (GTRTL), respectively). Further, provided are cyclic dinucleotides (e.g., c-di-GMP) that modulate STING. Another activating adjuvant includes interleukins such as IL-33. In some instances, the pharmaceutical compositions described herein are administered in conjunction with an adjuvant therapy.

### Kits

In some embodiments, the present invention provides kits comprising one or more oncolytic viruses as described herein, a nucleic acid sequence as described herein, a vector as described herein, and/or a host cell as described herein. In some embodiments, the kits comprise a pharmaceutical composition as described herein above, either alone or in combination with further therapeutic agents to be administered to an individual in need thereof.

In some embodiments, the present invention provides kits for the use of vectors and virus-producing cells according to the invention as drugs in therapeutic methods. In particular, the vectors and virus producing cells according to some embodiments of the invention are used for the therapy or treatment of solid tumors in a subject. In some embodiments, the therapeutic effect is caused by the oncolytic properties of the recombinant vectors and viruses as well as by the use of therapeutic genes.

In some embodiments, the present invention provides kits for use with methods and compositions. Some embodiments concern kits having vaccine compositions of use to reduce onset of or treat subjects having one or more solid tumors. Other embodiments concern kits for making and using molecular constructs described herein. In some instances, kits also include a suitable container, for example, vials, tubes, mini- or microfuge tubes, test tube, flask, bottle, syringe or other container. Where an additional component or agent is provided, the kit contains one or more additional containers into which this agent or component is placed. Kits herein also include a means for containing the constructs, vaccine compositions and any other reagent containers in close confinement for commercial sale. Such containers include injection or blow-molded plastic containers into which the desired vials are retained. Optionally, one or more additional agents such as other anti-viral agents, anti-fungal or anti-bacterial agents are needed for compositions described, for example, for compositions of use as a vaccine.

All publications, patents, and patent applications mentioned in this specification are herein incorporated by reference to the same extent as if each individual publication, patent, or patent application was specifically and individually indicated to be incorporated by reference.

### EXAMPLES

The examples below further illustrate the described embodiments without limiting the scope of the invention. While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1: Preparation of Pseudotyped VSV-G

The following protocol was adopted to prepare an exemplary pseudotyped VSV-G, by combining VSV-Glycoprotein (VSV-GP) with HIVI-gag and rev proteins.

Cell culture and transfection: DNA of the following packaging plasmids was mixed and prepared for transfection into 293T cells: pMDLg/pRRE expressing HIV-1 GAG/POL; pRSV/REV expressing HIV-1 REV; and pMD2.G 5 60 5.8 VSV glycoprotein. The DNA mix was added to 500 μL of pre-warmed Optimem II medium. A working stock of polyethyleneimine transfection reagent (PEI) was prepared at 1 μg/L in 1xPBS, pH 4.5, and 88 μL of the working stock was added to the mixture, maintaining a 4:1 v/w ratio of PEI:DNA. The mixture was vortexed briefly and left for 5-10 min at room temperature to form a PEI:DNA transfection complex. A total of 2.5×10⁶ low passage (less than P20) 293T cells were seeded per 15 cm dish in 15 mL DMEM supplemented with 10% serum and 1% Pen/Strep. 2 hours prior to transfection, the cell culture medium was aspirated and replaced with 15 mL of fresh pre-warmed growth medium (GM). The transfection complex was then added drop-wise to each 15 cm plate, swirled briefly to mix and incubated for 8 hrs in 10% CO₂, 35° C. After 8 hours, the medium was replaced with 10 mL of fresh growth medium containing 25 mM HEPES and 10% serum. The mixture was then incubated for 48 hrs post-transfection.

Virus collection: The medium from each dish was removed, pooled, and filtered through a 0.22 μm low protein binding/fast flow filter unit and stored at 4° C. A 5 mL volume of fresh growth medium was added to each dish and incubated overnight at 4° C. (60-72 hours post transfection). The second lot of medium from each dish was collected, as in the previous step, and pooled with previous media harvest. The plasmid carry-over is removed by digestion with DNASE-I (1 mg/mL stock). A 1 μg/mL solution the viral supernatant, supplemented with 1 μL of 1M MgCl₂, was incubated at room temperature for 30 min followed by 2-4 hrs at 4° C. The filtered supernatants can be used directly on cultured cells, or aliquoted and stored at −80° C. The pseudotyped VSV-G viral supernatant can be optionally concentrated and purified.

### Example 2: Construction of Pseudotyped VSV-G Expressing a CD28-CA125 Bispecific Antibody Engager Molecule

Pseudotyped VSV-G is prepared as described in Example 1 and further processed to express a nucleic acid encoding an engager polypeptide comprising an effector domain comprising an anti-CD28 molecule and a targeting domain comprising an anti-CA125 molecule, and a nucleic acid encoding an anti-PD1 immune modulatory peptide. The resulting oncolytic virus is a pseudotyped onlcolytic VSV-G virus encoding a CD28-CA125 engager molecule and an anti-PD1 therapeutic molecule (CD28-CA125-PD1 VSV-G).

### Example 3: CD28-CA125-PD1 VSV-G Activates Human T Cells and Exhibits Anti-Tumor Activity

Human T cells are infected with the pseudotyped CD28-CA125-PD1 VSV-G virus. 24 hrs to 48 hrs post viral infection, the T cell culture medium is collected and checked for the presence of proinflammatory cytokines. These results will show that T cells are activated by CD28-CA125-PD1 VSV-G, as evidenced by presence of proinflammatory cytokines such as IFN-3 and IL-2 in the cell culture supernatant of CD28-CA125-PD1 VSV-G infected human T cells.

EphA2-overexpressing gastric cancer cells, from KATO3 cell line, are infected with pseudotyped CD28-CA125-PD1 VSV-G or non-pseudotyped CD28-CA125-PD1 VSV virus and the cell proliferation is assessed. These results will show that cell proliferation is significantly reduced in cells KATO3 cells infected with pseudotyped CD28-CA125-PD1 VSV-G compared to KATO3 cells infected with non-pseudotyped CD28-CA125-PD1 VSV virus.

### Example 4: CD19-CD3, SIRP1α-CD3, and PDL1-CD3-Fc Engager Molecules Specifically Bind to T-Cells Via CD3

The binding of bipartite (CD19-CD3 and SIRP1α-CD3) and triparte (PDL1-CD3-Fc) engager molecules to T cells was assessed. Briefly, 25,000 T cells were stimulated with 200 U/mL IL-2 for 12 days. After 12 days, T cell were incubated with varying concentrations of engager molecules (500, 1000, or 2000 ng/mL for CD19-CD3 and SIRP1α-CD3; neat supernatant for PDL1-CD3-Fc) for 20 minutes at room temperature in triplicate. Cells were then washed twice, followed by staining with an anti-6XHis APC antibody at 500 ng/mL for an additional 20 minutes. Cells were washed again and treated with propidium iodide (PI) to exclude dead cells from further analysis. Stained cells were analyzed by flow cytometry on a BD LSR Fortesa cytometer and the percentage of the cell population positive for staining was set at 2% of the secondary only control.

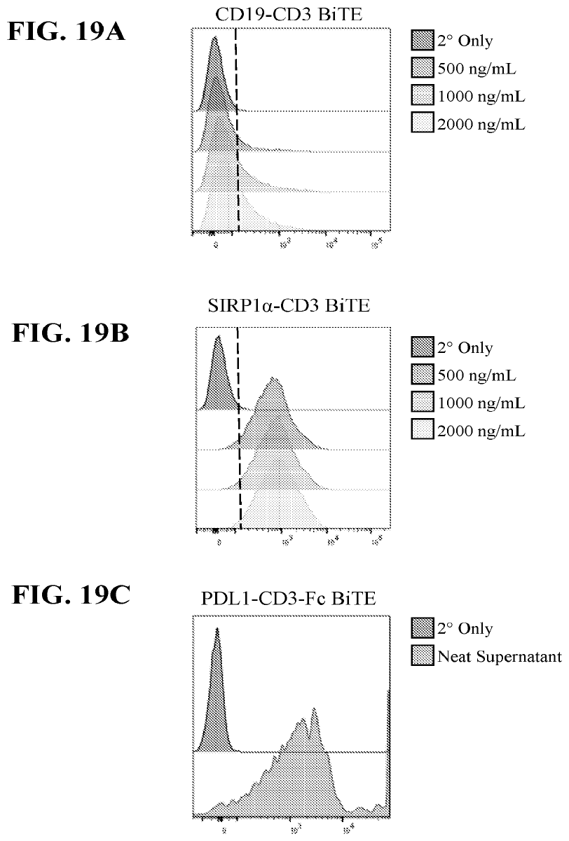

Results for CD19-CD3 (FIG. 19A), SIRP1α-CD3 (FIG. 19B), and PDL1-CD3-Fc (FIG. 19C) show that the CD3 binding moiety of each of these molecules functional binds to CD3-expressing 293F T cells, as indicated by an increase in the percentage of cells that are positive for the engager molecules compared to the secondary antibody alone. In particular, a dose dependent increase in the % positive cells is observed for CD19-CD3 (FIG. 19A), while the SIRP1α-CD3 construct demonstrated maximal binding at all concentrations. The amount of the neat PDL1-CD3-Fc supernatant used resulted in binding of the construct to the majority of T cells (FIG. 19C).

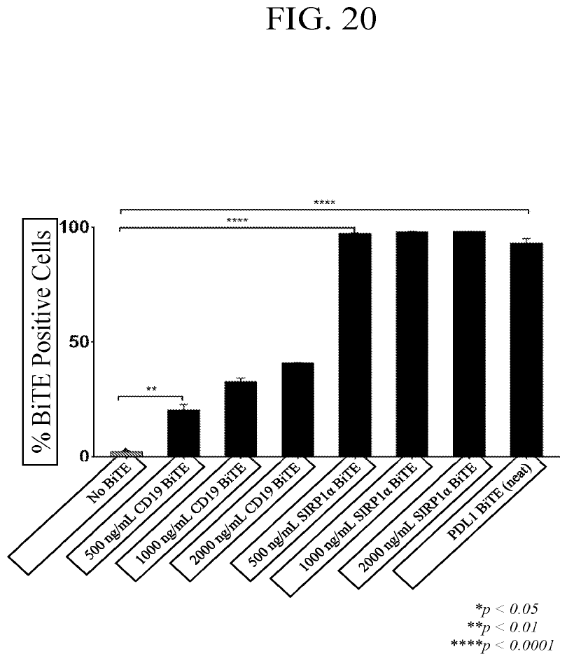

The results of this experiment are quantified in FIG. 20. In particular, all of the constructs demonstrated a significant increase in the % positive T cells compared to samples where no engager molecule was added.

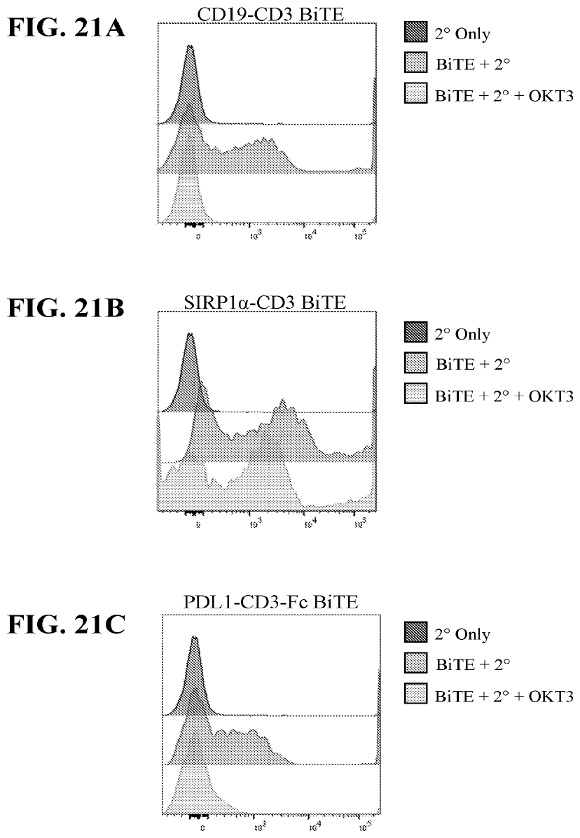

Additional experiments demonstrated that the binding of the CD19-CD3, SIRP1α-CD3, and PDL1-CD3-Fc was mediated by interactions of the anti-CD3 domain of the engager molecules with CD3 expressed by the T cells. Prior to exposure of T cells to the engager molecules, the T cells were incubated with an anti-CD3 monoclonal antibody (OKT3). Preincubation with the OKT3 inhibited binding of the CD19-CD3 engager, and substantially reduced binding of the PDL1-CD3-Fc engager. The lack of inhibition of binding of the SIRP1α-CD3 engager by preincubation with OKT3 (FIG. 21C) is likely due to an incomplete inhibition of CD3 by OKT3 in these samples.

### Example 5: SIRP1α-CD3 Constructs Specifically Bind to CD47

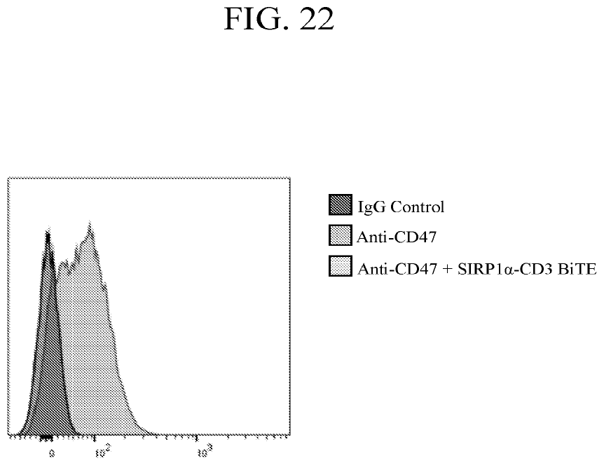

Experiments were performed to determine the binding specificity of the SIRP1α-CD3 engager constructs. Raji cells were preincubated with SIRP1α-CD3 engagers for 20 min at RT. Cells were then washed and incubated with a fluorescently labelled anti-CD47 monoclonal antibody for 20 min at RT, after which cells were washed and analyzed by flow cytometry. Raji cells that were not preincubated with the SIRP1α-CD3 engager showed significant binding of the anti-CD47 monoclonal antibody (FIG. 22, IgG control histogram vs. the anti-CD47 histogram). Preincubation of Raji cells with the SIRP1α-CD3 engager blocked binding of the anti-CD47 monoclonal antibody (FIG. 22, anti-CD47 histogram vs. anti-CD47+SIRP1α-CD3 histograph).

### Example 6: Binding of SIRP1α-CD3 and CD19-CD3 Engager Molecules to Target Cells

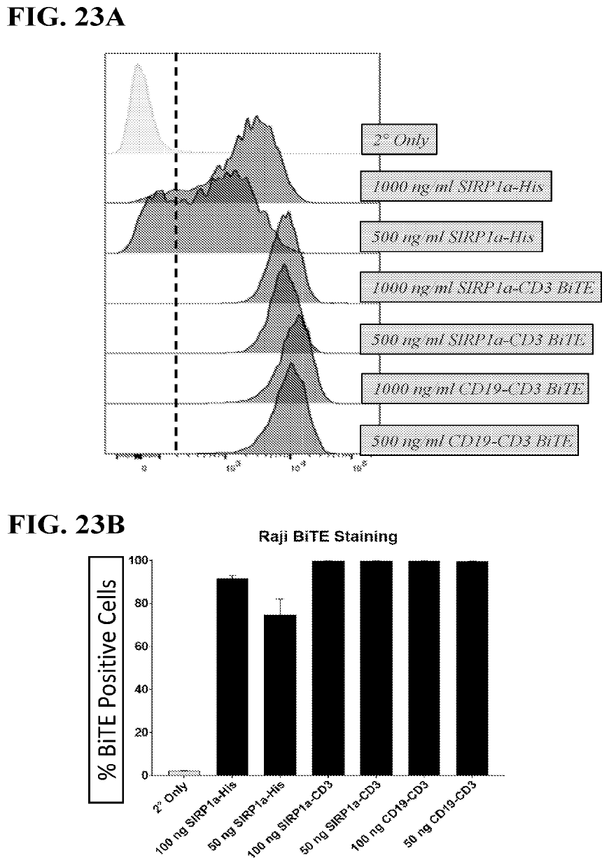

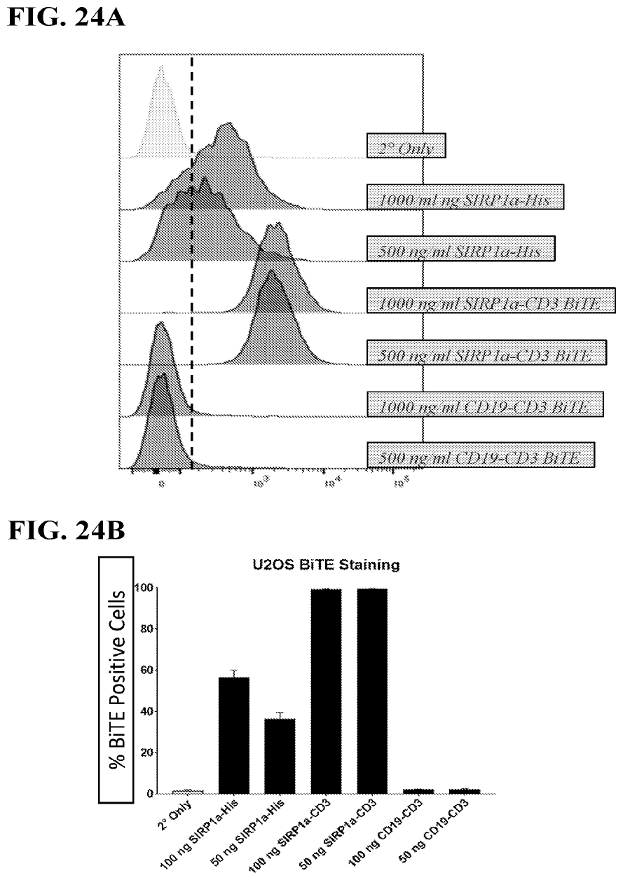

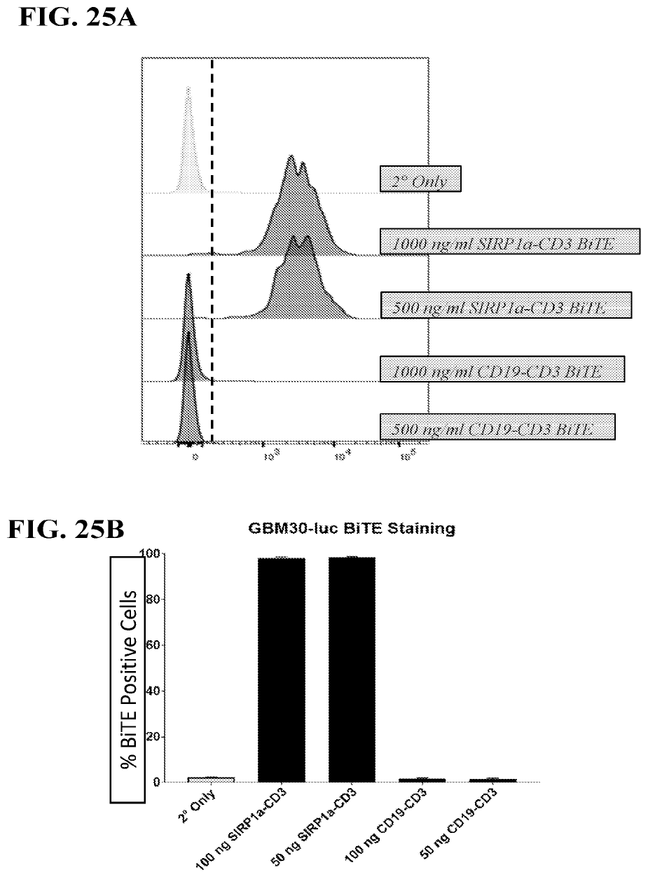

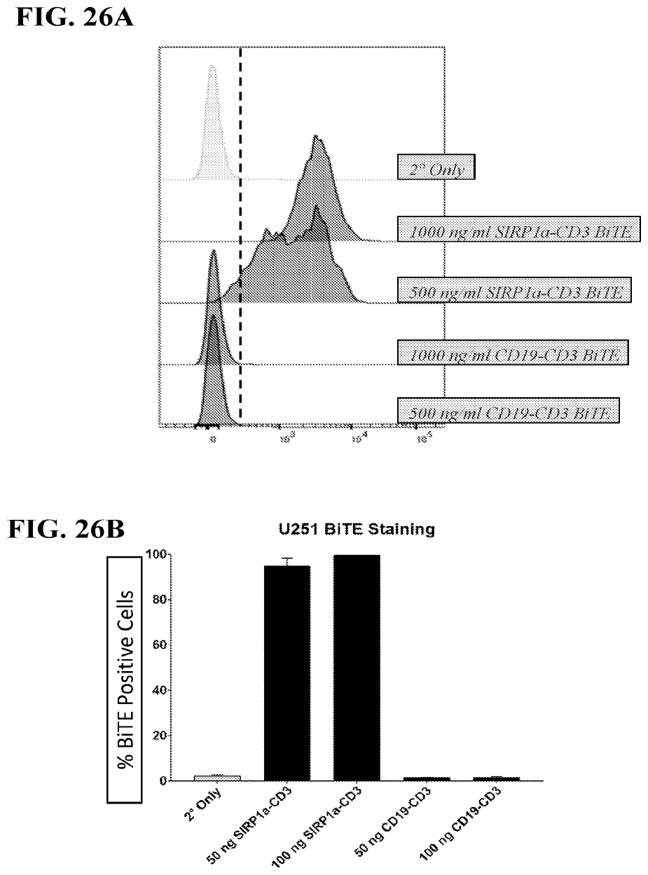

Experiments were performed to determine the ability of SIRP1α-CD3 and CD19-CD3 BiTEs to bind to Raji (CD19⁺CD47⁺, FIG. 23), U20S (CD19⁻CD47⁺, FIG. 24), GBM30-luc (CD19-CD47⁺, FIG. 25), and U251 (CD19⁻CD47⁺, FIG. 26) target cell types. For each target cell type, cells were treated with 500 or 1000 ng/mL of either (i) His-tagged soluble SIRP1α; (ii) SIRP1α-CD3 BiTE; or (iii) or CD19-CD3 BiTE. Cells were then stained with a fluorescently labelled anti-His antibody and analyzed by flow cytometry.

The results of SIRP1α-CD3 and CD19-CD3 binding to CD19⁺CD47⁺ Raji cells are shown in FIG. 23. Relative to the negative control Ig (2° only), soluble SIRP1α, SIRP1α-CD3 BiTE, and CD19-CD3 BiTE were able to bind to Raji cells, as indicated by a shift towards the right of the engager histograms compared to the IgG control histogram (FIG. 23A). Quantitation of the binding data showing percentage of BiTE positive cells is show in FIG. 23B.

The results of SIRP1α-CD3 and CD19-CD3 binding to CD19⁻CD47⁺U2OS cells are shown in FIG. 24. Relative to the negative control Ig (2° only), soluble SIRP1α, SIRP1α-CD3 BiTE were able to bind to U2OS cells at all concentrations used, as indicated by a shift towards the right of the engager histograms compared to the IgG control histogram (FIG. 24A). CD19-CD3 BiTEs were unable to bind to U2OS cells, which was expected based on the lack of CD19 expression by U2OS cells. Quantitation of these binding data showing percentage of BiTE positive cells is show in FIG. 24B.

The results of SIRP1α-CD3 and CD19-CD3 binding to CD19⁻CD47⁺ GBM30-luc cells are shown in FIG. 25. Relative to the negative control Ig (2° only), SIRP1α-CD3 BiTE were able to bind to GBM30-luc cells at all concentrations used, as indicated by a shift towards the right of the engager histograms compared to the IgG control histogram (FIG. 25A). In contrast, CD19-CD3 BiTEs were unable to bind to GBM30-luc cells, which was expected based on the lack of CD19 expression by GBM30-luc cells. Quantitation of these binding data showing percentage of BiTE positive cells is show in FIG. 25B.

The results of SIRP1α-CD3 and CD19-CD3 binding to CD19⁻CD47⁺ U251 cells are shown in FIG. 26. Relative to the negative control Ig (2⁰ only), SIRP1α-CD3 BiTE were able to bind to U251 cells at all concentrations used, as indicated by a shift towards the right of the engager histograms compared to the IgG control histogram (FIG. 26A). In contrast, CD19-CD3 BiTEs were unable to bind to U251 cells, which was expected based on the lack of CD19 expression by U251 cells. Quantitation of these binding data showing percentage of BiTE positive cells is show in FIG. 26B.

### Example 7: Binding of PDL1-CD3-Fc TiTEs to U251 Cells is Mediated by CD47, not FcγRs

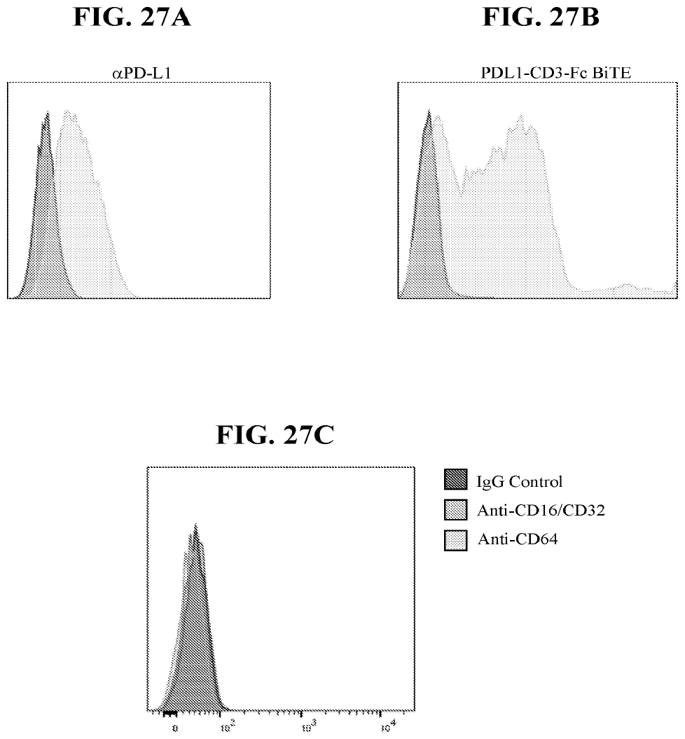

As the PDL1-CD3-Fc TiTE construct comprises 2 domains that are capable of binding to target cells (the anti-PDL1 and the Fc domain) experiments were performed to assess the binding specificity of these constructs. CD19⁻CD47T U251 cells were treated with 2 μg/mL of a fluorescently labeled anti-PDL1 antibody, an isotype control, or PDL1-CD3-Fc transfection supernant. Relative to negative control Ig, the PDL1-CD3-Fc TiTE bound to U251 cells (FIG. 27B). To assess whether this observed binding was due to interactions with CD47 or FcγRs expressed by U251 cells, the FEcγR expression on U251 cells was determined. Cells were incubated with 2 μg/mL of fluorophore-conjugated anti-CD16/32 (recognizing FEcγRIII/FcγRII) or anti-CD64 (recognizing FEcγRI) mAbs for 20 min at RT. Cells were then washed and analyzed by flow cytometry using a BD LSR Fortessa cytometer. As shown in FIG. 27C, U251 cells do not express FcγRI, FcγRII, or FEcγRIII, indicating the binding of the PDL1-CD3-Fc construct was mediated by interactions with CD47 and not FcγRs.

### Example 8: CD19-CD3, SIRP1α-CD3, and PDL1-CD3-Fc Constructs Stimulate CD8+ T Cell-Mediated Killing of Target Cells

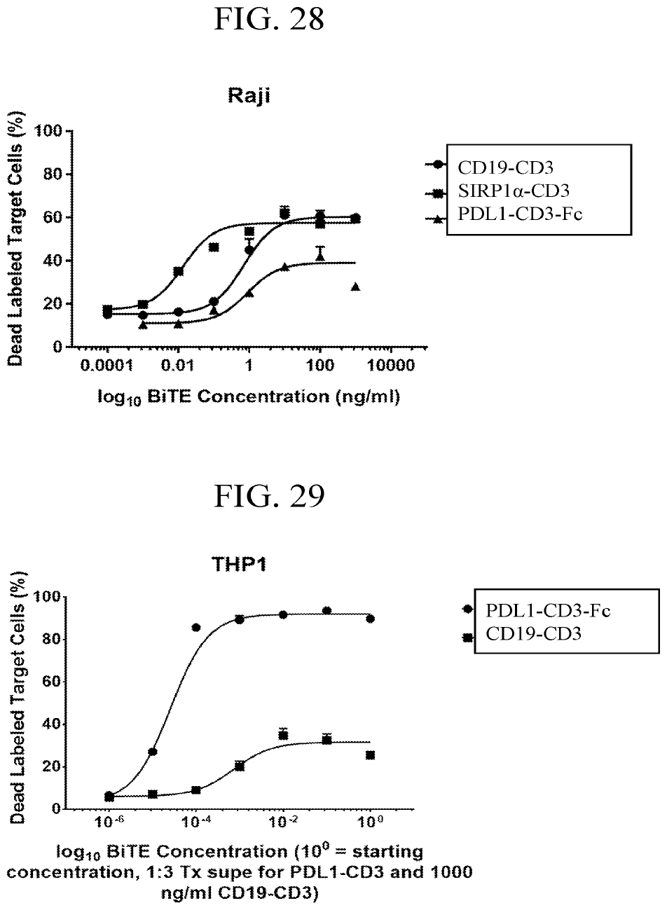

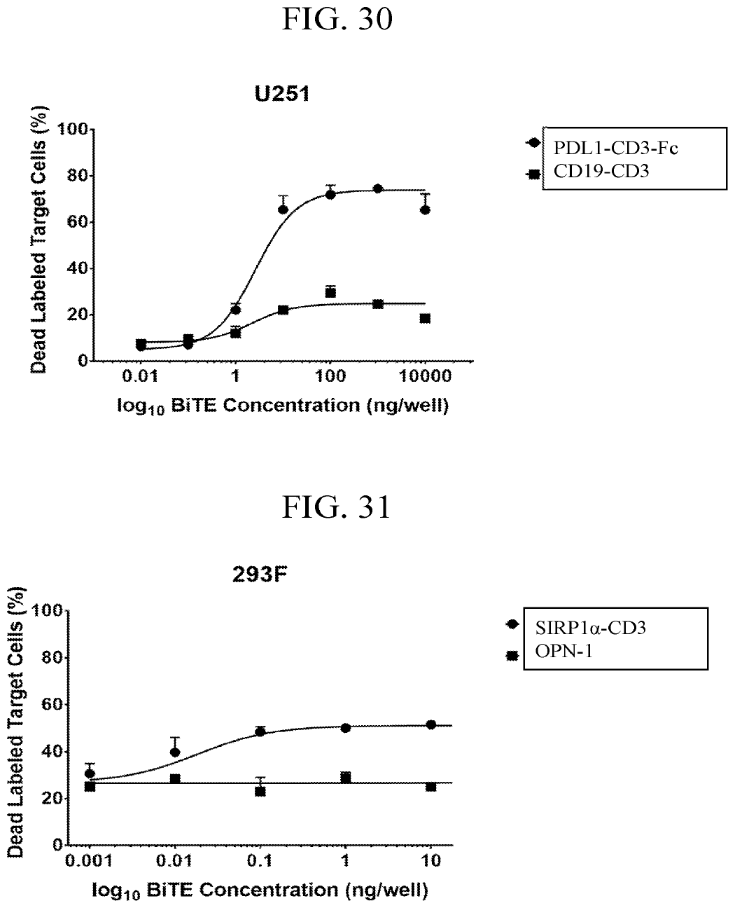

Experiments were performed to determine the ability of CD19-CD3, SIRP1α-CD3, and PDL1-CD3-Fc constructs to mediate killing of target cells. Briefly, CD8⁺ T cells were stimulated for 8-12 days in the presence of 200 U/mL IL-2 and Dynabeads. Prior to co-culture with target cells, all Dynabeads were removed by magnet and cells were washed to remove IL-2. Raji (FIG. 28), THP1 (FIG. 29), U251 (FIG. 30), and 293F (FIG. 31) target cells were labeled with the fluorescent membrane dye PKH67 green before plating. CD8+effector T cells were then co-cultured with target cells at an effector to target ratio of 1:1 along with 1000 ng/mL CD19-CD3 BiTE, SIRP1α-CD3 BiTEs, or a 1:3 dilution of PDL1-CD3-Fc transfection supernatant. Co-cultures of target and effector cells were incubated for 18 hours, after which they were stained with 7-AAD and live/dead analysis was performed by flow cytometry on a BD LSR Fortesa cytometer.

The results of these experiments indicate that the CD19-CD3, SIRP1α-CD3 and PDL1-CD3-Fc engager constructs were all capable of inducing effector cell-mediated death of Raji target cells (FIG. 28), The EC₅₀ for each of the CD19-CD3, SIRP1α-CD3 and PDL1-CD3-Fc engager molecules on Raji cells are shown below in Table 5.

TABLE 5
EC50 of engager molecules on Raji cells

| Engager Molecule | EC₅₀ (ng/mL) |
|---|---|
| CD19-CD3 | 0.6997 |
| SIRP1α-CD3 | 0.0137 |
| PDL1-CD3-Fc | 0.8907 |

The results of these experiments further indicate that the PDL1-CD3-Fc engager constructs, but not the CD19-CD3 constructs, were capable of inducing effector cell-mediated death of THP1 target cells (FIG. 29). This is likely due to the lack of/relatively low expression of CD19 by THP1 cells.

Further, the PDL1-CD3-Fc engager constructs were capable of inducing effector cell-mediated death of U251 target cells (FIG. 30), while the CD19-CD3 constructs did not induce effector cell-mediated death of U251 cells due to a lack of CD19 expression by U251 cells. The EC₅₀ for each of the CD19-CD3 and PDL1-CD3-Fc constructs on U251 cells are shown below in Table 6.

TABLE 6
EC₅₀ of engager molecules on U251 cells

| Engager Molecule | EC₅₀ (ng/mL) |
|---|---|
| CD19-CD3 | 2.247 |
| PDL1-CD3-Fc | 2.611 |

Further, the SIRP1α-CD3 engager constructs were capable of inducing effector cell-mediated death of 293F target cells (FIG. 31), indicated by the increase in cell death in SIRP1α-CD3 containing cultures compared to a control osteopontin-fusion protein (OPN 1). The EC₅₀ for SIRP1α-CD3 engager molecules on 293F cells is shown below in Table 7.

TABLE 7
EC₅₀ of SIRP1α-CD3 on 293F cells

| Engager Molecule | EC₅₀ (ng/mL) |
|---|---|
| SIRP1α-CD3 | 0.0184 |

### Example 9: PDL1-CD3-Fc BiTE Enhances Primary NK Cell Killing of U251 Cells

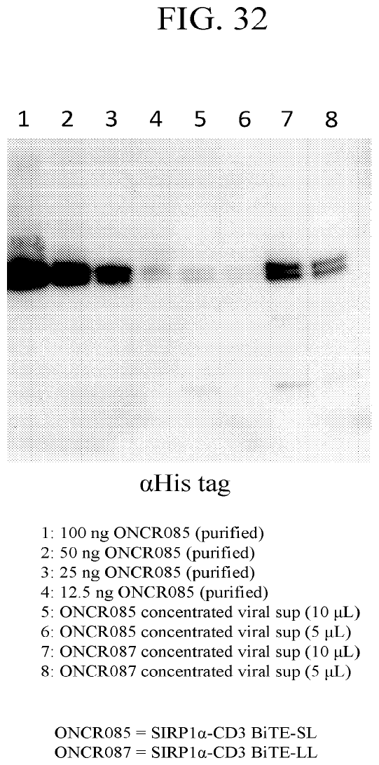

Experiments are performed to assess the ability of PDL1-CD3-Fc constructs to induce NK cell-mediated killing of target cells. Briefly, U251 cells are labelled with cell membrane dye PKH67 green, and then seeded and allowed to adhere to wells over night (FIG. 32). Primary NK cells (StemCell Technologies, Inc.) are then added to each well at an effector to target ratio of 1:1, along with varying amounts of virally produced PDL1-CD3-Fc protein. Effector/target cell co-culture are incubated at 37° C. for 6 hours prior to live/dead analysis by 7-AAD staining. Stained cells are analyzed by flow cytometry on a BD LSR Fortesa cytometer.

These results will demonstrate that virally produced PDL1-CD3-Fc compounds are able to stimulate NK cell-mediated death of target cells such as U251.

### Example 10: oHSV-Infected Vero Cells Express SIRP-1α-CD3 BiTEs

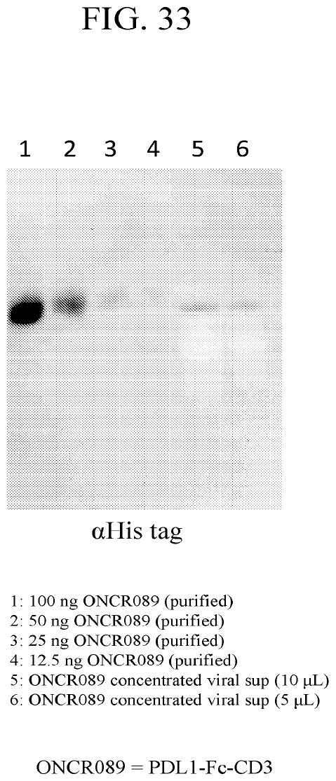

To demonstrate that the oncolytic viruses described here are capable or producing the engager molecules, Vero cells were infected with oHSV expressing SIRP1α-CD3 BiTEs (FIG. 32) with either a short linker (SL) (ONCR-085; 2A5B SIRP1α-CD3 (SL) BiTE) or long linker (LL) (ONCR-087; 2A5B SIRP1α-CD3 (LL) BiTE), or with oHSV expressing PDL1-CD3-Fc TiTEs (ONCR-089, FIG. 33). Cells were infected for 3 days, after which supernatants from infected cells were passed through a 100K MWCO ultrafiltration membrane to remove any viral particles. The flowthrough was concentrated with a 10K MWCO ultrafiltration membrane. Concentrated viral supernatants and 100 ng, 50 ng, 25 ng, or 12.5 ng of purified SIRP1α-CD3 or PDL1-CD3-Fc protein were then analyzed by PAGE followed by Western blotting with an anti-6xHis detection antibody in order to determine the amount of engager protein present in the viral supernatants.

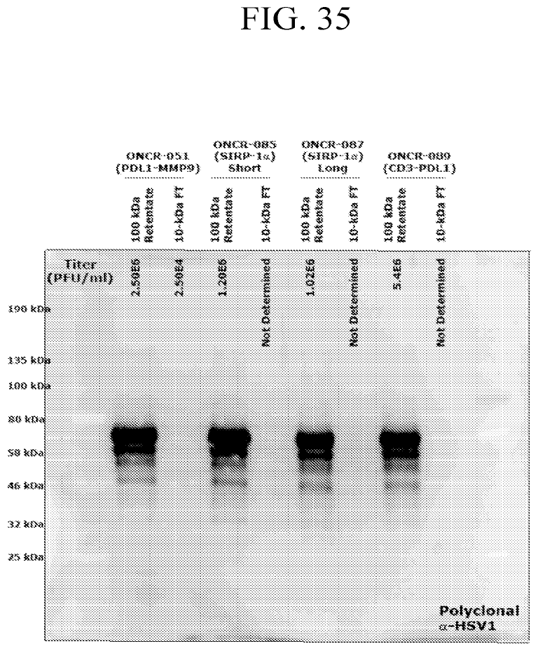

The results demonstrate that cells infected with either ONCR-085 or ONCR-087 produced the SIRP1α-CD3 (SL) and SIRP1α-CD3 (LL) protein, respectively (FIG. 32). Further, cells infected with ONCR-089 produced the PDL1-CD3-Fc protein (FIG. 33). The ability of the 100K and 10K Amicon filtration and concentration steps to remove remaining virus was assessed by Western blot. The workflow for clarifying viral supernatants comprises low-speed centrifugation of the supernatants followed by filtration through a 0.8 μm filter membrane. Supernatant filtrates are then passaged through an Amicon 100 kDa filter to entrain the virus, followed by passage of the filtrate through an Amicon 10 kDa filter to entrain remaining protein. Aliquots of supernatants from virally-infected cells were taken before and after processing with the Amicon filters and the presence of HSV was determined by blotting with an anti-HSV polyclonal antibody. These results show that the ultrafiltration steps used to purify the engager constructs effectively removed virus (FIG. 35). Therefore, any target cell killing observed in the presence of these engager constructs is due to the engager construct itself, and not a result of viral infection of the target cells.

### Example 11: Virally-Produced SIRP1α-CD3 and PDL1-CD3-Fc Engager Constructs Induced Effector-Cell Mediated Killing of Target Cells

Experiments were performed to assess the ability of virally-produced engager molecules (SIRP1α-CD3 and PDL1-CD3-Fc constructs) to mediate target cell killing. Briefly, SIRP1α-CD3 (SL), SIRP1α-CD3 (LL) and PDL1-CD3-Fc proteins were prepared from Vero cells as described in Example 10. 50 μL of the resulting SIRP1α-CD3 (SL), SIRP1α-CD3 (LL), and PDL1-CD3-Fc engager proteins protein samples were diluted 1:1 in tissue culture media containing 20% FBS. The diluted engager proteins were then incubated with activated CD8+effector T cells co-cultured with fluorescently labelled U251 target cells at a target to effector ratio of 1:1 for 18 hours. Cell death of U251 cells was assessed by flow cytometry on a BD LSR Fortesa cytometer.

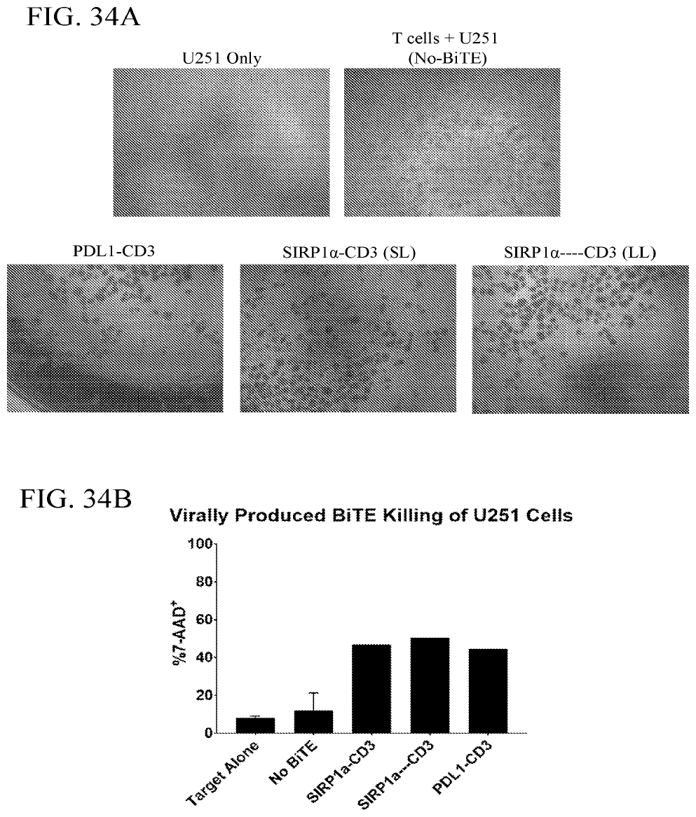

The results of this experiment demonstrate that virally-produced engager constructs direct T-cell mediated killing of U251 target cells (FIG. 34A). These results are quantified in FIG. 34B.

### Example 12: Expression of SIRP1α-CD3/PDL1-Fc Compounds from 293 T Cells

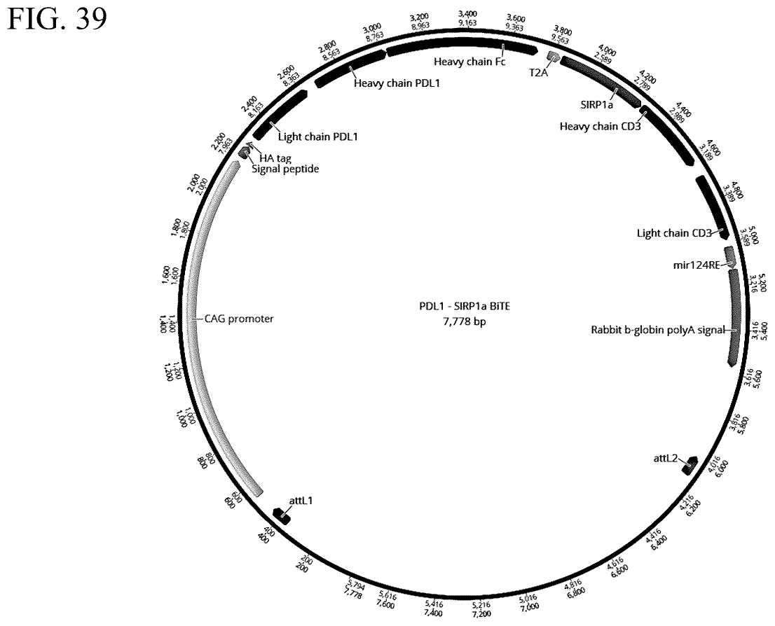

Two expression plasmids encoding a SIRP1α-CD3 engager molecule and a PDL1-Fc therapeutic molecule were generated. One construct comprised a first gene encoding an HA-tagged PDL1-Fc linked to a second gene encoding a His-tagged SIRP1α-CD3 BiTE. The SIRP1α amino acid sequence was linked to the anti-CD3 scFv by a single amino acid linker (i.e., a short linker) (SIRP1α-CD3/PDL1-Fc (SL), FIG. 37). The other construct comprised a first gene encoding a PDL1-Fc linked to a second gene encoding a SIRP1α-CD3 BiTE. The SIRP1α amino acid sequence was linked to the anti-CD3 scFv by a G4S linker (i.e., a long linker) (SIRP1-CD3/PDL1-Fc (LL), FIG. 38). The constructs were inserted into a plasmid (FIG. 39) and the resultant SIRP1α-CD3/PDL1-Fc expression plasmids were transfected into 293 Free Style T cells. Four days after plasmid transfection, culture supernatants were collected.

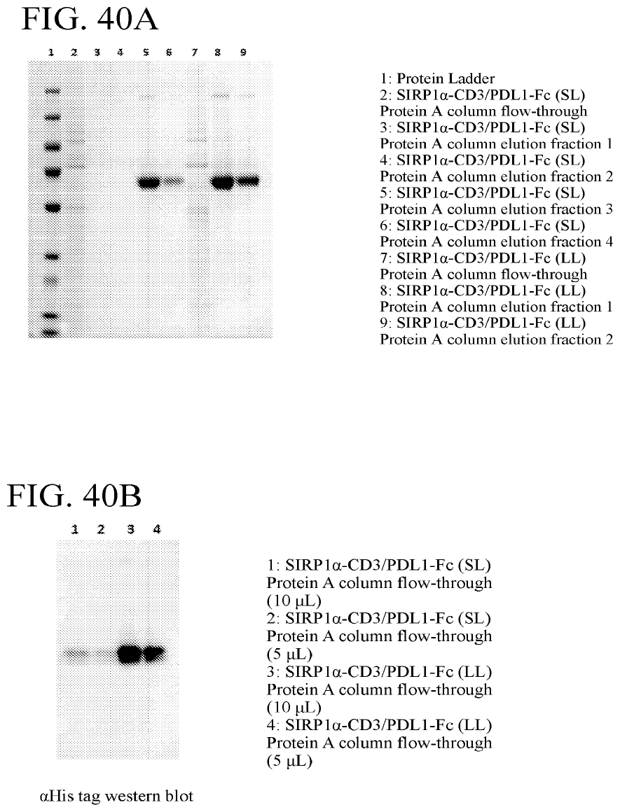

Anti-PDL1-Fc compounds were purified from the culture supernatants using a HiTrap MabSelect SuRe Protein A column HiTrap column (GE Healthcare). Briefly, supernatants from 293 T cells transfected with either the SIRP1α-CD3/PDL1-Fc (LL) or the SIRP1α-CD3/PDL1-Fc (LL) expression plasmids were loaded onto the column to purify the anti-PDL1-Fc compounds by binding of the HA-tag to the column. Flow through was collected for SIRP1α-CD3 BiTE detection by Western Blot using an anti-His antibody (FIG. 40B). Columns were washed with wash buffer (20 mM sodium phosphate, 150 mM NaCl, pH 7.4). Bound anti-PDL1-Fc protein was eluted with IgG elution buffer (pH 2.8, Pierce) and was immediately neutralized with a 1 M Tris-HCl buffer, pH 8.

The anti-PDL1-Fc protein content of different elution fractions then were visualized by Coomassie staning. Briefly, elution fractions were run on a 4%-12% Bis-Tris NuPAGE gel in MOPS buffer at 180 volts for 1 hour. Gels were stained for 1 hour in Simply Blue SafeStain followed by destaining with water. Anti-PDL1-Fc protein content for each elution fraction is show in FIG. 40A. After Coomassie analysis, elution fractions were combined and dialyzed against PBS at 4° C. Total anti-PDL1-Fc protein concentration was then determined by a BCA assay.

### Example 13: Isolated PDL1-Fc Proteins Stimulate T Cell-Mediated Death of Target Cells

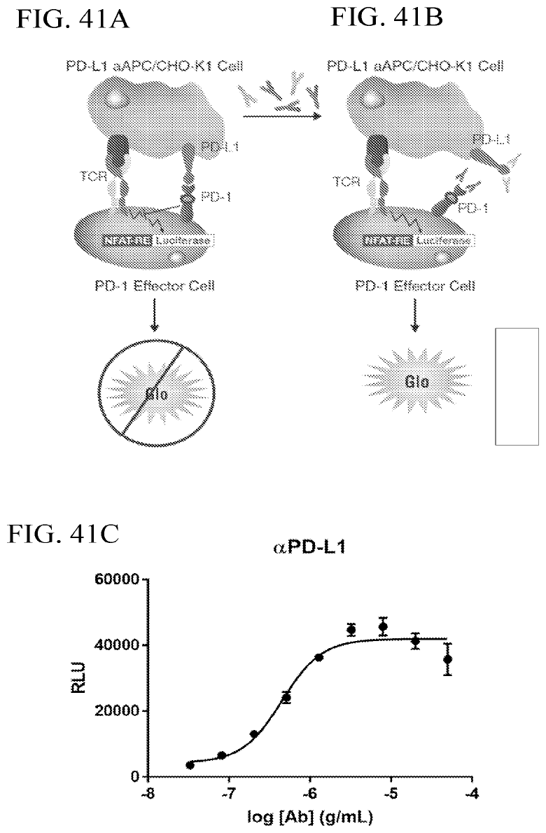

The ability of the anti-PDL1-Fc proteins to induce effector cell-mediated death of target cells was assessed by a PD1/PDL1 blockade assay. A general schematic of the assay is show in FIG. 41A-41B. Briefly, CD8⁺ T cells were co-cultured with PDL1-expressing target cells (CHO-K1 cells). Varying concentrations of the anti-PDL1-Fc protein isolated as described in Example 12 were then added to the culture. The highest concentration of anti-PDL1-Fc used was 50 μg/mL. 8, 2.5 fold serial dilutions were then performed to generate the remainder of the anti-PDL1-Fc concentrations. Cell death was analyzed by a CytoTox-Glo™ cytotoxicity assay in the presence (FIG. 41B) and absence (FIG. 41A) of the anti-PDL1-Fc. Results are quantified in FIG. 41C. The EC₅₀ of the anti-PDL1-Fc is shown in Table 8. These results demonstrate that the anti-PDL1-Fc therapeutic molecules produced from the expression constructs described herein are capable of mediating effector cell-mediated death of target cells.

TABLE 8
EC₅₀ of anti-PDL1-Fc compounds

| Compound | EC₅₀ |
|---|---|
| anti-PDL1-Fc | 0.45 μg/mL |

### Example 14: oHSV-Infected Vero Cells Express MMP9 and Anti-PDL1-Fc Therapeutic Molecules

In addition to producing the engager molecules as described in Example 10, experiments are performed to demonstrate that the oncolytic viruses described here are capable or producing the MMP9 and anti-PDL1-Fc therapeutic molecules. Vero cells are infected with oHSV expressing SIRP1α-CD3/PDL1-Fc constructs BiTEs (FIG. 37 and FIG. 38) or with oHSV expressing SIRP1α-CD3/MMP9 constructs (FIG. 18A and FIG. 18B). Cells are infected for 3 days, after which supernatants from infected cells are passed through a 100K MWCO ultrafiltration membrane to remove any viral particles. The flowthrough is concentrated with a 10K MWCO ultrafiltration membrane. MMP9 and anti-PDL1-Fc are purified from filtered, concentrated supernatants according to the protocol outlined in Example 11. Protein A-isolated MMP9 and anti-PDL1 fractions are analyzed by PAGE followed by Coomassie staining. SIRP1α-CD3 BiTEs present in the Protein A flowthrough are analyzed by Western blotting with an anti-6×His detection antibody.

The results will demonstrate that cells infected with oHSV vectors encoding either SIRP1α-CD3/PDL1-Fc constructs or SIRP1α-CD3/MMP9 constructs produce the SIRP1α-CD3 (SL) and SIRP1α-CD3 (LL) BITE protein, MMP9, and anti-PDL1-Fc.

### Example 15: Virally-Produced SIRP1α-CD3/MMP9 and SIRP1α-CD3/PDL1-Fc Engager Constructs Induce Effector-Cell Mediated Killing of Target Cells

Experiments are performed to assess the ability of virally-produced engager molecules (SIRP1α-CD3) and therapeutic molecules (MMP9 and anti-PDL1-Fc) to mediate target cell killing. Briefly, SIRP1α-CD3 (SL), SIRP1α-CD3 (LL), MMP9, and anti-PDL1-Fc proteins are prepared from Vero cells as described in Example 13. 50 μL of the resulting protein samples are diluted in tissue culture media containing 20% FBS. The diluted proteins are then incubated with activated CD8+effector T cells or NK effector cells and are co-cultured with fluorescently labelled target cells at a target to effector ratio of 1:1 for 18 hours. Cell death of target cells is assessed by flow cytometry on a BD LSR Fortesa cytometer.

The results of this experiment will demonstrate that virally-produced SIRP1α-CD3 engager constructs and therapeutic molecules NvMMP9 and anti-PDL1-Fc are able to direct T-cell and/or NK cell mediated killing of target cells.

### Example 16: Isolated FAP-CD3 Engager Molecules Bind FAP and T-Cells

The binding of bipartite (FAP-CD3) engager molecules to T cells will be assessed. Briefly, 25,000 T cells are stimulated with 200 U/mL IL-2 for 12 days. After 12 days, T cell are incubated with varying concentrations of engager molecules (500, 1000, or 2000 ng/mL) for 20 minutes at room temperature in triplicate. Cells are then washed twice, followed by staining with an anti-6XHis APC antibody at 500 ng/mL for an additional 20 minutes. Cells are washed again and treated with propidium iodide (PI) to exclude dead cells from further analysis. Stained cells are analyzed by flow cytometry on a BD LSR Fortesa cytometer and the percentage of the cell population positive for staining was set at 2% of the secondary only control.

Assessment of binding of bipartite (FAP-CD3) engager molecules to tumor cells. Briefly, HEK293 stably overexpressing FAB or negative control cells (H1EK293) are treated with 500 or 1000 ng/mL of either (i) His-tagged soluble FAP or (ii) His-tagged Fc negative control protein. Cells are stained with a fluorescently labelled anti-His antibody and analyze by flow cytometry.

### Example 17: Intravenous Delivery of Retargeted-oHSV Encoding FAP-CD3 Engager Molecules

The gD glycoprotein of HSV was modified to mediate viral entry via the EGFR receptor and engineered to encode for FAP-CD3. To test the ability of a retargeted ONCR-virus that encodes FAP-CD3 to induce tumor killing, 5×10⁶ A431 cells are implanted subcutaneously into the right flank Nude mice. When the flank tumors reach ˜100 mm³, mice are injected intravenously with a single dose or ONCR-EGFR-(FAP-CD3) at 1×10⁶ PFU; 3×10⁶ PFU; 1×10⁷ PFU, or vehicle (PBS). Tumor measurements are recorded every 2 to 3 days. Tumor growth inhibition is calculated against vehicle control treated cohort.

### Example 18: Intratumoral Delivery of oHSV Encoding FAP-CD3 Engager Molecules

Experiments were performed to assess the ability of an oHSV encoding a bipartite FAP-CD3 engager molecule to inhibit tumor growth in vivo. ONCR-148 virus that encodes s can induce tumor growth inhibition in vivo. Briefly, 5×10⁵ murine colon carcinoma cells expressing fibroblast activation protein (MC38-FAP) were implanted subcutaneously into both flanks of female C57BL/6 mice. When tumors reached a volume of ˜100 mm³, mice were divided into experimental groups and injected intratumorally (IT) with 3×10⁶ PFU of osHSV ONCR-148 (encoding bipartite FAP-CD3) or ONCR-146 (negative control virus encoding bipartite FAP-CD19). Vehicle controls were IT injected with PBS. Dosing was repeated every third day for a total number of 3 doses. Tumor measurements were performed twice weekly using electronic calipers, and tumor volume (TV) was calculated according to the modified ellipsoid formula: TV=½(length×width²). Inhibition of tumor growth was determined against vehicle-treated group.

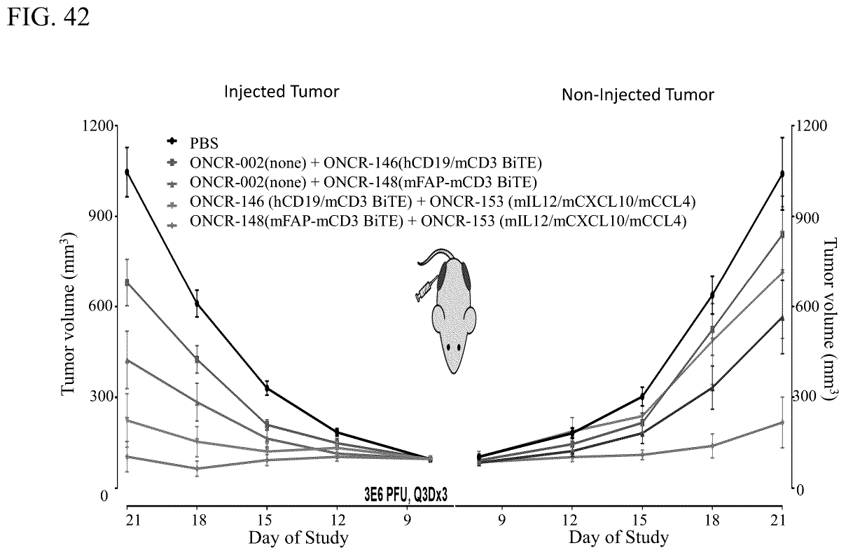

To assess the effect of FAP-CD3 engager molecule expression by oHSV on tumor growth, mice were divided into experimental groups and injected intratumorally (IT) with 3×10⁶ PFU of ONCR-148 (oHSV encoding bipartite FAP-CD3) or ONCR-146 (negative control virus encoding bipartite FAP-CD19). As shown in FIG. 42 mice treated with ONCR-148 demonstrated significant tumor growth inhibition in both injected (ipsilateral) and distant (contralateral) tumors.

Additional experiments were performed to determine additional effects of payloads that promote T cell activation and antigen presentation in tumor microenvironment (IL-12, CXCL10, and CCL4) on tumor growth inhibition. Mice were injected IT with vehicle control, 3×10⁶ PFU/dose ONCR-148 (oHSV encoding bipartite FAP-CD3), 3×10⁶ PFU/dose ONCR-153 (oHSV encoding IL-12, CXCL10 and CCL4), or a mixture of 1.5×10⁶ PFU each ONCR-148 and ONCR-153 (for a total viral dose of 3×10⁶ PFU/dose). Additional control groups were treated with ONCR-146 (negative control virus encoding bipartite FAP-CD19) or ONCR-146 and ONCR-153.

As depicted in FIG. 42, ONCR-148 and ONCR-153, when combined, elicited antitumor effect that was stronger than observed for either virus alone. Combination viral therapy resulted in 90% and 80% tumor growth inhibition in injected (ipsilateral) and distant (contralateral) tumors, respectively.

1.-57. (canceled)

58. An oncolytic virus comprising a first recombinant nucleic acid encoding an engager polypeptide comprising an effector domain specific for an antigen expressed on an effector cell and a targeting domain specific for an antigen expressed on a target cell, wherein the antigen expressed on the target cell is fibroblast activation protein (FAP) and a second recombinant nuclei acid encoding an immune modulator polypeptide selected from CCL4, CXCL10, IL-15 and IL-12.

59. The oncolytic virus of claim 58, comprising one or more micro RNA (miR) target sequences inserted into a locus of one or more genes required for viral replication.

60. The oncolytic virus of claim 59, wherein the one or more miR target sequences is recognized by miR-1, miR-10b, miR-17, miR-21, miR-106a, miR-125b, miR-145, miR-146a, miR-146b, miR-155, miR-96, miR-182, miR-183, miR-221, miR-222, miR-124, miR-143, or miR-1247-5p.

61. The oncolytic virus of claim 59, wherein the one or more miR target sequences is recognized by a plurality of microRNAs selected from miR-1, miR-10b, miR-17, miR-21, miR-106a, miR-125b, miR-145, miR-146a, miR-146b, miR-155, miR-96, miR-182, miR-183, miR-221, miR-222, miR-124, miR-143, and miR-1247-5p.

62. The oncolytic virus of claim 58, wherein the oncolytic virus is capable of preferential replication in a tumor cell.

63. The oncolytic virus of claim 58, wherein the engager polypeptide is a bipartite, tripartite or multipartite polypeptide and is comprised of an antibody, an antibody domain, a human immunoglobulin heavy chain variable domain, a dual-variable-domain, antibody (DVD-Ig), a Tandab, a diabody, a flexibody, a dock-and-lock antibody, a Scorpion polypeptide, a single chain variable fragment (scFv), a BiTE, a DuoBody, and Fc-engineered IgG, an Fcab, a Mab2, or DART polypeptide.

64. The oncolytic virus of claim 58, wherein the effector cell is a T cell or an NKT cell, an NK cell, or a macrophage.

65. The oncolytic virus of claim 58, wherein the antigen expressed on the effector cell is CD3, CD4, CD5, CD8, CD16, CD28, CD40, CD134, CD137 or NKG2D.

66. The oncolytic virus of claim 58, wherein the effector cell antigen is CD3.

67. The oncolytic virus of claim 66, wherein the engager polypeptide comprises or consists of a polypeptide sequence that is at least 90%, 95%, 96%, 97%, 98%, 99% or 100% identical to SEQ ID NO: 75.

68. The oncolytic virus of claim 66, wherein the first recombinant nucleic acid comprises or consists of a sequence that is at least 90%, 95%, 96%, 97%, 98%, 99%, or 100% identical to SEQ ID NO: 74.

69. The oncolytic virus of claim 58, wherein:
(i) the polypeptide sequence of IL-15 is at least 90%, 95%, 96%, 97%, 98%, 99%, or 100% identical to SEQ ID NO: 53;
(ii) the polypeptide sequence of IL-12 is at least 90%, 95%, 96%, 97%, 98%, 99%, or 100% identical to SEQ ID NO: 54;
(iii) the polypeptide sequence of CXCL10 is at least 90%, 95%, 96%, 97%, 98%, 99%, or 100% identical to SEQ ID NO: 55; and/or
(iv) the polypeptide sequence of CCL4 is at least 90%, 95%, 96%, 97%, 98%, 99%, or 100% identical to SEQ ID NO: 80.

70. The oncolytic virus of claim 58, wherein the oncolytic virus comprises the recombinant nucleic acids encoding two or more of CCL4, CXCL10, IL-15 and IL-12.

71. The oncolytic virus of claim 70, wherein the oncolytic virus comprises the recombinant nucleic acids encoding each of CCL4, CXCL10, and IL-12.

72. The oncolytic virus of claim 58, wherein the oncolytic virus is a pseudotyped oncolytic virus.

73. The oncolytic virus of claim 72, wherein the pseudotyped oncolytic virus comprises one or more modified glycoproteins involved in viral entry into a host cell, or wherein the pseudotyped oncolytic virus comprises a non-viral protein present on the viral surface.

74. The oncolytic virus of claim 73, wherein the one or more modified glycoproteins comprise one or more amino acid mutations, or wherein the non-viral protein binds to a protein expressed on the cell-surface of the target cell.

75. The oncolytic virus of claim 72, wherein the pseudotyped oncolytic virus:
(i) possesses an altered tropism relative to a non-pseudotyped virus;
(ii) yields reduced toxicity or reduced entry of non-tumor cells or tissue relative to a non-pseudotyped virus; and/or
(iii) yields increased toxicity or increased entry of tumor cells or cancerous tissue relative to a non-pseudotyped virus.

76. The oncolytic virus of claim 58, wherein the oncolytic virus is selected from or is derived from an adenovirus, a herpes simplex virus 1 (HSV1), a herpes simplex virus 2 (HSV2), a myxoma virus, a reovirus, a poliovirus, a vesicular stomatitis virus (VSV), a measles virus (MV), a lassa virus (LASV), or a Newcastle disease virus (NDV).

77. A pharmaceutical composition comprising the oncolytic virus of claim 58.

78. A method of (i) treating a cancer in a subject in need thereof or (ii) treating one or more tumors in the subject in need thereof using a therapeutically effective amount of the oncolytic virus of claim 58.

79. The method of claim 78, wherein the antigen expressed on the target cell is a tumor antigen expressed by the one or more tumors.

80. The method of claim 78, wherein the oncolytic virus is administered to a subject intravenously, intratumorally, systemically, intraperitoneally, subcutaneously, or intramuscularly.

81. The method of claim 78, wherein the oncolytic virus induces an immune response.

82. The method of claim 81, wherein the immune response is selectively cytotoxic to the target cell.

83. The method of claim 82, wherein the target cell is derived from a solid tumor or a hematologic cancer.

84. The method of claim 78, further comprising administering one or more additional therapies to the subject in need thereof,

85. The method of claim 84, wherein the one or more additional therapies comprise surgery, radiation, chemotherapy, immunotherapy, hormone therapy, or a combination thereof.

86. A method of delivering the engager polypeptide and the immune modulator polypeptide to a tumor site comprising administering the oncolytic virus of claim 58 to the tumor site.

87. The method of claim 86, wherein the tumor site is in a patient in need thereof.
